(19) United States
(12) Patent Application Publication
(10) Pub. No.: US 2023/0303537 A1
(43) Pub. Date: Sep. 28, 2023

(54) NITROGEN-CONTAINING COMPOUND, AND ELECTRONIC ELEMENT AND ELECTRONIC DEVICE THEREOF

(71) Applicant: Shaanxi Lighte Optoelectronics Material Co., Ltd., Xi’an (CN)

(72) Inventors: Lingang LI, Xi’an (CN); Peng NAN, Xi’an (CN)

(21) Appl. No.: 18/011,906

(22) PCT Filed: Jul. 30, 2021
(86) PCT No.: PCT/CN2021/109823

§ 371 Date: Dec. 21, 2022

(30) Foreign Application Priority Data
Aug. 5, 2020 (CN) 202010779851.6
Jun. 23, 2021 (CN) 202110698599.0

(51) Int. Cl. C07D 405/12; H10K 85/60; C07D 409/12; C07D 405/14; C07D 409/14; H10K 50/15

(52) U.S. Cl. CPC C07D 405/12; C07D 405/14; C07D 409/12; C07D 409/14; H10K 50/15; H10K 85/631; H10K 85/633; H10K 85/6572; H10K 85/6574; H10K 85/6576; C07B 2200/05

(57) ABSTRACT

The present disclosure belongs to the field of organic light-emitting materials, and in particular relates to a nitrogen-containing compound, and an electronic element and an electronic device comprising same. The nitrogen-containing compound has a structure as shown in formula 1, wherein X is selected from O or S; m is 0, 1 or 2, n is 0, 1 or 2, and 1≤m+n≤2. The nitrogen-containing compound is used in the electronic element, so that the performance of the electronic element can be improved.

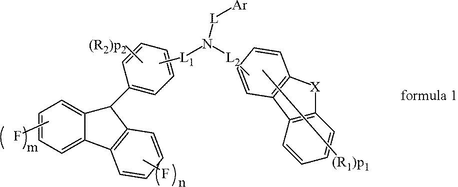

### CROSS REFERENCE TO RELATED APPLICATIONS

The present application claims the priority of Chinese Patent Application No. 202010779851.6, filed on Aug. 5, 2020, and Chinese Patent Application No. 202110698599.0, filed on Jun. 23, 2021, the contents of which are incorporated herein by reference in their entirety as a part of the present application.

### TECHNICAL FIELD

The present disclosure belongs to the technical field of organic light-emitting materials, and in particular to a nitrogen-containing compound, and an electronic element and an electronic device thereof.

### BACKGROUND

With the development of electronic technology and the progress of material science, electronic components for realizing electroluminescence or photoelectric conversion are more and more widely used. In recent years, organic electroluminescent devices (OLED) have gradually entered the field of view of people as a new generation of display technology. Such electronic component typically includes a cathode and an anode which are disposed oppositely, and a functional layer disposed between the cathode and the anode. The functional layer is composed of a plurality of organic or inorganic film layers, and generally includes an energy conversion layer, a hole transport layer between the energy conversion layer and the anode, and an electron transport layer between the energy conversion layer and the cathode. When a voltage is applied to the cathode and the anode, an electric field is generated between the two electrodes, under the action of the electric field, electrons on a cathode side move towards a light-emitting layer, and holes on an anode side also move towards the light-emitting layer, the electrons and the holes are combined in the light-emitting layer to form excitons, the excitons in an excited state release energy outwards, and a process of releasing energy from the excited state to a ground state emits light outwards. At present, the organic electroluminescent devices still have the problem of poor performance, in particular how to further improve the service life or efficiency of the device while ensuring a low driving voltage is still an urgent problem to be solved.

### SUMMARY

In view of the above-mentioned problems in the prior art, the present disclosure aims to provide a nitrogen-containing compound, and an electronic element and an electronic device comprising same. The nitrogen-containing compound is used in the electronic element, so that the performance of the electronic element can be improved.

In order to achieve the above purpose, in a first aspect, the present disclosure provides a nitrogen-containing compound, having a structure as shown in formula 1:

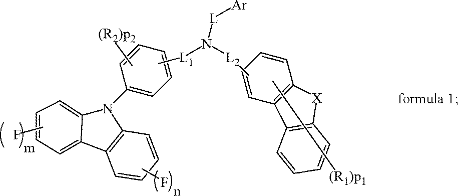

wherein X is selected from O or S;
Ar is selected from a substituted or unsubstituted aryl group with 6 to 30 carbon atoms, and a substituted or unsubstituted heteroaryl group with 3 to 30 carbon atoms;
L, L₁ and L₂ are the same or different, and are each independently selected from a single bond, a substituted or unsubstituted arylene group with 6 to 25 carbon atoms, and a substituted or unsubstituted heteroarylene group with 3 to 25 carbon atoms, and L₁ and L₂ are not simultaneously a single bond;
m is 0, 1 or 2, n is 0, 1 or 2, and 1≤m+n ≤2;
the substituents in Ar, L, L₁ and L₂, as well as R₁ and R₂, are the same or different, and are each independently selected from: deuterium, tritium, a halogen group, cyano, aryl with 6 to 18 carbon atoms, heteroaryl with 3 to 18 carbon atoms, trialkylsilyl with 3 to 12 carbon atoms, alkyl with 1 to 10 carbon atoms, haloalkyl with 1 to 10 carbon atoms, alkenyl with 2 to 10 carbon atoms, cycloalkyl with 3 to 10 carbon atoms, alkylthio with 1 to 10 carbon atoms, alkoxy with 1 to 10 carbon atoms, aryloxy with 6 to 18 carbon atoms, arylthio with 6 to 18 carbon atoms, and triphenylsilyl;
p₁ represents the number of R₁, and is selected from 0, 1, 2, 3, 4, 5, 6 or 7; when p₁ is greater than 1, any two R₁ are the same or different; and optionally, any two adjacent R₁ can be connected to form a ring; and
p₂ represents the number of R₂, and is selected from 0, 1, 2, 3 or 4; when p₂ is greater than 1, any two R₂ are the same or different; and optionally, any two adjacent R₂ can be connected to form a ring.

In a second aspect, the present disclosure provides an electronic element, including an anode and a cathode which are oppositely disposed, and a functional layer disposed between the anode and the cathode; and the functional layer includes the nitrogen-containing compound according to the first aspect of the present disclosure.

In a third aspect, the present disclosure provides an electronic device comprising the electronic element according to the second aspect of the present disclosure.

The inventors of the present disclosure found in research that in a structure formed by N-phenylcarbazole-substituted triarylamine, when one or two fluorines are connected to the carbazolyl, for the fluorines have a strong electron withdrawing ability, the electron cloud density of the parent core structure can be reduced; and meanwhile, specific groups such as dibenzofuranyl/dibenzothienyl are introduced on triarylamine, based on this, and at least one of the two groups which refer to N-phenylcarbazolyl and dibenzofuranyl/dibenzothienyl is controlled to be connected to a nitrogen atom via an aromatic group, so that the distortion degree of the whole molecular structure can be improved, and molecular configuration of the compounds can be improved, on one hand, as a result, the nitrogen-containing compound provided by the present disclosure has relatively high thermal stability, and on the other hand, the transport rate of electrons can be effectively reduced, hence the electrons can be blocked from passing through the device. The nitrogen-containing compound of the present disclosure is used as an electron blocking layer (also referred to as a “second hole transport layer”) material, so that the performance of OLEDs can be improved, and in particular, the luminous efficiency and service life of the device can be improved while ensuring a lower driving voltage of the device.

### BRIEF DESCRIPTION OF THE DRAWINGS

FIG. 1 is a structural schematic diagram of an organic electroluminescent device according to one example of the present disclosure.

FIG. 2 is a structural schematic diagram of a first electronic device according to one example of the present disclosure.

FIG. 3 is a structural schematic diagram of a photoelectric conversion device according to one example of the present disclosure.

FIG. 4 is a structural schematic diagram of a second electronic device according to one example of the present disclosure.

### DESCRIPTION OF REFERENCE SIGNS

100, anode; 200, cathode; 300, functional layer; 310, hole injection layer; 320, hole transport layer; 321, first hole transport layer; 322, second hole transport layer; 330, organic light-emitting layer; 340, electron transport layer; 350, electron injection layer; 360, photoelectric conversion layer; 400, first electronic device; and 500, second electronic device.

### DETAILED DESCRIPTION

The specific examples of the present disclosure will be described in detail below with reference to the accompanying drawings. It should be understood that the specific examples described here are only used to illustrate and explain the present disclosure, but are not intended to limit the present disclosure.

In a first aspect, the present disclosure provides a nitrogen-containing compound, having a structure as shown in formula 1:

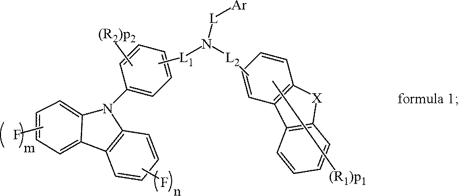

wherein X is selected from O or S;
Ar is selected from a substituted or unsubstituted aryl group with 6 to 30 carbon atoms, and a substituted or unsubstituted heteroaryl group with 3 to 30 carbon atoms;
L, L₁ and L₂ are the same or different, and are each independently selected from a single bond, a substituted or unsubstituted arylene group with 6 to 25 carbon atoms, and a substituted or unsubstituted heteroarylene group with 3 to 25 carbon atoms, and L₁ and L₂ are not simultaneously a single bond;
m is 0, 1 or 2, n is 0, 1 or 2, and 1≤m+n ≤2;
substituents in Ar, L, L₁ and L₂, as well as R₁ and R₂, are the same or different, and are each independently selected from: deuterium, tritium, a halogen group, cyano, aryl with 6 to 18 carbon atoms, heteroaryl with 3 to 18 carbon atoms, trialkylsilyl with 3 to 12 carbon atoms, alkyl with 1 to 10 carbon atoms, haloalkyl with 1 to 10 carbon atoms, alkenyl with 2 to 10 carbon atoms, cycloalkyl with 3 to 10 carbon atoms, alkylthio with 1 to 10 carbon atoms, alkoxy with 1 to 10 carbon atoms, aryloxy with 6 to 18 carbon atoms, arylthio with 6 to 18 carbon atoms, and triphenylsilyl;
p₁ represents the number of R₁, and is selected from 0, 1, 2, 3, 4, 5, 6 or 7; when p₁ is greater than 1, any two R₁ are the same or different; and optionally, any two adjacent R₁ can be connected to form a ring; and
p₂ represents the number of R₂, and is selected from 0, 1, 2, 3 or 4; when p₂ is greater than 1, any two R₂ are the same or different; and optionally, any two adjacent R₂ can be connected to form a ring.

In the present disclosure, m and n in the formula 1 represent the number of F, respectively.

In the present disclosure, the used descriptions modes “each...is independently”, “...is respectively and independently” and “...is independently selected from” can be interchanged, which should be understood in a broad sense, and may mean that specific options expressed by a same symbol in different groups do not influence each other, or that specific options expressed by a same symbol in a same group do not influence each other. For example, the meaning of “

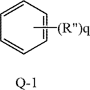

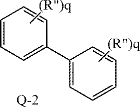

wherein each q is independently selected from 0, 1, 2 or 3 and each R″ is independently selected from hydrogen, deuterium, fluorine, and chlorine“ is as follows: a formula Q-1 represents that there are q substituents R″ on a benzene ring, each R″ may be the same or different, and the options of each R″ do not affect each other; and a formula Q-2 represents that there are q substituents R″ on each benzene ring of biphenyl, the number q of R″ substituents of the two benzene rings may be the same or different, R″ may be the same or different, and the options of each R″ do not affect each other.

In the present disclosure, the terms “optional” and “optionally” mean that the subsequently described event or circumstance may but need not occur, and that the description includes that the event or circumstance occurs or not. For example, “optionally, any two adjacent substituents xx can be connected to form a ring”, which means that the two substituents can, but need not, form a ring, including a scenario in which two adjacent substituents form a ring and a scenario in which two adjacent substituents do not form a ring.

In the present disclosure, the term such as “substituted or unsubstituted” means that a functional group described behind the term may or may not have a substituent (the substituent is collectively referred to as Rc below for ease of description). For example, “substituted or unsubstituted aryl” refers to aryl with a substituent Rc or unsubstituted aryl. The above substituent, i.e. Rc, can, for example, be deuterium, tritium, a halogen group, cyano, aryl, heteroaryl, trialkylsilyl, triphenylsilyl, alkyl, haloalkyl, alkenyl, cycloalkyl, alkylthio, alkoxy, or the like; when two substituents Rc are connected to a same atom, the two substituents Rc may independently exist or may be connected to each other to form a ring with the atom; and when two adjacent substituents Rc are present on a functional group, the two adjacent substituents Rc may independently be present or may be fused to form a ring with the functional group to which they are connected.

In the present disclosure, the number of carbon atoms of a substituted or unsubstituted functional group refers to the number of all carbon atoms. For example, if L is selected from substituted arylene with 12 carbon atoms, the number of all carbon atoms of the arylene and substituents on the arylene is 12.

In the present disclosure, aryl refers to an optional functional group or substituent derived from an aromatic carbocyclic ring. The aryl can be monocyclic aryl (e.g., phenyl) or polycyclic aryl, in other words, the aryl can be monocyclic aryl, fused aryl, two or more monocyclic aryl conjugatedly linked by carbon-carbon bonds, monocyclic aryl and fused aryl which are conjugatedly linked by a carbon-carbon bond, and two or more fused aryl conjugatedly linked by carbon-carbon bonds. That is, unless specified otherwise, two or more aromatic groups conjugatedly linked by carbon-carbon bonds can also be regarded as aryl of the present disclosure. Wherein the fused aryl may, for example, include bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. It should be noted that biphenyl, terphenyl, and 9,9-dimethylfluorenyl are all regarded as aryl in the present disclosure. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl, and the like. In the present disclosure, involved arylene refers to a divalent group formed by further loss of one hydrogen atom of the aryl.

In the present disclosure, substituted aryl can be that one or two or more hydrogen atoms in the aryl are substituted with groups such as deuterium, a halogen group, cyano, aryl, heteroaryl, trialkylsilyl, alkyl, cycloalkyl, alkoxy, alkylthio, and the like. Specific examples of heteroaryl-substituted aryl include, but are not limited to, dibenzofuranyl-substituted phenyl, dibenzothiophene-substituted phenyl, pyridine-substituted phenyl, and the like. It should be understood that the number of carbon atoms of the substituted aryl refers to the total number of carbon atoms of the aryl and substituents on the aryl, for example, substituted aryl with 18 carbon atoms means that the total number of carbon atoms of the aryl and its substituents is 18.

In the present disclosure, heteroaryl refers to a monovalent aromatic ring containing at least one heteroatom in the ring or its derivative, and the heteroatom may be at least one of B, O, N, P, Si, Se and S. The heteroaryl can be monocyclic heteroaryl or polycyclic heteroaryl, in other words, the heteroaryl can be a single aromatic ring system or a plurality of aromatic ring systems conjugatedly linked via carbon-carbon bonds, and any one aromatic ring system is a aromatic monocyclic ring or a aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl and the like, but is not limited to these. Among them, thienyl, furyl, phenanthrolinyl and etc. are the heteroaryl with a single aromatic ring system, N-phenylcarbazolyl and N-pyridylcarbazolyl are the heteroaryl with a plurality of aromatic ring systems which conjugatedly linked via carbon-carbon bonds. In the present disclosure, involved heteroarylene refers to a divalent group formed by further lossing one hydrogen atom from the heteroaryl.

In the present disclosure, substituted heteroaryl may be that one or two or more hydrogen atoms in the heteroaryl are substituted with groups such as deuterium, a halogen group, cyano, aryl, heteroaryl, trialkylsilyl, alkyl, cycloalkyl, alkoxy, alkylthio, and the like. Specific examples of aryl-substituted heteroaryl include, but are not limited to, phenyl-substituted dibenzofuranyl, phenyl-substituted dibenzothienyl, phenyl-substituted pyridyl, and the like. It should be understood that the number of carbon atoms of the substituted heteroaryl refers to the total number of carbon atoms of the heteroaryl and substituents on the heteroaryl.

In the present disclosure, “any adjacent” in “any two adjacent R form a ring” (R represents R₁ and R₂) may include a situation that a same atom has two R, and may also include a situation that there are two adjacent atoms and each of them has one R respectively; when the same atom has two R, the two R may form a saturated or unsaturated ring with the atom to which they are commonly connected, and may, for example, form a 5- to 15-membered saturated or unsaturated ring, such as cyclopentane, cyclohexane or a fluorene ring; and when the two adjacent atoms having one R respectively, the two R can be fused to form a ring, for example, the two R can be fused to form a benzene ring, a naphthalene ring, or the like.

In the present disclosure, an unpositioned connecting bond refers to a single bond “

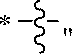

extending from a ring system, which means that one end of the connecting bond can be connected with any position in the ring system through which the bond penetrates, and the other end of the connecting bond is connected with the remaining part of a compound molecule.

For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1)-(f-10).

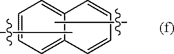

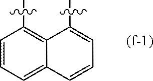

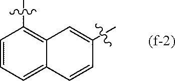

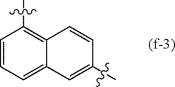

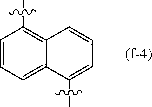

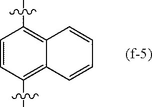

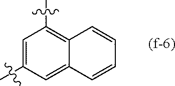

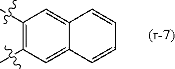

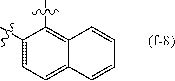

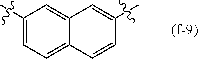

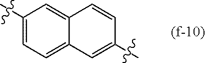

For another example, as shown in the following formula (X′), phenanthryl represented by the formula (X′) is connected with other positions of a molecule through one unpositioned connecting bond extending from the middle of a benzene ring on one side, and its meaning includes any one possible connecting mode represented by formulae (X′-1)-(X′-4).

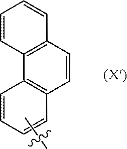

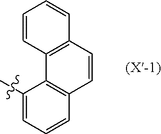

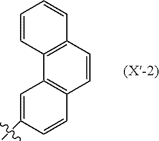

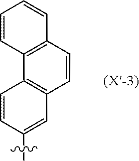

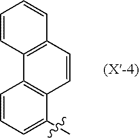

An unpositioned substituent in the present disclosure refers to a substituent connected through a single bond extending from the center of a ring system, which means that the substituent can be connected to any possible position in the ring system. For example, as shown in the following formula (Y), a substituent R′ represented in the formula (Y) is connected with a quinoline ring through one unpositioned connecting bond, and its meaning includes any one possible connecting mode represented by formulae (Y-1)-(Y-7).

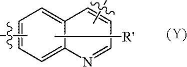

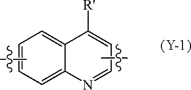

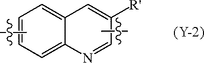

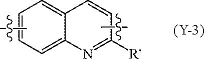

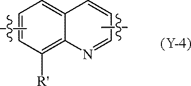

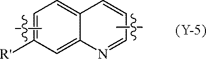

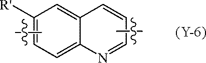

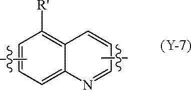

In the present disclosure, alkyl with 1 to 10 carbon atoms may include linear alkyl with 1 to 10 carbon atoms and branched alkyl with 3 to 10 carbon atoms, and the number of carbon atoms may be 1, 2, 3, 4, 5, 6, 7, 8, 9, or 10. Specific examples of the alkyl with 1 to 10 carbon atoms include, but are not limited to, methyl, ethyl, n-propyl, isopropyl, n-butyl, isobutyl, tert-butyl, n-pentyl, isopentyl, neopentyl, cyclopentyl, n-hexyl, heptyl, n-octyl, 2-ethylhexyl, nonyl, decyl, 3,7-dimethyloctyl, and the like.

In the present disclosure, the halogen group may include fluorine, iodine, bromine, chlorine, and the like.

In the present disclosure, the number of carbon atoms of aryl as a substituent is, for example, 6 to 18, 6 to 15, or the like, the number of carbon atoms is, for example, 6, 10, 12, 14, 15, 18, or the like, and specific examples of the aryl include, but are not limited to, phenyl, naphthyl, biphenyl, etc.

In the present disclosure, the number of carbon atoms of heteroaryl as a substituent is, for example, 3 to 18, 5 to 15, 5 to 12, or the like, the number of carbon atoms is, for example, 3, 4, 5, 6, 7, 8, 9, 10, 11, 12, 13, 14, 15, or the like, and specific examples of the heteroaryl include, but are not limited to, pyridyl, quinolyl, dibenzofuranyl, dibenzothienyl, carbazolyl, etc.

In the present disclosure, the number of carbon atoms of trialkylsilyl as a substituent may be 3 to 12, preferably 3 to 7, and specific examples include, but are not limited to, trimethylsilyl, ethyldimethylsilyl, triethylsilyl, and the like.

In the present disclosure, the number of carbon atoms of cycloalkyl as a substituent can be 3 to 10, preferably 5 to 10, and specific examples include, but are not limited to, cyclopentyl, cyclohexyl, adamantyl, and the like.

In the present disclosure, specific examples of haloalkyl include, but are not limited to, trifluoromethyl.

According to one example, the nitrogen-containing compound has a structure as shown in formula 1-1:

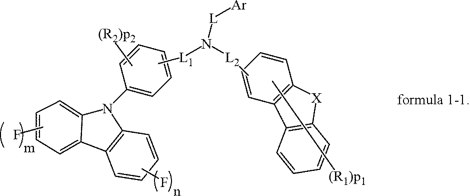

According to another example, the nitrogen-containing compound has a structure as shown in formula 1-2:

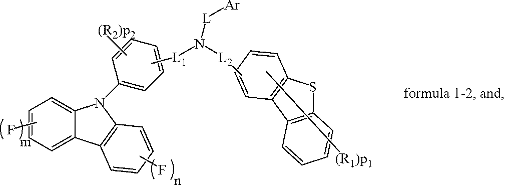

in the formula 1-2, L₂ is selected from a substituted or unsubstituted arylene group with 6 to 25 carbon atoms, and a substituted or unsubstituted heteroarylene group with 3 to 25 carbon atoms.

Optionally, R₁ and R₂ are the same or different, and are each independently selected from deuterium, fluorine, cyano, aryl with 6 to 15 carbon atoms, heteroaryl with 5 to 12 carbon atoms, trialkylsilyl with 3 to 7 carbon atoms, alkyl with 1 to 4 carbon atoms, fluoroalkyl with 1 to 4 carbon atoms, cycloalkyl with 5 to 10 carbon atoms, alkylthio with 1 to 4 carbon atoms, alkoxy with 1 to 4 carbon atoms, aryloxy with 6 to 12 carbon atoms, and arylthio with 6 to 12 carbon atoms.

Further optionally, R₁ and R₂ are the same or different, and are each independently selected from deuterium, fluorine, cyano, aryl with 6 to 12 carbon atoms, trialkylsilyl with 3 to 7 carbon atoms, alkyl with 1 to 4 carbon atoms, fluoroalkyl with 1 to 4 carbon atoms, and cycloalkyl with 5 to 10 carbon atoms. Specific examples of R₁ and R₂ respectively include, but are not limited to, deuterium, fluorine, cyano, phenyl, naphthyl, methyl, ethyl, isopropyl, tert-butyl, trifluoromethyl, cyclopentyl, cyclohexyl, and the like.

Optionally, Ar is selected from a substituted or unsubstituted aryl group with 6 to 25 carbon atoms, and substituted or a unsubstituted heteroaryl group with 5 to 24 carbon atoms. Specifically, Ar may be selected from substituted or unsubstituted aryl with 6, 7, 8, 9, 10, 11, 12, 13, 14, 15, 16, 17, 18, 19, 20, 21, 22, 23, 24, or 25 carbon atoms, and substituted or unsubstituted heteroaryl with 6, 7, 8, 9, 10, 11, 12, 13, 14, 15, 16, 17, 18, 19, 20, 21, 22, 23, or 24 carbon atoms.

Also optionally, Ar is selected from a substituted or unsubstituted aryl group with 6 to 24 carbon atoms, and a substituted or unsubstituted heteroaryl group with 5 to 20 carbon atoms.

In one example, Ar is selected from substituted or unsubstituted phenyl, substituted or unsubstituted biphenyl, substituted or unsubstituted naphthyl, substituted or unsubstituted fluorenyl, substituted or unsubstituted carbazolyl, substituted or unsubstituted dibenzofuranyl, substituted or unsubstituted dibenzothienyl, substituted or unsubstituted pyridyl, substituted or unsubstituted anthryl, substituted or unsubstituted phenanthryl, and substituted or unsubstituted quinolyl. Substituents in Ar are each independently selected from deuterium, fluoro, cyano, methyl, ethyl, n-propyl, isopropyl, tert-butyl, phenyl, pyridyl, naphthyl, dibenzofuranyl, dibenzothienyl, trimethylsilyl, and trifluoromethyl; and optionally, any two adjacent substituents form cyclopentane, cyclohexane or a fluorene ring. The number of carbon atoms of Ar is as described above.

Optionally, the substituents in Ar are selected from: deuterium, tritium, fluorine, cyano, aryl with 6 to 15 carbon atoms, heteroaryl with 3 to 12 carbon atoms, trialkylsilyl with 3 to 7 carbon atoms, alkyl with 1 to 4 carbon atoms, fluoroalkyl with 1 to 4 carbon atoms, cycloalkyl with 5 to 10 carbon atoms, alkylthio with 1 to 4 carbon atoms, alkoxy with 1 to 4 carbon atoms, aryloxy with 6 to 12 carbon atoms, arylthio with 6 to 12 carbon atoms, and triphenylsilyl.

Further optionally, the substituents in Ar are selected from deuterium, tritium, fluorine, cyano, aryl with 6 to 15 carbon atoms, heteroaryl with 5 to 12 carbon atoms, trialkylsilyl with 3 to 7 carbon atoms, alkyl with 1 to 4 carbon atoms, fluoroalkyl with 1 to 4 carbon atoms, cycloalkyl with 5 to 10 carbon atoms, and triphenylsilyl. Specific examples of the substituents in Ar include, but are not limited to, deuterium, tritium, fluorine, cyano, phenyl, naphthyl, dibenzofuranyl, dibenzothienyl, trimethylsilyl, methyl, ethyl, isopropyl, tert-butyl, trimethylsilyl, cyclohexyl, cyclopentyl, triphenylsilyl, and the like.

In some examples, Ar is selected from the group consisting of groups represented by formulae i-1 to i-15 below:

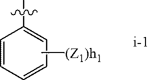

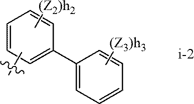

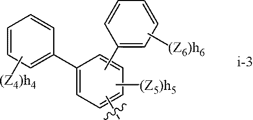

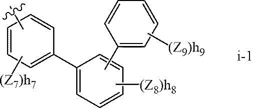

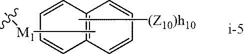

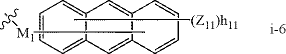

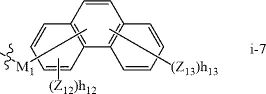

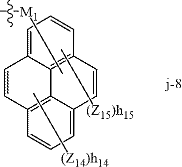

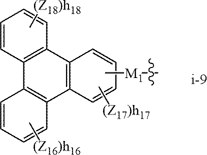

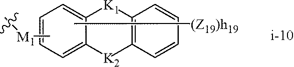

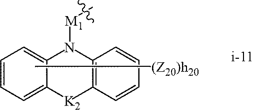

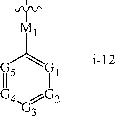

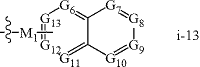

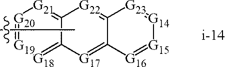

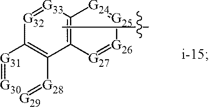

wherein,

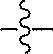

represents a chemical bond, and M₁ is selected from a single bond

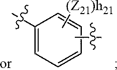

G₁-G₅ are each independently selected from N or C(F₁), and at least one of G₁-G₅ is selected from N; and when two or more of G₁-G₅ are selected from C(F₁), any two F₁ are the same or different;
G₆-G₁₃ are each independently selected from N or C(F₂), and at least one of G₆-G₁₃ is selected from N; and when two or more of G₆-G₁₃ are selected from C(F₂), any two F₂ are the same or different;
G₁₄-G₂₃ are each independently selected from N or C(F₃), and at least one of G₁₄-G₂₃ is selected from N; and when two or more of G₁₄-G₂₃ are selected from C(F₃), any two F₃ are the same or different;
G₂₄-G₃₃ are each independently selected from N or C(F₄), and at least one of G₂₄-G₃₃ is selected from N; and when two or more of G₂₄-G₃₃ are selected from C(F₄), any two F₄ are the same or different;
Z₁ is selected from hydrogen, deuterium, fluorine, chlorine, bromine, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, and triphenylsilyl;
Z₂-Z₉, and Z₂₁ are each independently selected from: hydrogen, deuterium, fluorine, chlorine, bromine, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, and heteroaryl with 3 to 15 carbon atoms;
Z₁₀-Z₂₀, and F₁-F₄ are each independently selected from: hydrogen, deuterium, fluorine, chlorine, bromine, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, aryl with 6 to 18 carbon atoms, and heteroaryl with 3 to 15 carbon atoms;
h₁-h₂₁ are represented by hₖ, Z₁-Z₂₁ are represented by Zₖ, k is a variable, and represents any integer from 1 to 21, and hₖ represents the number of a substituent Zₖ; when k is selected from 5 or 17, hₖ is selected from 1, 2 or 3; when k is selected from 2, 7, 8, 12, 15, 16, 18 or 21, hₖ is selected from 1, 2, 3 or 4; when k is selected from 1, 3, 4, 6, 9 or 14, hₖ is selected from 1, 2, 3, 4 or 5; when k is 13, hₖ is selected from 1, 2, 3, 4, 5, or 6; when k is selected from 10 or 19, hₖ is selected from 1, 2, 3, 4, 5, 6 or 7; when k is 20, hₖ is selected from 1, 2, 3, 4, 5, 6, 7, or 8; when k is 11, hₖ is selected from 1, 2, 3, 4, 5, 6, 7, 8 or 9; and when hₖ is greater than 1, any two Zₖ are the same or different; and optionally, any two adjacent Zₖ form a ring;
K₁ is selected from O, S, N(Z₂₂), C(Z₂₃Z₂₄), and Si(Z₂₃Z₂₄); wherein Z₂₂, Z₂₃, and Z₂₄ are each independently selected from hydrogen, aryl with 6 to 18 carbon atoms, heteroaryl with 3 to 15 carbon atoms, alkyl with 1 to 10 carbon atoms or cycloalkyl with 3 to 10 carbon atoms, or the Z₂₃ and the Z₂₄ are connected to each other to form a saturated or unsaturated ring with 5 to 15 carbon atoms together with the atoms to which they are commonly connected; and
K₂ is selected from a single bond, O, S, N(Z₂₅), C(Z₂₆Z₂₇), and Si(Z₂₆Z₂₇); wherein Z₂₅, Z₂₆, and Z₂₇ are each independently selected from hydrogen, aryl with 6 to 18 carbon atoms, heteroaryl with 3 to 15 carbon atoms, alkyl with 1 to 10 carbon atoms or cycloalkyl with 3 to 10 carbon atoms, or the Z₂₆ and the Z₂₇ are connected to each other to form a saturated or unsaturated ring with 5 to 15 carbon atoms together with the atoms to which they are commonly connected.

In the formulae i-13 to i-15, F₂-F₄ may be represented by Fᵢ, wherein i is a variable and represents 2, 3, or 4. For example, when i is 2, Fᵢ refers to F₂. It should be understood that Fᵢ in C(Fᵢ) is not present when an unpositioned connecting bond is connected to C(Fᵢ). For example, in the formula i-13, when “

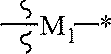

“ is connected to G₁₂, G₁₂ can only represent a C atom, i.e., a structure of the formula i-13 is specifically:

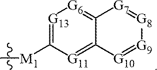

In the present disclosure, in both groups of the Z₂₃ and the Z₂₄, and the Z₂₆ and the Z₂₇, a ring formed by connecting two groups with each other in one group may be saturated or unsaturated, for example, a 5- to 15-membered saturated or unsaturated ring can be formed. For example, in the formula i-10, when K₂ and M₁ are both a single bond, Z₁₉ is hydrogen, h₁₉=7, and K₁ is C(Z₂₃Z₂₄), and Z₂₃ and Z₂₄ are connected to each other to form a 5-membered ring with the atoms to which they are commonly connected, the formula i-10 is

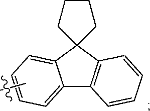

and likewise, the formula i-10 may also represent

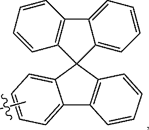

that is, Z₂₃ and Z₂₄ are connected to each other to form a partially unsaturated 13-membered ring with the atoms to which they are commonly connected.

Optionally, Ar is selected from a substituted or unsubstituted group V₁, and the unsubstituted group V₁ is selected from the group consisting of:

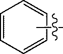

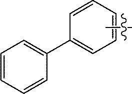

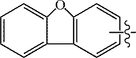

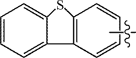

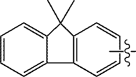

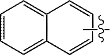

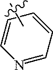

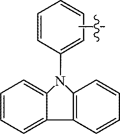

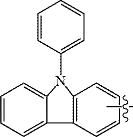

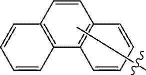

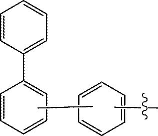

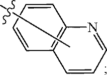

and the substituted group V₁ has one or two or more substituents, and the substituents are respectively and independently selected from deuterium, fluorine, cyano, alkyl with 1 to 4 carbon atoms, fluoroalkyl with 1 to 4 carbon atoms, cycloalkyl with 5 to 10 carbon atoms, trialkylsilyl with 3 to 7 carbon atoms, phenyl, naphthyl, pyridyl, and triphenylsilyl; and when the number of the substituents is greater than 1, the substituents are the same or different; and optionally, any two adjacent substituents form a ring, e.g. a fluorene ring, cyclohexane or cyclopentane.

Optionally, Ar is selected from the group consisting of:

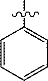

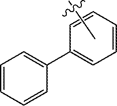

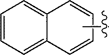

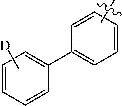

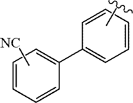

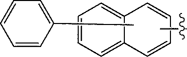

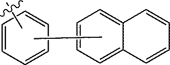

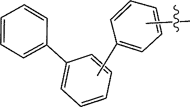

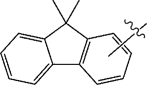

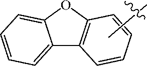

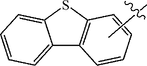

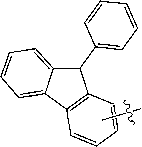

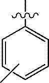

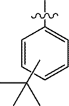

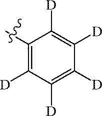

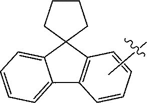

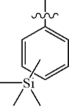

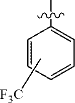

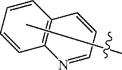

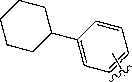

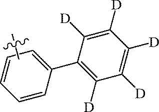

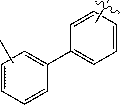

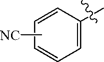

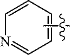

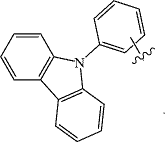

Further optionally, Ar is selected from the group consisting of:

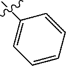

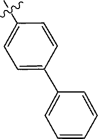

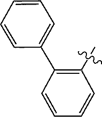

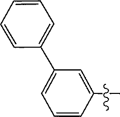

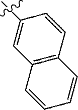

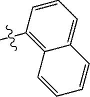

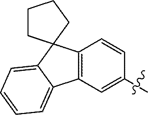

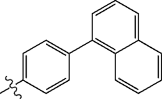

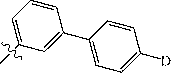

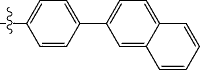

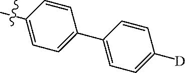

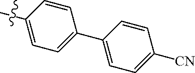

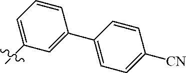

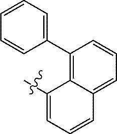

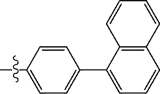

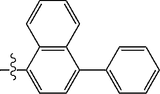

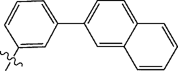

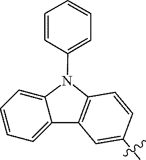

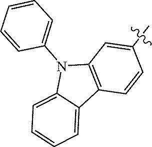

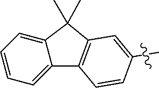

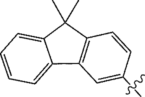

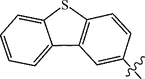

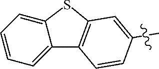

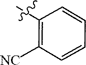

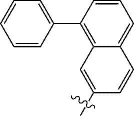

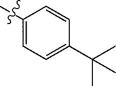

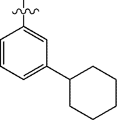

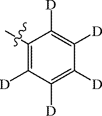

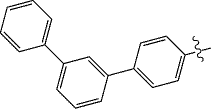

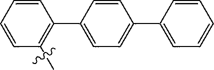

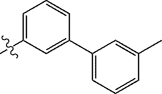

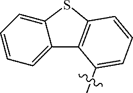

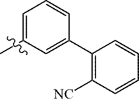

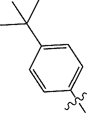

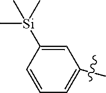

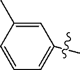

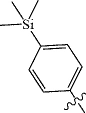

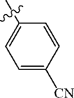

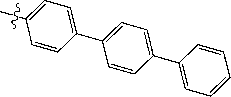

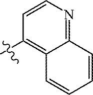

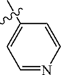

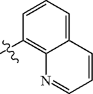

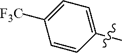

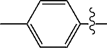

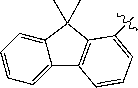

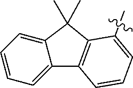

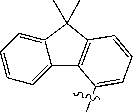

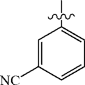

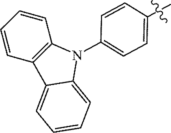

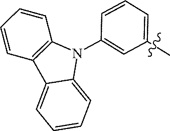

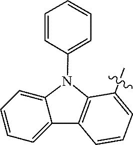

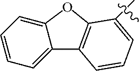

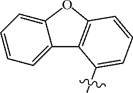

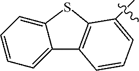

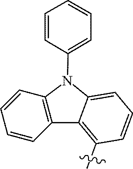

In one example, Ar is

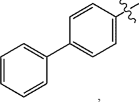

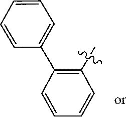

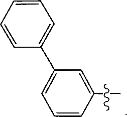

Preferably, Ar is

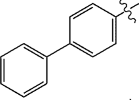

Optionally, L, L₁ and L₂ are the same or different, and are each independently selected from a single bond, substituted or unsubstituted arylene with 6 to 20 carbon atoms, and substituted or unsubstituted heteroarylene with 5 to 20 carbon atoms. Specifically, L, L₁ and L₂ may each independently be selected from a single bond, substituted or unsubstituted arylene with 6, 7, 8, 9, 10, 11, 12, 13, 14, 15, 16, 17, 18, 19, or 20 carbon atoms, and substituted or unsubstituted heteroarylene with 5, 7, 8, 9, 10, 11, 12, 13, 14, 15, 16, 17, 18, 19, or 20 carbon atoms.

Also optionally, L, L₁ and L₂ are the same or different, and are each independently selected from a single bond, substituted or unsubstituted arylene with 6 to 15 carbon atoms, and substituted or unsubstituted heteroarylene with 5 to 15 carbon atoms.

Optionally, substituents in L, L₁ and L₂ are each independently selected from deuterium, fluorine, cyano, alkyl with 1 to 4 carbon atoms, cyclopentyl, cyclohexyl, fluoroalkyl with 1 to 4 carbon atoms, methoxy, trimethylsilyl, triethylsilyl, and phenyl.

Further optionally, the substituents in L, L₁ and L₂ are each independently selected from deuterium, fluorine, cyano, methyl, ethyl, isopropyl, tert-butyl, cyclopentyl, cyclohexyl, trifluoromethyl, trimethylsilyl, and phenyl.

In some examples, L, L₁ and L₂ are the same or different, and are each independently selected from a single bond or selected from the group consisting of groups represented by formulae j-1 to j-13:

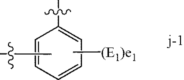

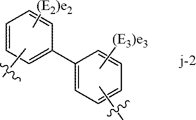

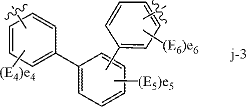

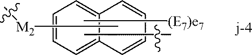

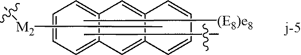

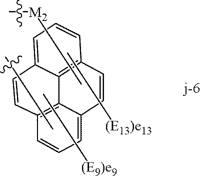

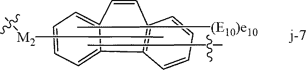

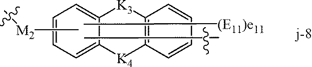

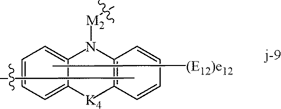

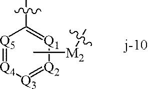

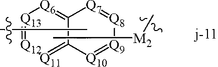

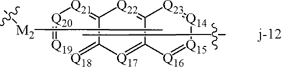

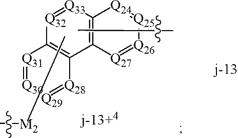

wherein M₂ is selected from a single bond or

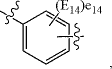

and

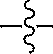

represents a chemical bond;
Q₁-Q₅ are each independently selected from N or C(Js), and at least one of Q₁-Q₅ is selected from N; and when two or more of Q₁-Q₅ are selected from C(Js), any two J₅ are the same or different;
Q₆-Q₁₃ are each independently selected from N or C(J₆), and at least one of Q₆-Q₁₃ is selected from N; and when two or more of Q₆-Q₁₃ are selected from C(J₆), any two J₆ are the same or different;
Q₁₄-Q₂₃ are each independently selected from N or C(J₇), and at least one of Q₁₄-Q₂₃ is selected from N; and when two or more of Q₁₄-Q₂₃ are selected from C(J₇), any two J₇ are the same or different;
Q₂₄-Q₃₂ are each independently selected from N or C(Js), and at least one of Q₂₄-Q₃₂ is selected from N; and when two or more of Q₂₄-Q₃₂ are selected from C(Js), any two J₈ are the same or different;
E₁-E₁₄, and J₅-J₉ are each independently selected from: hydrogen, deuterium, a halogen group, heteroaryl with 3 to 15 carbon atoms, aryl with 6 to 15 carbon atoms, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, aryloxy with 6 to 12 carbon atoms, and arylthio with 6 to 12 carbon atoms;
e₁-e₁₄ are represented by eᵣ, E₁-E₁₄ are represented by Eᵣ, r is a variable, and represents any integer from 1 to 14, and eᵣ represents the number of a substituent Eᵣ; when r is selected from 1, 2, 3, 4, 5, 6, 9, 13 or 14, eᵣ is selected from 1, 2, 3 or 4; when r is selected from 7 or 11, eᵣ is selected from 1, 2, 3, 4, 5 or 6; when r is 12, eᵣ is selected from 1, 2, 3, 4, 5, 6, or 7; when r is selected from 8 or 10, eᵣ is selected from 1, 2, 3, 4, 5, 6, 7, or 8; and when eᵣ is greater than 1, any two Eᵣ are the same or different;
K₃ is selected from O, S, Se, N(E₁₅), C(E₁₆E₁₇), and Si(E₁₆E₁₇); wherein E₁₅, E₁₆, and E₁₇ are each independently selected from aryl with 6 to 15 carbon atoms, heteroaryl with 3 to 15 carbon atoms, alkyl with 1 to 10 carbon atoms, and cycloalkyl with 3 to 10 carbon atoms, or E₁₆ and E₁₇ are connected to each other to form a saturated or unsaturated ring with 5 to 15 carbon atoms together with the atoms to which they are commonly connected; and
K₄ is selected from a single bond, O, S, Se, N(E₁₈), C(E₁₉E₂₀), and Si(E₁₉E₂₀); wherein E₁₈ to E₂₀ are each independently selected from aryl with 6 to 15 carbon atoms, heteroaryl with 3 to 15 carbon atoms, alkyl with 1 to 10 carbon atoms, and cycloalkyl with 3 to 10 carbon atoms, or E₁₉ and E₂₀ are connected to each other to form a saturated or unsaturated ring with 5 to 15 carbon atoms together with the atoms to which they are commonly connected.

Optionally, L, L₁ and L₂ are the same or different, and are each independently selected from a single bond, substituted or unsubstituted phenylene, substituted or unsubstituted naphthylene, substituted or unsubstituted pyridylene, substituted or unsubstituted 9,9-dimethylfluorenylidene, substituted or unsubstituted dibenzofurylene, substituted or unsubstituted dibenzothienylene, and substituted or unsubstituted carbazolylene; and substituents are independently selected from deuterium, fluorine, cyano, methyl, ethyl, n-propyl, isopropyl, tert-butyl, phenyl, naphthyl, pyridyl, trifluoromethyl, trimethylsilyl, methoxy, methylthio, cyclohexyl, and cyclopentyl. The number of carbon atoms of L, L₁ and L₂ is as shown above.

In one example, L, L₁ and L₂ are the same or different, and are each independently selected from a single bond, and a substituted or unsubstituted group V₂, and the unsubstituted group V₂ is selected from the group consisting of:

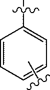

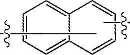

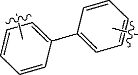

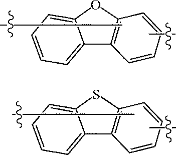

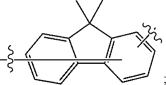

and the substituted group V₂ has one or two or more substituents, and the substituents are respectively and independently selected from deuterium, fluorine, cyano, alkyl with 1 to 4 carbon atoms, fluoroalkyl with 1 to 4 carbon atoms, cycloalkyl with 5 to 10 carbon atoms, trialkylsilyl with 3 to 7 carbon atoms, phenyl, and naphthyl; and when the number of the substituents is greater than 1, the substituents are the same or different.

Optionally, L₁ is selected from a single bond or the group consisting of:

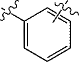

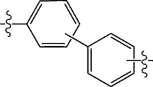

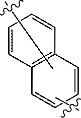

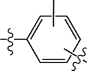

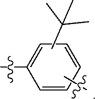

Optionally,

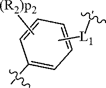

in the formula 1 is selected from the group consisting of:

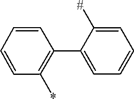

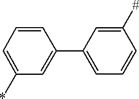

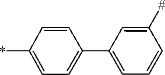

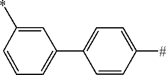

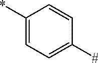

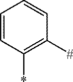

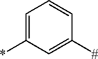

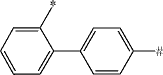

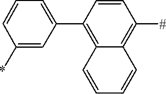

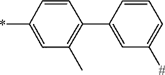

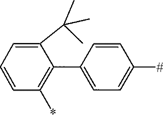

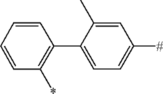

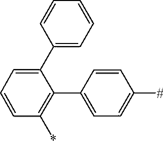

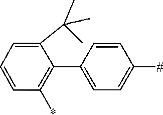

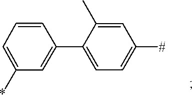

wherein, * indicates a site for connecting with

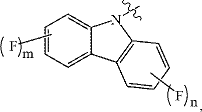

and # indicates a site for connecting with

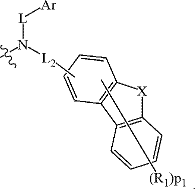

Preferably,

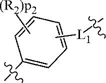

in the formula 1 is selected from

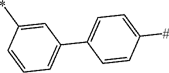

or

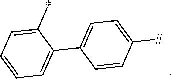

Optionally, L and L₂ are each independently selected from a single bond or the group consisting of:

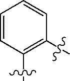

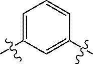

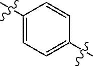

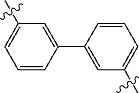

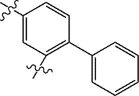

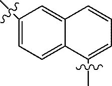

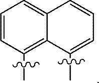

In one example, the total number of carbon atoms of groups L and Ar is not more than 25, and preferably, the total number of carbon atoms of the groups L and Ar is 10 to 22.

Optionally, L₂ is a single bond or phenylene.

Optionally,

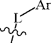

in the formula 1 is selected from the group consisting of:

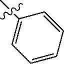

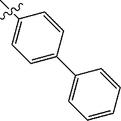

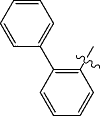

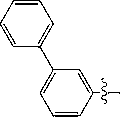

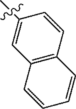

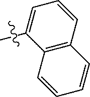

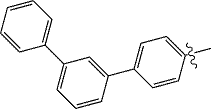

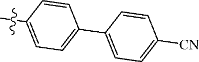

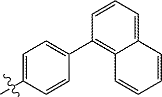

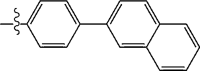

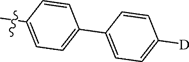

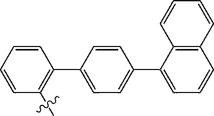

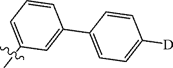

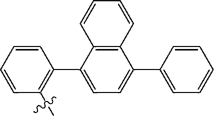

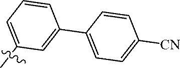

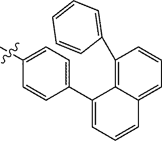

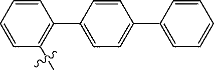

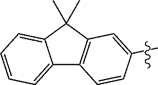

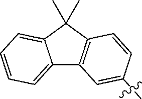

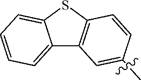

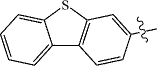

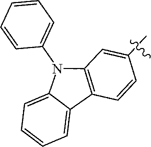

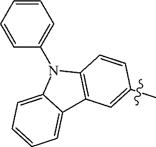

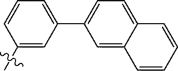

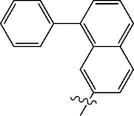

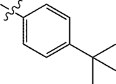

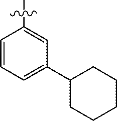

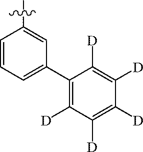

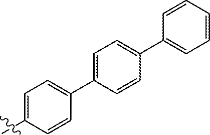

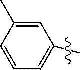

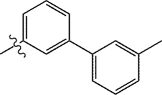

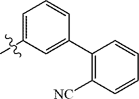

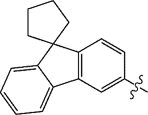

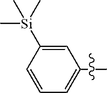

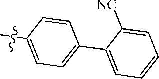

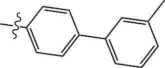

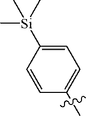

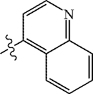

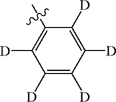

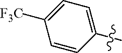

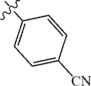

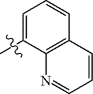

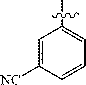

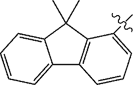

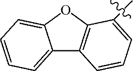

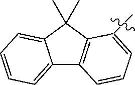

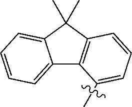

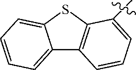

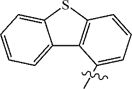

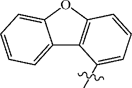

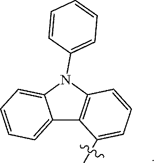

In the present disclosure,

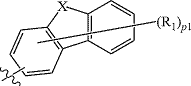

in the formula 1 may specifically be selected from the group consisting of the following structures:

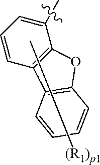

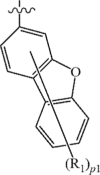

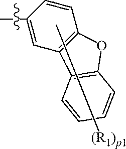

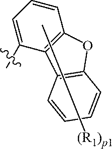

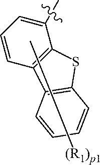

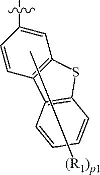

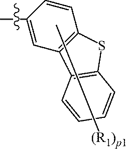

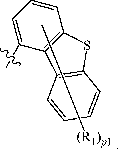

Preferably,

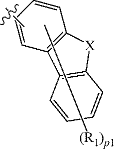

is selected from

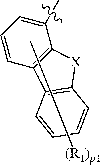

or

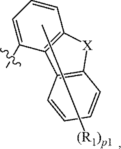

that is,

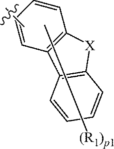

is selected from the group consisting of:

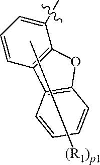

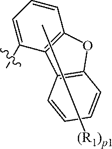

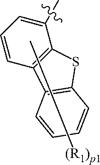

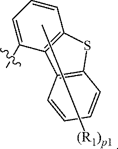

In the present disclosure,

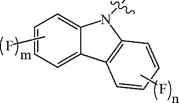

in the formula 1 may specifically be selected from the group consisting of:

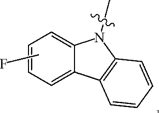

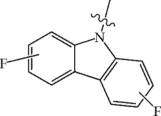

or

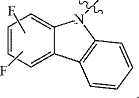

Preferably, m=1 and n=1 or 0, i.e.

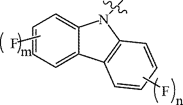

is selected from

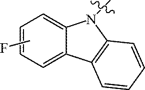

or

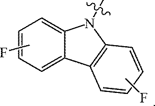

According to one example, L₁ is phenylene.

According to one preferred example, the structure of the nitrogen-containing compound is selected from the group consisting of structures represented by formulae 1-A to 1-D:

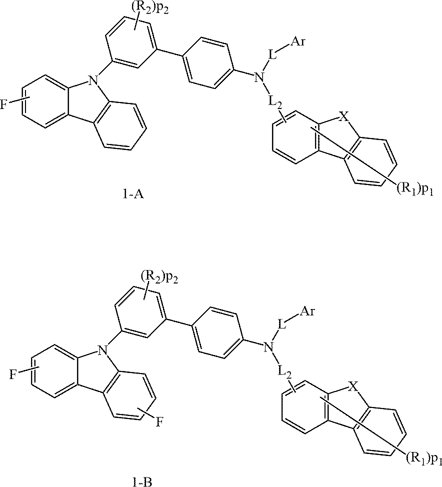

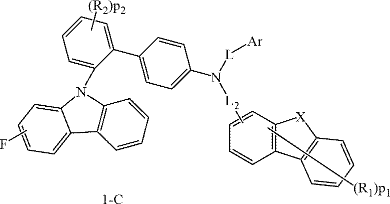

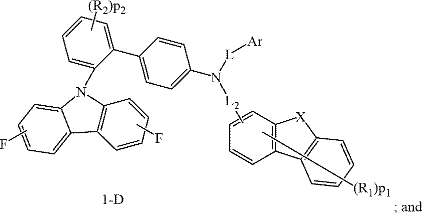

in this preferred example, the nitrogen-containing compound is applied to OLEDs, so that the service life of the device can be further prolonged.

Further preferably, the structure of the nitrogen-containing compound is selected from the group consisting of the following structures:

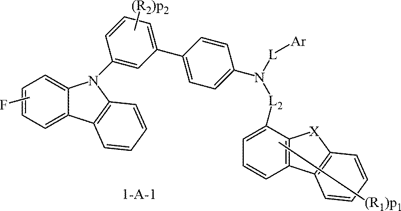

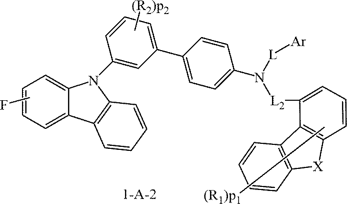

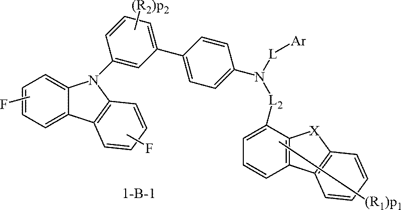

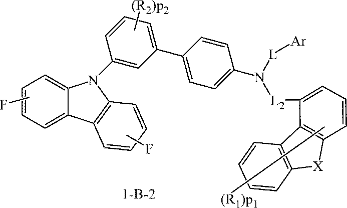

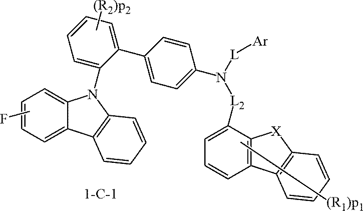

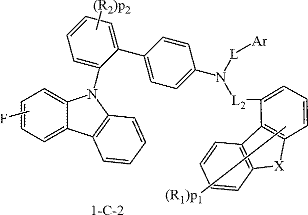

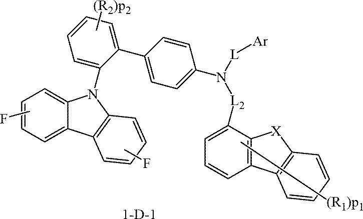

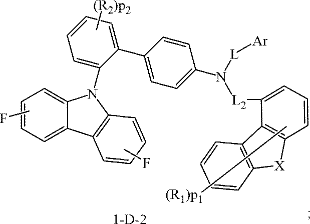

in this case, the nitrogen-containing compound of the present disclosure has a better configuration, three groups on arylamine have higher compatibility, and the interaction between the groups can be fully realized, so that the performance of the device can be further improved. More preferably, X is O.

Optionally, the nitrogen-containing compound is selected from the group consisting of the following compounds:

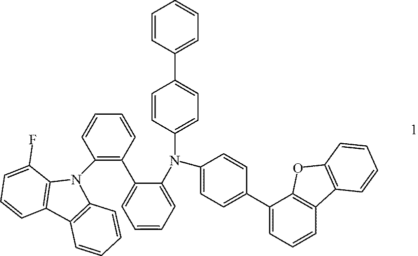 [SMILES: Fc1cccc2c3ccccc3n(-c3ccccc3-c3ccccc3N(c3ccc(-c4ccccc4)cc3)c3ccc(-c4cccc5c4oc4ccccc45)cc3)c12]

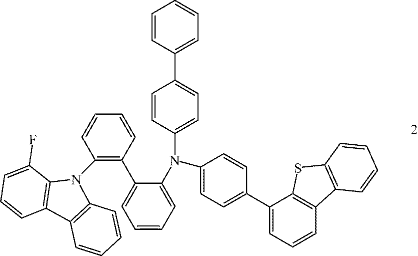 [SMILES: Fc1cccc2c3ccccc3n(-c3ccccc3-c3ccccc3N(c3ccc(-c4ccccc4)cc3)c3ccc(-c4cccc5c4sc4ccccc45)cc3)c12]

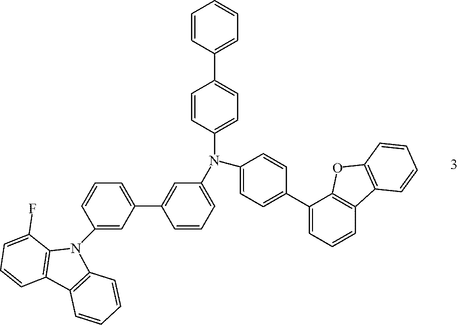 [SMILES: Fc1cccc2c3ccccc3n(-c3cccc(-c4cccc(N(c5ccc(-c6ccccc6)cc5)c5ccc(-c6cccc7c6oc6ccccc67)cc5)c4)c3)c12]

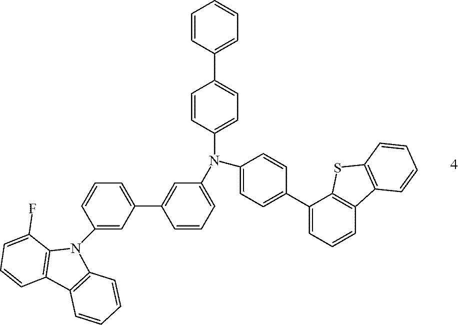 [SMILES: Fc1cccc2c3ccccc3n(-c3cccc(-c4cccc(N(c5ccc(-c6ccccc6)cc5)c5ccc(-c6cccc7c6sc6ccccc67)cc5)c4)c3)c12]

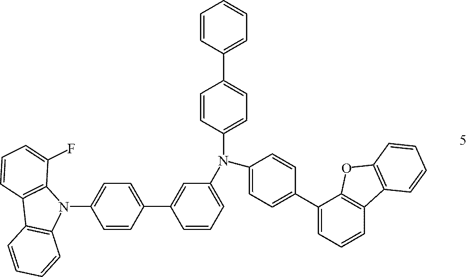 [SMILES: Fc1cccc2c3ccccc3n(-c3ccc(-c4cccc(N(c5ccc(-c6ccccc6)cc5)c5ccc(-c6cccc7c6oc6ccccc67)cc5)c4)cc3)c12]

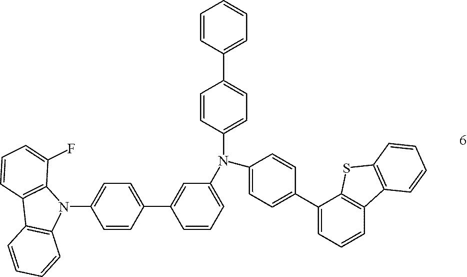 [SMILES: Fc1cccc2c3ccccc3n(-c3ccc(-c4cccc(N(c5ccc(-c6ccccc6)cc5)c5ccc(-c6cccc7c6sc6ccccc67)cc5)c4)cc3)c12]

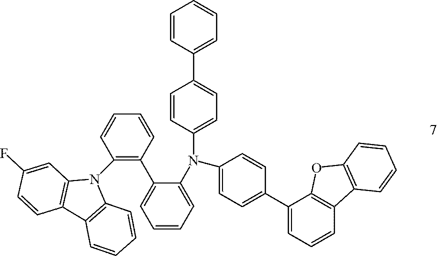 [SMILES: Fc1ccc2c3ccccc3n(-c3ccccc3-c3ccccc3N(c3ccc(-c4ccccc4)cc3)c3ccc(-c4cccc5c4oc4ccccc45)cc3)c2c1]

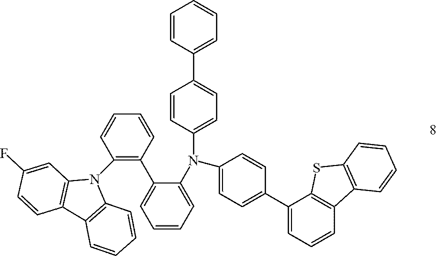 [SMILES: Fc1ccc2c3ccccc3n(-c3ccccc3-c3ccccc3N(c3ccc(-c4ccccc4)cc3)c3ccc(-c4cccc5c4sc4ccccc45)cc3)c2c1]

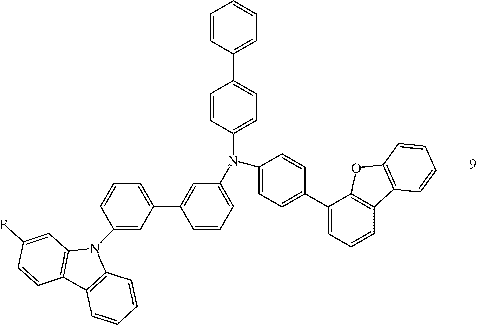 [SMILES: Fc1ccc2c3ccccc3n(-c3cccc(-c4cccc(N(c5ccc(-c6ccccc6)cc5)c5ccc(-c6cccc7c6oc6ccccc67)cc5)c4)c3)c2c1]

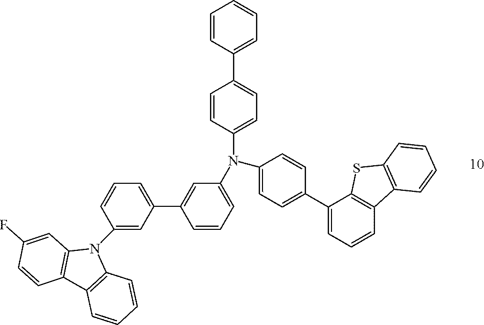 [SMILES: Fc1ccc2c3ccccc3n(-c3cccc(-c4cccc(N(c5ccc(-c6ccccc6)cc5)c5ccc(-c6cccc7c6sc6ccccc67)cc5)c4)c3)c2c1]

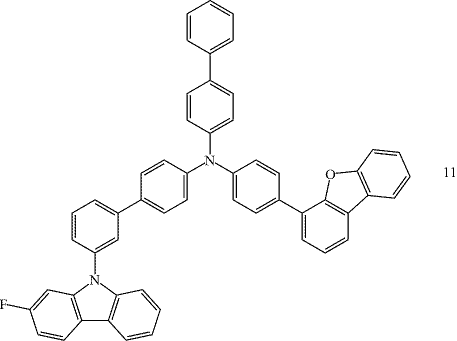 [SMILES: Fc1ccc2c3ccccc3n(-c3cccc(-c4ccc(N(c5ccc(-c6ccccc6)cc5)c5ccc(-c6cccc7c6oc6ccccc67)cc5)cc4)c3)c2c1]

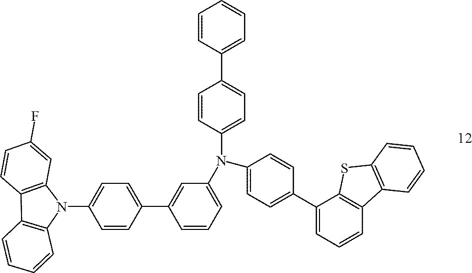 [SMILES: Fc1ccc2c3ccccc3n(-c3ccc(-c4cccc(N(c5ccc(-c6ccccc6)cc5)c5ccc(-c6cccc7c6sc6ccccc67)cc5)c4)cc3)c2c1]

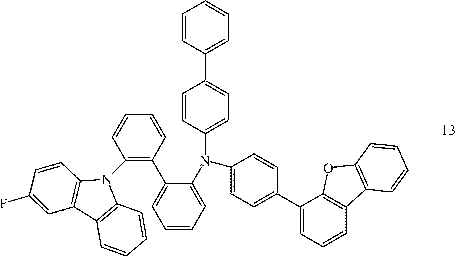 [SMILES: Fc1ccc2c(c1)c1ccccc1n2-c1ccccc1-c1ccccc1N(c1ccc(-c2ccccc2)cc1)c1ccc(-c2cccc3c2oc2ccccc23)cc1]

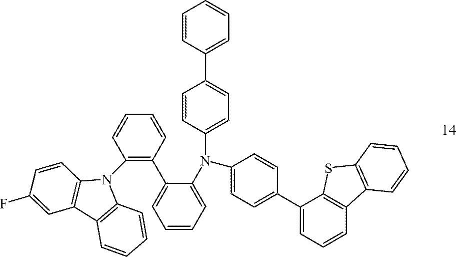 [SMILES: Fc1ccc2c(c1)c1ccccc1n2-c1ccccc1-c1ccccc1N(c1ccc(-c2ccccc2)cc1)c1ccc(-c2cccc3c2sc2ccccc23)cc1]

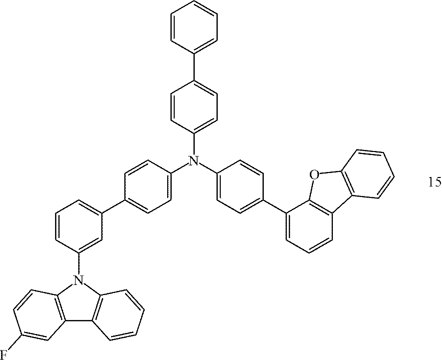 [SMILES: Fc1ccc2c(c1)c1ccccc1n2-c1cccc(-c2ccc(N(c3ccc(-c4ccccc4)cc3)c3ccc(-c4cccc5c4oc4ccccc45)cc3)cc2)c1]

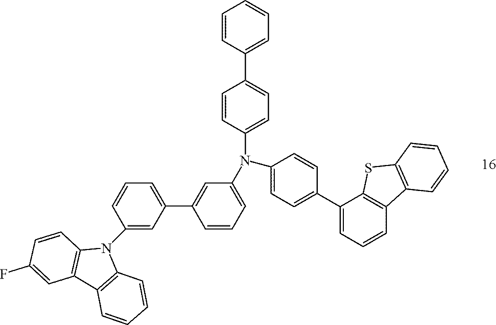 [SMILES: Fc1ccc2c(c1)c1ccccc1n2-c1cccc(-c2cccc(N(c3ccc(-c4ccccc4)cc3)c3ccc(-c4cccc5c4sc4ccccc45)cc3)c2)c1]

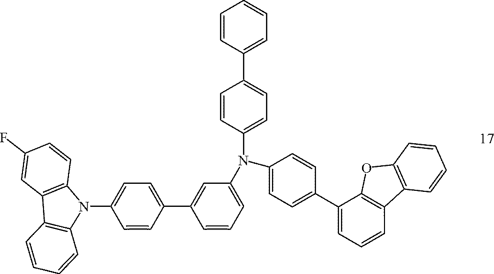 [SMILES: Fc1ccc2c(c1)c1ccccc1n2-c1ccc(-c2cccc(N(c3ccc(-c4ccccc4)cc3)c3ccc(-c4cccc5c4oc4ccccc45)cc3)c2)cc1]

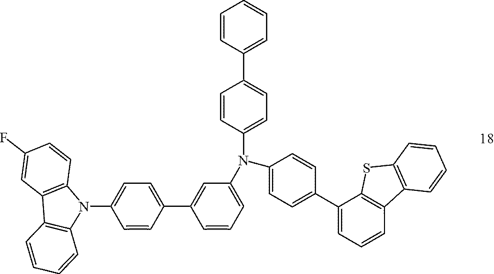 [SMILES: Fc1ccc2c(c1)c1ccccc1n2-c1ccc(-c2cccc(N(c3ccc(-c4ccccc4)cc3)c3ccc(-c4cccc5c4sc4ccccc45)cc3)c2)cc1]

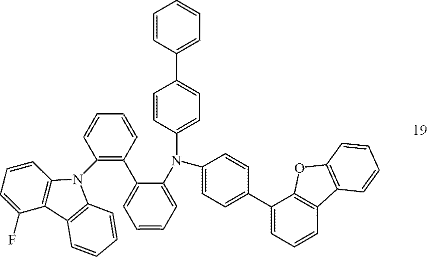 [SMILES: Fc1cccc2c1c1ccccc1n2-c1ccccc1-c1ccccc1N(c1ccc(-c2ccccc2)cc1)c1ccc(-c2cccc3c2oc2ccccc23)cc1]

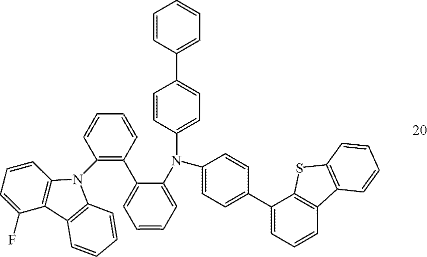 [SMILES: Fc1cccc2c1c1ccccc1n2-c1ccccc1-c1ccccc1N(c1ccc(-c2ccccc2)cc1)c1ccc(-c2cccc3c2sc2ccccc23)cc1]

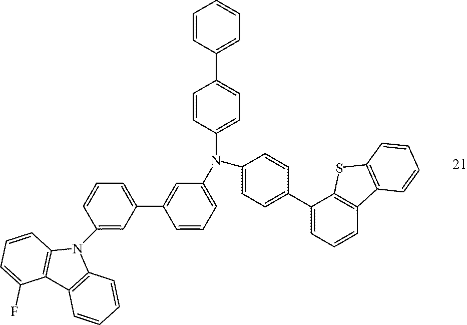 [SMILES: Fc1cccc2c1c1ccccc1n2-c1cccc(-c2cccc(N(c3ccc(-c4ccccc4)cc3)c3ccc(-c4cccc5c4sc4ccccc45)cc3)c2)c1]

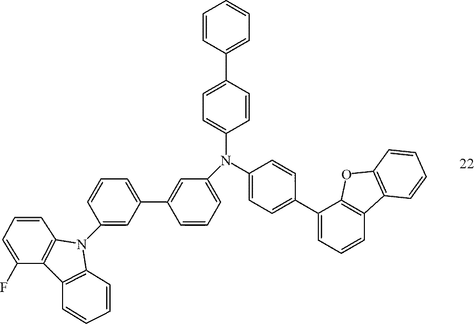 [SMILES: Fc1cccc2c1c1ccccc1n2-c1cccc(-c2cccc(N(c3ccc(-c4ccccc4)cc3)c3ccc(-c4cccc5c4oc4ccccc45)cc3)c2)c1]

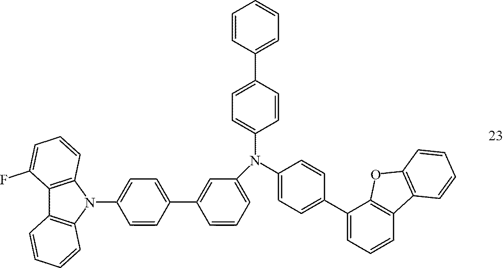 [SMILES: Fc1cccc2c1c1ccccc1n2-c1ccc(-c2cccc(N(c3ccc(-c4ccccc4)cc3)c3ccc(-c4cccc5c4oc4ccccc45)cc3)c2)cc1]

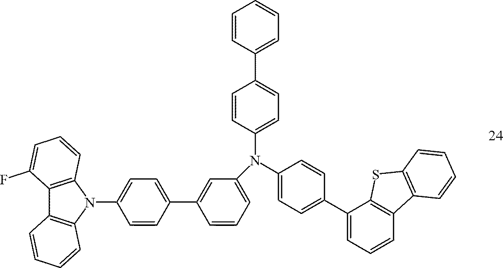 [SMILES: Fc1cccc2c1c1ccccc1n2-c1ccc(-c2cccc(N(c3ccc(-c4ccccc4)cc3)c3ccc(-c4cccc5c4sc4ccccc45)cc3)c2)cc1]

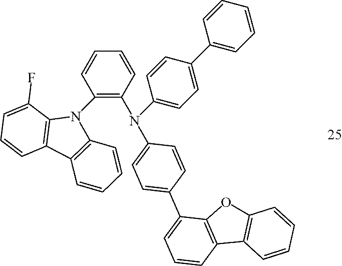 [SMILES: Fc1cccc2c3ccccc3n(-c3ccccc3N(c3ccc(-c4ccccc4)cc3)c3ccc(-c4cccc5c4oc4ccccc45)cc3)c12]

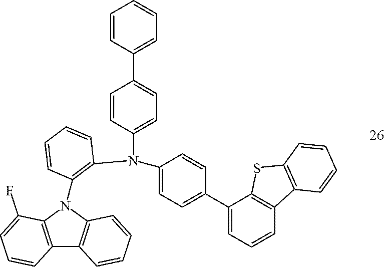 [SMILES: Fc1cccc2c3ccccc3n(-c3ccccc3N(c3ccc(-c4ccccc4)cc3)c3ccc(-c4cccc5c4sc4ccccc45)cc3)c12]

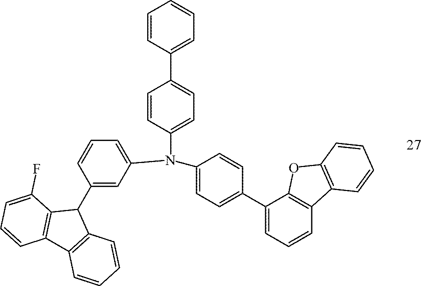 [SMILES: Fc1cccc2c1C(c1cccc(N(c3ccc(-c4ccccc4)cc3)c3ccc(-c4cccc5c4oc4ccccc45)cc3)c1)c1ccccc1-2]

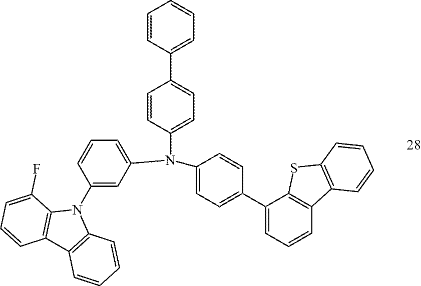 [SMILES: Fc1cccc2c3ccccc3n(-c3cccc(N(c4ccc(-c5ccccc5)cc4)c4ccc(-c5cccc6c5sc5ccccc56)cc4)c3)c12]

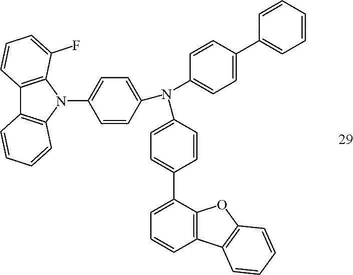 [SMILES: Fc1cccc2c3ccccc3n(-c3ccc(N(c4ccc(-c5ccccc5)cc4)c4ccc(-c5cccc6c5oc5ccccc56)cc4)cc3)c12]

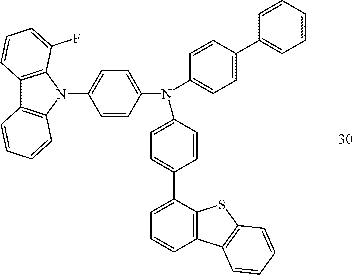 [SMILES: Fc1cccc2c3ccccc3n(-c3ccc(N(c4ccc(-c5ccccc5)cc4)c4ccc(-c5cccc6c5sc5ccccc56)cc4)cc3)c12]

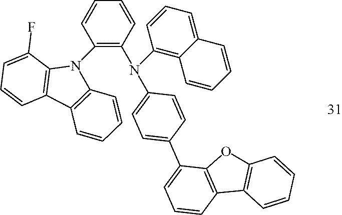 [SMILES: Fc1cccc2c3ccccc3n(-c3ccccc3N(c3ccc(-c4cccc5c4oc4ccccc45)cc3)c3cccc4ccccc34)c12]

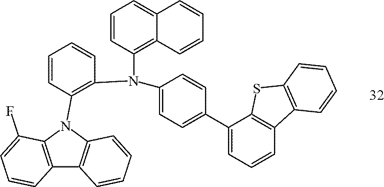 [SMILES: Fc1cccc2c3ccccc3n(-c3ccccc3N(c3ccc(-c4cccc5c4sc4ccccc45)cc3)c3cccc4ccccc34)c12]

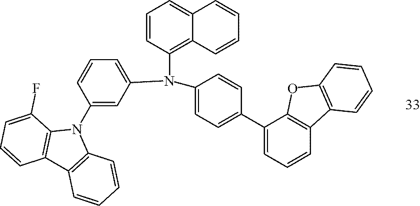 [SMILES: Fc1cccc2c3ccccc3n(-c3cccc(N(c4ccc(-c5cccc6c5oc5ccccc56)cc4)c4cccc5ccccc45)c3)c12]

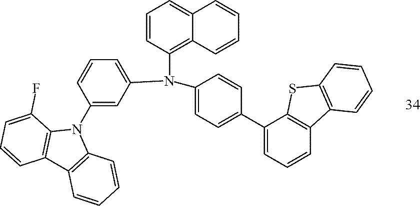 [SMILES: Fc1cccc2c3ccccc3n(-c3cccc(N(c4ccc(-c5cccc6c5sc5ccccc56)cc4)c4cccc5ccccc45)c3)c12]

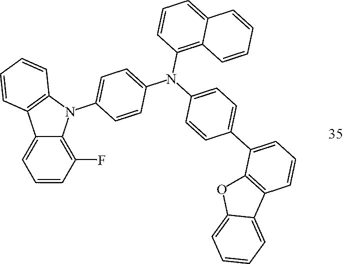 [SMILES: Fc1cccc2c3ccccc3n(-c3ccc(N(c4ccc(-c5cccc6c5oc5ccccc56)cc4)c4cccc5ccccc45)cc3)c12]

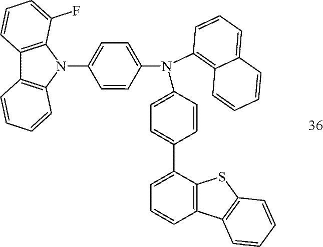 [SMILES: Fc1cccc2c3ccccc3n(-c3ccc(N(c4ccc(-c5cccc6c5sc5ccccc56)cc4)c4cccc5ccccc45)cc3)c12]

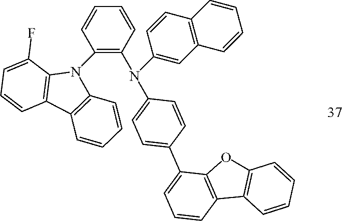 [SMILES: Fc1cccc2c3ccccc3n(-c3ccccc3N(c3ccc(-c4cccc5c4oc4ccccc45)cc3)c3ccc4ccccc4c3)c12]

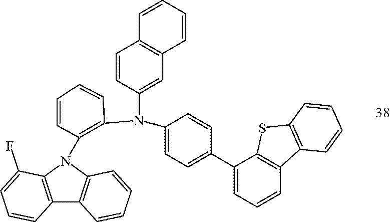 [SMILES: Fc1cccc2c3ccccc3n(-c3ccccc3N(c3ccc(-c4cccc5c4sc4ccccc45)cc3)c3ccc4ccccc4c3)c12]

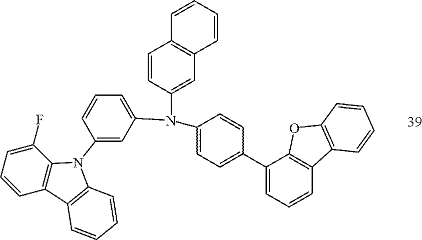 [SMILES: Fc1cccc2c3ccccc3n(-c3cccc(N(c4ccc(-c5cccc6c5oc5ccccc56)cc4)c4ccc5ccccc5c4)c3)c12]

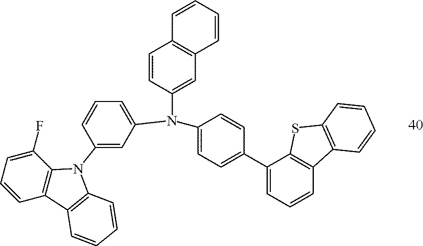 [SMILES: Fc1cccc2c3ccccc3n(-c3cccc(N(c4ccc(-c5cccc6c5sc5ccccc56)cc4)c4ccc5ccccc5c4)c3)c12]

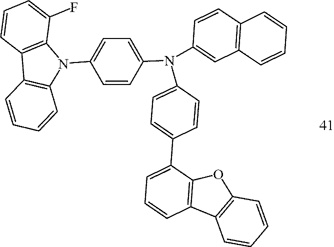 [SMILES: Fc1cccc2c3ccccc3n(-c3ccc(N(c4ccc(-c5cccc6c5oc5ccccc56)cc4)c4ccc5ccccc5c4)cc3)c12]

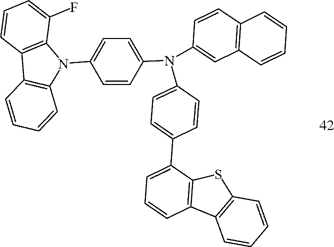 [SMILES: Fc1cccc2c3ccccc3n(-c3ccc(N(c4ccc(-c5cccc6c5sc5ccccc56)cc4)c4ccc5ccccc5c4)cc3)c12]

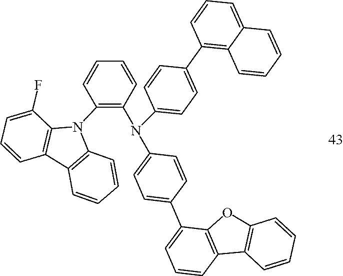 [SMILES: Fc1cccc2c3ccccc3n(-c3ccccc3N(c3ccc(-c4cccc5ccccc45)cc3)c3ccc(-c4cccc5c4oc4ccccc45)cc3)c12]

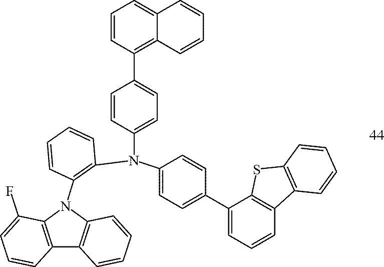 [SMILES: Fc1cccc2c3ccccc3n(-c3ccccc3N(c3ccc(-c4cccc5ccccc45)cc3)c3ccc(-c4cccc5c4sc4ccccc45)cc3)c12]

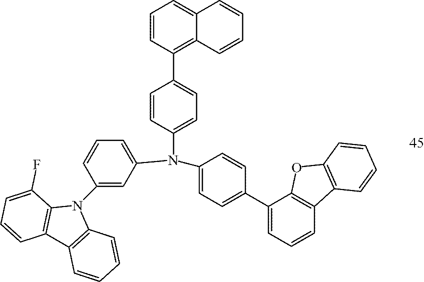 [SMILES: Fc1cccc2c3ccccc3n(-c3cccc(N(c4ccc(-c5cccc6ccccc56)cc4)c4ccc(-c5cccc6c5oc5ccccc56)cc4)c3)c12]

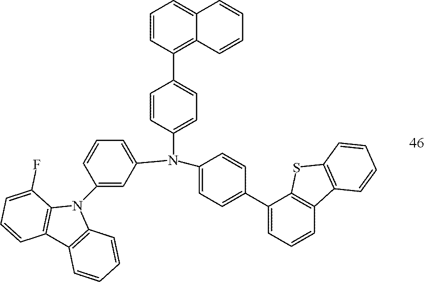 [SMILES: Fc1cccc2c3ccccc3n(-c3cccc(N(c4ccc(-c5cccc6ccccc56)cc4)c4ccc(-c5cccc6c5sc5ccccc56)cc4)c3)c12]

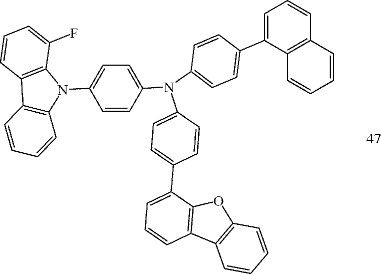 [SMILES: Fc1cccc2c3ccccc3n(-c3ccc(N(c4ccc(-c5cccc6ccccc56)cc4)c4ccc(-c5cccc6c5oc5ccccc56)cc4)cc3)c12]

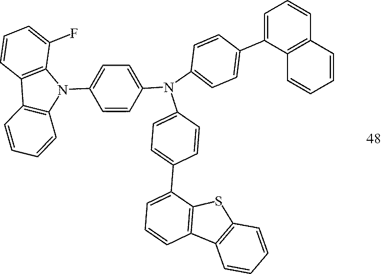 [SMILES: Fc1cccc2c3ccccc3n(-c3ccc(N(c4ccc(-c5cccc6ccccc56)cc4)c4ccc(-c5cccc6c5sc5ccccc56)cc4)cc3)c12]

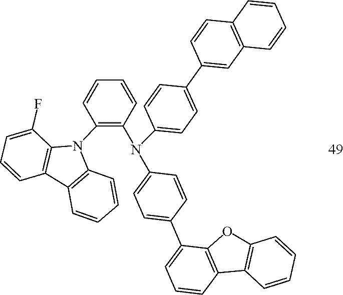 [SMILES: Fc1cccc2c3ccccc3n(-c3ccccc3N(c3ccc(-c4ccc5ccccc5c4)cc3)c3ccc(-c4cccc5c4oc4ccccc45)cc3)c12]

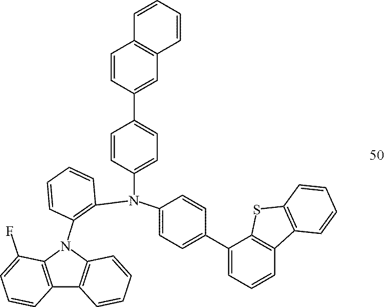 [SMILES: Fc1cccc2c3ccccc3n(-c3ccccc3N(c3ccc(-c4ccc5ccccc5c4)cc3)c3ccc(-c4cccc5c4sc4ccccc45)cc3)c12]

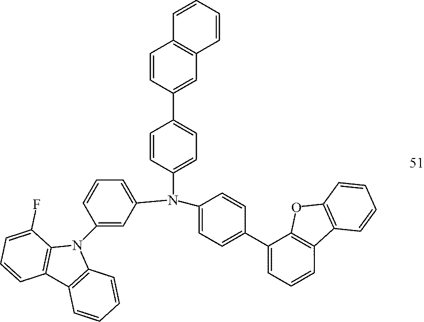 [SMILES: Fc1cccc2c3ccccc3n(-c3cccc(N(c4ccc(-c5ccc6ccccc6c5)cc4)c4ccc(-c5cccc6c5oc5ccccc56)cc4)c3)c12]

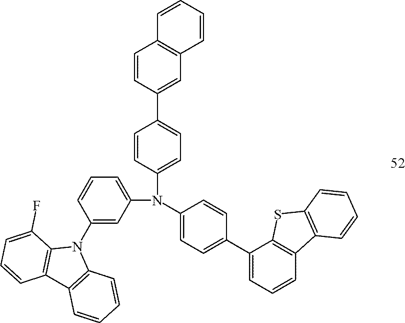 [SMILES: Fc1cccc2c3ccccc3n(-c3cccc(N(c4ccc(-c5ccc6ccccc6c5)cc4)c4ccc(-c5cccc6c5sc5ccccc56)cc4)c3)c12]

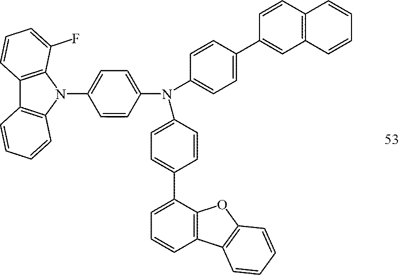 [SMILES: Fc1cccc2c3ccccc3n(-c3ccc(N(c4ccc(-c5ccc6ccccc6c5)cc4)c4ccc(-c5cccc6c5oc5ccccc56)cc4)cc3)c12]

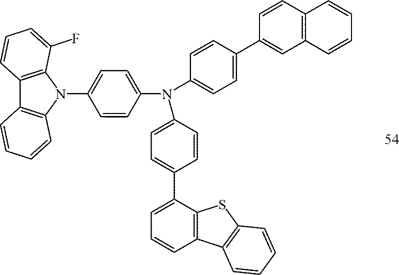 [SMILES: Fc1cccc2c3ccccc3n(-c3ccc(N(c4ccc(-c5ccc6ccccc6c5)cc4)c4ccc(-c5cccc6c5sc5ccccc56)cc4)cc3)c12]

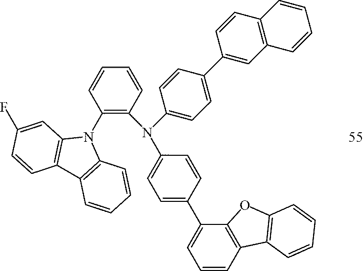 [SMILES: Fc1ccc2c3ccccc3n(-c3ccccc3N(c3ccc(-c4ccc5ccccc5c4)cc3)c3ccc(-c4cccc5c4oc4ccccc45)cc3)c2c1]

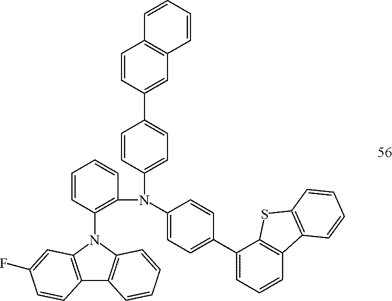 [SMILES: Fc1ccc2c3ccccc3n(-c3ccccc3N(c3ccc(-c4ccc5ccccc5c4)cc3)c3ccc(-c4cccc5c4sc4ccccc45)cc3)c2c1]

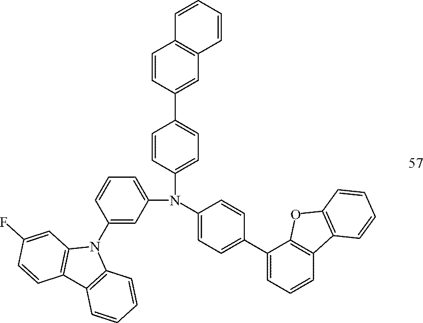 [SMILES: Fc1ccc2c3ccccc3n(-c3cccc(N(c4ccc(-c5ccc6ccccc6c5)cc4)c4ccc(-c5cccc6c5oc5ccccc56)cc4)c3)c2c1]

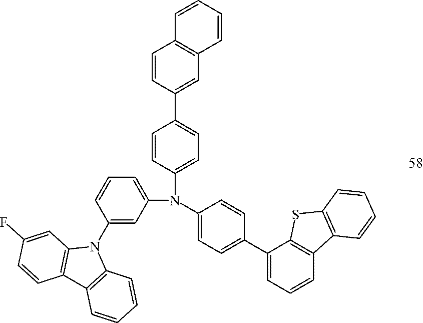 [SMILES: Fc1ccc2c3ccccc3n(-c3cccc(N(c4ccc(-c5ccc6ccccc6c5)cc4)c4ccc(-c5cccc6c5sc5ccccc56)cc4)c3)c2c1]

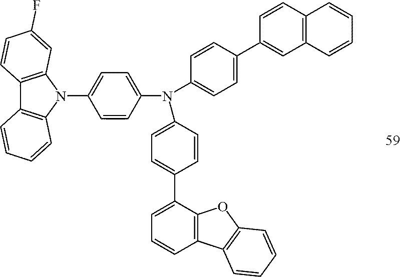 [SMILES: Fc1ccc2c3ccccc3n(-c3ccc(N(c4ccc(-c5ccc6ccccc6c5)cc4)c4ccc(-c5cccc6c5oc5ccccc56)cc4)cc3)c2c1]

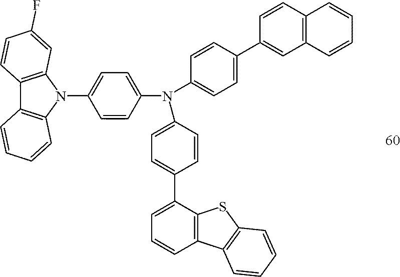 [SMILES: Fc1ccc2c3ccccc3n(-c3ccc(N(c4ccc(-c5ccc6ccccc6c5)cc4)c4ccc(-c5cccc6c5sc5ccccc56)cc4)cc3)c2c1]

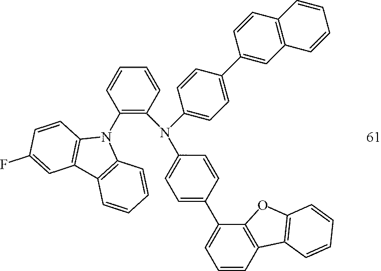 [SMILES: Fc1ccc2c(c1)c1ccccc1n2-c1ccccc1N(c1ccc(-c2ccc3ccccc3c2)cc1)c1ccc(-c2cccc3c2oc2ccccc23)cc1]

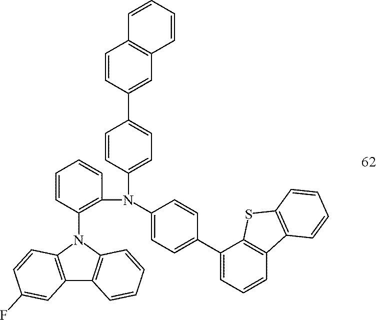 [SMILES: Fc1ccc2c(c1)c1ccccc1n2-c1ccccc1N(c1ccc(-c2ccc3ccccc3c2)cc1)c1ccc(-c2cccc3c2sc2ccccc23)cc1]

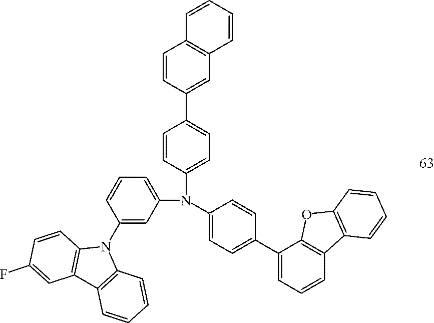 [SMILES: Fc1ccc2c(c1)c1ccccc1n2-c1cccc(N(c2ccc(-c3ccc4ccccc4c3)cc2)c2ccc(-c3cccc4c3oc3ccccc34)cc2)c1]

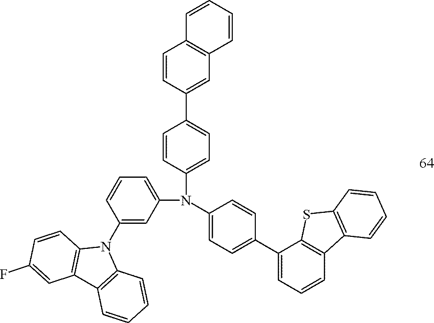 [SMILES: Fc1ccc2c(c1)c1ccccc1n2-c1cccc(N(c2ccc(-c3ccc4ccccc4c3)cc2)c2ccc(-c3cccc4c3sc3ccccc34)cc2)c1]

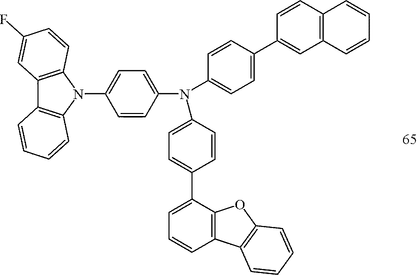 [SMILES: Fc1ccc2c(c1)c1ccccc1n2-c1ccc(N(c2ccc(-c3ccc4ccccc4c3)cc2)c2ccc(-c3cccc4c3oc3ccccc34)cc2)cc1]

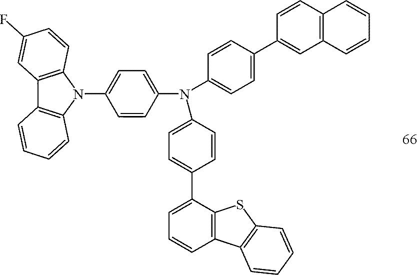 [SMILES: Fc1ccc2c(c1)c1ccccc1n2-c1ccc(N(c2ccc(-c3ccc4ccccc4c3)cc2)c2ccc(-c3cccc4c3sc3ccccc34)cc2)cc1]

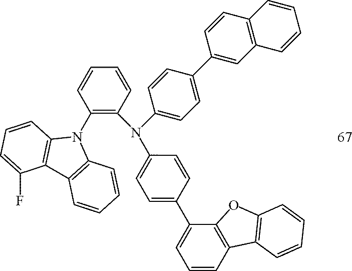 [SMILES: Fc1cccc2c1c1ccccc1n2-c1ccccc1N(c1ccc(-c2ccc3ccccc3c2)cc1)c1ccc(-c2cccc3c2oc2ccccc23)cc1]

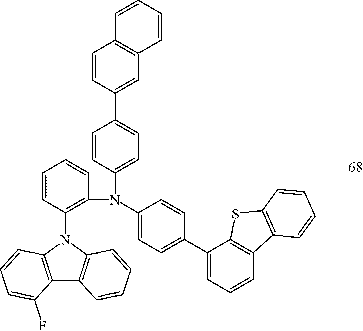 [SMILES: Fc1cccc2c1c1ccccc1n2-c1ccccc1N(c1ccc(-c2ccc3ccccc3c2)cc1)c1ccc(-c2cccc3c2sc2ccccc23)cc1]

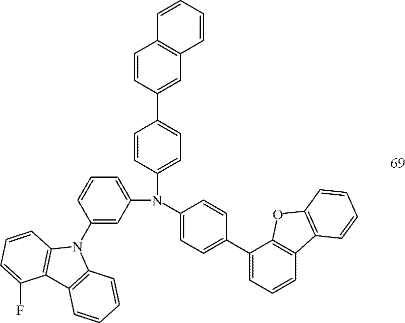 [SMILES: Fc1cccc2c1c1ccccc1n2-c1cccc(N(c2ccc(-c3ccc4ccccc4c3)cc2)c2ccc(-c3cccc4c3oc3ccccc34)cc2)c1]

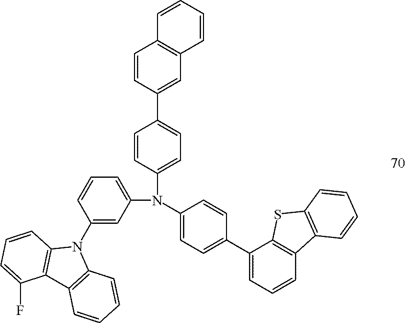 [SMILES: Fc1cccc2c1c1ccccc1n2-c1cccc(N(c2ccc(-c3ccc4ccccc4c3)cc2)c2ccc(-c3cccc4c3sc3ccccc34)cc2)c1]

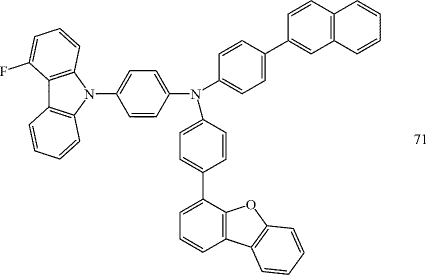 [SMILES: Fc1cccc2c1c1ccccc1n2-c1ccc(N(c2ccc(-c3ccc4ccccc4c3)cc2)c2ccc(-c3cccc4c3oc3ccccc34)cc2)cc1]

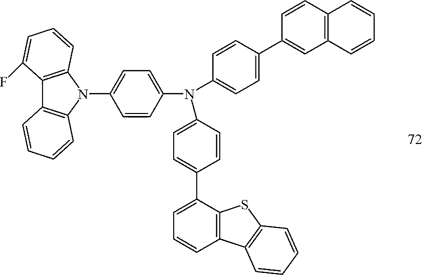 [SMILES: Fc1cccc2c1c1ccccc1n2-c1ccc(N(c2ccc(-c3ccc4ccccc4c3)cc2)c2ccc(-c3cccc4c3sc3ccccc34)cc2)cc1]

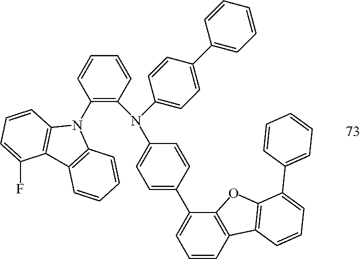 [SMILES: Fc1cccc2c1c1ccccc1n2-c1ccccc1N(c1ccc(-c2ccccc2)cc1)c1ccc(-c2cccc3c2oc2c(-c4ccccc4)cccc23)cc1]

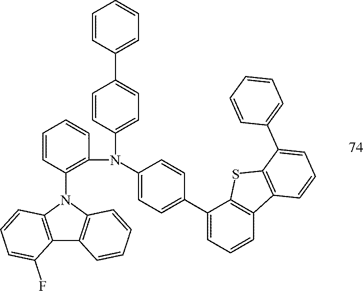 [SMILES: Fc1cccc2c1c1ccccc1n2-c1ccccc1N(c1ccc(-c2ccccc2)cc1)c1ccc(-c2cccc3c2sc2c(-c4ccccc4)cccc23)cc1]

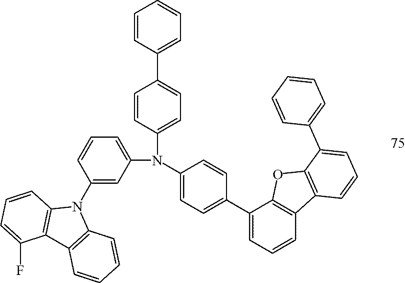 [SMILES: Fc1cccc2c1c1ccccc1n2-c1cccc(N(c2ccc(-c3ccccc3)cc2)c2ccc(-c3cccc4c3oc3c(-c5ccccc5)cccc34)cc2)c1]

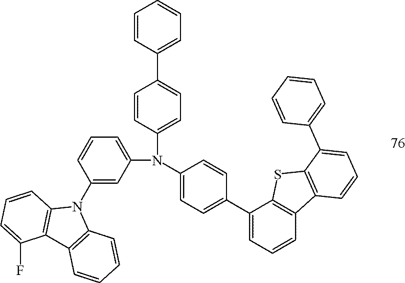 [SMILES: Fc1cccc2c1c1ccccc1n2-c1cccc(N(c2ccc(-c3ccccc3)cc2)c2ccc(-c3cccc4c3sc3c(-c5ccccc5)cccc34)cc2)c1]

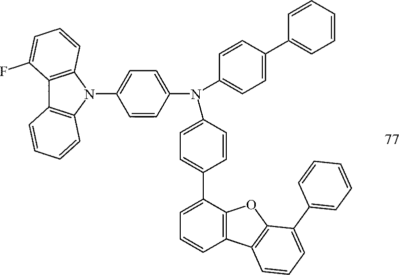 [SMILES: Fc1cccc2c1c1ccccc1n2-c1ccc(N(c2ccc(-c3ccccc3)cc2)c2ccc(-c3cccc4c3oc3c(-c5ccccc5)cccc34)cc2)cc1]

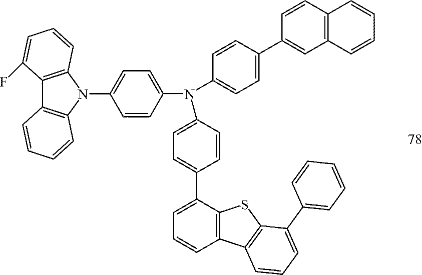 [SMILES: Fc1cccc2c1c1ccccc1n2-c1ccc(N(c2ccc(-c3ccc4ccccc4c3)cc2)c2ccc(-c3cccc4c3sc3c(-c5ccccc5)cccc34)cc2)cc1]

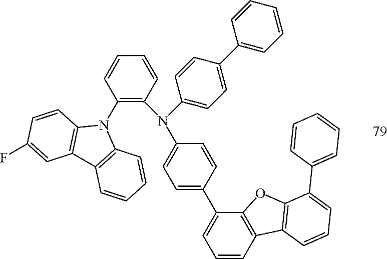 [SMILES: Fc1ccc2c(c1)c1ccccc1n2-c1ccccc1N(c1ccc(-c2ccccc2)cc1)c1ccc(-c2cccc3c2oc2c(-c4ccccc4)cccc23)cc1]

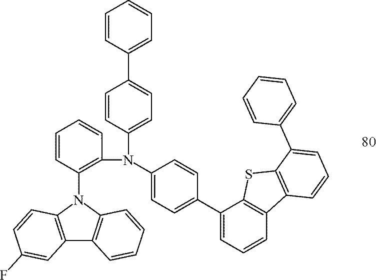 [SMILES: Fc1ccc2c(c1)c1ccccc1n2-c1ccccc1N(c1ccc(-c2ccccc2)cc1)c1ccc(-c2cccc3c2sc2c(-c4ccccc4)cccc23)cc1]

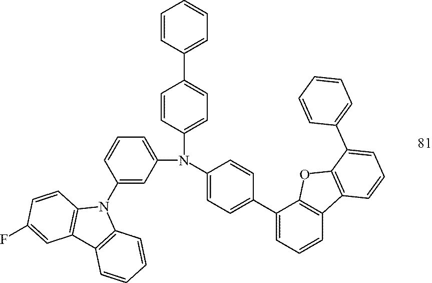 [SMILES: Fc1ccc2c(c1)c1ccccc1n2-c1cccc(N(c2ccc(-c3ccccc3)cc2)c2ccc(-c3cccc4c3oc3c(-c5ccccc5)cccc34)cc2)c1]

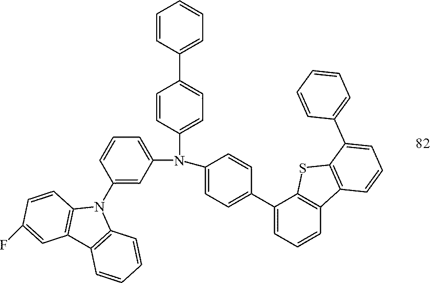 [SMILES: Fc1ccc2c(c1)c1ccccc1n2-c1cccc(N(c2ccc(-c3ccccc3)cc2)c2ccc(-c3cccc4c3sc3c(-c5ccccc5)cccc34)cc2)c1]

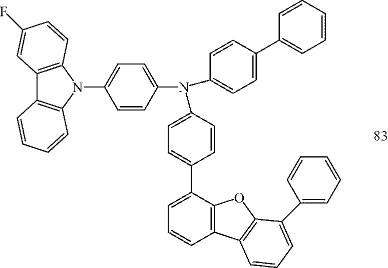 [SMILES: Fc1ccc2c(c1)c1ccccc1n2-c1ccc(N(c2ccc(-c3ccccc3)cc2)c2ccc(-c3cccc4c3oc3c(-c5ccccc5)cccc34)cc2)cc1]

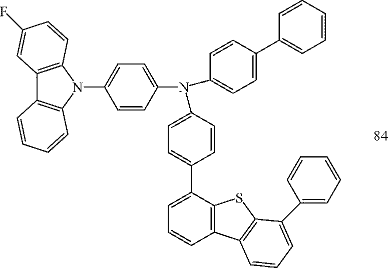 [SMILES: Fc1ccc2c(c1)c1ccccc1n2-c1ccc(N(c2ccc(-c3ccccc3)cc2)c2ccc(-c3cccc4c3sc3c(-c5ccccc5)cccc34)cc2)cc1]

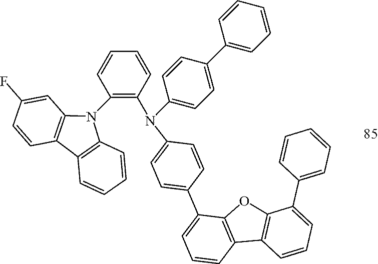 [SMILES: Fc1ccc2c3ccccc3n(-c3ccccc3N(c3ccc(-c4ccccc4)cc3)c3ccc(-c4cccc5c4oc4c(-c6ccccc6)cccc45)cc3)c2c1]

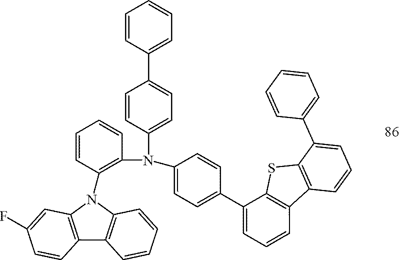 [SMILES: Fc1ccc2c3ccccc3n(-c3ccccc3N(c3ccc(-c4ccccc4)cc3)c3ccc(-c4cccc5c4sc4c(-c6ccccc6)cccc45)cc3)c2c1]

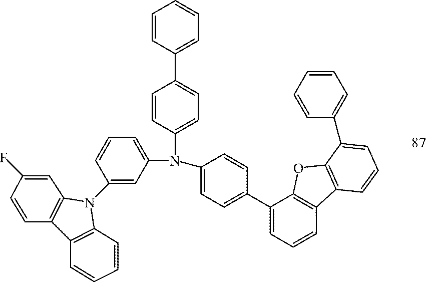 [SMILES: Fc1ccc2c3ccccc3n(-c3cccc(N(c4ccc(-c5ccccc5)cc4)c4ccc(-c5cccc6c5oc5c(-c7ccccc7)cccc56)cc4)c3)c2c1]

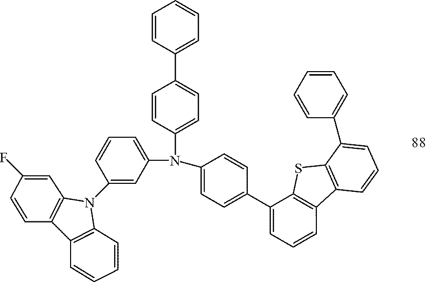 [SMILES: Fc1ccc2c3ccccc3n(-c3cccc(N(c4ccc(-c5ccccc5)cc4)c4ccc(-c5cccc6c5sc5c(-c7ccccc7)cccc56)cc4)c3)c2c1]

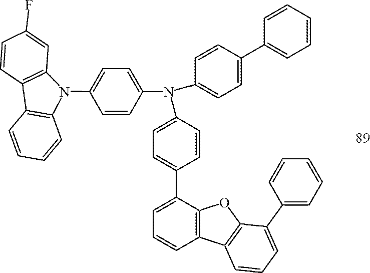 [SMILES: Fc1ccc2c3ccccc3n(-c3ccc(N(c4ccc(-c5ccccc5)cc4)c4ccc(-c5cccc6c5oc5c(-c7ccccc7)cccc56)cc4)cc3)c2c1]

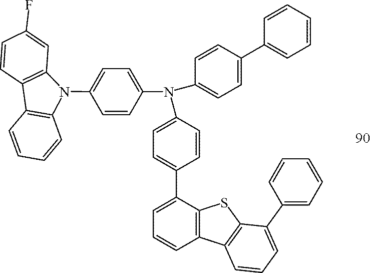 [SMILES: Fc1ccc2c3ccccc3n(-c3ccc(N(c4ccc(-c5ccccc5)cc4)c4ccc(-c5cccc6c5sc5c(-c7ccccc7)cccc56)cc4)cc3)c2c1]

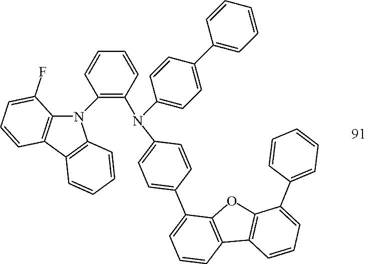 [SMILES: Fc1cccc2c3ccccc3n(-c3ccccc3N(c3ccc(-c4ccccc4)cc3)c3ccc(-c4cccc5c4oc4c(-c6ccccc6)cccc45)cc3)c12]

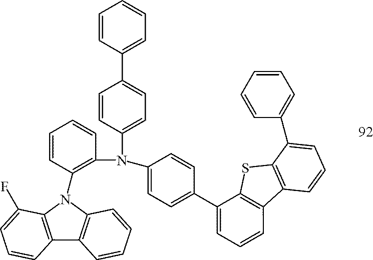 [SMILES: Fc1cccc2c3ccccc3n(-c3ccccc3N(c3ccc(-c4ccccc4)cc3)c3ccc(-c4cccc5c4sc4c(-c6ccccc6)cccc45)cc3)c12]

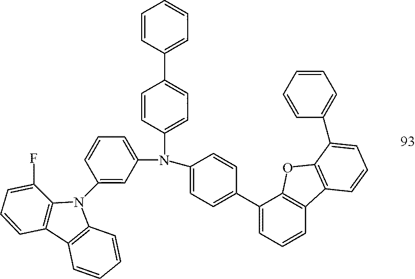 [SMILES: Fc1cccc2c3ccccc3n(-c3cccc(N(c4ccc(-c5ccccc5)cc4)c4ccc(-c5cccc6c5oc5c(-c7ccccc7)cccc56)cc4)c3)c12]

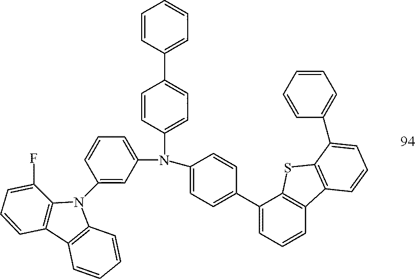 [SMILES: Fc1cccc2c3ccccc3n(-c3cccc(N(c4ccc(-c5ccccc5)cc4)c4ccc(-c5cccc6c5sc5c(-c7ccccc7)cccc56)cc4)c3)c12]

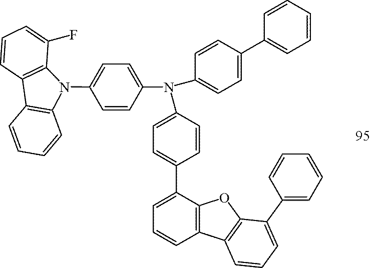 [SMILES: Fc1cccc2c3ccccc3n(-c3ccc(N(c4ccc(-c5ccccc5)cc4)c4ccc(-c5cccc6c5oc5c(-c7ccccc7)cccc56)cc4)cc3)c12]

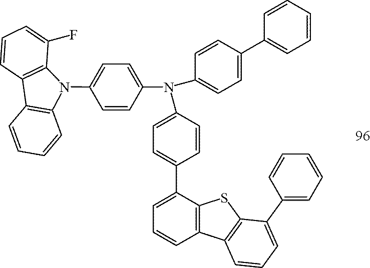 [SMILES: Fc1cccc2c3ccccc3n(-c3ccc(N(c4ccc(-c5ccccc5)cc4)c4ccc(-c5cccc6c5sc5c(-c7ccccc7)cccc56)cc4)cc3)c12]

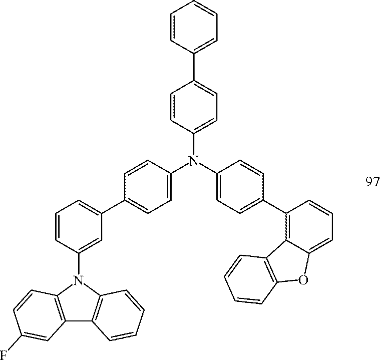 [SMILES: Fc1ccc2c(c1)c1ccccc1n2-c1cccc(-c2ccc(N(c3ccc(-c4ccccc4)cc3)c3ccc(-c4cccc5oc6ccccc6c45)cc3)cc2)c1]

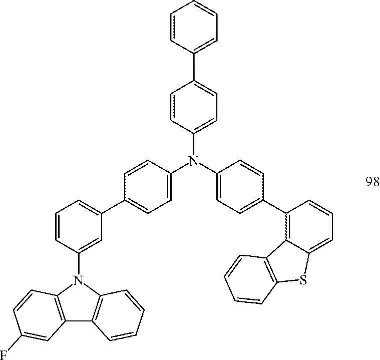 [SMILES: Fc1ccc2c(c1)c1ccccc1n2-c1cccc(-c2ccc(N(c3ccc(-c4ccccc4)cc3)c3ccc(-c4cccc5sc6ccccc6c45)cc3)cc2)c1]

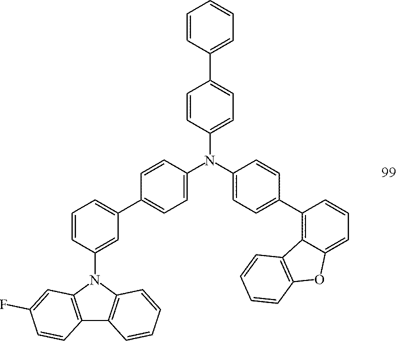 [SMILES: Fc1ccc2c3ccccc3n(-c3cccc(-c4ccc(N(c5ccc(-c6ccccc6)cc5)c5ccc(-c6cccc7oc8ccccc8c67)cc5)cc4)c3)c2c1]

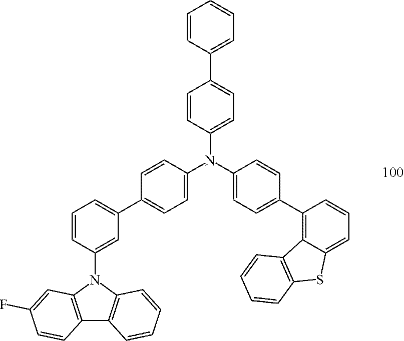 [SMILES: Fc1ccc2c3ccccc3n(-c3cccc(-c4ccc(N(c5ccc(-c6ccccc6)cc5)c5ccc(-c6cccc7sc8ccccc8c67)cc5)cc4)c3)c2c1]

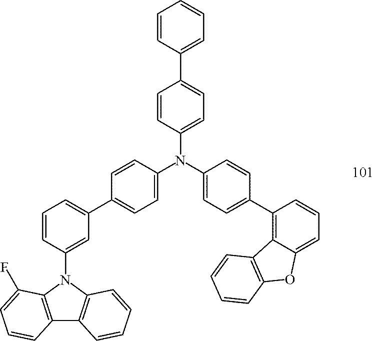 [SMILES: Fc1cccc2c3ccccc3n(-c3cccc(-c4ccc(N(c5ccc(-c6ccccc6)cc5)c5ccc(-c6cccc7oc8ccccc8c67)cc5)cc4)c3)c12]

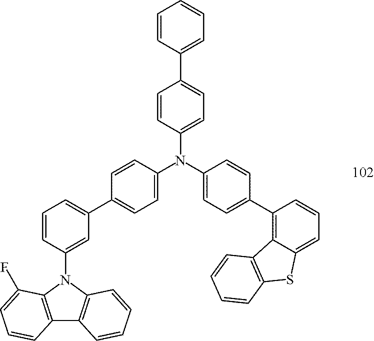 [SMILES: Fc1cccc2c3ccccc3n(-c3cccc(-c4ccc(N(c5ccc(-c6ccccc6)cc5)c5ccc(-c6cccc7sc8ccccc8c67)cc5)cc4)c3)c12]

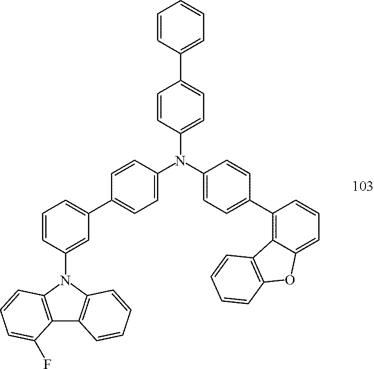 [SMILES: Fc1cccc2c1c1ccccc1n2-c1cccc(-c2ccc(N(c3ccc(-c4ccccc4)cc3)c3ccc(-c4cccc5oc6ccccc6c45)cc3)cc2)c1]

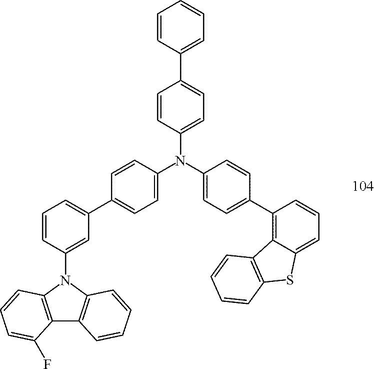 [SMILES: Fc1cccc2c1c1ccccc1n2-c1cccc(-c2ccc(N(c3ccc(-c4ccccc4)cc3)c3ccc(-c4cccc5sc6ccccc6c45)cc3)cc2)c1]

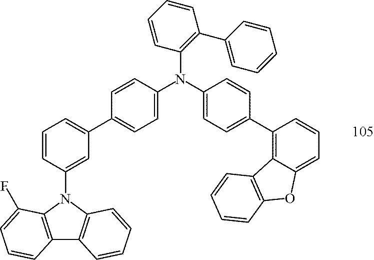 [SMILES: Fc1cccc2c3ccccc3n(-c3cccc(-c4ccc(N(c5ccc(-c6cccc7oc8ccccc8c67)cc5)c5ccccc5-c5ccccc5)cc4)c3)c12]

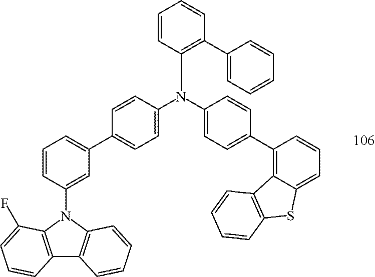 [SMILES: Fc1cccc2c3ccccc3n(-c3cccc(-c4ccc(N(c5ccc(-c6cccc7sc8ccccc8c67)cc5)c5ccccc5-c5ccccc5)cc4)c3)c12]

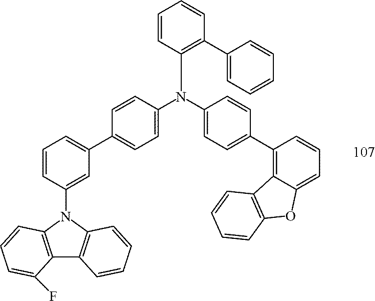 [SMILES: Fc1cccc2c1c1ccccc1n2-c1cccc(-c2ccc(N(c3ccc(-c4cccc5oc6ccccc6c45)cc3)c3ccccc3-c3ccccc3)cc2)c1]

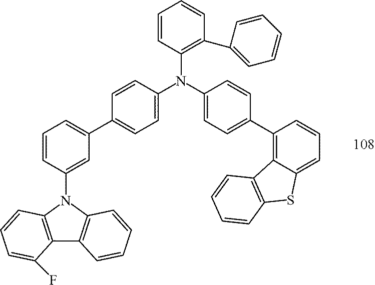 [SMILES: Fc1cccc2c1c1ccccc1n2-c1cccc(-c2ccc(N(c3ccc(-c4cccc5sc6ccccc6c45)cc3)c3ccccc3-c3ccccc3)cc2)c1]

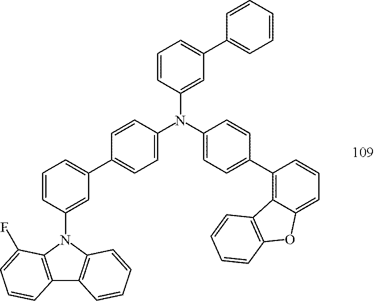 [SMILES: Fc1cccc2c3ccccc3n(-c3cccc(-c4ccc(N(c5ccc(-c6cccc7oc8ccccc8c67)cc5)c5cccc(-c6ccccc6)c5)cc4)c3)c12]

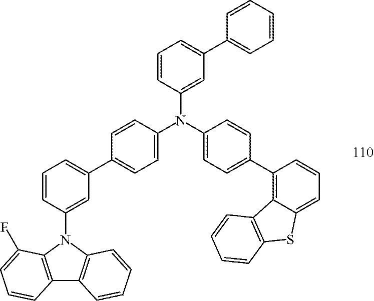 [SMILES: Fc1cccc2c3ccccc3n(-c3cccc(-c4ccc(N(c5ccc(-c6cccc7sc8ccccc8c67)cc5)c5cccc(-c6ccccc6)c5)cc4)c3)c12]

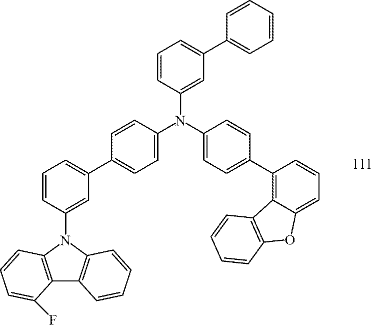 [SMILES: Fc1cccc2c1c1ccccc1n2-c1cccc(-c2ccc(N(c3ccc(-c4cccc5oc6ccccc6c45)cc3)c3cccc(-c4ccccc4)c3)cc2)c1]

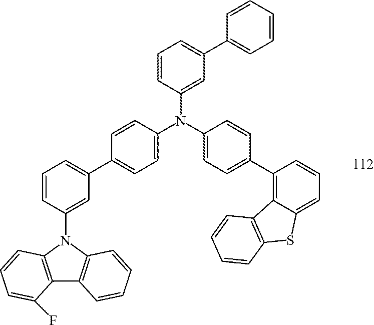 [SMILES: Fc1cccc2c1c1ccccc1n2-c1cccc(-c2ccc(N(c3ccc(-c4cccc5sc6ccccc6c45)cc3)c3cccc(-c4ccccc4)c3)cc2)c1]

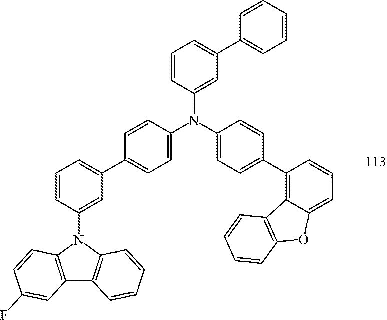 [SMILES: Fc1ccc2c(c1)c1ccccc1n2-c1cccc(-c2ccc(N(c3ccc(-c4cccc5oc6ccccc6c45)cc3)c3cccc(-c4ccccc4)c3)cc2)c1]

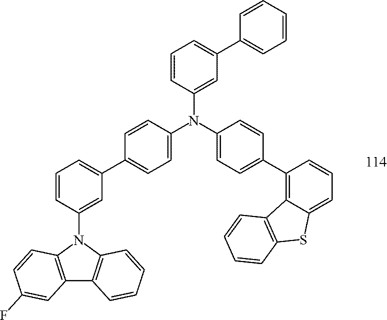 [SMILES: Fc1ccc2c(c1)c1ccccc1n2-c1cccc(-c2ccc(N(c3ccc(-c4cccc5sc6ccccc6c45)cc3)c3cccc(-c4ccccc4)c3)cc2)c1]

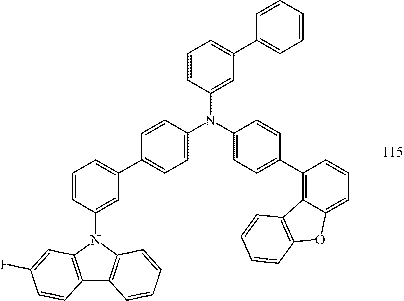 [SMILES: Fc1ccc2c3ccccc3n(-c3cccc(-c4ccc(N(c5ccc(-c6cccc7oc8ccccc8c67)cc5)c5cccc(-c6ccccc6)c5)cc4)c3)c2c1]

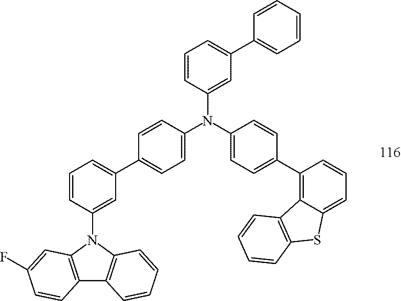 [SMILES: Fc1ccc2c3ccccc3n(-c3cccc(-c4ccc(N(c5ccc(-c6cccc7sc8ccccc8c67)cc5)c5cccc(-c6ccccc6)c5)cc4)c3)c2c1]

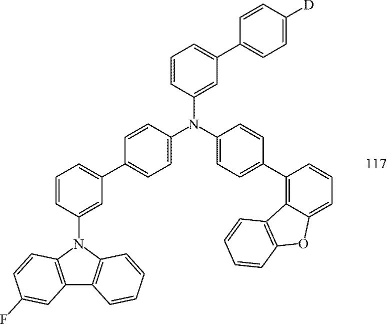 [SMILES: [2H]c1ccc(-c2cccc(N(c3ccc(-c4cccc(-n5c6ccccc6c6cc(F)ccc65)c4)cc3)c3ccc(-c4cccc5oc6ccccc6c45)cc3)c2)cc1]

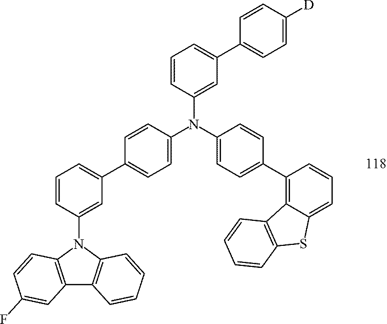 [SMILES: [2H]c1ccc(-c2cccc(N(c3ccc(-c4cccc(-n5c6ccccc6c6cc(F)ccc65)c4)cc3)c3ccc(-c4cccc5sc6ccccc6c45)cc3)c2)cc1]

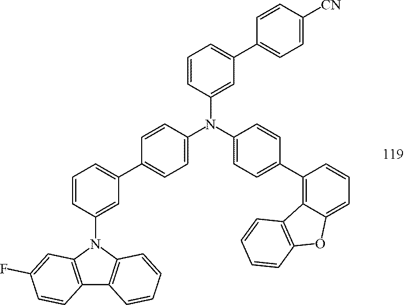 [SMILES: N#Cc1ccc(-c2cccc(N(c3ccc(-c4cccc(-n5c6ccccc6c6ccc(F)cc65)c4)cc3)c3ccc(-c4cccc5oc6ccccc6c45)cc3)c2)cc1]

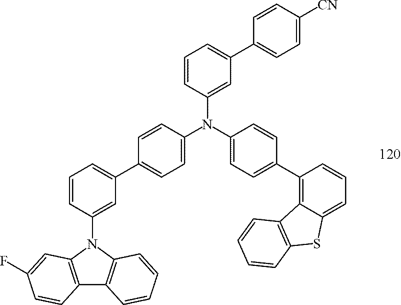 [SMILES: N#Cc1ccc(-c2cccc(N(c3ccc(-c4cccc(-n5c6ccccc6c6ccc(F)cc65)c4)cc3)c3ccc(-c4cccc5sc6ccccc6c45)cc3)c2)cc1]

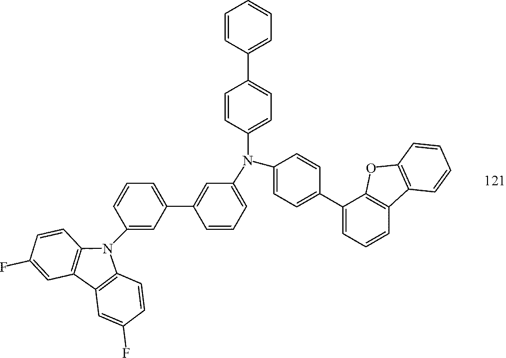 [SMILES: Fc1ccc2c(c1)c1cc(F)ccc1n2-c1cccc(-c2cccc(N(c3ccc(-c4ccccc4)cc3)c3ccc(-c4cccc5c4oc4ccccc45)cc3)c2)c1]

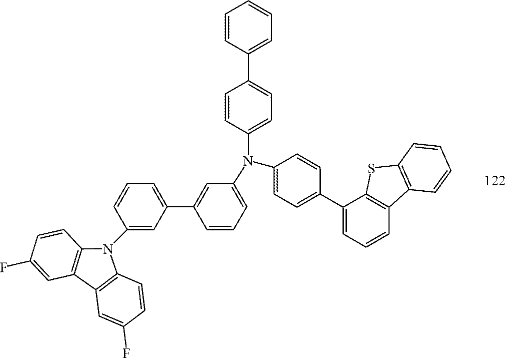 [SMILES: Fc1ccc2c(c1)c1cc(F)ccc1n2-c1cccc(-c2cccc(N(c3ccc(-c4ccccc4)cc3)c3ccc(-c4cccc5c4sc4ccccc45)cc3)c2)c1]

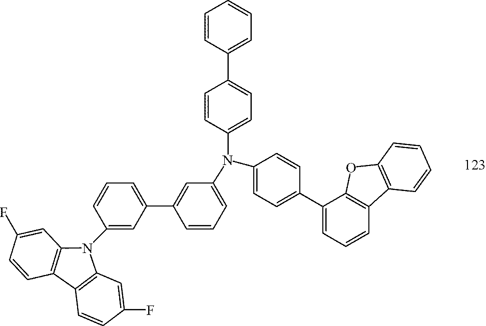 [SMILES: Fc1ccc2c3ccc(F)cc3n(-c3cccc(-c4cccc(N(c5ccc(-c6ccccc6)cc5)c5ccc(-c6cccc7c6oc6ccccc67)cc5)c4)c3)c2c1]

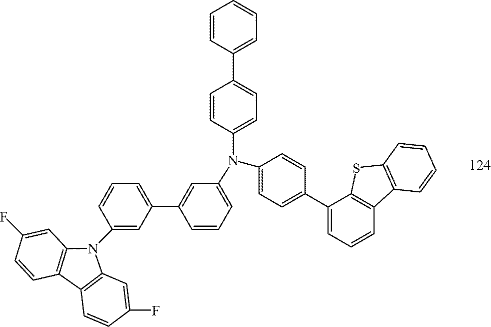 [SMILES: Fc1ccc2c3ccc(F)cc3n(-c3cccc(-c4cccc(N(c5ccc(-c6ccccc6)cc5)c5ccc(-c6cccc7c6sc6ccccc67)cc5)c4)c3)c2c1]

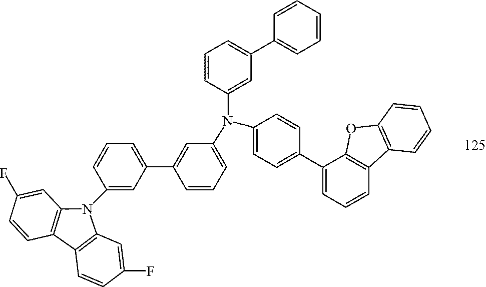 [SMILES: Fc1ccc2c3ccc(F)cc3n(-c3cccc(-c4cccc(N(c5ccc(-c6cccc7c6oc6ccccc67)cc5)c5cccc(-c6ccccc6)c5)c4)c3)c2c1]

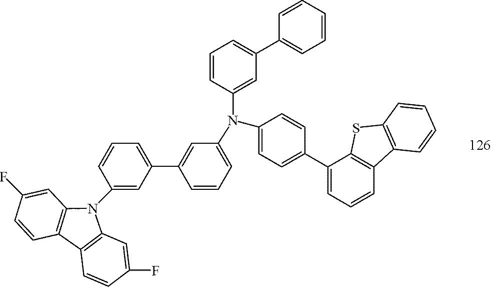 [SMILES: Fc1ccc2c3ccc(F)cc3n(-c3cccc(-c4cccc(N(c5ccc(-c6cccc7c6sc6ccccc67)cc5)c5cccc(-c6ccccc6)c5)c4)c3)c2c1]

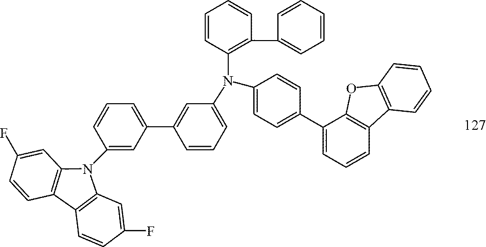 [SMILES: Fc1ccc2c3ccc(F)cc3n(-c3cccc(-c4cccc(N(c5ccc(-c6cccc7c6oc6ccccc67)cc5)c5ccccc5-c5ccccc5)c4)c3)c2c1]

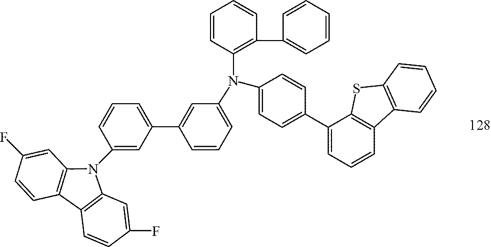 [SMILES: Fc1ccc2c3ccc(F)cc3n(-c3cccc(-c4cccc(N(c5ccc(-c6cccc7c6sc6ccccc67)cc5)c5ccccc5-c5ccccc5)c4)c3)c2c1]

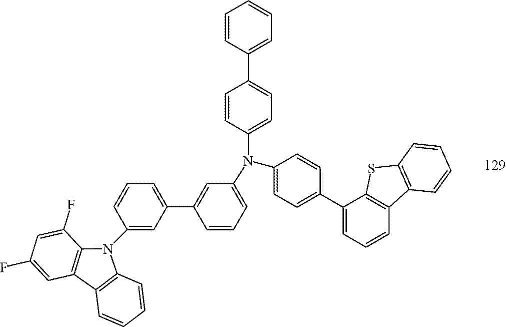 [SMILES: Fc1cc(F)c2c(c1)c1ccccc1n2-c1cccc(-c2cccc(N(c3ccc(-c4ccccc4)cc3)c3ccc(-c4cccc5c4sc4ccccc45)cc3)c2)c1]

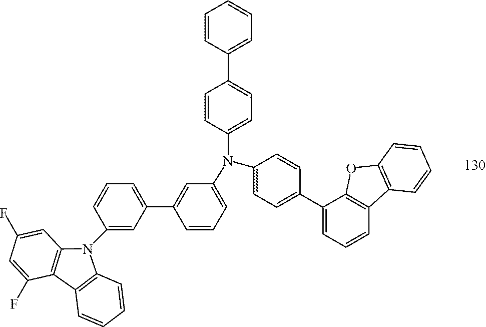 [SMILES: Fc1cc(F)c2c3ccccc3n(-c3cccc(-c4cccc(N(c5ccc(-c6ccccc6)cc5)c5ccc(-c6cccc7c6oc6ccccc67)cc5)c4)c3)c2c1]

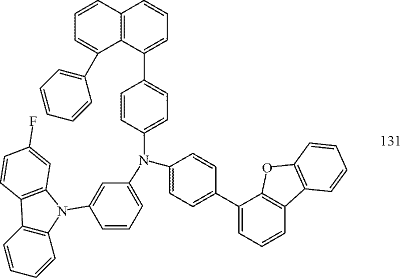 [SMILES: Fc1ccc2c3ccccc3n(-c3cccc(N(c4ccc(-c5cccc6c5oc5ccccc56)cc4)c4ccc(-c5cccc6cccc(-c7ccccc7)c56)cc4)c3)c2c1]

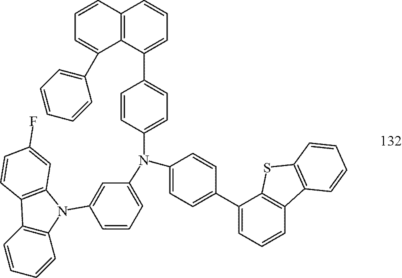 [SMILES: Fc1ccc2c3ccccc3n(-c3cccc(N(c4ccc(-c5cccc6c5sc5ccccc56)cc4)c4ccc(-c5cccc6cccc(-c7ccccc7)c56)cc4)c3)c2c1]

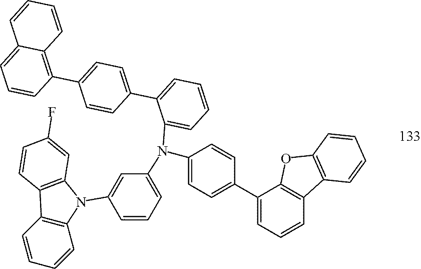 [SMILES: Fc1ccc2c3ccccc3n(-c3cccc(N(c4ccc(-c5cccc6c5oc5ccccc56)cc4)c4ccccc4-c4ccc(-c5cccc6ccccc56)cc4)c3)c2c1]

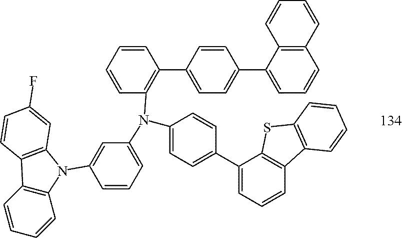 [SMILES: Fc1ccc2c3ccccc3n(-c3cccc(N(c4ccc(-c5cccc6c5sc5ccccc56)cc4)c4ccccc4-c4ccc(-c5cccc6ccccc56)cc4)c3)c2c1]

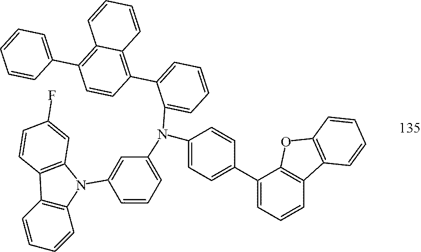 [SMILES: Fc1ccc2c3ccccc3n(-c3cccc(N(c4ccc(-c5cccc6c5oc5ccccc56)cc4)c4ccccc4-c4ccc(-c5ccccc5)c5ccccc45)c3)c2c1]

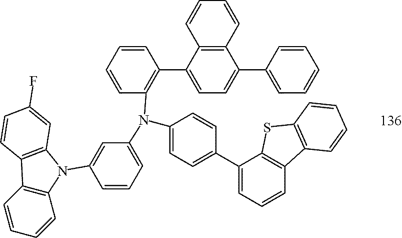 [SMILES: Fc1ccc2c3ccccc3n(-c3cccc(N(c4ccc(-c5cccc6c5sc5ccccc56)cc4)c4ccccc4-c4ccc(-c5ccccc5)c5ccccc45)c3)c2c1]

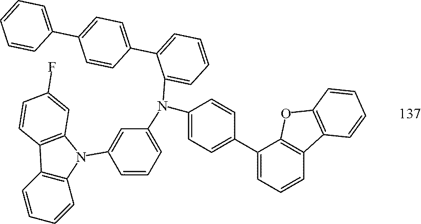 [SMILES: Fc1ccc2c3ccccc3n(-c3cccc(N(c4ccc(-c5cccc6c5oc5ccccc56)cc4)c4ccccc4-c4ccc(-c5ccccc5)cc4)c3)c2c1]

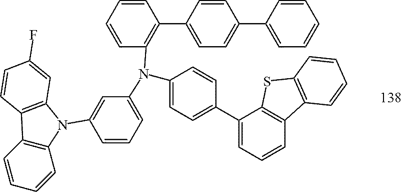 [SMILES: Fc1ccc2c3ccccc3n(-c3cccc(N(c4ccc(-c5cccc6c5sc5ccccc56)cc4)c4ccccc4-c4ccc(-c5ccccc5)cc4)c3)c2c1]

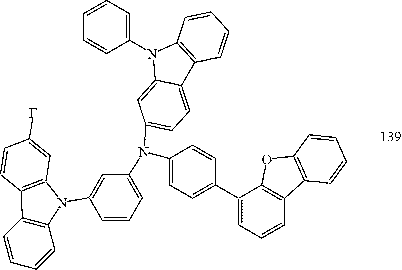 [SMILES: Fc1ccc2c3ccccc3n(-c3cccc(N(c4ccc(-c5cccc6c5oc5ccccc56)cc4)c4ccc5c6ccccc6n(-c6ccccc6)c5c4)c3)c2c1]

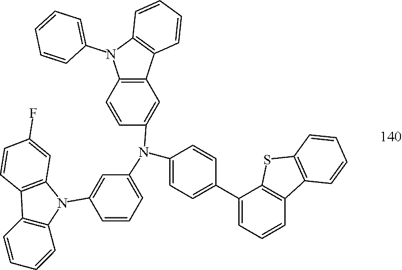 [SMILES: Fc1ccc2c3ccccc3n(-c3cccc(N(c4ccc(-c5cccc6c5sc5ccccc56)cc4)c4ccc5c(c4)c4ccccc4n5-c4ccccc4)c3)c2c1]

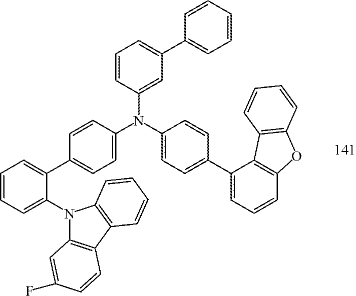 [SMILES: Fc1ccc2c3ccccc3n(-c3ccccc3-c3ccc(N(c4ccc(-c5cccc6oc7ccccc7c56)cc4)c4cccc(-c5ccccc5)c4)cc3)c2c1]

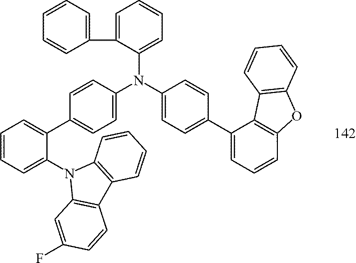 [SMILES: Fc1ccc2c3ccccc3n(-c3ccccc3-c3ccc(N(c4ccc(-c5cccc6oc7ccccc7c56)cc4)c4ccccc4-c4ccccc4)cc3)c2c1]

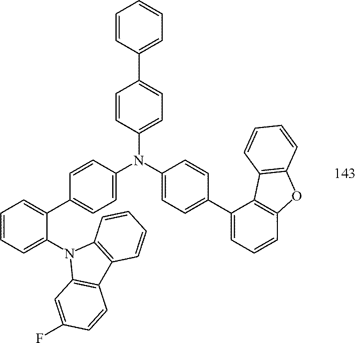 [SMILES: Fc1ccc2c3ccccc3n(-c3ccccc3-c3ccc(N(c4ccc(-c5ccccc5)cc4)c4ccc(-c5cccc6oc7ccccc7c56)cc4)cc3)c2c1]

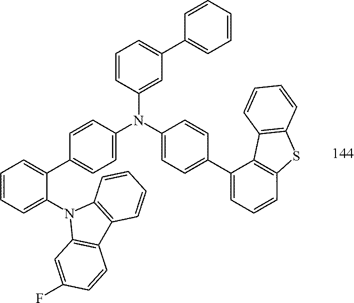 [SMILES: Fc1ccc2c3ccccc3n(-c3ccccc3-c3ccc(N(c4ccc(-c5cccc6sc7ccccc7c56)cc4)c4cccc(-c5ccccc5)c4)cc3)c2c1]

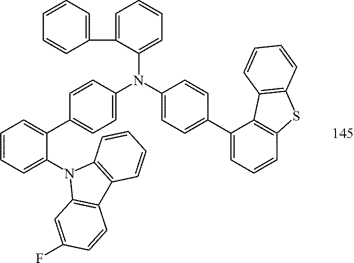 [SMILES: Fc1ccc2c3ccccc3n(-c3ccccc3-c3ccc(N(c4ccc(-c5cccc6sc7ccccc7c56)cc4)c4ccccc4-c4ccccc4)cc3)c2c1]

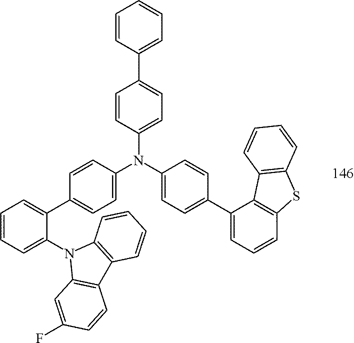 [SMILES: Fc1ccc2c3ccccc3n(-c3ccccc3-c3ccc(N(c4ccc(-c5ccccc5)cc4)c4ccc(-c5cccc6sc7ccccc7c56)cc4)cc3)c2c1]

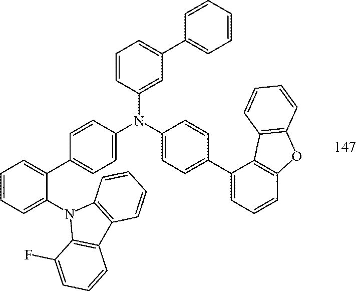 [SMILES: Fc1cccc2c3ccccc3n(-c3ccccc3-c3ccc(N(c4ccc(-c5cccc6oc7ccccc7c56)cc4)c4cccc(-c5ccccc5)c4)cc3)c12]

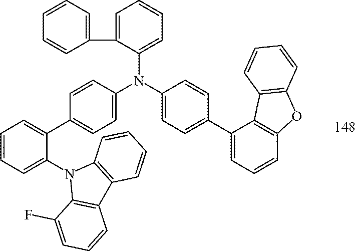 [SMILES: Fc1cccc2c3ccccc3n(-c3ccccc3-c3ccc(N(c4ccc(-c5cccc6oc7ccccc7c56)cc4)c4ccccc4-c4ccccc4)cc3)c12]

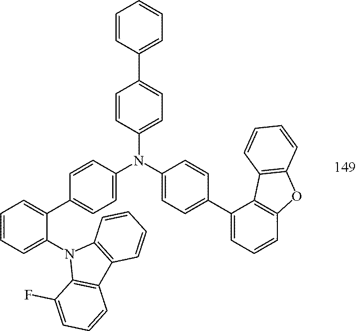 [SMILES: Fc1cccc2c3ccccc3n(-c3ccccc3-c3ccc(N(c4ccc(-c5ccccc5)cc4)c4ccc(-c5cccc6oc7ccccc7c56)cc4)cc3)c12]

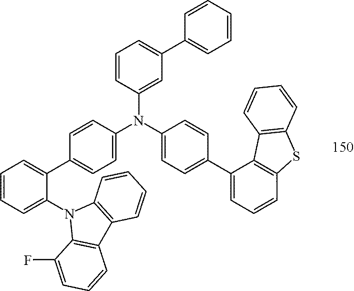 [SMILES: Fc1cccc2c3ccccc3n(-c3ccccc3-c3ccc(N(c4ccc(-c5cccc6sc7ccccc7c56)cc4)c4cccc(-c5ccccc5)c4)cc3)c12]

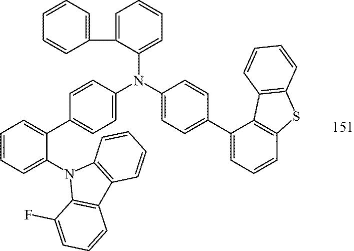 [SMILES: Fc1cccc2c3ccccc3n(-c3ccccc3-c3ccc(N(c4ccc(-c5cccc6sc7ccccc7c56)cc4)c4ccccc4-c4ccccc4)cc3)c12]

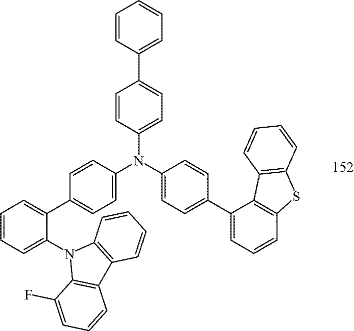 [SMILES: Fc1cccc2c3ccccc3n(-c3ccccc3-c3ccc(N(c4ccc(-c5ccccc5)cc4)c4ccc(-c5cccc6sc7ccccc7c56)cc4)cc3)c12]

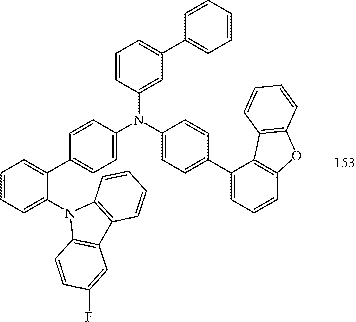 [SMILES: Fc1ccc2c(c1)c1ccccc1n2-c1ccccc1-c1ccc(N(c2ccc(-c3cccc4oc5ccccc5c34)cc2)c2cccc(-c3ccccc3)c2)cc1]

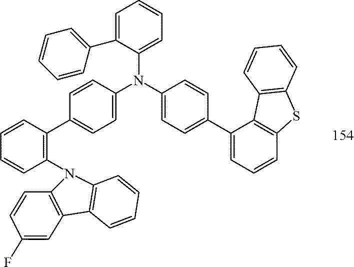 [SMILES: Fc1ccc2c(c1)c1ccccc1n2-c1ccccc1-c1ccc(N(c2ccc(-c3cccc4sc5ccccc5c34)cc2)c2ccccc2-c2ccccc2)cc1]

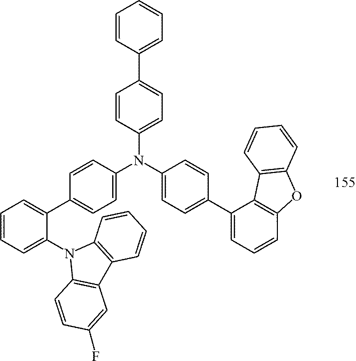 [SMILES: Fc1ccc2c(c1)c1ccccc1n2-c1ccccc1-c1ccc(N(c2ccc(-c3ccccc3)cc2)c2ccc(-c3cccc4oc5ccccc5c34)cc2)cc1]

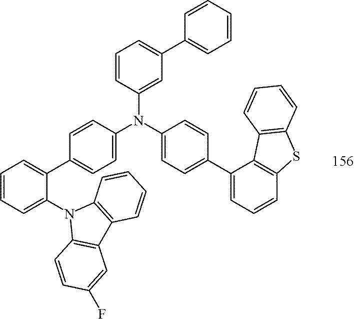 [SMILES: Fc1ccc2c(c1)c1ccccc1n2-c1ccccc1-c1ccc(N(c2ccc(-c3cccc4sc5ccccc5c34)cc2)c2cccc(-c3ccccc3)c2)cc1]

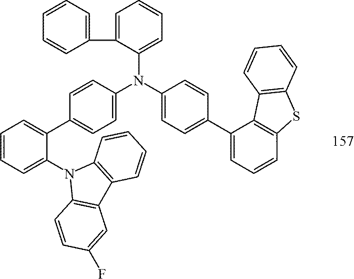 [SMILES: Fc1ccc2c(c1)c1ccccc1n2-c1ccccc1-c1ccc(N(c2ccc(-c3cccc4sc5ccccc5c34)cc2)c2ccccc2-c2ccccc2)cc1]

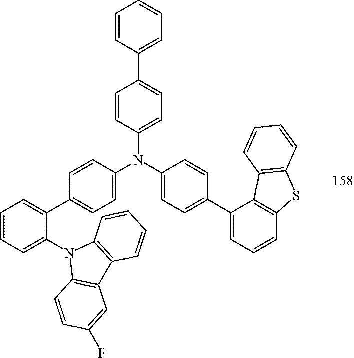 [SMILES: Fc1ccc2c(c1)c1ccccc1n2-c1ccccc1-c1ccc(N(c2ccc(-c3ccccc3)cc2)c2ccc(-c3cccc4sc5ccccc5c34)cc2)cc1]

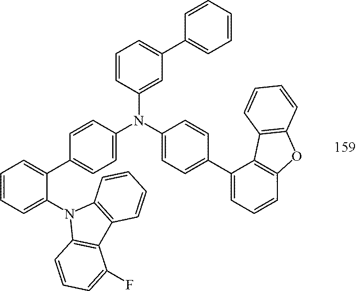 [SMILES: Fc1cccc2c1c1ccccc1n2-c1ccccc1-c1ccc(N(c2ccc(-c3cccc4oc5ccccc5c34)cc2)c2cccc(-c3ccccc3)c2)cc1]

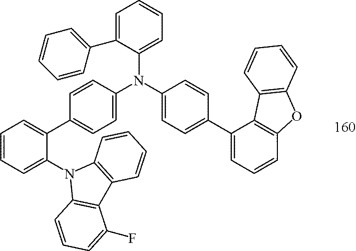 [SMILES: Fc1cccc2c1c1ccccc1n2-c1ccccc1-c1ccc(N(c2ccc(-c3cccc4oc5ccccc5c34)cc2)c2ccccc2-c2ccccc2)cc1]

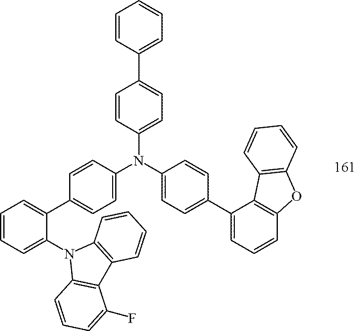 [SMILES: Fc1cccc2c1c1ccccc1n2-c1ccccc1-c1ccc(N(c2ccc(-c3ccccc3)cc2)c2ccc(-c3cccc4oc5ccccc5c34)cc2)cc1]

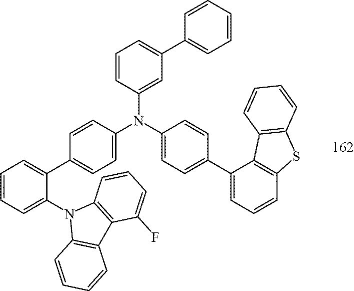 [SMILES: Fc1cccc2c1c1ccccc1n2-c1ccccc1-c1ccc(N(c2ccc(-c3cccc4sc5ccccc5c34)cc2)c2cccc(-c3ccccc3)c2)cc1]

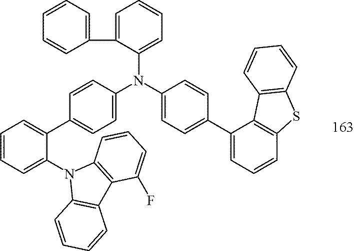 [SMILES: Fc1cccc2c1c1ccccc1n2-c1ccccc1-c1ccc(N(c2ccc(-c3cccc4sc5ccccc5c34)cc2)c2ccccc2-c2ccccc2)cc1]

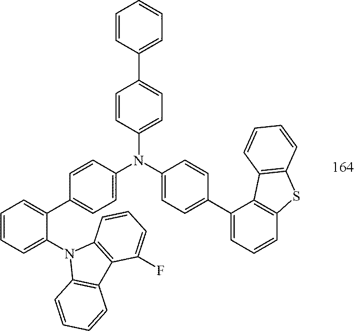 [SMILES: Fc1cccc2c1c1ccccc1n2-c1ccccc1-c1ccc(N(c2ccc(-c3ccccc3)cc2)c2ccc(-c3cccc4sc5ccccc5c34)cc2)cc1]

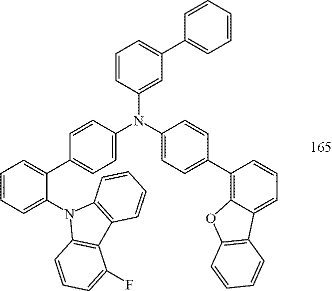 [SMILES: Fc1cccc2c1c1ccccc1n2-c1ccccc1-c1ccc(N(c2ccc(-c3cccc4c3oc3ccccc34)cc2)c2cccc(-c3ccccc3)c2)cc1]

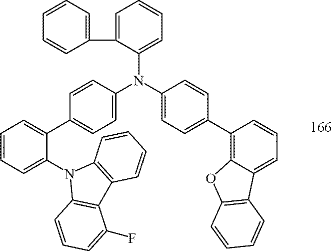 [SMILES: Fc1cccc2c1c1ccccc1n2-c1ccccc1-c1ccc(N(c2ccc(-c3cccc4c3oc3ccccc34)cc2)c2ccccc2-c2ccccc2)cc1]

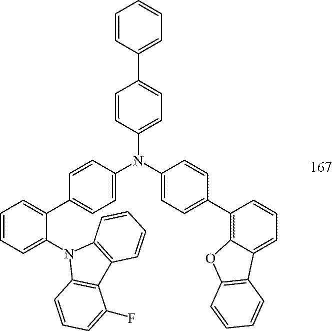 [SMILES: Fc1cccc2c1c1ccccc1n2-c1ccccc1-c1ccc(N(c2ccc(-c3ccccc3)cc2)c2ccc(-c3cccc4c3oc3ccccc34)cc2)cc1]

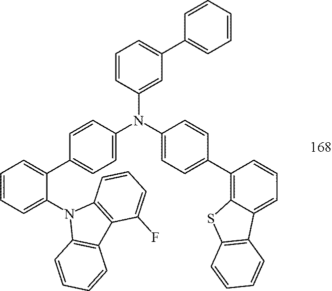 [SMILES: Fc1cccc2c1c1ccccc1n2-c1ccccc1-c1ccc(N(c2ccc(-c3cccc4c3sc3ccccc34)cc2)c2cccc(-c3ccccc3)c2)cc1]

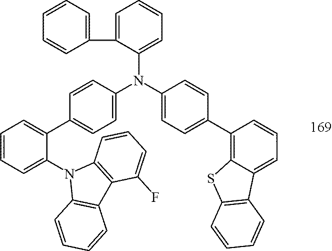 [SMILES: Fc1cccc2c1c1ccccc1n2-c1ccccc1-c1ccc(N(c2ccc(-c3cccc4c3sc3ccccc34)cc2)c2ccccc2-c2ccccc2)cc1]

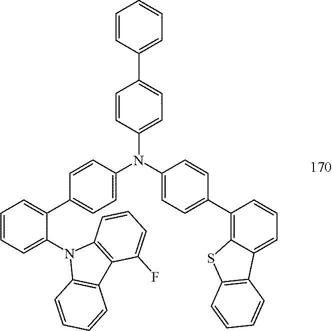 [SMILES: Fc1cccc2c1c1ccccc1n2-c1ccccc1-c1ccc(N(c2ccc(-c3ccccc3)cc2)c2ccc(-c3cccc4c3sc3ccccc34)cc2)cc1]

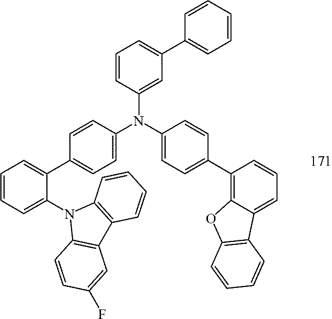 [SMILES: Fc1ccc2c(c1)c1ccccc1n2-c1ccccc1-c1ccc(N(c2ccc(-c3cccc4c3oc3ccccc34)cc2)c2cccc(-c3ccccc3)c2)cc1]

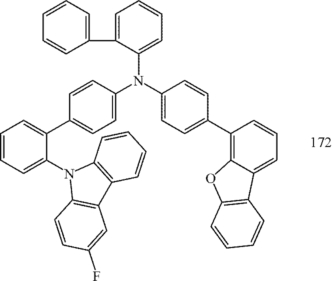 [SMILES: Fc1ccc2c(c1)c1ccccc1n2-c1ccccc1-c1ccc(N(c2ccc(-c3cccc4c3oc3ccccc34)cc2)c2ccccc2-c2ccccc2)cc1]

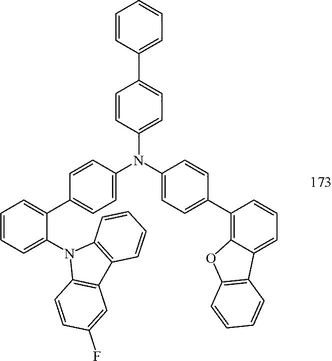 [SMILES: Fc1ccc2c(c1)c1ccccc1n2-c1ccccc1-c1ccc(N(c2ccc(-c3ccccc3)cc2)c2ccc(-c3cccc4c3oc3ccccc34)cc2)cc1]

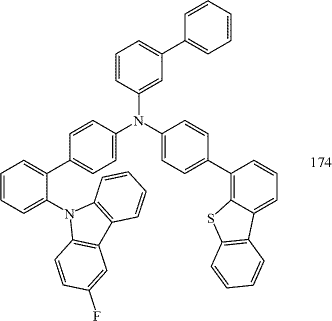 [SMILES: Fc1ccc2c(c1)c1ccccc1n2-c1ccccc1-c1ccc(N(c2ccc(-c3cccc4c3sc3ccccc34)cc2)c2cccc(-c3ccccc3)c2)cc1]

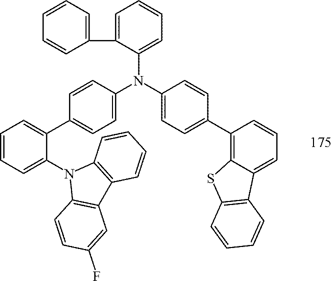 [SMILES: Fc1ccc2c(c1)c1ccccc1n2-c1ccccc1-c1ccc(N(c2ccc(-c3cccc4c3sc3ccccc34)cc2)c2ccccc2-c2ccccc2)cc1]

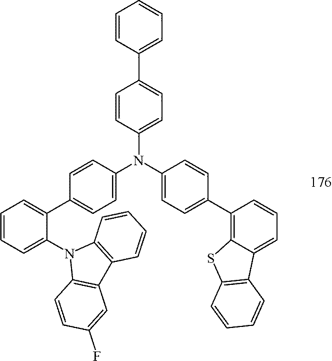 [SMILES: Fc1ccc2c(c1)c1ccccc1n2-c1ccccc1-c1ccc(N(c2ccc(-c3ccccc3)cc2)c2ccc(-c3cccc4c3sc3ccccc34)cc2)cc1]

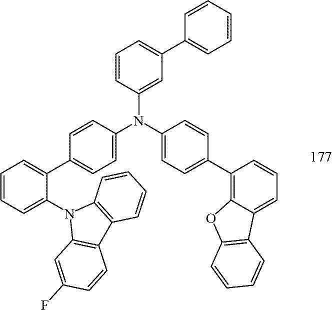 [SMILES: Fc1ccc2c3ccccc3n(-c3ccccc3-c3ccc(N(c4ccc(-c5cccc6c5oc5ccccc56)cc4)c4cccc(-c5ccccc5)c4)cc3)c2c1]

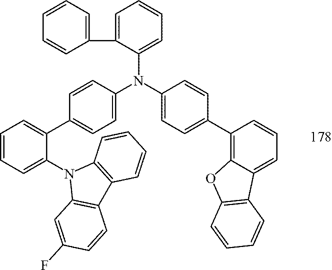 [SMILES: Fc1ccc2c3ccccc3n(-c3ccccc3-c3ccc(N(c4ccc(-c5cccc6c5oc5ccccc56)cc4)c4ccccc4-c4ccccc4)cc3)c2c1]

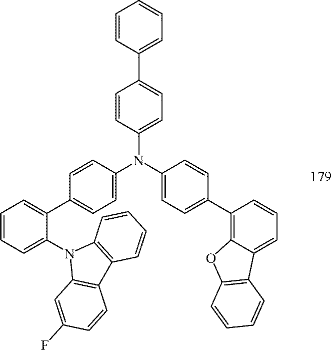 [SMILES: Fc1ccc2c3ccccc3n(-c3ccccc3-c3ccc(N(c4ccc(-c5ccccc5)cc4)c4ccc(-c5cccc6c5oc5ccccc56)cc4)cc3)c2c1]

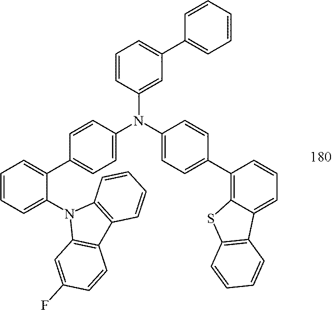 [SMILES: Fc1ccc2c3ccccc3n(-c3ccccc3-c3ccc(N(c4ccc(-c5cccc6c5sc5ccccc56)cc4)c4cccc(-c5ccccc5)c4)cc3)c2c1]

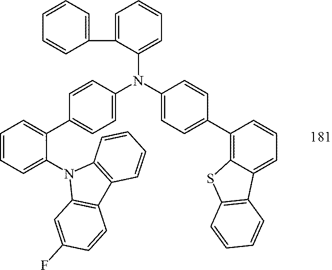 [SMILES: Fc1ccc2c3ccccc3n(-c3ccccc3-c3ccc(N(c4ccc(-c5cccc6c5sc5ccccc56)cc4)c4ccccc4-c4ccccc4)cc3)c2c1]

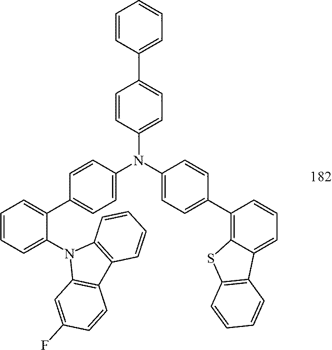 [SMILES: Fc1ccc2c3ccccc3n(-c3ccccc3-c3ccc(N(c4ccc(-c5ccccc5)cc4)c4ccc(-c5cccc6c5sc5ccccc56)cc4)cc3)c2c1]

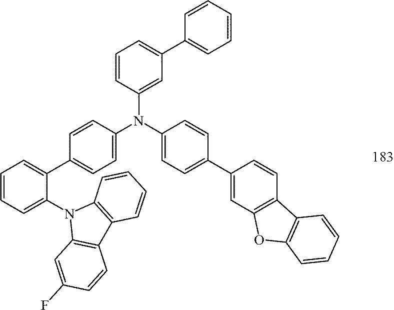 [SMILES: Fc1ccc2c3ccccc3n(-c3ccccc3-c3ccc(N(c4ccc(-c5ccc6c(c5)oc5ccccc56)cc4)c4cccc(-c5ccccc5)c4)cc3)c2c1]

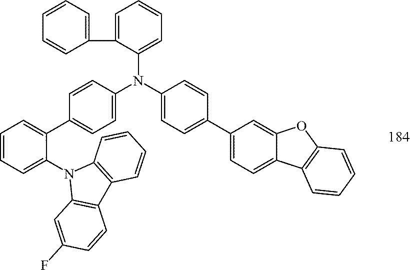 [SMILES: Fc1ccc2c3ccccc3n(-c3ccccc3-c3ccc(N(c4ccc(-c5ccc6c(c5)oc5ccccc56)cc4)c4ccccc4-c4ccccc4)cc3)c2c1]

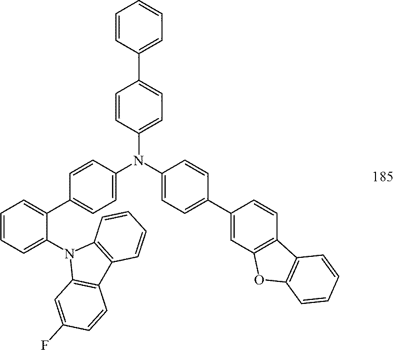 [SMILES: Fc1ccc2c3ccccc3n(-c3ccccc3-c3ccc(N(c4ccc(-c5ccccc5)cc4)c4ccc(-c5ccc6c(c5)oc5ccccc56)cc4)cc3)c2c1]

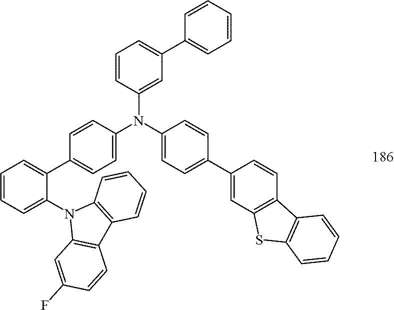 [SMILES: Fc1ccc2c3ccccc3n(-c3ccccc3-c3ccc(N(c4ccc(-c5ccc6c(c5)sc5ccccc56)cc4)c4cccc(-c5ccccc5)c4)cc3)c2c1]

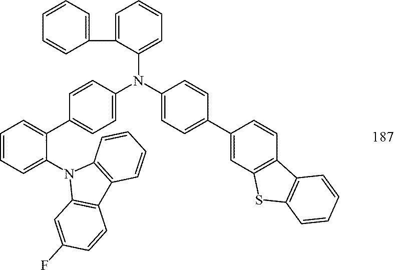 [SMILES: Fc1ccc2c3ccccc3n(-c3ccccc3-c3ccc(N(c4ccc(-c5ccc6c(c5)sc5ccccc56)cc4)c4ccccc4-c4ccccc4)cc3)c2c1]

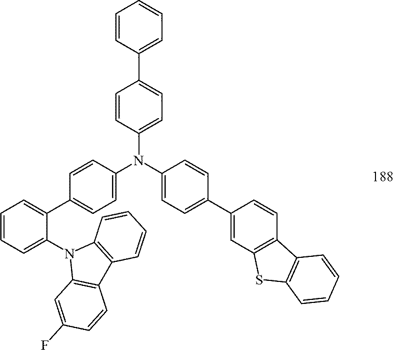 [SMILES: Fc1ccc2c3ccccc3n(-c3ccccc3-c3ccc(N(c4ccc(-c5ccccc5)cc4)c4ccc(-c5ccc6c(c5)sc5ccccc56)cc4)cc3)c2c1]

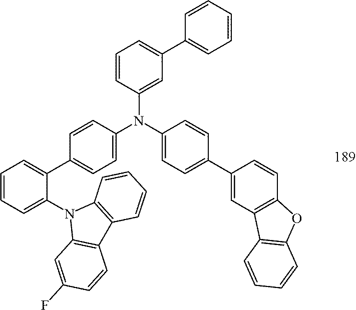 [SMILES: Fc1ccc2c3ccccc3n(-c3ccccc3-c3ccc(N(c4ccc(-c5ccc6oc7ccccc7c6c5)cc4)c4cccc(-c5ccccc5)c4)cc3)c2c1]

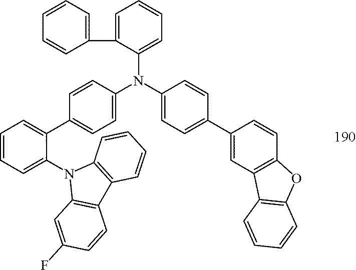 [SMILES: Fc1ccc2c3ccccc3n(-c3ccccc3-c3ccc(N(c4ccc(-c5ccc6oc7ccccc7c6c5)cc4)c4ccccc4-c4ccccc4)cc3)c2c1]

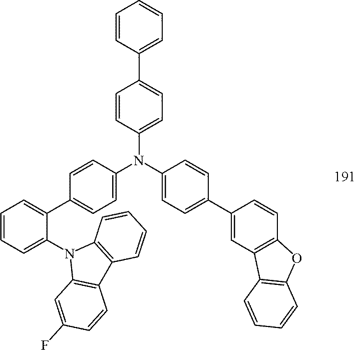 [SMILES: Fc1ccc2c3ccccc3n(-c3ccccc3-c3ccc(N(c4ccc(-c5ccccc5)cc4)c4ccc(-c5ccc6oc7ccccc7c6c5)cc4)cc3)c2c1]

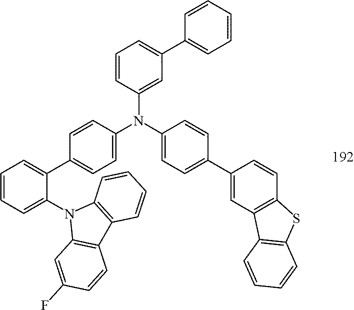 [SMILES: Fc1ccc2c3ccccc3n(-c3ccccc3-c3ccc(N(c4ccc(-c5ccc6sc7ccccc7c6c5)cc4)c4cccc(-c5ccccc5)c4)cc3)c2c1]

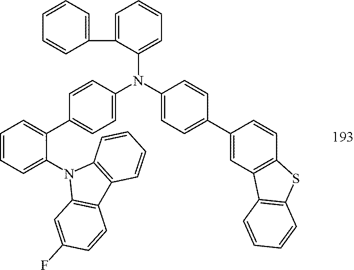 [SMILES: Fc1ccc2c3ccccc3n(-c3ccccc3-c3ccc(N(c4ccc(-c5ccc6sc7ccccc7c6c5)cc4)c4ccccc4-c4ccccc4)cc3)c2c1]

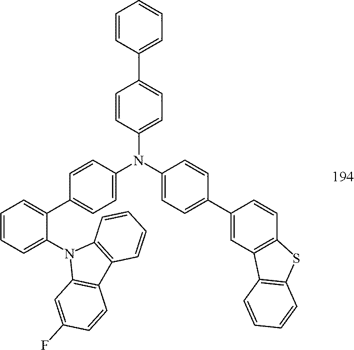 [SMILES: Fc1ccc2c3ccccc3n(-c3ccccc3-c3ccc(N(c4ccc(-c5ccccc5)cc4)c4ccc(-c5ccc6sc7ccccc7c6c5)cc4)cc3)c2c1]

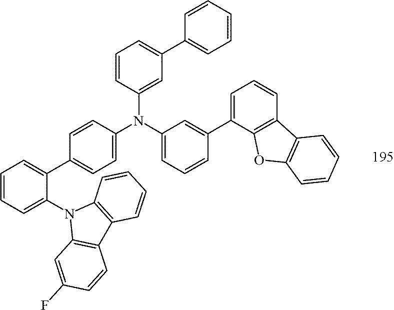 [SMILES: Fc1ccc2c3ccccc3n(-c3ccccc3-c3ccc(N(c4cccc(-c5ccccc5)c4)c4cccc(-c5cccc6c5oc5ccccc56)c4)cc3)c2c1]

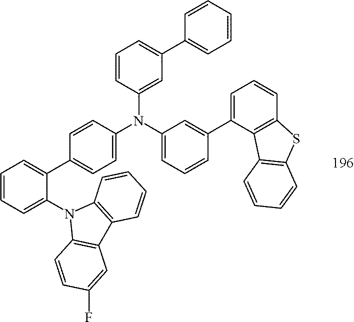 [SMILES: Fc1ccc2c(c1)c1ccccc1n2-c1ccccc1-c1ccc(N(c2cccc(-c3ccccc3)c2)c2cccc(-c3cccc4sc5ccccc5c34)c2)cc1]

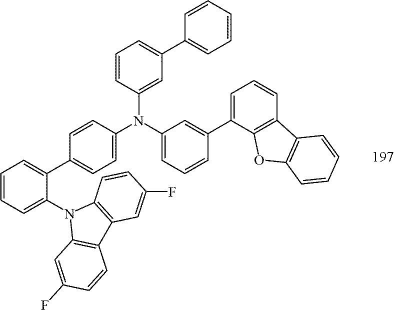 [SMILES: Fc1ccc2c(c1)c1ccc(F)cc1n2-c1ccccc1-c1ccc(N(c2cccc(-c3ccccc3)c2)c2cccc(-c3cccc4c3oc3ccccc34)c2)cc1]

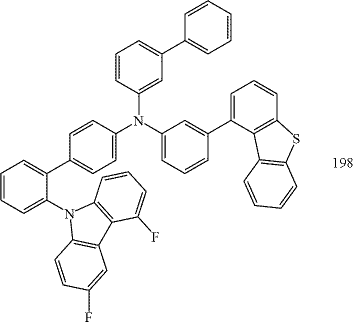 [SMILES: Fc1ccc2c(c1)c1c(F)cccc1n2-c1ccccc1-c1ccc(N(c2cccc(-c3ccccc3)c2)c2cccc(-c3cccc4sc5ccccc5c34)c2)cc1]

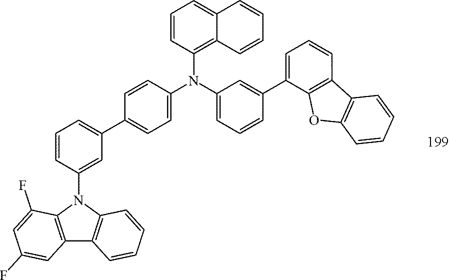 [SMILES: Fc1cc(F)c2c(c1)c1ccccc1n2-c1cccc(-c2ccc(N(c3cccc(-c4cccc5c4oc4ccccc45)c3)c3cccc4ccccc34)cc2)c1]

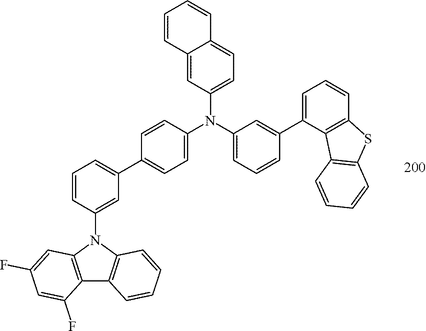 [SMILES: Fc1cc(F)c2c3ccccc3n(-c3cccc(-c4ccc(N(c5cccc(-c6cccc7sc8ccccc8c67)c5)c5ccc6ccccc6c5)cc4)c3)c2c1]

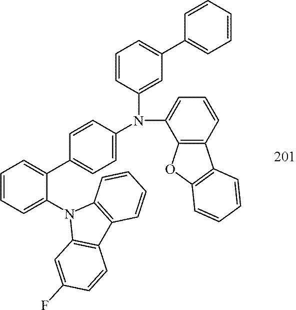 [SMILES: Fc1ccc2c3ccccc3n(-c3ccccc3-c3ccc(N(c4cccc(-c5ccccc5)c4)c4cccc5c4oc4ccccc45)cc3)c2c1]

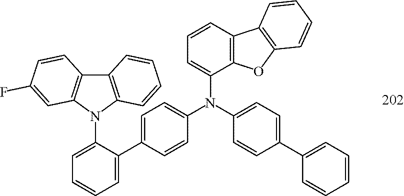 [SMILES: Fc1ccc2c3ccccc3n(-c3ccccc3-c3ccc(N(c4ccc(-c5ccccc5)cc4)c4cccc5c4oc4ccccc45)cc3)c2c1]

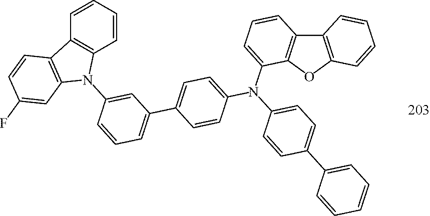 [SMILES: Fc1ccc2c3ccccc3n(-c3cccc(-c4ccc(N(c5ccc(-c6ccccc6)cc5)c5cccc6c5oc5ccccc56)cc4)c3)c2c1]

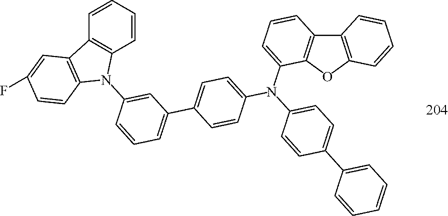 [SMILES: Fc1ccc2c(c1)c1ccccc1n2-c1cccc(-c2ccc(N(c3ccc(-c4ccccc4)cc3)c3cccc4c3oc3ccccc34)cc2)c1]

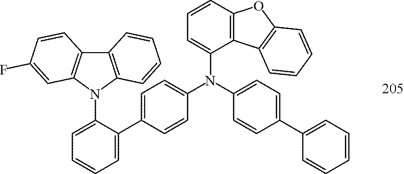 [SMILES: Fc1ccc2c3ccccc3n(-c3ccccc3-c3ccc(N(c4ccc(-c5ccccc5)cc4)c4cccc5oc6ccccc6c45)cc3)c2c1]

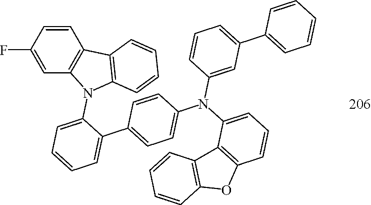 [SMILES: Fc1ccc2c3ccccc3n(-c3ccccc3-c3ccc(N(c4cccc(-c5ccccc5)c4)c4cccc5oc6ccccc6c45)cc3)c2c1]

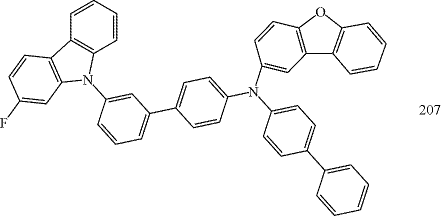 [SMILES: Fc1ccc2c3ccccc3n(-c3cccc(-c4ccc(N(c5ccc(-c6ccccc6)cc5)c5ccc6oc7ccccc7c6c5)cc4)c3)c2c1]

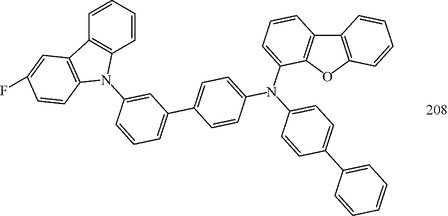 [SMILES: Fc1ccc2c(c1)c1ccccc1n2-c1cccc(-c2ccc(N(c3ccc(-c4ccccc4)cc3)c3cccc4c3oc3ccccc34)cc2)c1]

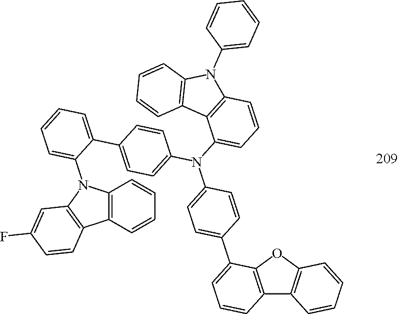 [SMILES: Fc1ccc2c3ccccc3n(-c3ccccc3-c3ccc(N(c4ccc(-c5cccc6c5oc5ccccc56)cc4)c4cccc5c4c4ccccc4n5-c4ccccc4)cc3)c2c1]

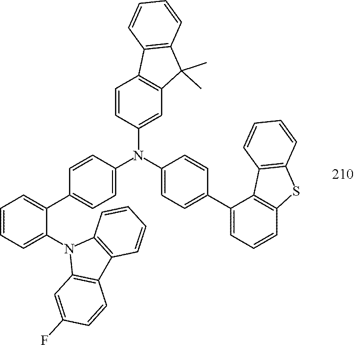 [SMILES: CC1(C)c2ccccc2-c2ccc(N(c3ccc(-c4ccccc4-n4c5ccccc5c5ccc(F)cc54)cc3)c3ccc(-c4cccc5sc6ccccc6c45)cc3)cc21]

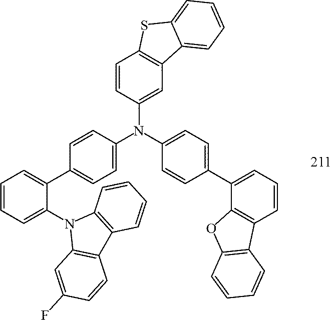 [SMILES: Fc1ccc2c3ccccc3n(-c3ccccc3-c3ccc(N(c4ccc(-c5cccc6c5oc5ccccc56)cc4)c4ccc5sc6ccccc6c5c4)cc3)c2c1]

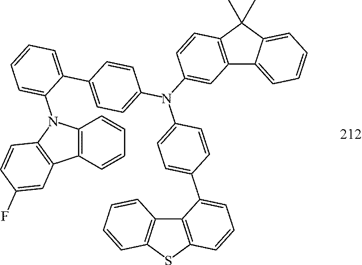 [SMILES: CC1(C)c2ccccc2-c2cc(N(c3ccc(-c4ccccc4-n4c5ccccc5c5cc(F)ccc54)cc3)c3ccc(-c4cccc5sc6ccccc6c45)cc3)ccc21]

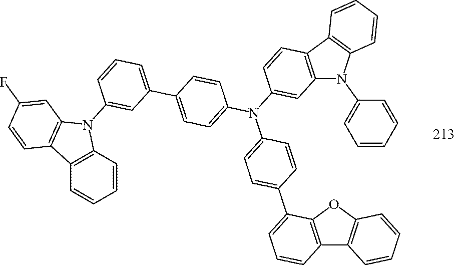 [SMILES: Fc1ccc2c3ccccc3n(-c3cccc(-c4ccc(N(c5ccc(-c6cccc7c6oc6ccccc67)cc5)c5ccc6c7ccccc7n(-c7ccccc7)c6c5)cc4)c3)c2c1]

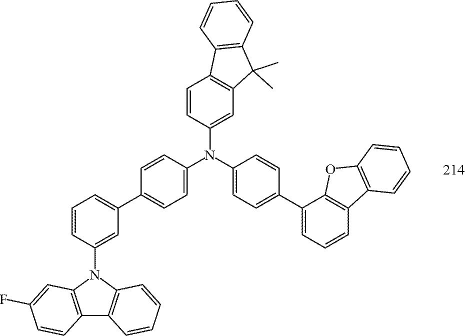 [SMILES: CC1(C)c2ccccc2-c2ccc(N(c3ccc(-c4cccc(-n5c6ccccc6c6ccc(F)cc65)c4)cc3)c3ccc(-c4cccc5c4oc4ccccc45)cc3)cc21]

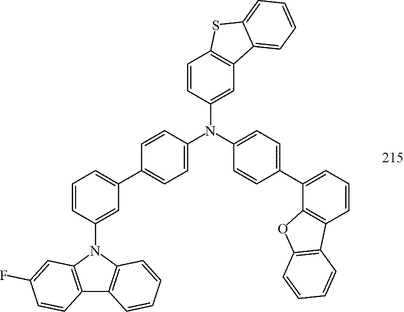 [SMILES: Fc1ccc2c3ccccc3n(-c3cccc(-c4ccc(N(c5ccc(-c6cccc7c6oc6ccccc67)cc5)c5ccc6sc7ccccc7c6c5)cc4)c3)c2c1]

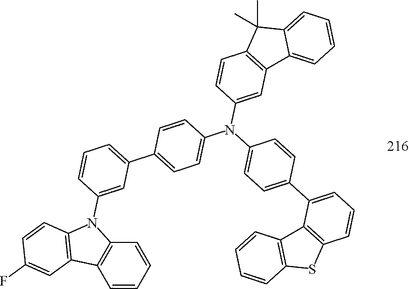 [SMILES: CC1(C)c2ccccc2-c2cc(N(c3ccc(-c4cccc(-n5c6ccccc6c6cc(F)ccc65)c4)cc3)c3ccc(-c4cccc5sc6ccccc6c45)cc3)ccc21]

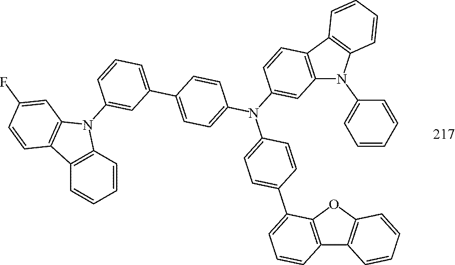 [SMILES: Fc1ccc2c3ccccc3n(-c3cccc(-c4ccc(N(c5ccc(-c6cccc7c6oc6ccccc67)cc5)c5ccc6c7ccccc7n(-c7ccccc7)c6c5)cc4)c3)c2c1]

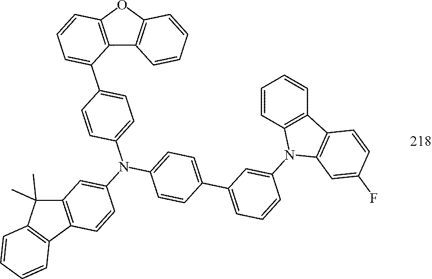 [SMILES: CC1(C)c2ccccc2-c2ccc(N(c3ccc(-c4cccc(-n5c6ccccc6c6ccc(F)cc65)c4)cc3)c3ccc(-c4cccc5oc6ccccc6c45)cc3)cc21]

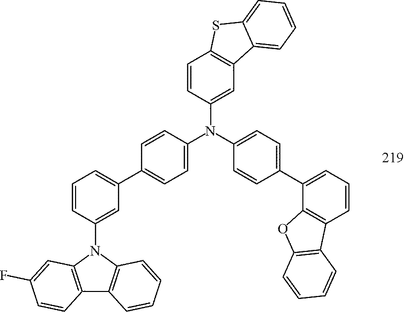 [SMILES: Fc1ccc2c3ccccc3n(-c3cccc(-c4ccc(N(c5ccc(-c6cccc7c6oc6ccccc67)cc5)c5ccc6sc7ccccc7c6c5)cc4)c3)c2c1]

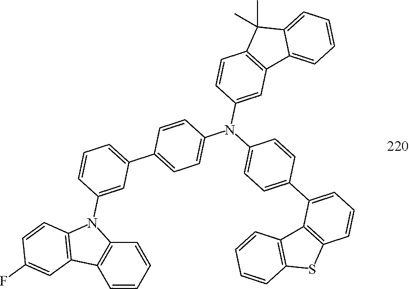 [SMILES: CC1(C)c2ccccc2-c2cc(N(c3ccc(-c4cccc(-n5c6ccccc6c6cc(F)ccc65)c4)cc3)c3ccc(-c4cccc5sc6ccccc6c45)cc3)ccc21]

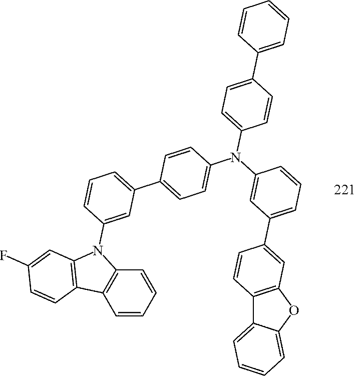 [SMILES: Fc1ccc2c3ccccc3n(-c3cccc(-c4ccc(N(c5ccc(-c6ccccc6)cc5)c5cccc(-c6ccc7c(c6)oc6ccccc67)c5)cc4)c3)c2c1]

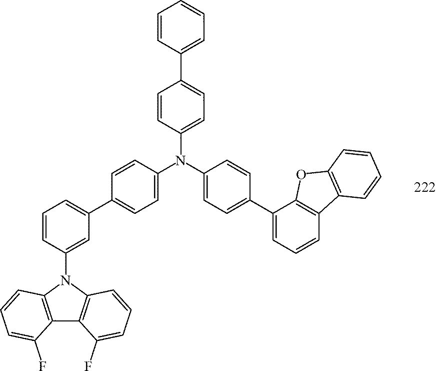 [SMILES: Fc1cccc2c1c1c(F)cccc1n2-c1cccc(-c2ccc(N(c3ccc(-c4ccccc4)cc3)c3ccc(-c4cccc5c4oc4ccccc45)cc3)cc2)c1]

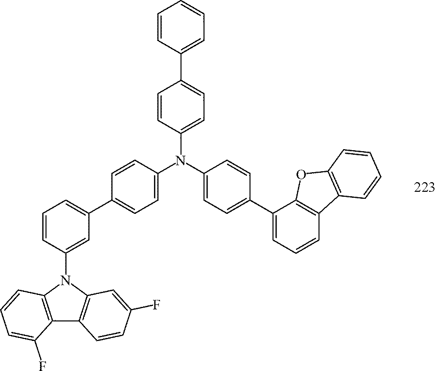 [SMILES: Fc1ccc2c3c(F)cccc3n(-c3cccc(-c4ccc(N(c5ccc(-c6ccccc6)cc5)c5ccc(-c6cccc7c6oc6ccccc67)cc5)cc4)c3)c2c1]

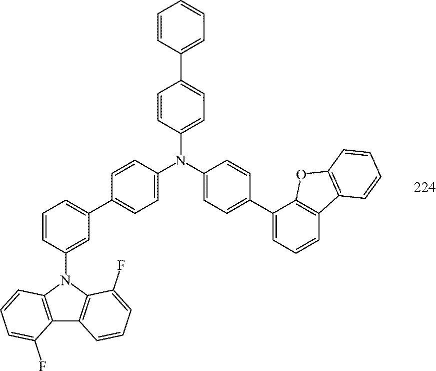 [SMILES: Fc1cccc2c1c1cccc(F)c1n2-c1cccc(-c2ccc(N(c3ccc(-c4ccccc4)cc3)c3ccc(-c4cccc5c4oc4ccccc45)cc3)cc2)c1]

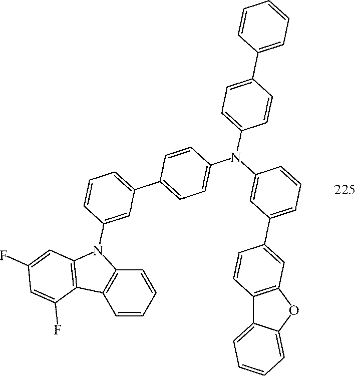 [SMILES: Fc1cc(F)c2c3ccccc3n(-c3cccc(-c4ccc(N(c5ccc(-c6ccccc6)cc5)c5cccc(-c6ccc7c(c6)oc6ccccc67)c5)cc4)c3)c2c1]

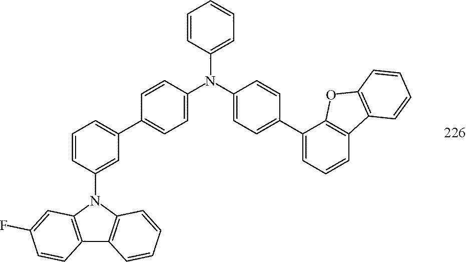 [SMILES: Fc1ccc2c3ccccc3n(-c3cccc(-c4ccc(N(c5ccccc5)c5ccc(-c6cccc7c6oc6ccccc67)cc5)cc4)c3)c2c1]

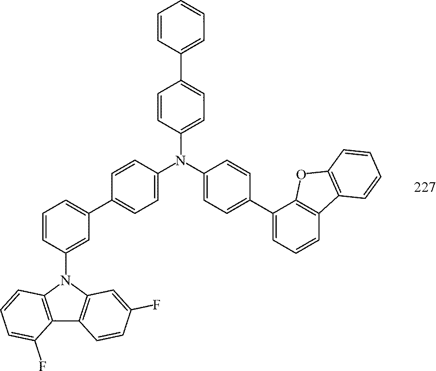 [SMILES: Fc1ccc2c3c(F)cccc3n(-c3cccc(-c4ccc(N(c5ccc(-c6ccccc6)cc5)c5ccc(-c6cccc7c6oc6ccccc67)cc5)cc4)c3)c2c1]

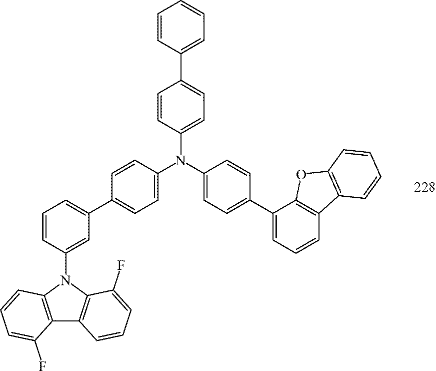 [SMILES: Fc1cccc2c1c1cccc(F)c1n2-c1cccc(-c2ccc(N(c3ccc(-c4ccccc4)cc3)c3ccc(-c4cccc5c4oc4ccccc45)cc3)cc2)c1]

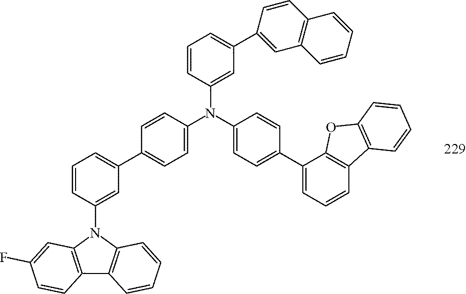 [SMILES: Fc1ccc2c3ccccc3n(-c3cccc(-c4ccc(N(c5ccc(-c6cccc7c6oc6ccccc67)cc5)c5cccc(-c6ccc7ccccc7c6)c5)cc4)c3)c2c1]

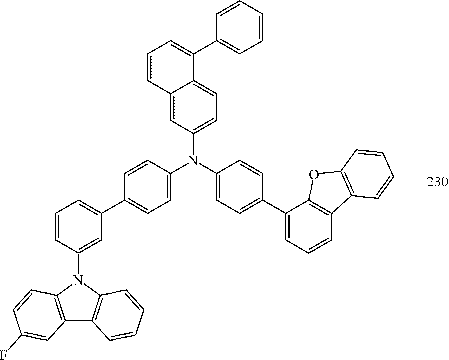 [SMILES: Fc1ccc2c(c1)c1ccccc1n2-c1cccc(-c2ccc(N(c3ccc(-c4cccc5c4oc4ccccc45)cc3)c3ccc4c(-c5ccccc5)cccc4c3)cc2)c1]

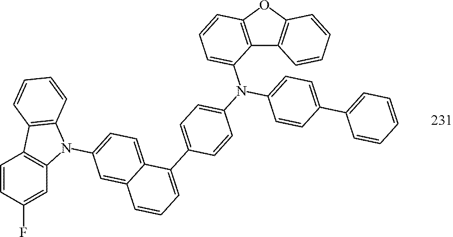 [SMILES: Fc1ccc2c3ccccc3n(-c3ccc4c(-c5ccc(N(c6ccc(-c7ccccc7)cc6)c6cccc7oc8ccccc8c67)cc5)cccc4c3)c2c1]

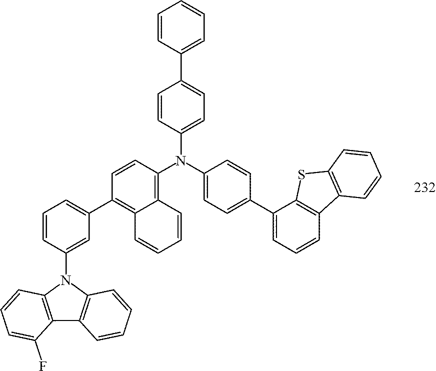 [SMILES: Fc1cccc2c1c1ccccc1n2-c1cccc(-c2ccc(N(c3ccc(-c4ccccc4)cc3)c3ccc(-c4cccc5c4sc4ccccc45)cc3)c3ccccc23)c1]

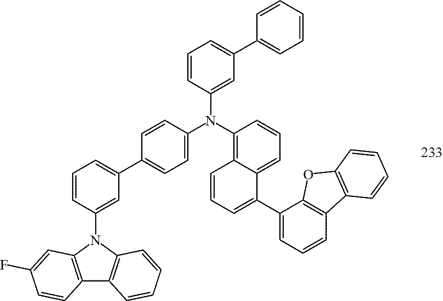 [SMILES: Fc1ccc2c3ccccc3n(-c3cccc(-c4ccc(N(c5cccc(-c6ccccc6)c5)c5cccc6c(-c7cccc8c7oc7ccccc78)cccc56)cc4)c3)c2c1]

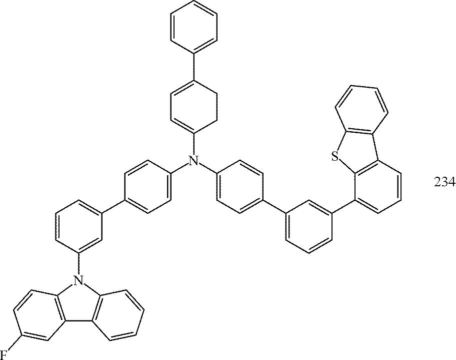 [SMILES: Fc1ccc2c(c1)c1ccccc1n2-c1cccc(-c2ccc(N(C3=CC=C(c4ccccc4)CC3)c3ccc(-c4cccc(-c5cccc6c5sc5ccccc56)c4)cc3)cc2)c1]

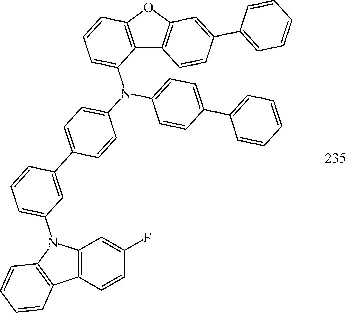 [SMILES: Fc1ccc2c3ccccc3n(-c3cccc(-c4ccc(N(c5ccc(-c6ccccc6)cc5)c5cccc6oc7cc(-c8ccccc8)ccc7c56)cc4)c3)c2c1]

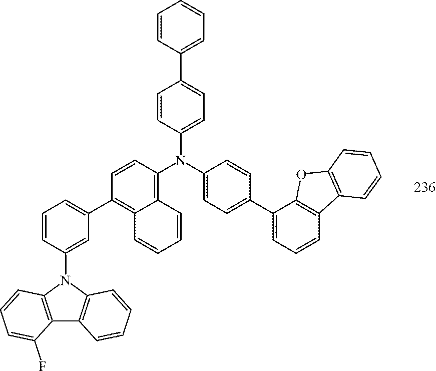 [SMILES: Fc1cccc2c1c1ccccc1n2-c1cccc(-c2ccc(N(c3ccc(-c4ccccc4)cc3)c3ccc(-c4cccc5c4oc4ccccc45)cc3)c3ccccc23)c1]

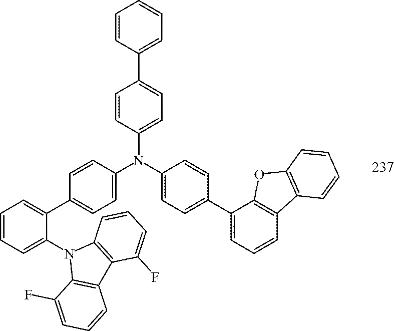 [SMILES: Fc1cccc2c1c1cccc(F)c1n2-c1ccccc1-c1ccc(N(c2ccc(-c3ccccc3)cc2)c2ccc(-c3cccc4c3oc3ccccc34)cc2)cc1]

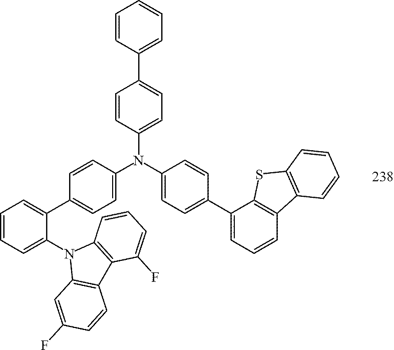 [SMILES: Fc1ccc2c3c(F)cccc3n(-c3ccccc3-c3ccc(N(c4ccc(-c5ccccc5)cc4)c4ccc(-c5cccc6c5sc5ccccc56)cc4)cc3)c2c1]

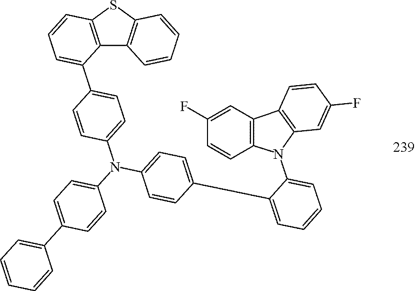 [SMILES: Fc1ccc2c(c1)c1ccc(F)cc1n2-c1ccccc1-c1ccc(N(c2ccc(-c3ccccc3)cc2)c2ccc(-c3cccc4sc5ccccc5c34)cc2)cc1]

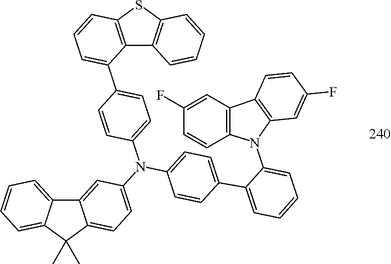 [SMILES: CC1(C)c2ccccc2-c2cc(N(c3ccc(-c4ccccc4-n4c5ccc(F)cc5c5ccc(F)cc54)cc3)c3ccc(-c4cccc5sc6ccccc6c45)cc3)ccc21]

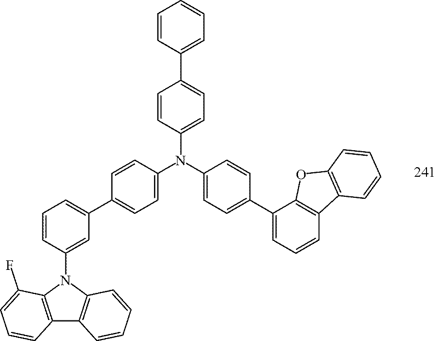 [SMILES: Fc1cccc2c3ccccc3n(-c3cccc(-c4ccc(N(c5ccc(-c6ccccc6)cc5)c5ccc(-c6cccc7c6oc6ccccc67)cc5)cc4)c3)c12]

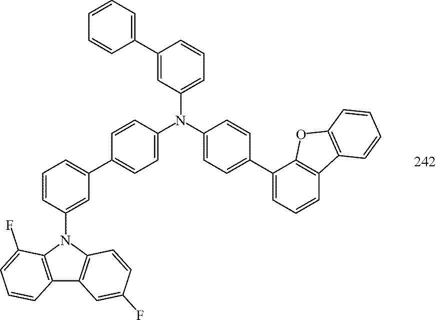 [SMILES: Fc1ccc2c(c1)c1cccc(F)c1n2-c1cccc(-c2ccc(N(c3ccc(-c4cccc5c4oc4ccccc45)cc3)c3cccc(-c4ccccc4)c3)cc2)c1]

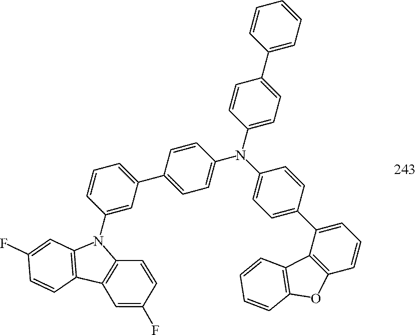 [SMILES: Fc1ccc2c(c1)c1ccc(F)cc1n2-c1cccc(-c2ccc(N(c3ccc(-c4ccccc4)cc3)c3ccc(-c4cccc5oc6ccccc6c45)cc3)cc2)c1]

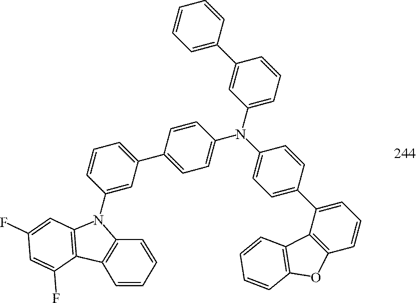 [SMILES: Fc1cc(F)c2c3ccccc3n(-c3cccc(-c4ccc(N(c5ccc(-c6cccc7oc8ccccc8c67)cc5)c5cccc(-c6ccccc6)c5)cc4)c3)c2c1]

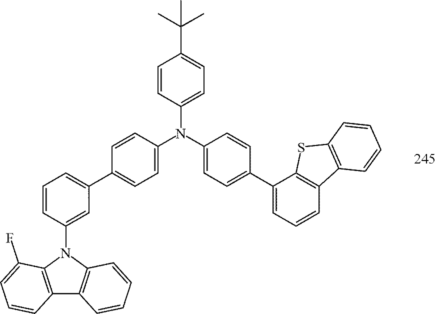 [SMILES: CC(C)(C)c1ccc(N(c2ccc(-c3cccc(-n4c5ccccc5c5cccc(F)c54)c3)cc2)c2ccc(-c3cccc4c3sc3ccccc34)cc2)cc1]

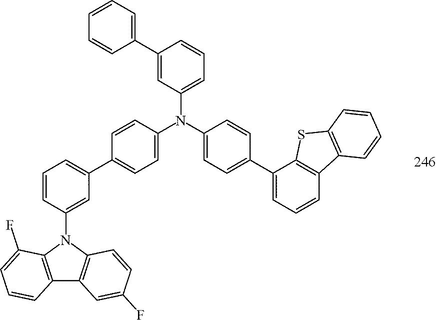 [SMILES: Fc1ccc2c(c1)c1cccc(F)c1n2-c1cccc(-c2ccc(N(c3ccc(-c4cccc5c4sc4ccccc45)cc3)c3cccc(-c4ccccc4)c3)cc2)c1]

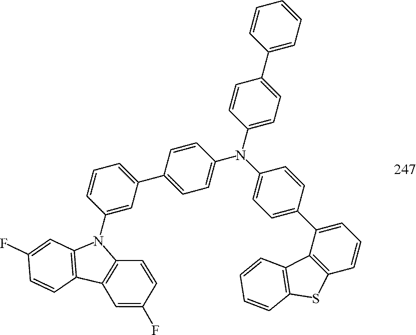 [SMILES: Fc1ccc2c(c1)c1ccc(F)cc1n2-c1cccc(-c2ccc(N(c3ccc(-c4ccccc4)cc3)c3ccc(-c4cccc5sc6ccccc6c45)cc3)cc2)c1]

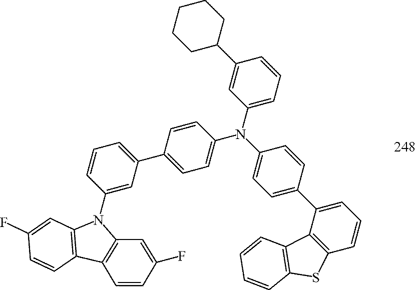 [SMILES: Fc1ccc2c3ccc(F)cc3n(-c3cccc(-c4ccc(N(c5ccc(-c6cccc7sc8ccccc8c67)cc5)c5cccc(C6CCCCC6)c5)cc4)c3)c2c1]

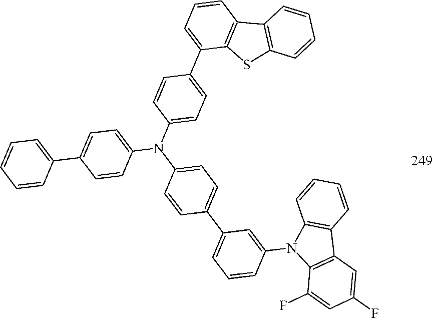 [SMILES: Fc1cc(F)c2c(c1)c1ccccc1n2-c1cccc(-c2ccc(N(c3ccc(-c4ccccc4)cc3)c3ccc(-c4cccc5c4sc4ccccc45)cc3)cc2)c1]

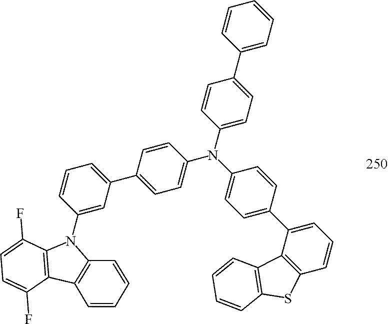 [SMILES: Fc1ccc(F)c2c1c1ccccc1n2-c1cccc(-c2ccc(N(c3ccc(-c4ccccc4)cc3)c3ccc(-c4cccc5sc6ccccc6c45)cc3)cc2)c1]

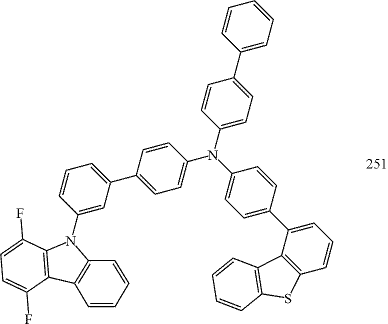 [SMILES: Fc1ccc(F)c2c1c1ccccc1n2-c1cccc(-c2ccc(N(c3ccc(-c4ccccc4)cc3)c3ccc(-c4cccc5sc6ccccc6c45)cc3)cc2)c1]

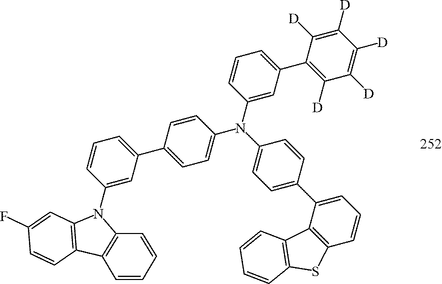 [SMILES: [2H]c1c([2H])c([2H])c(-c2cccc(N(c3ccc(-c4cccc(-n5c6ccccc6c6ccc(F)cc65)c4)cc3)c3ccc(-c4cccc5sc6ccccc6c45)cc3)c2)c([2H])c1[2H]]

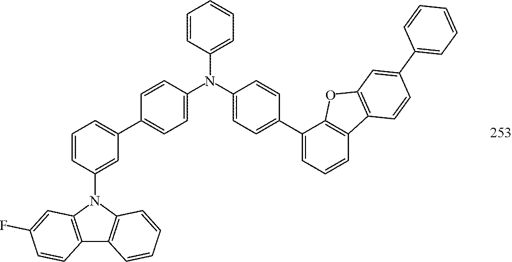 [SMILES: Fc1ccc2c3ccccc3n(-c3cccc(-c4ccc(N(c5ccccc5)c5ccc(-c6cccc7c6oc6cc(-c8ccccc8)ccc67)cc5)cc4)c3)c2c1]

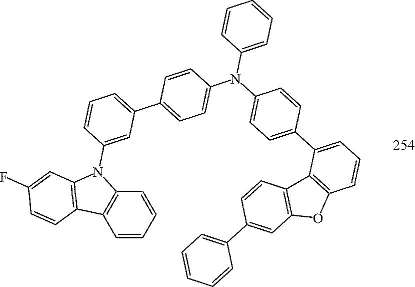 [SMILES: Fc1ccc2c3ccccc3n(-c3cccc(-c4ccc(N(c5ccccc5)c5ccc(-c6cccc7oc8cc(-c9ccccc9)ccc8c67)cc5)cc4)c3)c2c1]

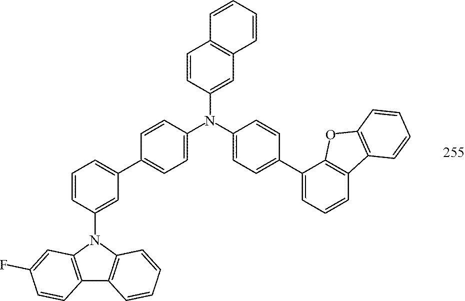 [SMILES: Fc1ccc2c3ccccc3n(-c3cccc(-c4ccc(N(c5ccc(-c6cccc7c6oc6ccccc67)cc5)c5ccc6ccccc6c5)cc4)c3)c2c1]

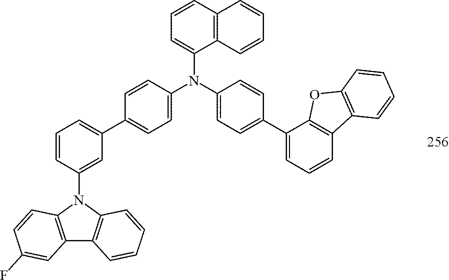 [SMILES: Fc1ccc2c(c1)c1ccccc1n2-c1cccc(-c2ccc(N(c3ccc(-c4cccc5c4oc4ccccc45)cc3)c3cccc4ccccc34)cc2)c1]

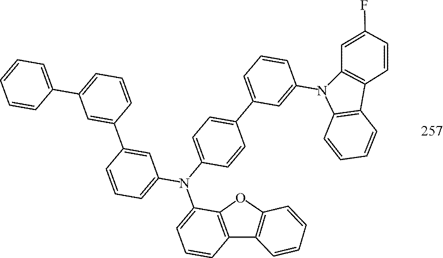 [SMILES: Fc1ccc2c3ccccc3n(-c3cccc(-c4ccc(N(c5cccc(-c6cccc(-c7ccccc7)c6)c5)c5cccc6c5oc5ccccc56)cc4)c3)c2c1]

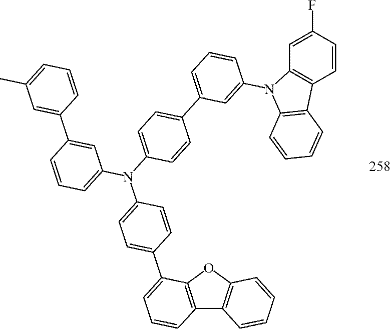 [SMILES: Cc1cccc(-c2cccc(N(c3ccc(-c4cccc(-n5c6ccccc6c6ccc(F)cc65)c4)cc3)c3ccc(-c4cccc5c4oc4ccccc45)cc3)c2)c1]

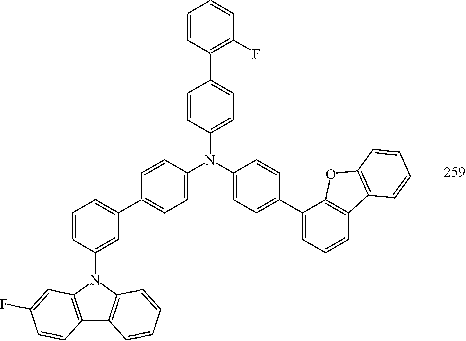 [SMILES: Fc1ccc2c3ccccc3n(-c3cccc(-c4ccc(N(c5ccc(-c6ccccc6F)cc5)c5ccc(-c6cccc7c6oc6ccccc67)cc5)cc4)c3)c2c1]

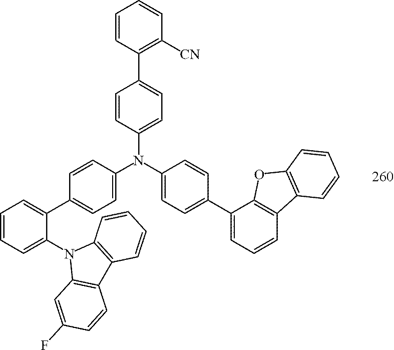 [SMILES: N#Cc1ccccc1-c1ccc(N(c2ccc(-c3ccccc3-n3c4ccccc4c4ccc(F)cc43)cc2)c2ccc(-c3cccc4c3oc3ccccc34)cc2)cc1]

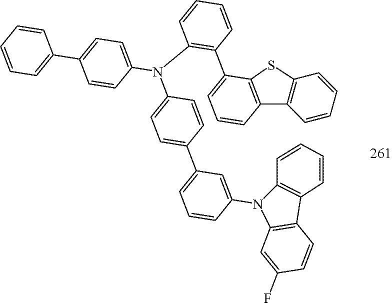 [SMILES: Fc1ccc2c3ccccc3n(-c3cccc(-c4ccc(N(c5ccc(-c6ccccc6)cc5)c5ccccc5-c5cccc6c5sc5ccccc56)cc4)c3)c2c1]

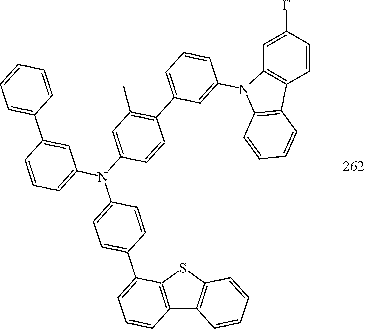 [SMILES: Cc1cc(N(c2ccc(-c3cccc4c3sc3ccccc34)cc2)c2cccc(-c3ccccc3)c2)ccc1-c1cccc(-n2c3ccccc3c3ccc(F)cc32)c1]

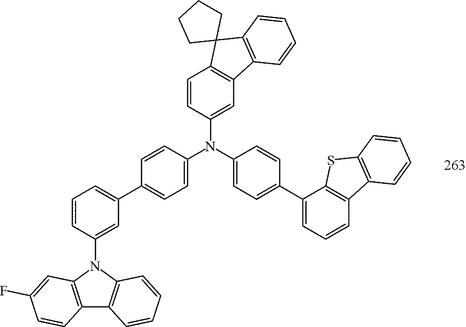 [SMILES: Fc1ccc2c3ccccc3n(-c3cccc(-c4ccc(N(c5ccc(-c6cccc7c6sc6ccccc67)cc5)c5ccc6c(c5)-c5ccccc5C65CCCC5)cc4)c3)c2c1]

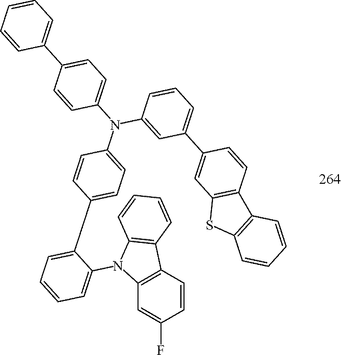 [SMILES: Fc1ccc2c3ccccc3n(-c3ccccc3-c3ccc(N(c4ccc(-c5ccccc5)cc4)c4cccc(-c5ccc6c(c5)sc5ccccc56)c4)cc3)c2c1]

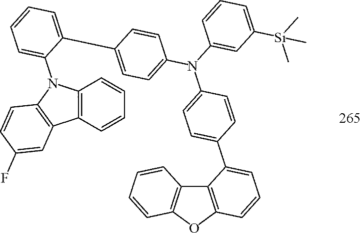 [SMILES: C[Si](C)(C)c1cccc(N(c2ccc(-c3ccccc3-n3c4ccccc4c4cc(F)ccc43)cc2)c2ccc(-c3cccc4oc5ccccc5c34)cc2)c1]

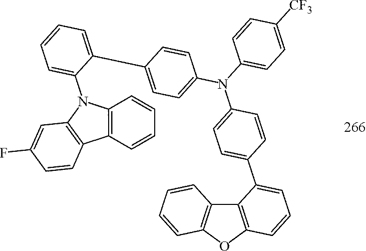 [SMILES: Fc1ccc2c3ccccc3n(-c3ccccc3-c3ccc(N(c4ccc(-c5cccc6oc7ccccc7c56)cc4)c4ccc(C(F)(F)F)cc4)cc3)c2c1]

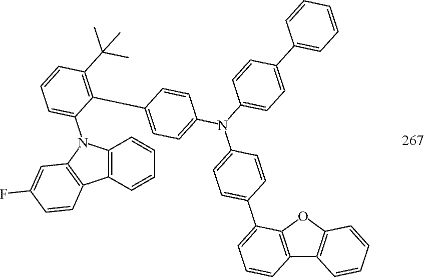 [SMILES: CC(C)(C)c1cccc(-n2c3ccccc3c3ccc(F)cc32)c1-c1ccc(N(c2ccc(-c3ccccc3)cc2)c2ccc(-c3cccc4c3oc3ccccc34)cc2)cc1]

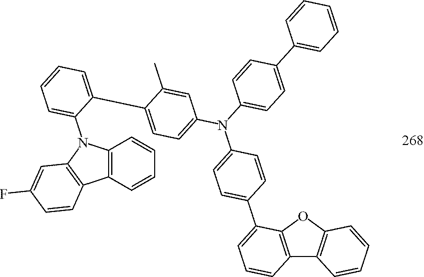 [SMILES: Cc1cc(N(c2ccc(-c3ccccc3)cc2)c2ccc(-c3cccc4c3oc3ccccc34)cc2)ccc1-c1ccccc1-n1c2ccccc2c2ccc(F)cc21]

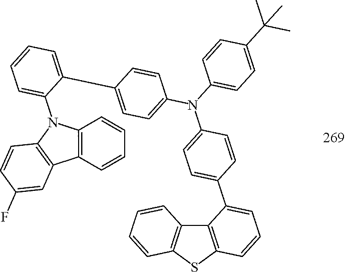 [SMILES: CC(C)(C)c1ccc(N(c2ccc(-c3ccccc3-n3c4ccccc4c4cc(F)ccc43)cc2)c2ccc(-c3cccc4sc5ccccc5c34)cc2)cc1]

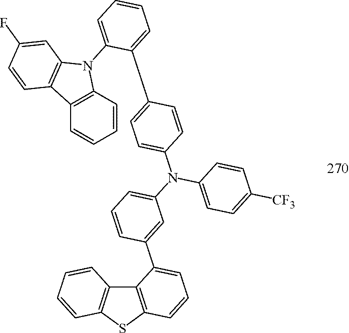 [SMILES: Fc1ccc2c3ccccc3n(-c3ccccc3-c3ccc(N(c4ccc(C(F)(F)F)cc4)c4cccc(-c5cccc6sc7ccccc7c56)c4)cc3)c2c1]

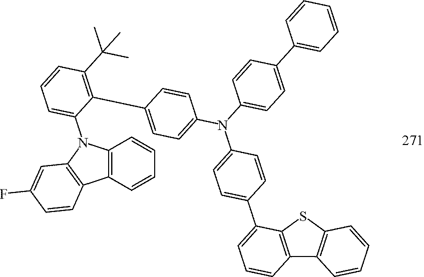 [SMILES: CC(C)(C)c1cccc(-n2c3ccccc3c3ccc(F)cc32)c1-c1ccc(N(c2ccc(-c3ccccc3)cc2)c2ccc(-c3cccc4c3sc3ccccc34)cc2)cc1]

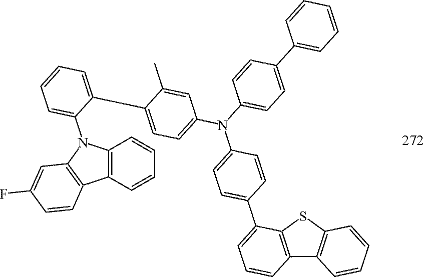 [SMILES: Cc1cc(N(c2ccc(-c3ccccc3)cc2)c2ccc(-c3cccc4c3sc3ccccc34)cc2)ccc1-c1ccccc1-n1c2ccccc2c2ccc(F)cc21]

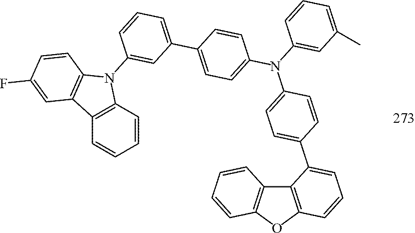 [SMILES: Cc1cccc(N(c2ccc(-c3cccc(-n4c5ccccc5c5cc(F)ccc54)c3)cc2)c2ccc(-c3cccc4oc5ccccc5c34)cc2)c1]

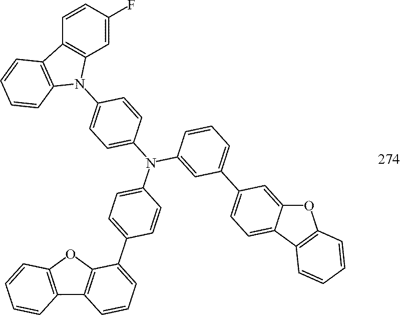 [SMILES: Fc1ccc2c3ccccc3n(-c3ccc(N(c4ccc(-c5cccc6c5oc5ccccc56)cc4)c4cccc(-c5ccc6c(c5)oc5ccccc56)c4)cc3)c2c1]

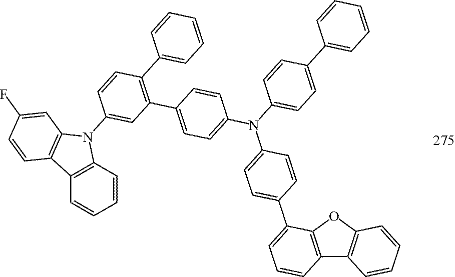 [SMILES: Fc1ccc2c3ccccc3n(-c3ccc(-c4ccccc4)c(-c4ccc(N(c5ccc(-c6ccccc6)cc5)c5ccc(-c6cccc7c6oc6ccccc67)cc5)cc4)c3)c2c1]

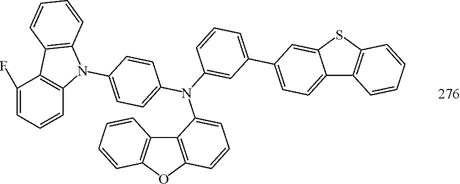 [SMILES: Fc1cccc2c1c1ccccc1n2-c1ccc(N(c2cccc(-c3ccc4c(c3)sc3ccccc34)c2)c2cccc3oc4ccccc4c23)cc1]

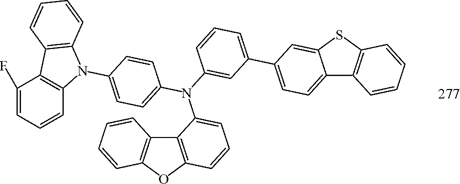 [SMILES: Fc1cccc2c1c1ccccc1n2-c1ccc(N(c2cccc(-c3ccc4c(c3)sc3ccccc34)c2)c2cccc3oc4ccccc4c23)cc1]

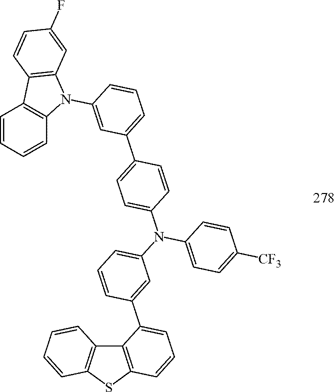 [SMILES: Fc1ccc2c3ccccc3n(-c3cccc(-c4ccc(N(c5ccc(C(F)(F)F)cc5)c5cccc(-c6cccc7sc8ccccc8c67)c5)cc4)c3)c2c1]

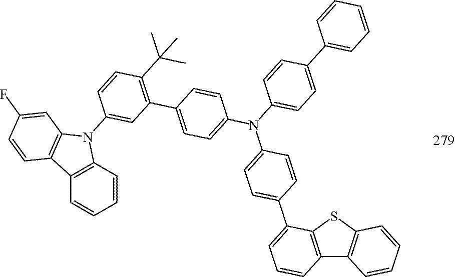 [SMILES: CC(C)(C)c1ccc(-n2c3ccccc3c3ccc(F)cc32)cc1-c1ccc(N(c2ccc(-c3ccccc3)cc2)c2ccc(-c3cccc4c3sc3ccccc34)cc2)cc1]

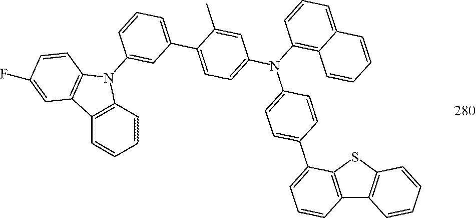 [SMILES: Cc1cc(N(c2ccc(-c3cccc4c3sc3ccccc34)cc2)c2cccc3ccccc23)ccc1-c1cccc(-n2c3ccccc3c3cc(F)ccc32)c1]

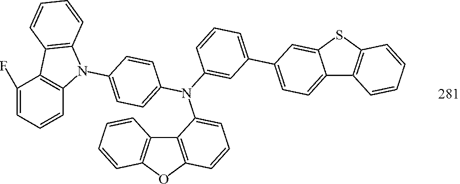 [SMILES: Fc1cccc2c1c1ccccc1n2-c1ccc(N(c2cccc(-c3ccc4c(c3)sc3ccccc34)c2)c2cccc3oc4ccccc4c23)cc1]

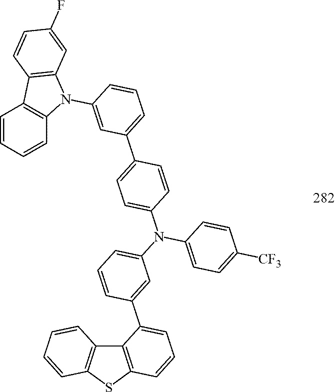 [SMILES: Fc1ccc2c3ccccc3n(-c3cccc(-c4ccc(N(c5ccc(C(F)(F)F)cc5)c5cccc(-c6cccc7sc8ccccc8c67)c5)cc4)c3)c2c1]

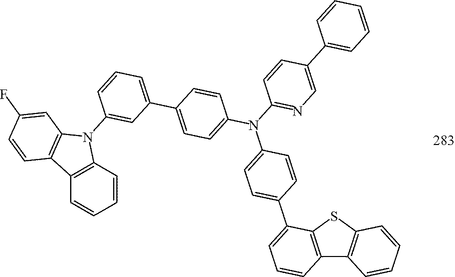 [SMILES: Fc1ccc2c3ccccc3n(-c3cccc(-c4ccc(N(c5ccc(-c6cccc7c6sc6ccccc67)cc5)c5ccc(-c6ccccc6)cn5)cc4)c3)c2c1]

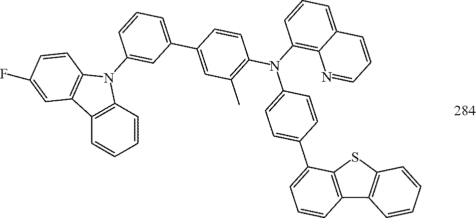 [SMILES: Cc1cc(-c2cccc(-n3c4ccccc4c4cc(F)ccc43)c2)ccc1N(c1ccc(-c2cccc3c2sc2ccccc23)cc1)c1cccc2cccnc12]

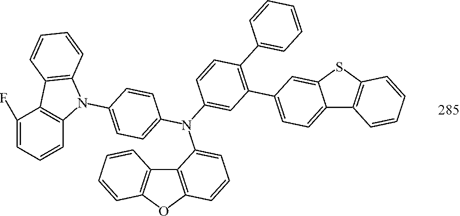 [SMILES: Fc1cccc2c1c1ccccc1n2-c1ccc(N(c2ccc(-c3ccccc3)c(-c3ccc4c(c3)sc3ccccc34)c2)c2cccc3oc4ccccc4c23)cc1]

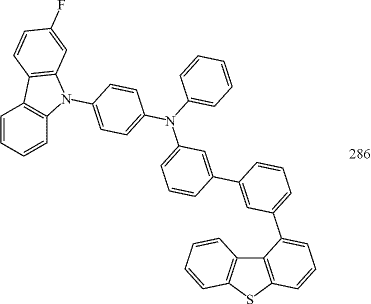 [SMILES: Fc1ccc2c3ccccc3n(-c3ccc(N(c4ccccc4)c4cccc(-c5cccc(-c6cccc7sc8ccccc8c67)c5)c4)cc3)c2c1]

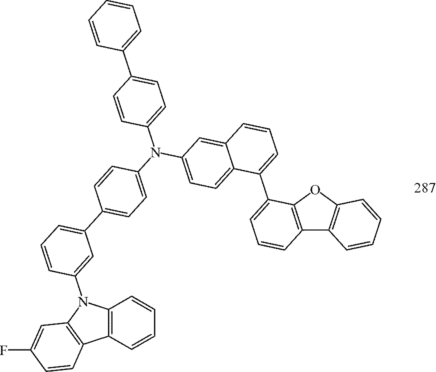 [SMILES: Fc1ccc2c3ccccc3n(-c3cccc(-c4ccc(N(c5ccc(-c6ccccc6)cc5)c5ccc6c(-c7cccc8c7oc7ccccc78)cccc6c5)cc4)c3)c2c1]

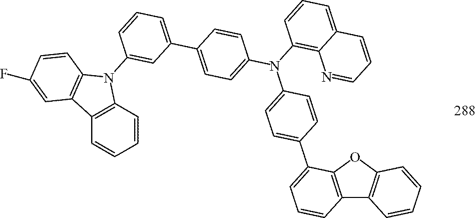 [SMILES: Fc1ccc2c(c1)c1ccccc1n2-c1cccc(-c2ccc(N(c3ccc(-c4cccc5c4oc4ccccc45)cc3)c3cccc4cccnc34)cc2)c1]

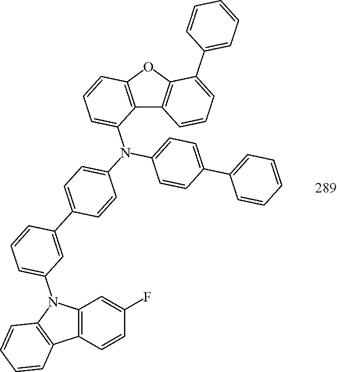 [SMILES: Fc1ccc2c3ccccc3n(-c3cccc(-c4ccc(N(c5ccc(-c6ccccc6)cc5)c5cccc6oc7c(-c8ccccc8)cccc7c56)cc4)c3)c2c1]

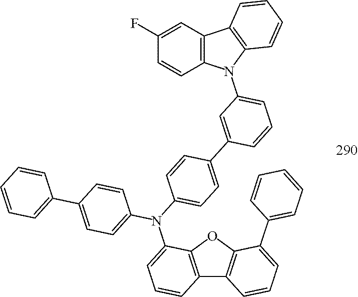 [SMILES: Fc1ccc2c(c1)c1ccccc1n2-c1cccc(-c2ccc(N(c3ccc(-c4ccccc4)cc3)c3cccc4c3oc3c(-c5ccccc5)cccc34)cc2)c1]

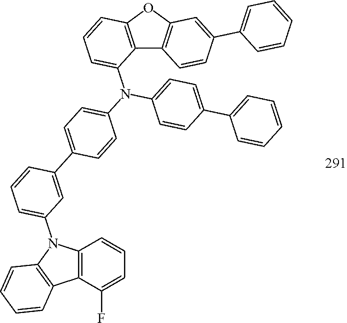 [SMILES: Fc1cccc2c1c1ccccc1n2-c1cccc(-c2ccc(N(c3ccc(-c4ccccc4)cc3)c3cccc4oc5cc(-c6ccccc6)ccc5c34)cc2)c1]

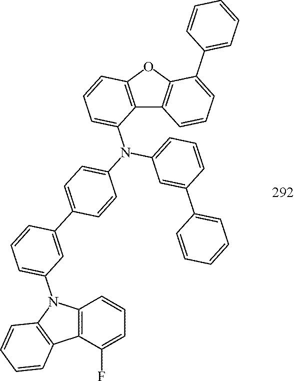 [SMILES: Fc1cccc2c1c1ccccc1n2-c1cccc(-c2ccc(N(c3cccc(-c4ccccc4)c3)c3cccc4oc5c(-c6ccccc6)cccc5c34)cc2)c1]

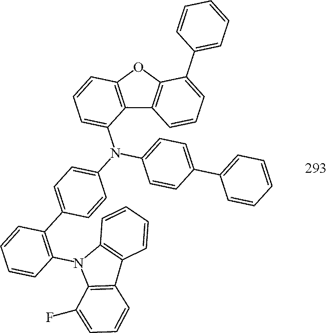 [SMILES: Fc1cccc2c3ccccc3n(-c3ccccc3-c3ccc(N(c4ccc(-c5ccccc5)cc4)c4cccc5oc6c(-c7ccccc7)cccc6c45)cc3)c12]

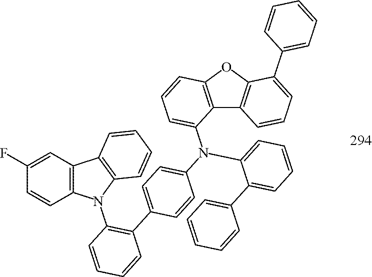 [SMILES: Fc1ccc2c(c1)c1ccccc1n2-c1ccccc1-c1ccc(N(c2ccccc2-c2ccccc2)c2cccc3oc4c(-c5ccccc5)cccc4c23)cc1]

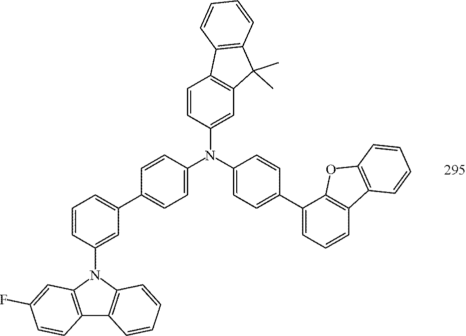 [SMILES: CC1(C)c2ccccc2-c2ccc(N(c3ccc(-c4cccc(-n5c6ccccc6c6ccc(F)cc65)c4)cc3)c3ccc(-c4cccc5c4oc4ccccc45)cc3)cc21]

A synthesis method of the nitrogen-containing compound provided is not particularly limited in the present disclosure, and those skilled in the art can determine a suitable synthesis method according to the nitrogen-containing compound of the present disclosure in combination with preparation methods provided in synthesis examples. In other words, the synthesis examples of the present disclosure exemplarily provide preparation methods for the nitrogen-containing compounds, and raw materials used may be commercially obtained or obtained by a method well known in the art. Those skilled in the art can obtain all nitrogen-containing compounds provided by the present disclosure according to these exemplary preparation methods, and all specific preparation methods for the nitrogen-containing compounds are not described in detail here, and those skilled in the art should not understand as limiting the present disclosure.

In a second aspect, the present disclosure provides an electronic element, including an anode and a cathode which are oppositely disposed, and a functional layer disposed between the anode and the cathode; and the functional layer containis the nitrogen-containing compound according to the first aspect of the present disclosure.

The nitrogen-containing compound provided by the present disclosure can be used to form at least one organic film layer in the functional layer to improve the properties such as the service life of the electronic element.

Optionally, the functional layer includes a hole transport layer, and the hole transport layer contains the nitrogen-containing compound provided by the present disclosure. The hole transport layer may be composed of the nitrogen-containing compound provided by the present disclosure or may be composed of the nitrogen-containing compound provided by the present disclosure together with other materials. The hole transport layer may be one layer or two or more layers.

Optionally, the electronic element is an organic electroluminescent device or a photoelectric conversion device. The organic electroluminescent device may be a green light device, a red light device or a blue light device.

According to one example, the electronic element is the organic electroluminescent device, the hole transport layer includes a first hole transport layer and a second hole transport layer (also referred to as an “electron blocking layer”), the first hole transport layer is closer to the anode than the second hole transport layer, and the second hole transport layer contains the nitrogen-containing compound, i.e. the electron blocking layer contains the nitrogen-containing compound.

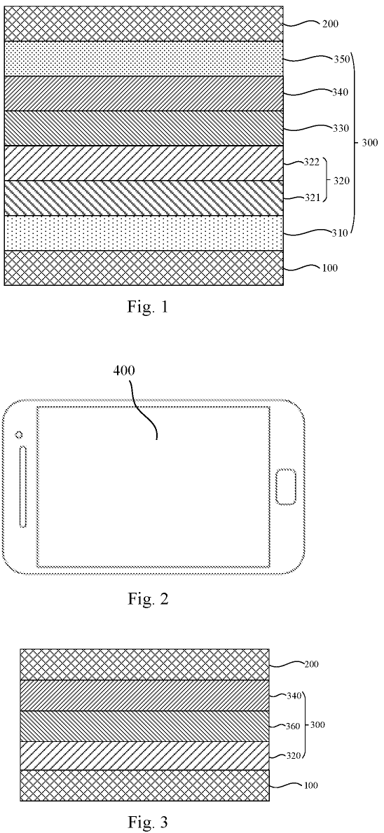

According to one specific example, the electronic element is the organic electroluminescent device. As shown in FIG. 1, the organic electroluminescent device may include an anode 100, a first hole transport layer 321, a second hole transport layer 322, an organic light-emitting layer 330 as an energy conversion layer, an electron transport layer 340, and a cathode 200 which are stacked in sequence.

Optionally, the anode 100 includes an anode material, which is preferably a material with a large work function that facilitates hole injection into the functional layer. Specific examples of the anode material include metals such as nickel, platinum, vanadium, chromium, copper, zinc, and gold, or their alloys; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene] (PEDT), polypyrrole, and polyaniline, but are not limited to these. A transparent electrode containing indium tin oxide (ITO) as the anode is preferably included.

Optionally, the first hole transport layer 321 may include one or more hole transport materials, and the hole transport materials may be selected from a carbazole polymers, carbazole-linked triarylamine compounds, or other types of compounds, which are not particularly limited in the present disclosure. For example, the first hole transport layer 321 may be composed of a compound NPB.

Optionally, the organic light-emitting layer 330 may be composed of a single light-emitting material and may also include a host material and a guest material. Optionally, the organic light-emitting layer 330 is composed of the host material and the guest material, and holes injected into the organic light-emitting layer 330 and electrons injected into the organic light-emitting layer 330 may be recombined in the organic light-emitting layer 330 to form excitons, the excitons transfer energy to the host material, and the host material transfers energy to the guest material, thus enabling the guest material to emit light.

The host material of the organic light-emitting layer 330 may be a metal chelate compound, a bis-styryl derivative, an aromatic amine derivative, a dibenzofuran derivative, or other types of materials, which is not particularly limited in the present disclosure. For example, the host material may be α,β-ADN.

The guest material of the organic light-emitting layer 330 may be a compound having a condensed aryl ring or its derivative, a compound having a heteroaryl ring or its derivative, an aromatic amine derivative, or other materials, which is not particularly limited in the present disclosure. For example, the guest material may be BD-1 (the structure is shown below).

The electron transport layer 340 may be of a single-layer structure or a multi-layer structure and may include one or more electron transport materials, and the electron transport materials can be selected from, but are not limited to, a benzimidazole derivative, an oxadiazole derivative, a quinoxaline derivative, or other electron transport materials. In one example of the present disclosure, the electron transport layer 340 may be composed of TPBi and LiQ.

In the present disclosure, the cathode 200 may include a cathode material, which is the material having a small work function that facilitates electron injection into the functional layer. Specific examples of the cathode material include, but are not limited to, metals such as magnesium, calcium, sodium, potassium, titanium, indium, yttrium, lithium, gadolinium, aluminum, silver, tin, and lead or their alloys; or multilayer materials such as LiF/Al, Liq/Al, LiO₂/Al, LiF/Ca, LiF/Al, and BaF₂/Ca. A metal electrode containing magnesium and silver as the cathode is preferably included.

Optionally, as shown in FIG. 1, a hole injection layer 310 may also be arranged between the anode 100 and the first hole transport layer 321 to enhance the ability to inject holes into the first hole transport layer 321. The hole injection layer 310 can be made of a biphenylamine derivative, a starburst arylamine compound, a phthalocyanine derivative or other materials, which is not specially limited in the present disclosure. For example, the hole injection layer 310 may be composed of F4-TCNQ.

Optionally, as shown in FIG. 1, an electron injection layer 350 may also be arranged between the cathode 200 and the electron transport layer 340 to enhance the ability to inject electrons into the electron transport layer 340. The electron injection layer 350 may include an inorganic material such as an alkali metal sulfide and an alkali metal halide, or may include a complex of alkali metal and organic substance. For example, the electron injection layer 350 may include LiQ.

According to one example, the organic electroluminescent device is a blue light device.

According to another example, the electronic element may be a photoelectric conversion device. As shown in FIG. 3, the photoelectric conversion device may include an anode 100 and a cathode 200 which are disposed oppositely, and a functional layer 300 disposed between the anode 100 and the cathode 200; and the functional layer 300 contains the nitrogen-containing compound provided by the present disclosure.

According to one example, as shown in FIG. 3, the functional layer 300 includes a hole transport layer 320, and the hole transport layer 320 contains the nitrogen-containing compound of the present disclosure. The hole transport layer 320 may be composed of the nitrogen-containing compound provided by the present disclosure, or may be composed of the nitrogen-containing compound provided by the present disclosure together with other materials.

Optionally, the hole transport layer 320 may further include an inorganic doping material to improve the hole transport properties of the hole transport layer 320.

According to one specific example, as shown in FIG. 3, the photoelectric conversion device may include an anode 100, a hole transport layer 320, a photoelectric conversion layer 360, an electron transport layer 340, and a cathode 200 which are stacked in sequence.

Optionally, the photoelectric conversion device may be a solar cell, and in particular may be an organic thin film solar cell. For example, in one example of the present disclosure, the solar cell may include an anode, a hole transport layer, a photoelectric conversion layer, an electron transport layer, and a cathode which are stacked in sequence, and the hole transport layer includes the nitrogen-containing compound provided by the present disclosure.

In a third aspect, the present disclosure provides an electronic device, including the electronic element according to the second aspect of the present disclosure.

According to one example, as shown in FIG. 2, the electronic device is a first electronic device 400 including the organic electroluminescent device described above. The first electronic device 400 may, for example, be a display device, a lighting device, an optical communication device, or other types of electronic devices, and may include, for example, but is not limited to, a computer screen, a mobile phone screen, a television, electronic paper, an emergency lighting lamp, an optical module, and the like.

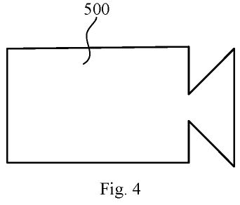

According to another example, as shown in FIG. 4, the electronic device is a second electronic device 500 including the photoelectric conversion device described above. The second electronic device 500 may, for example, be a solar power plant, a light detector, a fingerprint recognition device, an optical module, a CCD camera, or other types of electronic devices.

Compounds of which synthesis methods are not mentioned in the present disclosure are all commercially available raw material products.

The present disclosure is further illustrated below by way of examples, but the present disclosure is not limited in any way.

Synthesis examples are used to illustrate the synthesis of the nitrogen-containing compounds of the present disclosure.

Synthetic route:

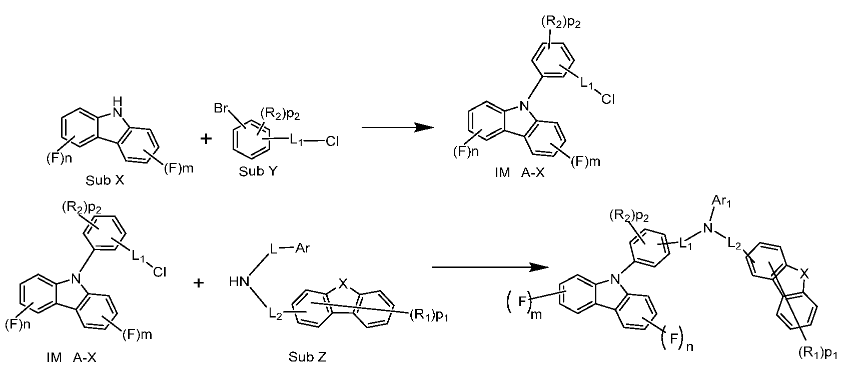 [SMILES: Fc1ccc2c(c1)[nH]c1ccccc12]

Raw materials Sub X, Sub Y and Sub Z can be commercially obtained or can be obtained by methods well known in the art, and specific preparation methods are well known in the art, which will not be repeated here.

### 1. Synthesis of Intermediates IM A-X

### 1) The Synthesis of IM A-X Was Illustrated by Taking IM A-1 as an Example

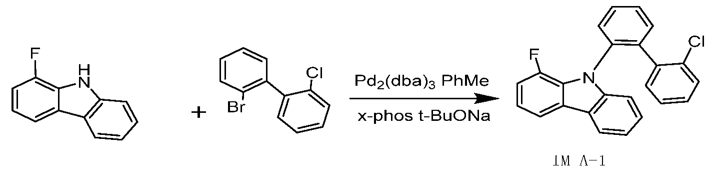 [SMILES: Clc1ccccc1-c1ccccc1Br]

1-fluoro-9H-carbazole (4.63 g, 25.00 mmol), 2-bromo-2′-chloro-1,1′-biphenyl (6.68 g, 25.00 mmol), tris(dibenzylideneacetone)dipalladium (0.23 g, 0.25 mmol), 2-dicyclohexylphosphino-2‘,4′,6′-triisopropylbiphenyl (0.24 g, 0.50 mmol) and sodium tert-butoxide (4.8 g, 50.0 mmol) were added to toluene (50 mL), and the mixture was heated to 108° C. under nitrogen protection, and stirred for 3h for reaction; after the reaction and cooling down to room temperature, the resulting reaction solution was washed with water, dried over magnesium sulfate, and then filtered out the magnesium sulfate to obtain filtrate, and a solvent was removed from the obtained filtrate under reduced pressure to obtain a crude product; and the crude product was purified by recrystallization using toluene (1 g of the crude product:8 mL of toluene) to gain a white intermediate IM A-1 (4.83 g, yield: 52%).

2) Intermediates IM A-X listed in Table 1 were synthesized with reference to the synthesis method for IM A-1 except that a raw material Sub X was used to instead of 1-fluoro-9H-carbazole, and a raw material Sub Y was used to instead of 2-bromo-2′-chloro-1,1′-biphenyl, and the used main raw materials, the synthesized intermediates and their yields are as shown in Table 1.

TABLE 1

| Sub X | Sub Y | IM A-X | Yield/% |
|---|---|---|---|
| 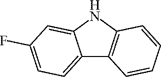 | 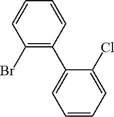 [SMILES: Fc1ccc2c(c1)[nH]c1ccccc12] | 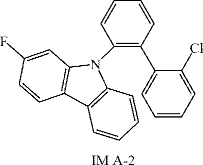 | 67 |
| 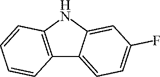 | 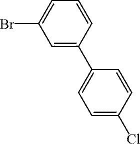 [SMILES: Fc1ccc2[nH]c3ccccc3c2c1] | 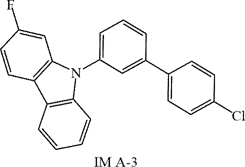 | 70 |
| 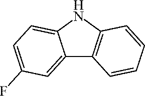 | 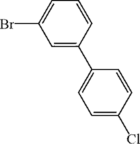 [SMILES: Fc1cccc2[nH]c3ccccc3c12] | 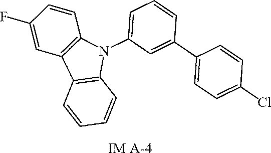 | 71 |
| 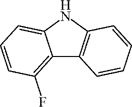 | 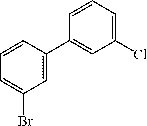 [SMILES: Fc1cccc2c1[nH]c1ccccc12] | 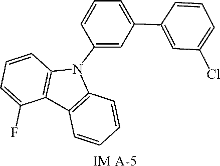 | 69 |
| 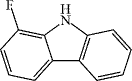 | 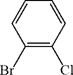 [SMILES: Fc1cccc2c1[nH]c1ccccc12] | 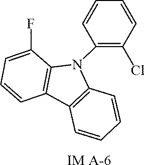 | 67 |
| 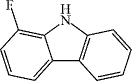 | 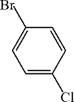 [SMILES: Fc1ccc2c(c1)[nH]c1ccccc12] | 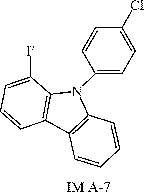 | 68 |
| 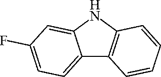 | 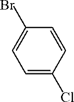 [SMILES: Fc1cccc2[nH]c3ccccc3c12] | 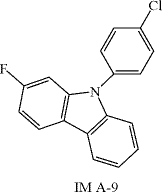 | 75 |
| 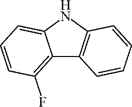 | 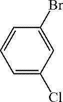 [SMILES: Fc1ccc2c(c1)[nH]c1ccccc12] | 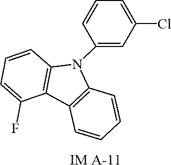 | 69 |
| 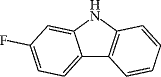 | 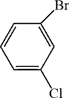 [SMILES: Fc1ccc2c(c1)[nH]c1ccccc12] | 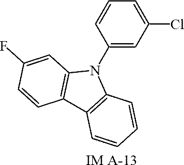 | 73 |
| 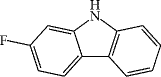 | 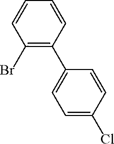 [SMILES: Fc1ccc2[nH]c3ccccc3c2c1] | 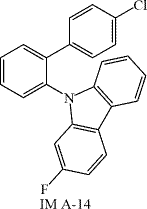 | 70 |
| 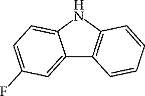 | 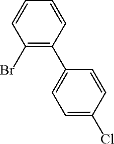 [SMILES: Fc1cccc2[nH]c3ccccc3c12] | 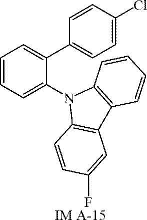 | 71 |
| 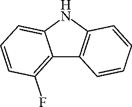 | 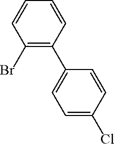 | 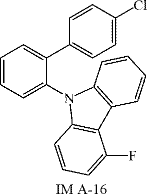 | 71 |

### 2. Synthesis of Compounds

### Synthesis Example 1: Synthesis of Compound 1

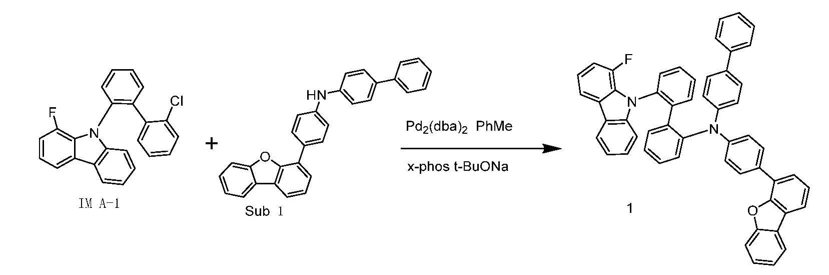

The intermediate IM A-1 (18.6 g, 50.00 mmol), a raw material Sub 1 (20.6 g, 50.00 mmol), tris(dibenzylideneacetone)dipalladium (0.46 g, 0.50 mmol), 2-dicyclohexylphosphino-2′,4′,6′-triisopropylbiphenyl (0.48 g, 1.00 mmol) and sodium tert-butoxide (9.61 g, 100.00 mmol) were added to toluene (150 mL), and the mixture was heated to 105° C. under nitrogen protection, and stirred for 4h; after stirred and cooled down to room temperature to obtain a resulting reaction solution, the resulting reaction solution was washed with water, dried over magnesium sulfate and filtered to obtain filtrate, and a solvent was removed from the obtained filtrate under reduced pressure to obtain a crude product; and a crude product was purified by recrystallization using toluene (1 g of the crude product: 10 mL of toluene) to gain a compound 1 (20.9 g, yield: 56%) as a white solid, mass spectrum: m/z =747.3 [M+H]⁺.

### Synthesis Examples 2 to 22

Compounds listed in Table 2 were synthesized with reference to the method in Synthesis example 1 except that intermediates IM A-X were used to instead of IM A-1, and a raw material Sub Z was used to instead of Sub 1, and the used main raw materials as well as the corresponding synthesized compounds, the compound yields and mass spectrometry characterization are as shown in Table 2.

TABLE 2

| Synthesis example | IM A-X | Sub Z | Compound | Yield /% | Mass spectrum (m/z), [M+H]⁺ |
|---|---|---|---|---|---|
| 2 | 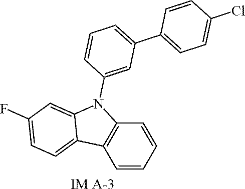 | 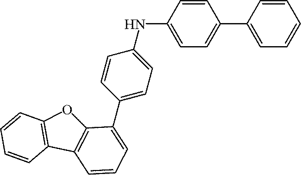 [SMILES: c1ccc(-c2ccc(Nc3ccc(-c4cccc5c4oc4ccccc45)cc3)cc2)cc1] | 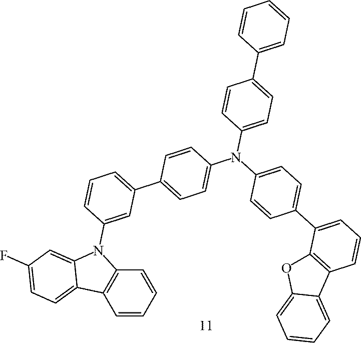 | 70 | 747.3 |
| 3 | 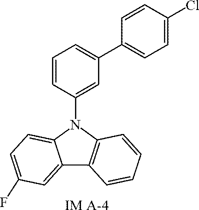 | 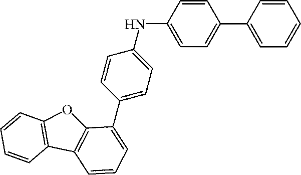 [SMILES: c1ccc(-c2ccc(Nc3ccc(-c4cccc5c4sc4ccccc45)cc3)cc2)cc1] | 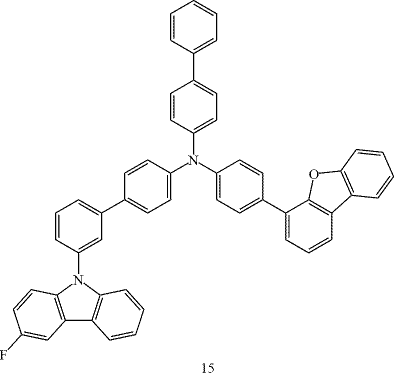 [SMILES: Fc1cccc2c1c1ccccc1n2-c1cccc(-c2cccc(N(c3ccc(-c4ccccc4)cc3)c3ccc(-c4cccc5c4sc4ccccc45)cc3)c2)c1 ; "21"] | 71 | 747.3 |
| 4 | 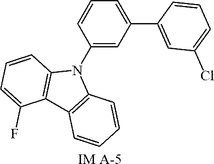 | 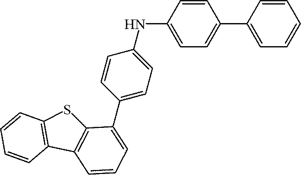 [SMILES: c1ccc2c(Nc3ccc(-c4cccc5c4oc4ccccc45)cc3)cccc2c1] | 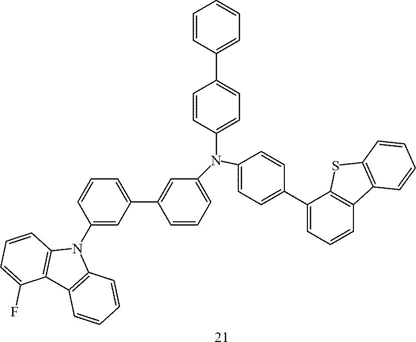 | 68 | 763.2 |
| 5 | 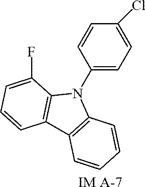 | 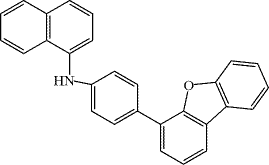 [SMILES: c1ccc2c(-c3ccc(Nc4ccc(-c5cccc6c5sc5ccccc56)cc4)cc3)cccc2c1] | 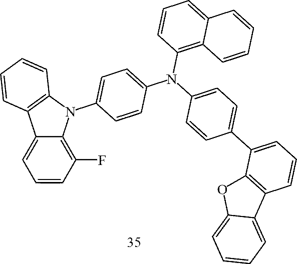 | 71 | 645.2 |
| 6 | 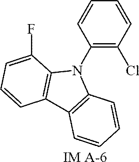 | 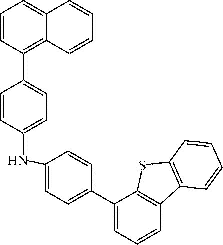 [SMILES: c1ccc2cc(-c3ccc(Nc4ccc(-c5cccc6c5oc5ccccc56)cc4)cc3)ccc2c1] | 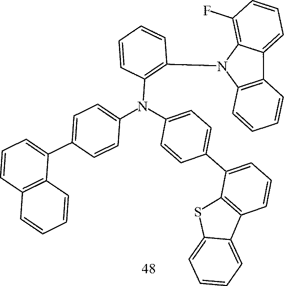 | 47 | 737.2 |
| 7 | 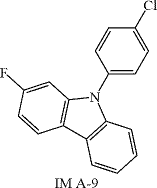 | 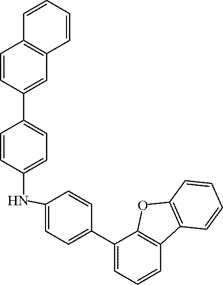 [SMILES: c1ccc(-c2ccc(Nc3ccc(-c4cccc5c4sc4c(-c6ccccc6)cccc45)cc3)cc2)cc1] | 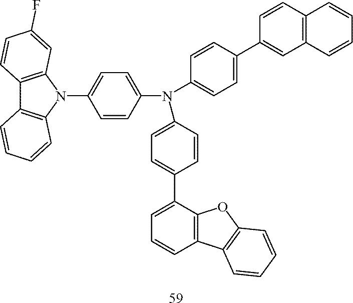 | 72 | 721.3 |
| 8 | 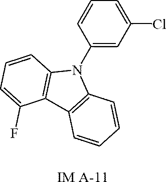 | 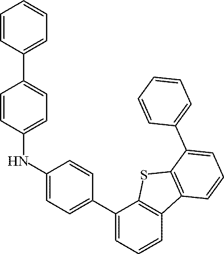 [SMILES: c1ccc(-c2cccc(Nc3cccc(-c4cccc5c4oc4ccccc45)c3)c2)cc1] | 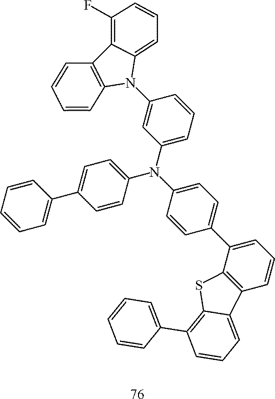 | 66 | 763.3 |
| 9 | 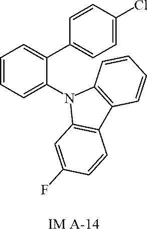 | 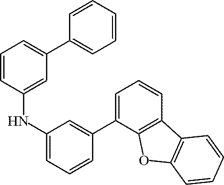 [SMILES: c1ccc(-c2ccc(Nc3cccc4oc5ccccc5c34)cc2)cc1] | 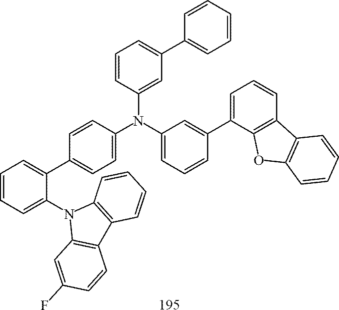 | 69 | 747.3 |
| 10 | 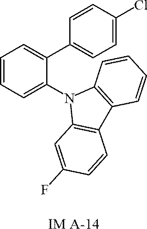 | 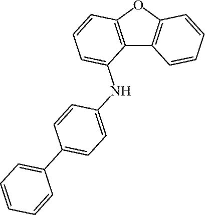 [SMILES: c1ccc(-c2cccc3cccc(-c4ccc(Nc5ccc(-c6cccc7c6sc6ccccc67)cc5)cc4)c23)cc1] | 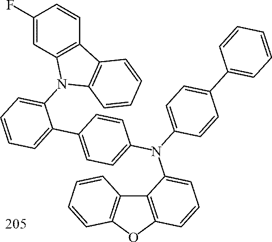 | 64 | 671.2 |
| 11 | 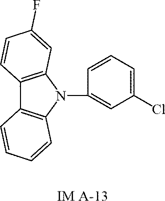 | 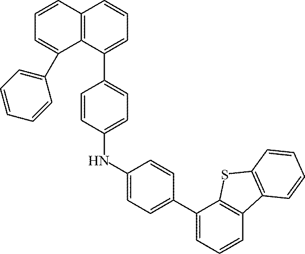 [SMILES: c1ccc(-c2ccc(-c3cccc4ccccc34)cc2)c(Nc2ccc(-c3cccc4c3oc3ccccc34)cc2)c1] | 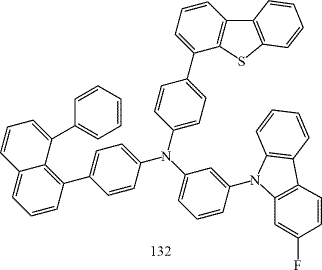 | 45 | 813.3 |
| 12 | 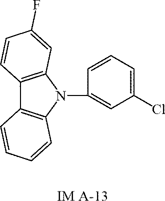 | 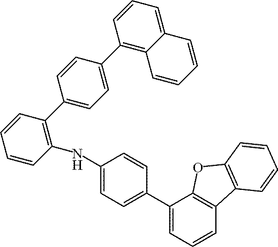 [SMILES: c1ccc(-c2ccccc2Nc2ccc(-c3cccc4sc5ccccc5c34)cc2)cc1] | 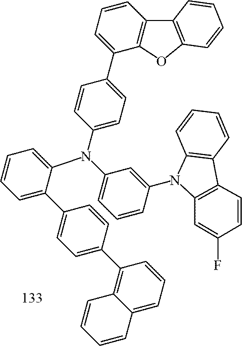 | 46 | 797.3 |
| 13 | 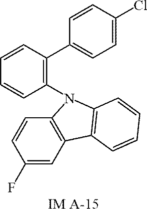 | 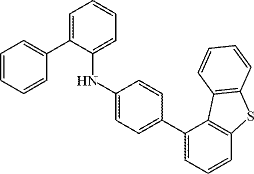 [SMILES: c1ccc(-c2ccc(Nc3ccc(-c4cccc5oc6ccccc6c45)cc3)cc2)cc1] | 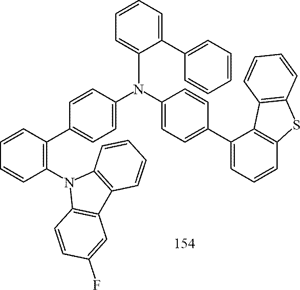 | 45 | 763.3 |
| 14 | 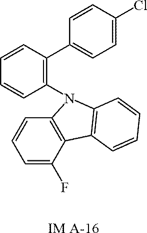 | 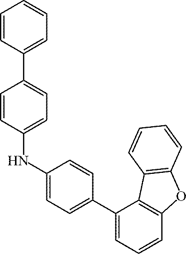 [SMILES: c1ccc(-c2ccccc2Nc2ccc(-c3cccc4c3sc3ccccc34)cc2)cc1] | 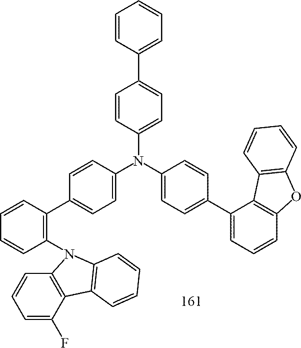 | 44 | 747.3 |
| 15 | 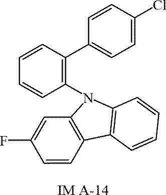 | 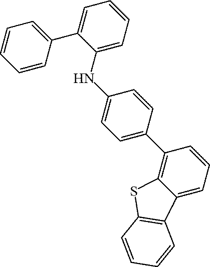 [SMILES: c1ccc(-c2ccccc2Nc2ccc(-c3ccc4c(c3)oc3ccccc34)cc2)cc1] | 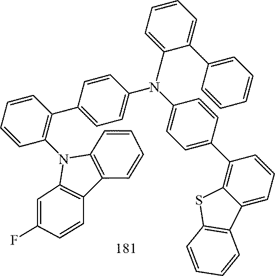 | 43 | 763.3 |
| 16 | 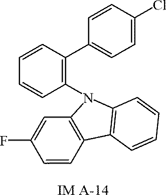 | 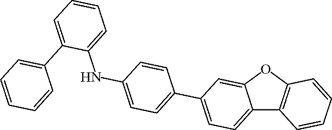 [SMILES: c1ccc(-c2cccc(Nc3ccc(-c4ccc5sc6ccccc6c5c4)cc3)c2)cc1] | 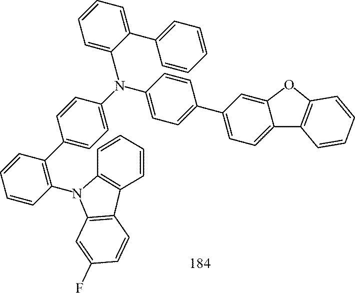 | 45 | 747.3 |
| 17 | 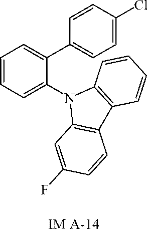 | 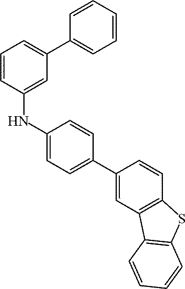 [SMILES: c1ccc(-n2c3ccccc3c3ccc(Nc4ccc(-c5cccc6c5oc5ccccc56)cc4)cc32)cc1] | 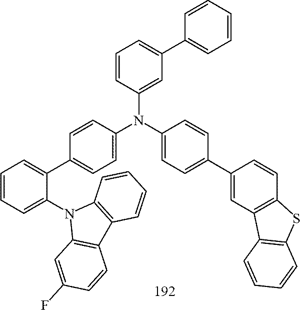 | 63 | 763.3 |
| 18 | 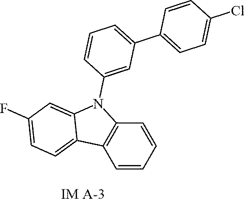 | 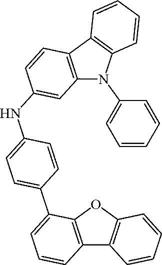 [SMILES: c1ccc(-c2ccc(Nc3cccc4c3oc3c(-c5ccccc5)cccc34)cc2)cc1] | 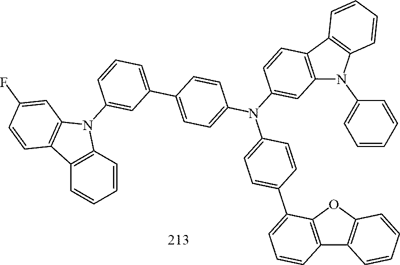 | 70 | 836.3 |
| 19 | 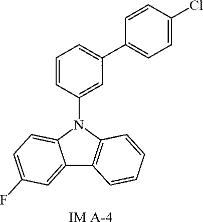 | 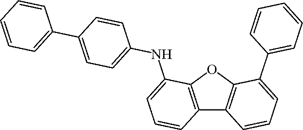 [SMILES: c1ccc(-c2ccccc2Nc2cccc3oc4c(-c5ccccc5)cccc4c23)cc1] | 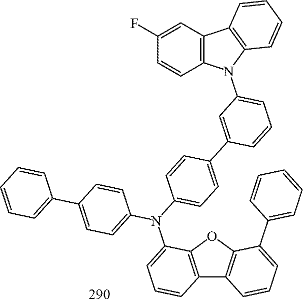 | 64 | 747.3 |
| 20 | 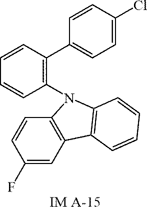 | 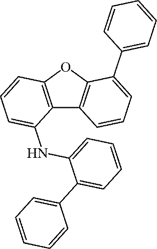 [SMILES: CC1(C)c2ccccc2-c2ccc(Nc3ccc(-c4cccc5oc6ccccc6c45)cc3)cc21] | 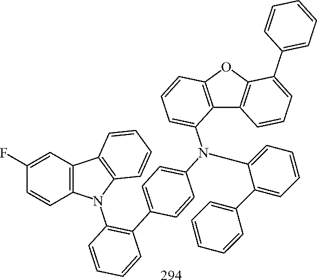 | 68 | 747.3 |
| 21 | 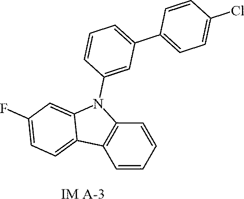 | 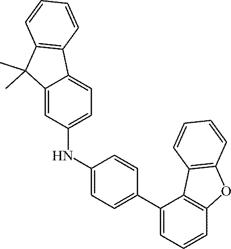 [SMILES: c1ccc2c(c1)oc1c(-c3ccc(Nc4ccc5sc6ccccc6c5c4)cc3)cccc12] | 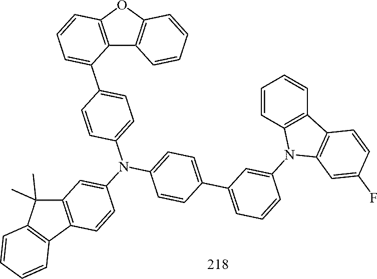 | 62 | 787.3 |
| 22 | 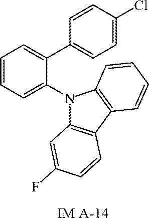 | 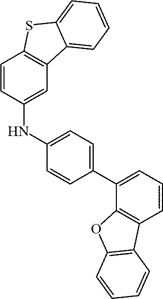 [SMILES: Clc1cccc(-c2cccc(Br)c2)c1] | 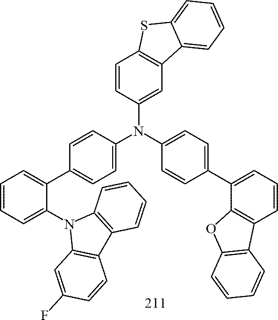 | 60 | 777.2 |

NMR data for a compound 11: ¹H NMR (600 MHz, CD₂Cl₂), 8.12-8.80 (m, 2H), 8.03 (d, 1H), 7.95 (d, 1H), 7.90 (d, 2H), 7.81 (s, 1H), 7.76 (d, 1H), 7.70 (t, 1H), 7.65-7.61 (m, 6H), 7.59 (d, 2H), 7.52-7.37 (m. 8H), 7.34-7.29 (m, 8H), 7.16 (d, 1H), 7.04 (t, 1H); and
NMR data for a compound 15: ¹H NMR (600 MHz, CD₂Cl₂), 8.12 (d, 1H), 8.03 (d, 1H), 7.95 (d, 1H), 7.90 (d, 2H), 7.83-7.81 (m, 2H), 7.75 (d, 1H), 7.70 (t, 1H), 7.64-7.61 (m, 6H), 7.59 (d, 2H), 7.53-7.42 (m, 8H), 7.39 (t, 1H), 7.34-7.29 (m, 8H), 7.18 (t, 1H).

### 3. Synthesis of Intermediates IM A-X (X>16)

### 1) Synthesis of IM A-X (X>16) Was Illustrated by Taking IM A-17 as an Example

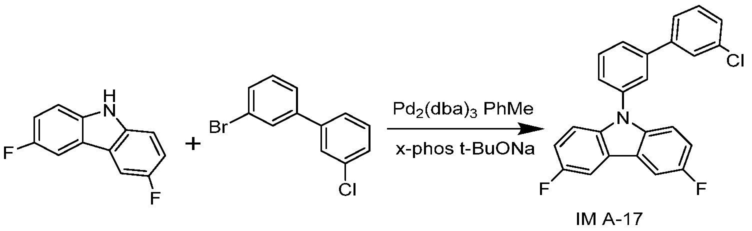 [SMILES: Fc1cc(F)c2[nH]c3ccccc3c2c1]

3,6-difluoro-9H-carbazole (4.06 g, 20.00 mmol), 3′-chloro-3-bromobiphenyl (5.35 g, 20.00 mmol), tris(dibenzylideneacetone)dipalladium (0.18 g, 0.20 mmol), 2-dicyclohexylphosphino-2′,4′,6′-triisopropylbiphenyl (0.19 g, 0.40 mmol) and sodium tert-butoxide (3.84 g, 40.0 mmol) were added to toluene (50 mL), and the mixture was heated to 108° C. under nitrogen protection, and stirred for 3h; after stirred and cooled down to room temperature, the resulting reaction solution was washed with water, dried over magnesium sulfate, and filtered to obtain filtrate, and a solvent was removed from the obtained filtrate under reduced pressure to obtain a crude product; and a crude product was purified by recrystallization using toluene (1 g: 6 mL of toluene) to gain an intermediate IM A-17 (5.13 g, yield: 66%) as a white solid.

2) Intermediates IM A-X listed in Table 3 were synthesized with reference to the method for IM A-17 except that a raw material Sub X was used to instead of 3,6-difluoro-9H-carbazole, and a raw material Sub Y was used to instead of 3′-chloro-3-bromobiphenyl, and the used main raw materials, the synthesized intermediates and their yields are as shown in Table 3.

TABLE 3

| Sub X | Sub Y | 1 M A-X | Yield/% |
|---|---|---|---|
| 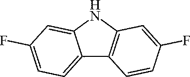 [SMILES: Clc1cccc(-c2cccc(Br)c2)c1] | 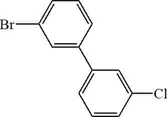 | 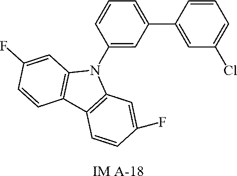 [SMILES: Fc1cc(F)c2c(c1)[nH]c1ccccc12] | 70 |
| 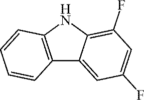 [SMILES: Clc1ccc(-c2cccc(Br)c2)cc1] | 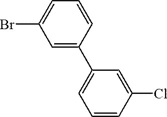 | 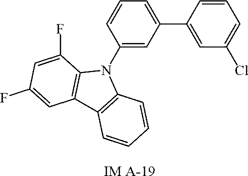 [SMILES: Fc1ccc2c(c1)[nH]c1ccc(F)cc12] | 62 |
| 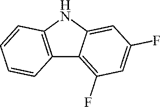 [SMILES: Clc1ccc(-c2ccccc2Br)cc1] | 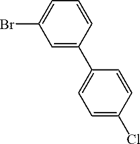 | 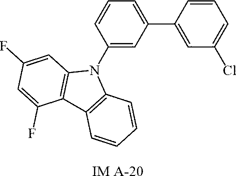 [SMILES: Fc1cc(F)c2c(c1)[nH]c1ccccc12] | 64 |
| 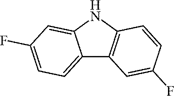 [SMILES: Clc1ccc(-c2ccccc2Br)cc1] | 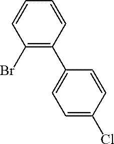 | 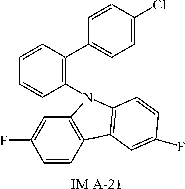 | 67 |
| 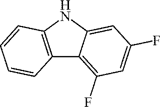 | 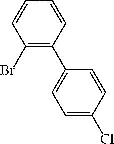 [SMILES: c1ccc(-c2ccc(Nc3ccc(-c4cccc5c4sc4ccccc45)cc3)cc2)cc1] | 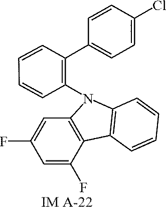 | 63 |

### 4. Synthesis of Compounds

### Synthetic Example 23: Synthesis of Compound 121

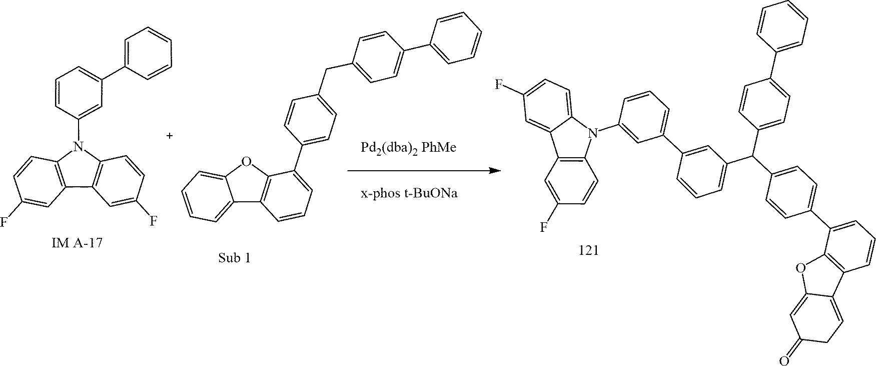

The intermediate IM A-17 (9.74 g, 25.00 mmol), a raw material Sub 1 (10.28 g, 25.00 mmol), tris(dibenzylideneacetone)dipalladium (0.23 g, 0.25 mmol), 2-dicyclohexylphosphino-2′,4′,6′-triisopropylbiphenyl (0.24 g, 0.50 mmol) and sodium tert-butoxide (4.8 g, 50.0 mmol) were added to toluene (150 mL), and the mixture was heated to 108° C. under nitrogen protection, and stirred for 3h; after stirred and cooled down to room temperature to obtain a resulting reaction solution, the resulting reaction solution was washed with water, dried over magnesium sulfate and filtered to obtain filtrate, and a solvent was removed from the obtained filtrate under reduced pressure to obtain a crude product; and a crude product was purified by recrystallization using toluene (1 g of the crude product: 15 mL of toluene) to gain a compound 121 (9.57 g, yield: 50%) as white solid, mass spectrum: m/z=765.3 [M+H]⁺.

### Synthesis Examples 24 to 28

Compounds listed in Table 4 were synthesized with reference to the method in Synthesis example 23 except that intermediates IM A-X were used to instead of the intermediate IM A-17, and a raw material Sub Z was used to instead of Sub 1, and the used main raw materials as well as the corresponding synthesized compounds, the compound yields and mass spectrometry characterization are as shown in Table 4.

TABLE 4

| Synthesis example | IM A-X | Sub Z | Compound | Yield/% | Mass spectrum (m/z), [M+H]⁺ |
|---|---|---|---|---|---|
| 24 | 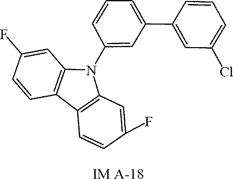 | 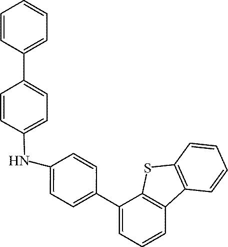 | 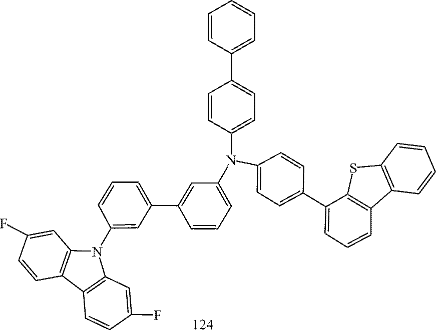 | 62 | 781.2 |
| 25 | 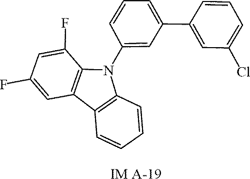 | 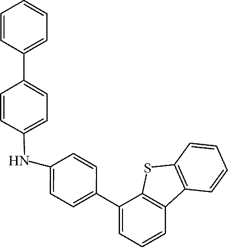 | 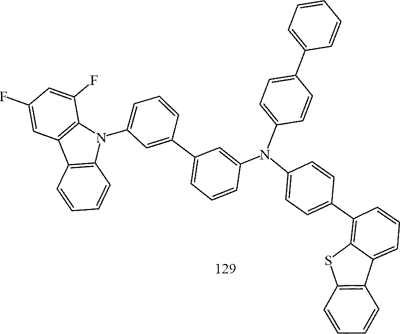 | 48 | 781.2 |
| 26 | 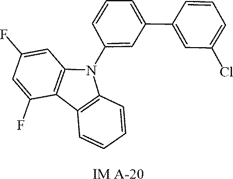 | 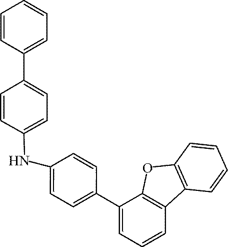 | 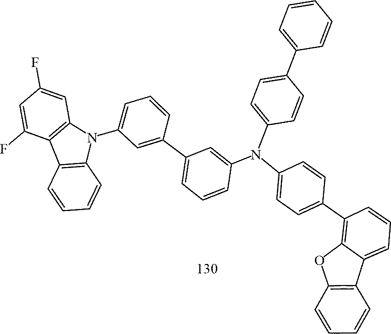 | 46 | 765.3 |
| 27 | 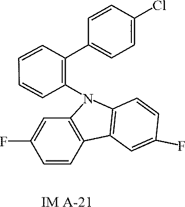 | 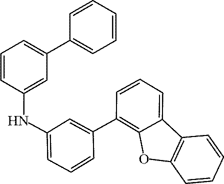 | 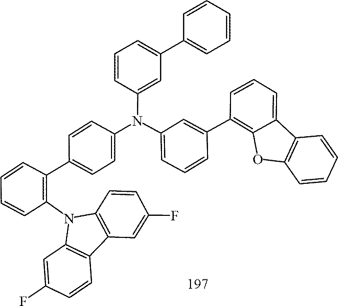 | 52 | 765.3 |
| 28 | 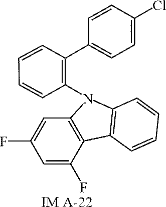 | 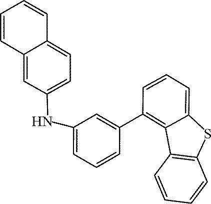 | 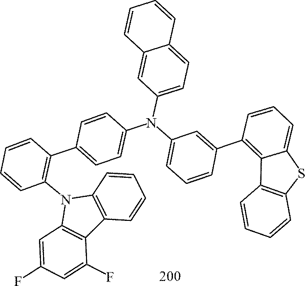 | 55 | 755.2 |

### Example 1: Blue Organic Electroluminescent Device

An anode was prepared by the following process: an ITO substrate (manufactured by Coming) with thickness of 1500 Å was cut into a dimension of 40 mm×40 mm×0.7 mm, after that continued to be prepared into an experimental substrate which includes a cathode, an anode and an insulating layer pattern by adopting a photoetching process, and for the experimental substrate surface treatment was performed by utilizing ultraviolet ozone and O₂:N₂ plasma so as to increase the work function of the anode (the experimental substrate) and remove scum.

F4-TCNQ was vacuum-evaporated on the experimental substrate (the anode) to form a hole injection layer (HIL) having a thickness of 100 Å, and NPB was evaporated on the hole injection layer to form a first hole transport layer having a thickness of 1150 Å.

A compound 1 was vacuum-evaporated on the first hole transport layer to form an electron blocking layer (EBL) having a thickness of 100 Å.

α,β-AND was used as a host and was doped simultaneously with BD-1 at a film thickness ratio of 100:3 on the electron blocking layer to form a light-emitting layer (EML) having a thickness of 200 Å.

TPBi and LiQ were co-evaporated at a film thickness ratio of 1:1 to form an electron transport layer (ETL) having a thickness of 300 Å, LiQ was evaporated on the electron transport layer to form an electron injection layer (EIL) having a thickness of 10 Å, and then magnesium (Mg) and silver (Ag) were mixed and vacuum-evaporated on the electron injection layer at an evaporation rate of 1:9 to form a cathode having a thickness of 115 Å.

In addition, CP-1 having a thickness of 700 Å was evaporated on the cathode to form an organic capping layer (CPL), thus completing the manufacture of an organic light-emitting device.

### Examples 2 to 28

An organic electroluminescent device was manufactured by the same method as that in Example 1 except that compounds shown in the following Table 5 were used to instead of the compound 1 when the electron blocking layer was formed.

### Comparative Examples 1 to 4

An organic electroluminescent device was manufactured by the same method as that in Example 1 except that a compound A, a compound B, a compound C, and a compound D were respectively used to instead of the compound 1 when the electron blocking layer was formed.

The structures of main materials used in the above examples and comparative examples are shown below:

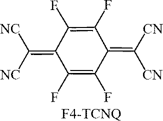

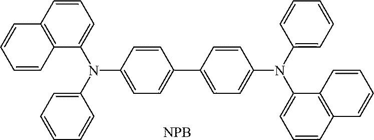

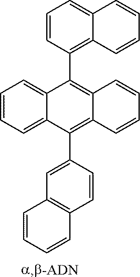 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; "CP-1"]

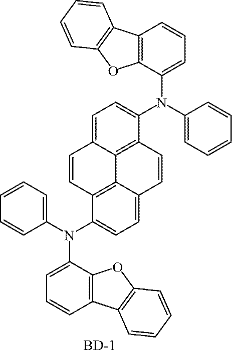

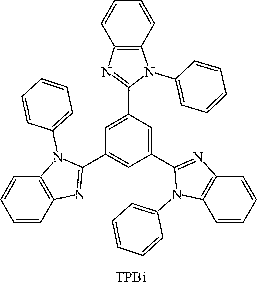

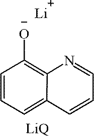

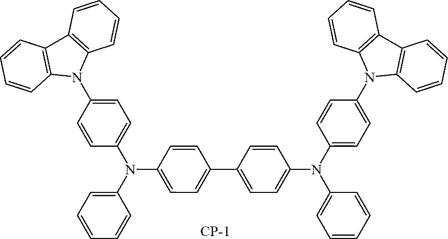

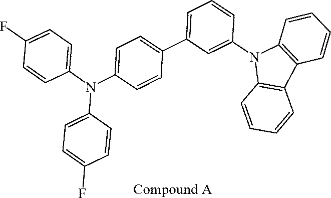

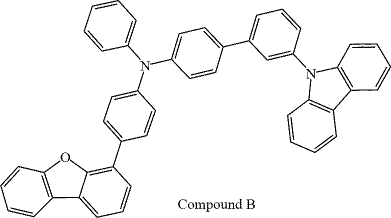

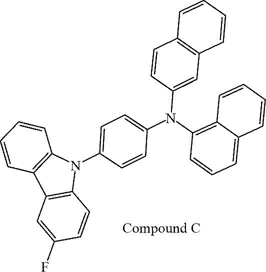

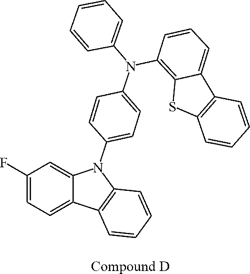

For the manufactured organic electroluminescent devices, the performance of the device was analyzed under a condition of 20 mA/cm², and the results are shown in the following Table 5:

TABLE 5

| Example | EBL | Volt (V) | Cd/A | lm/W | CIE-x | CIE-y | EQE% | T95 (hrs)@20 mA/cm² |
|---|---|---|---|---|---|---|---|---|
| Example 1 | Compound 1 | 3.97 | 6.74 | 5.33 | 0.14 | 0.05 | 13.86 | 203 |
| Example 2 | Compound 11 | 3.83 | 6.51 | 5.26 | 0.14 | 0.05 | 13.39 | 256 |
| Example 3 | Compound 15 | 3.85 | 6.62 | 5.23 | 0.14 | 0.05 | 13.62 | 252 |
| Example 4 | Compound 21 | 3.96 | 6.65 | 5.28 | 0.14 | 0.05 | 13.68 | 205 |
| Example 5 | Compound 35 | 3.89 | 6.49 | 5.24 | 0.14 | 0.05 | 13.35 | 193 |
| Example 6 | Compound 59 | 3.95 | 6.59 | 5.24 | 0.14 | 0.05 | 13.56 | 201 |
| Example 7 | Compound 76 | 3.93 | 6.61 | 5.28 | 0.14 | 0.05 | 13.62 | 211 |
| Example 8 | Compound 205 | 3.89 | 6.67 | 5.37 | 0.14 | 0.05 | 13.72 | 233 |
| Example 9 | Compound 132 | 3.98 | 6.58 | 5.19 | 0.14 | 0.05 | 13.54 | 206 |
| Example 10 | Compound 154 | 3.94 | 6.49 | 5.15 | 0.14 | 0.05 | 13.35 | 230 |
| Example 11 | Compound 161 | 3.86 | 6.58 | 5.27 | 0.14 | 0.05 | 13.54 | 251 |
| Example 12 | Compound 121 | 3.95 | 6.62 | 5.26 | 0.14 | 0.05 | 13.62 | 203 |
| Example 13 | Compound 129 | 3.94 | 6.54 | 5.21 | 0.14 | 0.05 | 13.45 | 209 |
| Example 14 | Compound 192 | 3.88 | 6.49 | 5.24 | 0.14 | 0.05 | 13.35 | 225 |
| Example 15 | Compound 197 | 3.92 | 6.61 | 5.22 | 0.14 | 0.05 | 13.59 | 235 |
| Example 16 | Compound 213 | 3.89 | 6.63 | 5.31 | 0.14 | 0.05 | 13.64 | 230 |
| Example 17 | Compound 48 | 3.92 | 6.51 | 5.21 | 0.14 | 0.05 | 13.37 | 212 |
| Example 18 | Compound 124 | 3.93 | 6.68 | 5.36 | 0.14 | 0.05 | 13.78 | 217 |
| Example 19 | Compound 130 | 3.91 | 6.52 | 5.23 | 0.14 | 0.05 | 13.39 | 208 |
| Example 20 | Compound 133 | 3.89 | 6.68 | 5.39 | 0.14 | 0.05 | 13.74 | 213 |
| Example 21 | Compound 181 | 3.90 | 6.66 | 5.34 | 0.14 | 0.05 | 13.64 | 232 |
| Example 22 | Compound 184 | 3.88 | 6.63 | 5.31 | 0.14 | 0.05 | 13.49 | 224 |
| Example 23 | Compound 195 | 3.85 | 6.60 | 5.36 | 0.14 | 0.05 | 13.56 | 241 |
| Example 24 | Compound 200 | 3.89 | 6.53 | 5.25 | 0.14 | 0.05 | 13.40 | 231 |
| Example 25 | Compound 290 | 3.92 | 6.79 | 5.41 | 0.14 | 0.05 | 13.88 | 240 |
| Example 26 | Compound 294 | 3.98 | 6.75 | 5.30 | 0.14 | 0.05 | 13.82 | 237 |
| Example 27 | Compound 218 | 3.83 | 6.68 | 5.45 | 0.14 | 0.05 | 13.68 | 232 |
| Example 28 | Compound 211 | 3.86 | 6.72 | 5.48 | 0.14 | 0.05 | 13.78 | 235 |
| Comparative example 1 | Compound A | 3.97 | 5.94 | 4.74 | 0.14 | 0.05 | 12.32 | 154 |
| Comparative example 2 | Compound B | 3.91 | 5.95 | 4.75 | 0.14 | 0.05 | 12.29 | 157 |
| Comparative example 3 | Compound C | 3.89 | 6.12 | 4.83 | 0.14 | 0.05 | 12.42 | 160 |
| Comparative example 4 | Compound D | 3.93 | 6.05 | 4.78 | 0.14 | 0.05 | 12.18 | 156 |

From the results of Table 5, it can be seen that by comparing Examples 1 to 28 in which the compounds were used as the electron blocking layer (also referred to as the “second hole transport layer”) with Comparative examples 1-4 using the well-known compounds A, B, C and D, for the organic electroluminescent devices manufactured by using the compounds of the present disclosure as the electron blocking layer, the current efficiency (Cd/A) is improved by at least 6.05%, the power efficiency (lm/W) is improved by at least 6.63%, the external quantum efficiency (EQE) is improved by at least 7.49%, and the service life (T95) is improved by at least 20.63%; and at the same time, the organic electroluminescent devices manufactured in Examples 1-28 also have a lower driving voltage.

In addition, by comparing Examples 1 to 28 with Comparative examples 1 to 4, it can be seen that according to the compounds of the present disclosure, one or two fluorines are introduced on carbazolyl as a substituent, so that the service life and luminous efficiency of the device can be improved, and the reason may be that the fluorines can efficiently attract electrons, so that the fluorine atom prevents the electrons of a host material in the organic light-emitting layer which is adjacent to the electron blocking layer from “escaping”; at the same time, fluorine is introduced onto carbazolyl, so that the fluorine atom can be interacted with the dibenzofuran/dibenzothiophene group on arylamine, and at least one of the two groups N-phenylcarbazolyl and dibenzofuranyl/dibenzothienyl is controlled to be connected to the nitrogen atom via the aromatic group, as a result, the distortion degree of the whole molecule can be improved, and the whole molecule has a better configuration; while the molecular weight of the compound D in Comparative Example 4 is too small, in that the nitrogen atom is directly adjacent to N-phenylcarbazolyl and dibenzothiophene/dibenzofuran which are bonded to the fluorine(s), so that the entire molecular structure is too flat, and the molecular weight is too low, making the compound D unable to act effectively in the device. In addition, in examples 2, 3, 11, 15, etc., the corresponding Li is controlled to have a specific structure, and the obtained compounds have higher thermal stability, in addition, the fluorine on the carbazole group and the other two substituents on the arylamine have higher compatibility, thus the role of each group is fully reflected, based on which can more effectively reduce the transport rate of electrons, block the electrons from passing through the device, and significantly improve the service life of the device.

Preferred examples of the present disclosure have been described above in detail with reference to the accompanying drawings, but the present disclosure is not limited to the specific details in the above-described examples, and many simple modifications may be made to the technical solutions of the present disclosure within the technical idea of the present disclosure, and these simple modifications are within the scope of protection of the present disclosure.

What is claimed is:

1. A nitrogen-containing compound, having a structure as shown in formula 1: 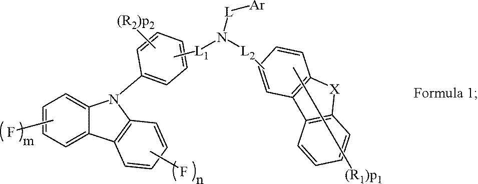
wherein X is selected from O or S;
Ar is selected from a substituted or unsubstituted aryl group with 6 to 30 carbon atoms, and substituted or a unsubstituted heteroaryl group with 3 to 30 carbon atoms;
L, L₁ and L₂ are the same or different, and are each independently selected from a single bond, a substituted or unsubstituted arylene group with 6 to 25 carbon atoms, and substituted or a unsubstituted heteroarylene group with 3 to 25 carbon atoms, and L₁ and L₂ are not simultaneously a single bond;
m is 0, 1 or 2, n is 0, 1 or 2, and 1≤m+n ≤2;
substituents in Ar, L, L₁ and L₂, as well as R₁ and R₂, are the same or different, and are each independently selected from: deuterium, tritium, a halogen group, cyano, aryl with 6 to 18 carbon atoms, heteroaryl with 3 to 18 carbon atoms, trialkylsilyl with 3 to 12 carbon atoms, alkyl with 1 to 10 carbon atoms, haloalkyl with 1 to 10 carbon atoms, alkenyl with 2 to 10 carbon atoms, cycloalkyl with 3 to 10 carbon atoms, alkylthio with 1 to 10 carbon atoms, alkoxy with 1 to 10 carbon atoms, aryloxy with 6 to 18 carbon atoms, arylthio with 6 to 18 carbon atoms, and triphenylsilyl;
pi represents the number of R₁, and is selected from 0, 1, 2, 3, 4, 5, 6 or 7; in response to determining that pi is greater than 1, any two R₁ are the same or different; and optionally, any two adjacent R₁ form a ring; and
p₂ represents the number of R₂, and is selected from 0, 1, 2, 3 or 4; in response to determining that p₂ is greater than 1, any two R₂ are the same or different; and optionally, any two adjacent R₂ form a ring.

2. The nitrogen-containing compound according to claim 1, wherein the nitrogen-containing compound has a structure as shown in formula 1-1 or formula 1-2: 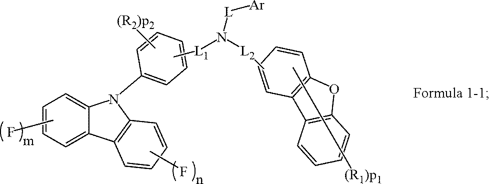 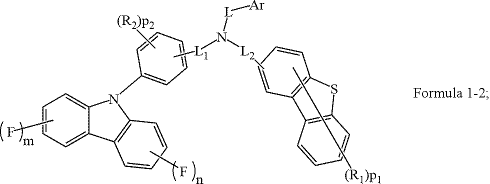
and in the formula 1-2, L₂ is selected from a substituted or unsubstituted arylene group with 6 to 25 carbon atoms, and a substituted or unsubstituted heteroarylene group with 3 to 25 carbon atoms.

3. The nitrogen-containing compound according to claim 1, wherein R₁ and R₂ are the same or different, and are each independently selected from deuterium, fluorine, cyano, aryl with 6 to 15 carbon atoms, heteroaryl with 5 to 12 carbon atoms, trialkylsilyl with 3 to 7 carbon atoms, alkyl with 1 to 4 carbon atoms, fluoroalkyl with 1 to 4 carbon atoms, cycloalkyl with 5 to 10 carbon atoms, alkylthio with 1 to 4 carbon atoms, alkoxy with 1 to 4 carbon atoms, aryloxy with 6 to 12 carbon atoms, and arylthio with 6 to 12 carbon atoms.

4. The nitrogen-containing compound according to claim 1, wherein Ar is selected from a substituted or unsubstituted aryl group with 6 to 25 carbon atoms, and a substituted or unsubstituted heteroaryl group with 5 to 24 carbon atoms; and
substituents in Ar are each independently selected from: deuterium, tritium, fluorine, cyano, aryl with 6 to 15 carbon atoms, heteroaryl with 3 to 12 carbon atoms, trialkylsilyl with 3 to 7 carbon atoms, alkyl with 1 to 4 carbon atoms, fluoroalkyl with 1 to 4 carbon atoms, cycloalkyl with 5 to 10 carbon atoms, alkylthio with 1 to 4 carbon atoms, alkoxy with 1 to 4 carbon atoms, aryloxy with 6 to 12 carbon atoms, arylthio with 6 to 12 carbon atoms, and triphenylsilyl.

5. The nitrogen-containing compound according to claim 1, wherein Ar is selected from a substituted or unsubstituted group V₁, and the unsubstituted group V₁ is selected from the group consisting of: 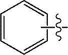 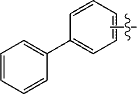 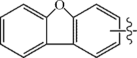 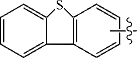 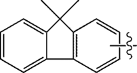 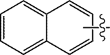 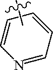 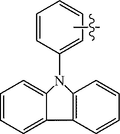 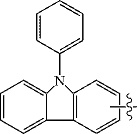 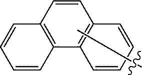 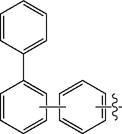 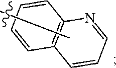
and
the substituted group V₁ has one or two or more substituents, and the substituents are respectively and independently selected from deuterium, fluorine, cyano, alkyl with 1 to 4 carbon atoms, fluoroalkyl with 1 to 4 carbon atoms, cycloalkyl with 5 to 10 carbon atoms, trialkylsilyl with 3 to 7 carbon atoms, phenyl, naphthyl, pyridyl, and triphenylsilyl; and when the number of the substituents is greater than 1, the substituents are the same or different; and optionally, any two adjacent substituents form a ring.

6. The nitrogen-containing compound according to claim 1, wherein Ar is selected from the group consisting of: 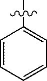 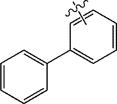 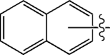 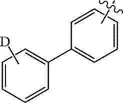 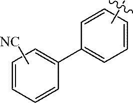 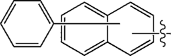 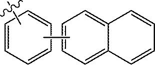 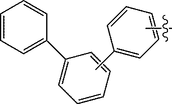 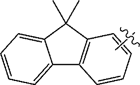 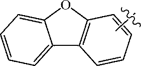 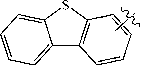 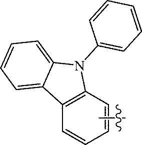 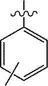 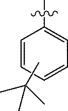 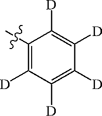 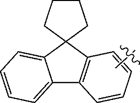 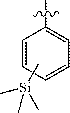 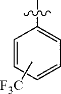 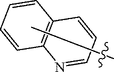 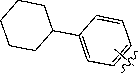 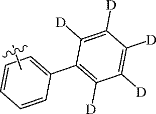 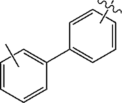 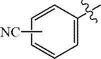 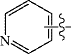 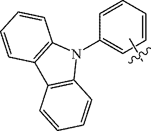
.

7. The nitrogen-containing compound according to claim 1, wherein L, L₁ and L₂ are the same or different, and are each independently selected from a single bond, a substituted or unsubstituted arylene group with 6 to 20 carbon atoms, and a substituted or unsubstituted heteroarylene group with 5 to 20 carbon atoms; and
substituents in L, L₁ and L₂ are each independently selected from deuterium, fluorine, cyano, alkyl with 1 to 4 carbon atoms, cyclopentyl, cyclohexyl, fluoroalkyl with 1 to 4 carbon atoms, methoxy, trimethylsilyl, triethylsilyl, and phenyl.

8. The nitrogen-containing compound according to claim 1, wherein L, L₁ and L₂ are the same or different, and are each independently selected from a single bond, and a substituted or unsubstituted group V₂, and the unsubstituted group V₂ is selected from the group consisting of: 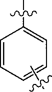 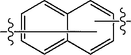 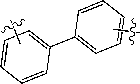 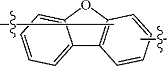 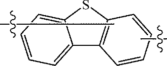 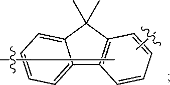
and
the substituted group V₂ has one or two or more substituents, and the substituents are respectively and independently selected from deuterium, fluorine, cyano, alkyl with 1 to 4 carbon atoms, fluoroalkyl with 1 to 4 carbon atoms, cycloalkyl with 5 to 10 carbon atoms, trialkylsilyl with 3 to 7 carbon atoms, phenyl, and naphthyl; and when that the number of the substituents is greater than 1, the substituents are the same or different.

9. The nitrogen-containing compound according to claim 1, wherein L₁ is selected from a single bond or the group consisting of: 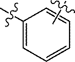 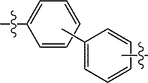 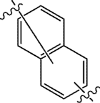 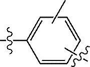 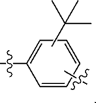
.

10. The nitrogen-containing compound according to claim 1, wherein 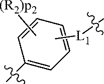
in the formula 1 is selected from the group consisting of: 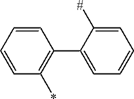 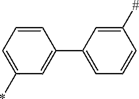 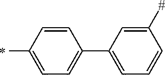 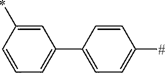 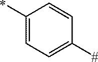 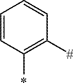 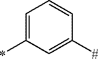 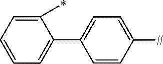 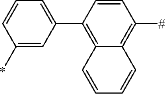 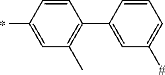 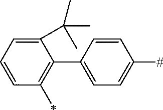 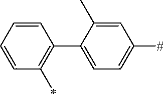 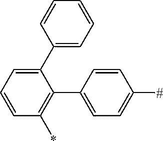 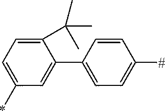 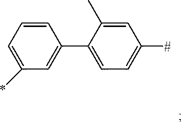
wherein ^{∗} indicates a site for connecting with 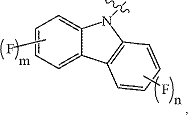
and # indicates a site for connecting with 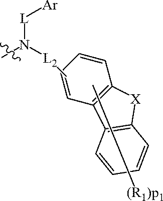 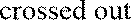 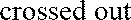 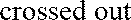
.

11. The nitrogen-containing compound according to claim 1, wherein L and L₂ are each independently selected from a single bond or the group consisting of: 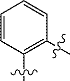 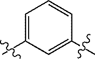 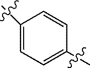 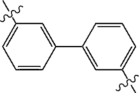 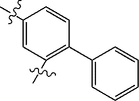 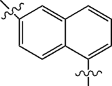 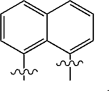 [SMILES: Fc1cccc2c3ccccc3n(-c3ccccc3-c3ccccc3N(c3ccc(-c4ccccc4)cc3)c3ccc(-c4cccc5c4oc4ccccc45)cc3)c12]
.

12. The nitrogen-containing compound according to claim 1, wherein 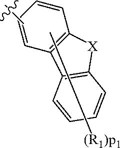 [SMILES: Fc1cccc2c3ccccc3n(-c3ccccc3-c3ccccc3N(c3ccc(-c4ccccc4)cc3)c3ccc(-c4cccc5c4sc4ccccc45)cc3)c12]
in the formula 1 is selected from 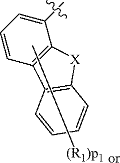 [SMILES: Fc1cccc2c3ccccc3n(-c3cccc(-c4cccc(N(c5ccc(-c6ccccc6)cc5)c5ccc(-c6cccc7c6oc6ccccc67)cc5)c4)c3)c12] 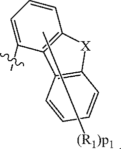 [SMILES: Fc1cccc2c3ccccc3n(-c3cccc(-c4cccc(N(c5ccc(-c6ccccc6)cc5)c5ccc(-c6cccc7c6sc6ccccc67)cc5)c4)c3)c12]
.

13. The nitrogen-containing compound according to claim 1, wherein the nitrogen-containing compound is selected from the group consisting of the following compounds: 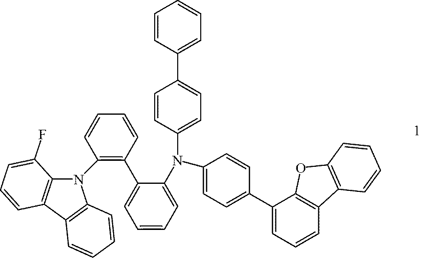 [SMILES: Fc1cccc2c3ccccc3n(-c3ccc(-c4cccc(N(c5ccc(-c6ccccc6)cc5)c5ccc(-c6cccc7c6oc6ccccc67)cc5)c4)cc3)c12] 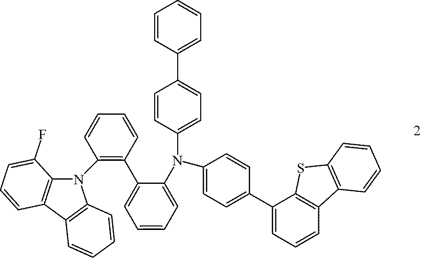 [SMILES: Fc1cccc2c3ccccc3n(-c3ccc(-c4cccc(N(c5ccc(-c6ccccc6)cc5)c5ccc(-c6cccc7c6sc6ccccc67)cc5)c4)cc3)c12] 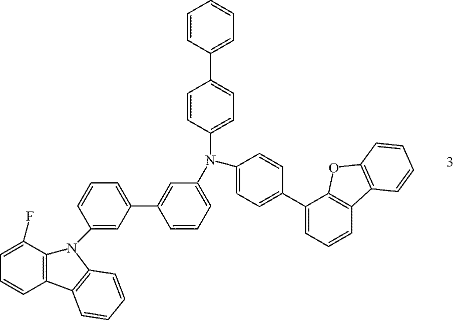 [SMILES: Fc1ccc2c3ccccc3n(-c3ccccc3-c3ccccc3N(c3ccc(-c4ccccc4)cc3)c3ccc(-c4cccc5c4oc4ccccc45)cc3)c2c1] 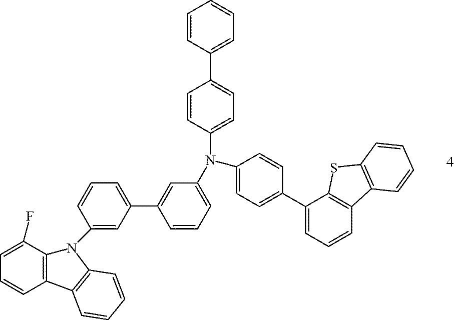 [SMILES: Fc1ccc2c3ccccc3n(-c3ccccc3-c3ccccc3N(c3ccc(-c4ccccc4)cc3)c3ccc(-c4cccc5c4sc4ccccc45)cc3)c2c1] 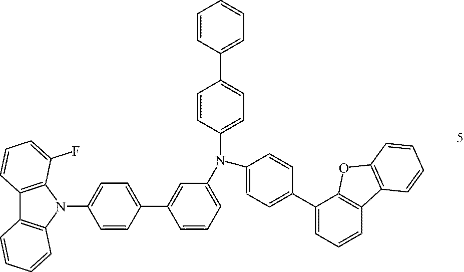 [SMILES: Fc1ccc2c3ccccc3n(-c3cccc(-c4cccc(N(c5ccc(-c6ccccc6)cc5)c5ccc(-c6cccc7c6oc6ccccc67)cc5)c4)c3)c2c1] 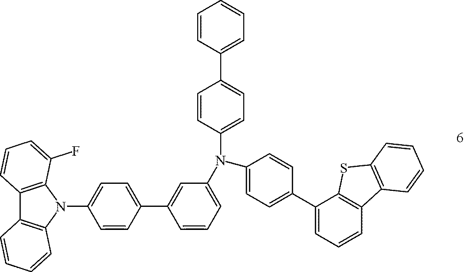 [SMILES: Fc1ccc2c3ccccc3n(-c3cccc(-c4cccc(N(c5ccc(-c6ccccc6)cc5)c5ccc(-c6cccc7c6sc6ccccc67)cc5)c4)c3)c2c1] 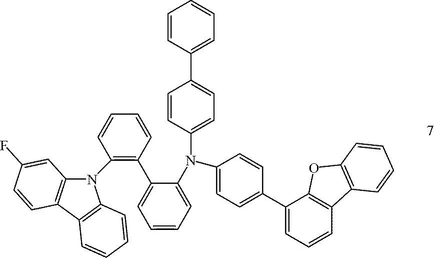 [SMILES: Fc1ccc2c3ccccc3n(-c3cccc(-c4ccc(N(c5ccc(-c6ccccc6)cc5)c5ccc(-c6cccc7c6oc6ccccc67)cc5)cc4)c3)c2c1] 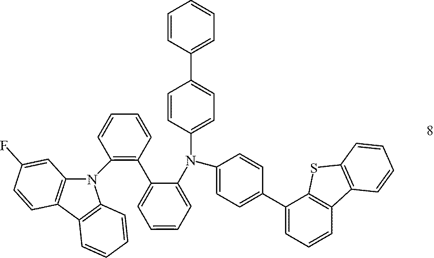 [SMILES: Fc1ccc2c3ccccc3n(-c3ccc(-c4cccc(N(c5ccc(-c6ccccc6)cc5)c5ccc(-c6cccc7c6sc6ccccc67)cc5)c4)cc3)c2c1] 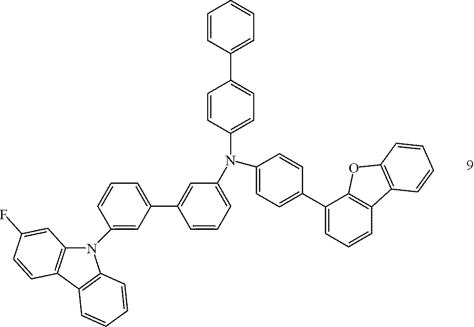 [SMILES: Fc1ccc2c(c1)c1ccccc1n2-c1ccccc1-c1ccccc1N(c1ccc(-c2ccccc2)cc1)c1ccc(-c2cccc3c2oc2ccccc23)cc1] 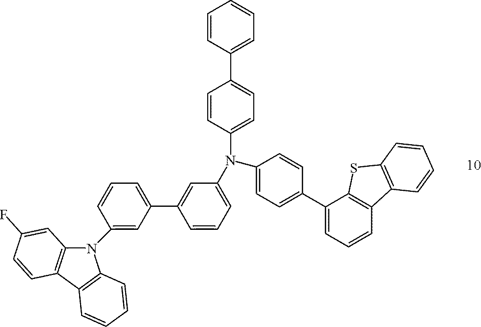 [SMILES: Fc1ccc2c(c1)c1ccccc1n2-c1ccccc1-c1ccccc1N(c1ccc(-c2ccccc2)cc1)c1ccc(-c2cccc3c2sc2ccccc23)cc1] 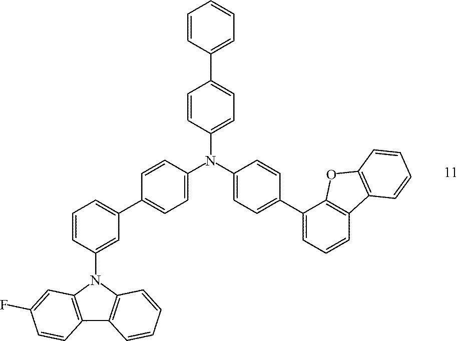 [SMILES: Fc1ccc2c(c1)c1ccccc1n2-c1cccc(-c2ccc(N(c3ccc(-c4ccccc4)cc3)c3ccc(-c4cccc5c4oc4ccccc45)cc3)cc2)c1] 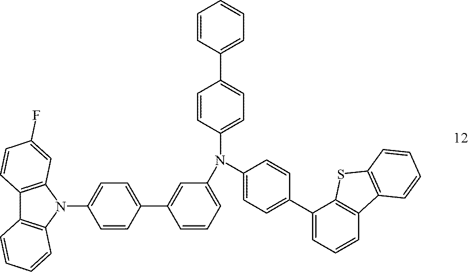 [SMILES: Fc1ccc2c(c1)c1ccccc1n2-c1cccc(-c2cccc(N(c3ccc(-c4ccccc4)cc3)c3ccc(-c4cccc5c4sc4ccccc45)cc3)c2)c1] 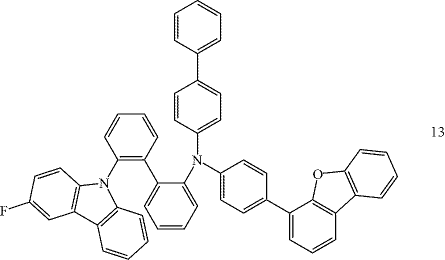 [SMILES: Fc1ccc2c(c1)c1ccccc1n2-c1ccc(-c2cccc(N(c3ccc(-c4ccccc4)cc3)c3ccc(-c4cccc5c4oc4ccccc45)cc3)c2)cc1] 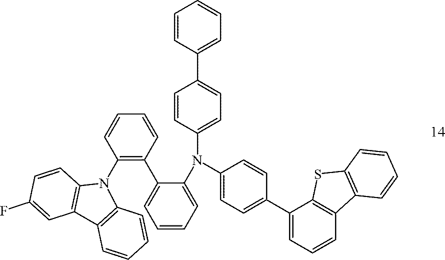 [SMILES: Fc1ccc2c(c1)c1ccccc1n2-c1ccc(-c2cccc(N(c3ccc(-c4ccccc4)cc3)c3ccc(-c4cccc5c4sc4ccccc45)cc3)c2)cc1] 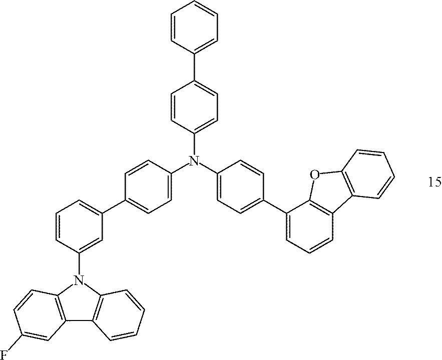 [SMILES: Fc1cccc2c1c1ccccc1n2-c1ccccc1-c1ccccc1N(c1ccc(-c2ccccc2)cc1)c1ccc(-c2cccc3c2oc2ccccc23)cc1] 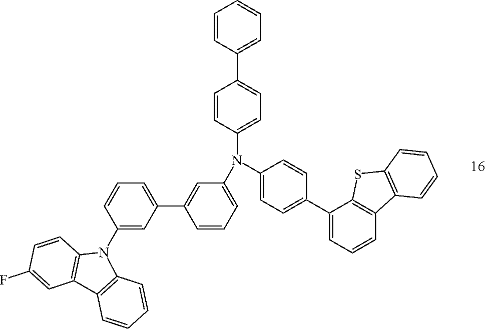 [SMILES: Fc1cccc2c1c1ccccc1n2-c1ccccc1-c1ccccc1N(c1ccc(-c2ccccc2)cc1)c1ccc(-c2cccc3c2sc2ccccc23)cc1] 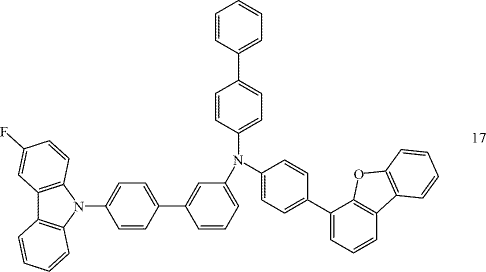 [SMILES: Fc1cccc2c1c1ccccc1n2-c1cccc(-c2cccc(N(c3ccc(-c4ccccc4)cc3)c3ccc(-c4cccc5c4sc4ccccc45)cc3)c2)c1] 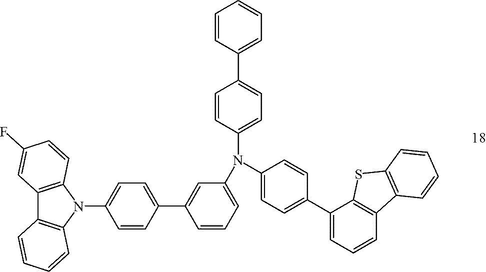 [SMILES: Fc1cccc2c1c1ccccc1n2-c1cccc(-c2cccc(N(c3ccc(-c4ccccc4)cc3)c3ccc(-c4cccc5c4oc4ccccc45)cc3)c2)c1] 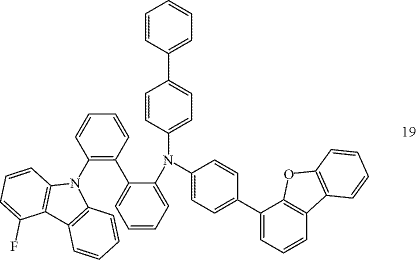 [SMILES: Fc1cccc2c1c1ccccc1n2-c1ccc(-c2cccc(N(c3ccc(-c4ccccc4)cc3)c3ccc(-c4cccc5c4oc4ccccc45)cc3)c2)cc1] 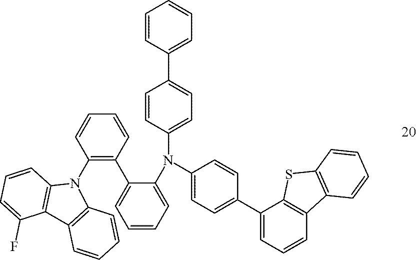 [SMILES: Fc1cccc2c1c1ccccc1n2-c1ccc(-c2cccc(N(c3ccc(-c4ccccc4)cc3)c3ccc(-c4cccc5c4sc4ccccc45)cc3)c2)cc1] 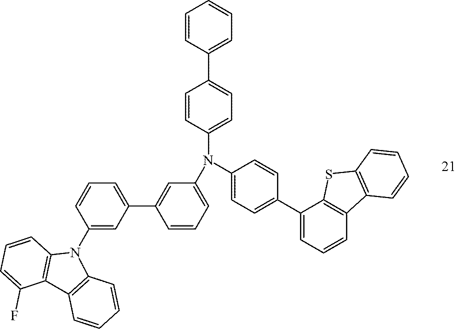 [SMILES: Fc1cccc2c3ccccc3n(-c3ccccc3N(c3ccc(-c4ccccc4)cc3)c3ccc(-c4cccc5c4oc4ccccc45)cc3)c12] 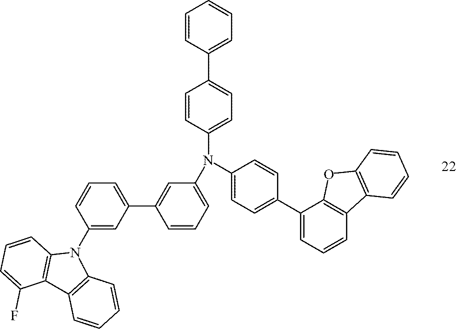 [SMILES: Fc1cccc2c3ccccc3n(-c3ccccc3N(c3ccc(-c4ccccc4)cc3)c3ccc(-c4cccc5c4sc4ccccc45)cc3)c12] 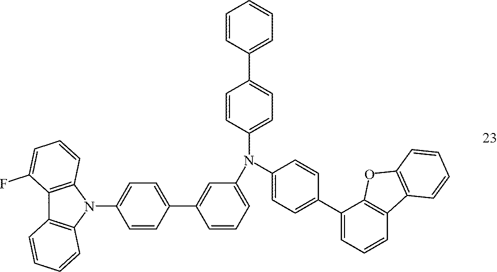 [SMILES: Fc1cccc2c3ccccc3n(-c3cccc(N(c4ccc(-c5ccccc5)cc4)c4ccc(-c5cccc6c5oc5ccccc56)cc4)c3)c12] 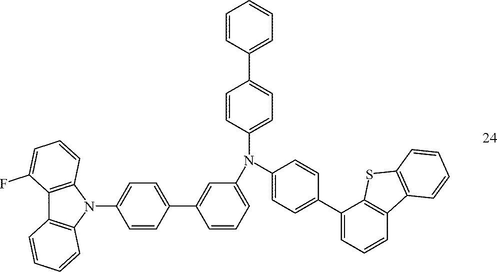 [SMILES: Fc1cccc2c3ccccc3n(-c3cccc(N(c4ccc(-c5ccccc5)cc4)c4ccc(-c5cccc6c5sc5ccccc56)cc4)c3)c12] 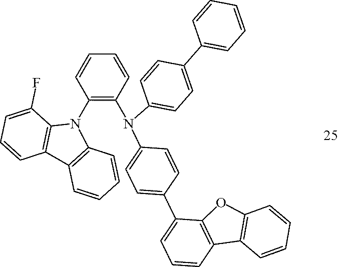 [SMILES: Fc1cccc2c3ccccc3n(-c3ccc(N(c4ccc(-c5ccccc5)cc4)c4ccc(-c5cccc6c5oc5ccccc56)cc4)cc3)c12] 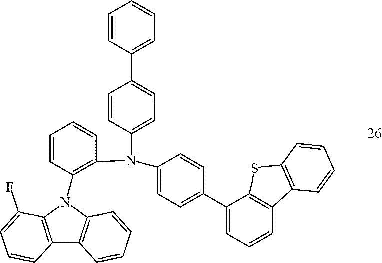 [SMILES: Fc1cccc2c3ccccc3n(-c3ccc(N(c4ccc(-c5ccccc5)cc4)c4ccc(-c5cccc6c5sc5ccccc56)cc4)cc3)c12] 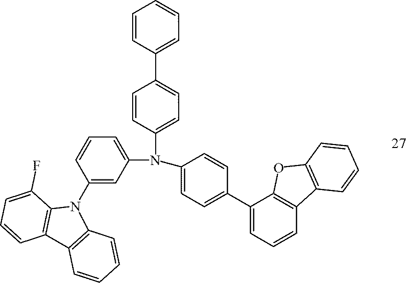 [SMILES: Fc1cccc2c3ccccc3n(-c3ccccc3N(c3ccc(-c4cccc5c4oc4ccccc45)cc3)c3cccc4ccccc34)c12] 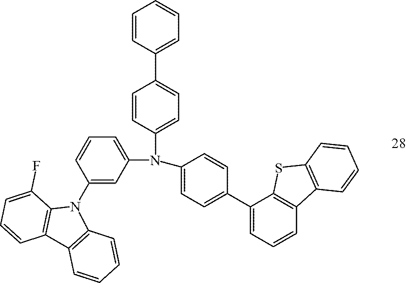 [SMILES: Fc1cccc2c3ccccc3n(-c3ccccc3N(c3ccc(-c4cccc5c4sc4ccccc45)cc3)c3cccc4ccccc34)c12] 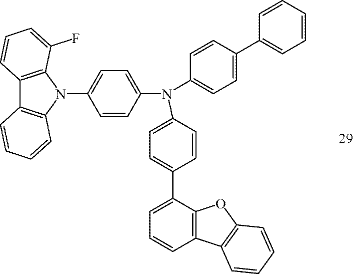 [SMILES: Fc1cccc2c3ccccc3n(-c3cccc(N(c4ccc(-c5cccc6c5oc5ccccc56)cc4)c4cccc5ccccc45)c3)c12] 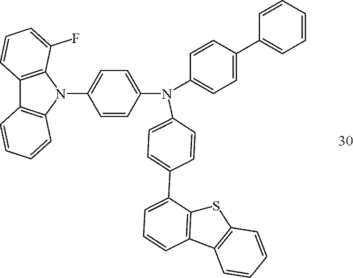 [SMILES: Fc1cccc2c3ccccc3n(-c3cccc(N(c4ccc(-c5cccc6c5sc5ccccc56)cc4)c4cccc5ccccc45)c3)c12] 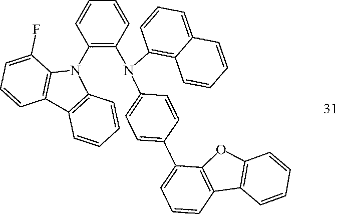 [SMILES: Fc1cccc2c3ccccc3n(-c3ccc(N(c4ccc(-c5cccc6c5oc5ccccc56)cc4)c4cccc5ccccc45)cc3)c12] 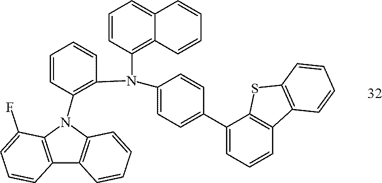 [SMILES: Fc1cccc2c3ccccc3n(-c3ccc(N(c4ccc(-c5cccc6c5sc5ccccc56)cc4)c4cccc5ccccc45)cc3)c12] 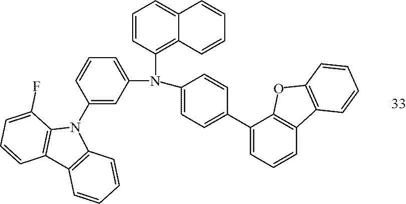 [SMILES: Fc1cccc2c3ccccc3n(-c3ccccc3N(c3ccc(-c4cccc5c4oc4ccccc45)cc3)c3ccc4ccccc4c3)c12] 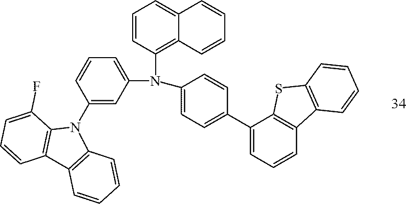 [SMILES: Fc1cccc2c3ccccc3n(-c3ccccc3N(c3ccc(-c4cccc5c4sc4ccccc45)cc3)c3ccc4ccccc4c3)c12] 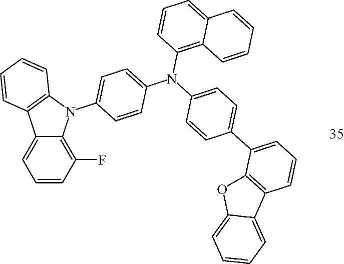 [SMILES: Fc1cccc2c3ccccc3n(-c3cccc(N(c4ccc(-c5cccc6c5oc5ccccc56)cc4)c4ccc5ccccc5c4)c3)c12] 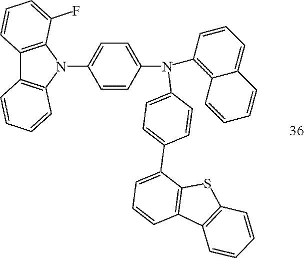 [SMILES: Fc1cccc2c3ccccc3n(-c3cccc(N(c4ccc(-c5cccc6c5sc5ccccc56)cc4)c4ccc5ccccc5c4)c3)c12] 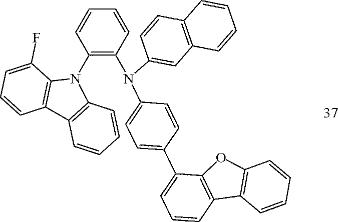 [SMILES: Fc1cccc2c3ccccc3n(-c3ccc(N(c4ccc(-c5cccc6c5oc5ccccc56)cc4)c4ccc5ccccc5c4)cc3)c12] 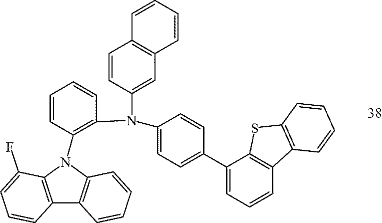 [SMILES: Fc1cccc2c3ccccc3n(-c3ccc(N(c4ccc(-c5cccc6c5sc5ccccc56)cc4)c4ccc5ccccc5c4)cc3)c12] 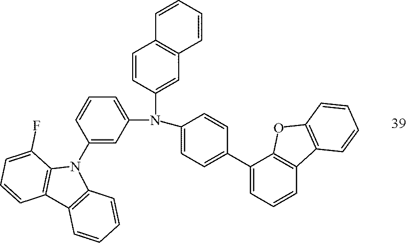 [SMILES: Fc1cccc2c1C(c1ccccc1N(c1ccc(-c3cccc4ccccc34)cc1)c1ccc(-c3cccc4c3oc3ccccc34)cc1)c1ccccc1-2] 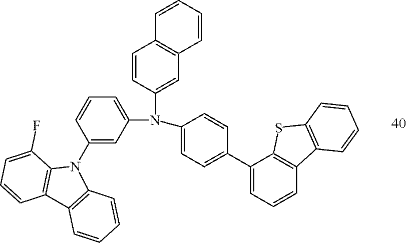 [SMILES: Fc1cccc2c3ccccc3n(-c3ccccc3N(c3ccc(-c4cccc5ccccc45)cc3)c3ccc(-c4cccc5c4sc4ccccc45)cc3)c12] 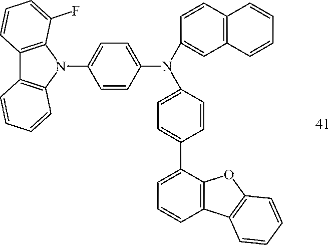 [SMILES: Fc1cccc2c3ccccc3n(-c3cccc(N(c4ccc(-c5cccc6ccccc56)cc4)c4ccc(-c5cccc6c5oc5ccccc56)cc4)c3)c12] 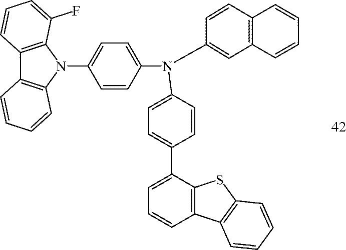 [SMILES: Fc1cccc2c3ccccc3n(-c3cccc(N(c4ccc(-c5cccc6ccccc56)cc4)c4ccc(-c5cccc6c5sc5ccccc56)cc4)c3)c12] 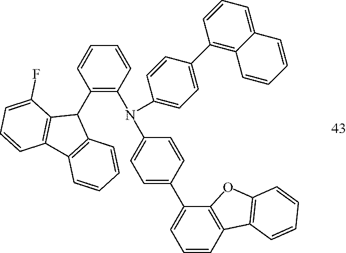 [SMILES: Fc1cccc2c3ccccc3n(-c3ccc(N(c4ccc(-c5cccc6ccccc56)cc4)c4ccc(-c5cccc6c5oc5ccccc56)cc4)cc3)c12] 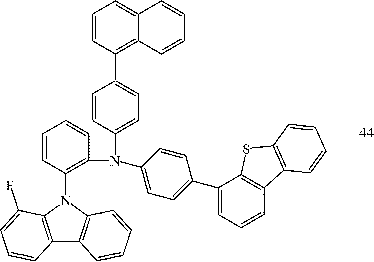 [SMILES: Fc1cccc2c3ccccc3n(-c3ccc(N(c4ccc(-c5cccc6ccccc56)cc4)c4ccc(-c5cccc6c5sc5ccccc56)cc4)cc3)c12] 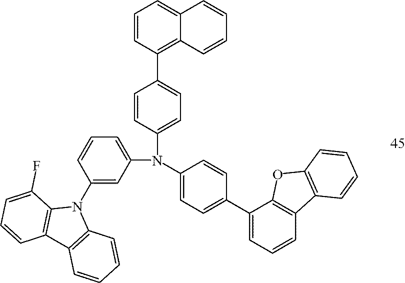 [SMILES: Fc1cccc2c3ccccc3n(-c3ccccc3N(c3ccc(-c4ccc5ccccc5c4)cc3)c3ccc(-c4cccc5c4oc4ccccc45)cc3)c12] 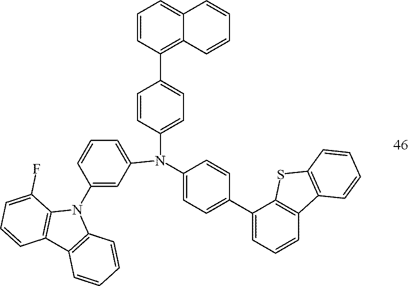 [SMILES: Fc1cccc2c3ccccc3n(-c3ccccc3N(c3ccc(-c4ccc5ccccc5c4)cc3)c3ccc(-c4cccc5c4sc4ccccc45)cc3)c12] 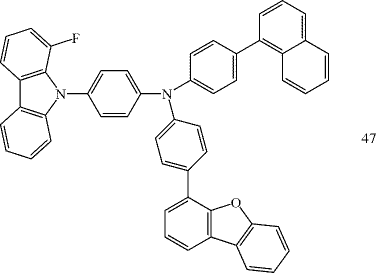 [SMILES: Fc1cccc2c3ccccc3n(-c3cccc(N(c4ccc(-c5ccc6ccccc6c5)cc4)c4ccc(-c5cccc6c5oc5ccccc56)cc4)c3)c12] 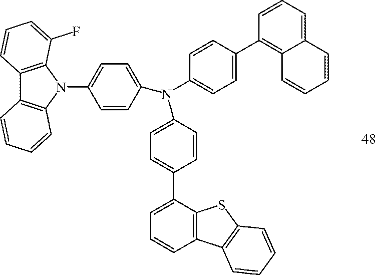 [SMILES: Fc1cccc2c3ccccc3n(-c3cccc(N(c4ccc(-c5ccc6ccccc6c5)cc4)c4ccc(-c5cccc6c5oc5ccccc56)cc4)c3)c12] 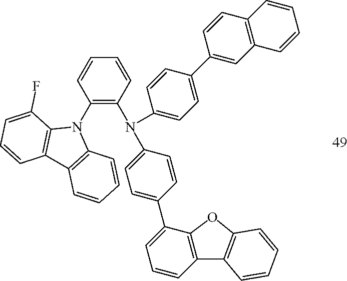 [SMILES: Fc1cccc2c3ccccc3n(-c3ccc(N(c4ccc(-c5ccc6ccccc6c5)cc4)c4ccc(-c5cccc6c5oc5ccccc56)cc4)cc3)c12] 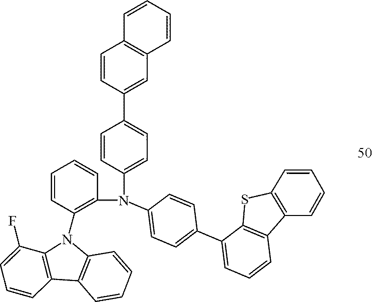 [SMILES: Fc1cccc2c3ccccc3n(-c3ccc(N(c4ccc(-c5ccc6ccccc6c5)cc4)c4ccc(-c5cccc6c5oc5ccccc56)cc4)cc3)c12] 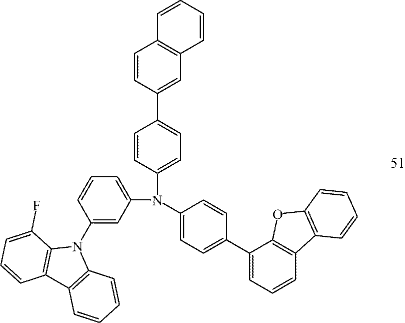 [SMILES: Fc1ccc2c3ccccc3n(-c3ccccc3N(c3ccc(-c4ccc5ccccc5c4)cc3)c3ccc(-c4cccc5c4oc4ccccc45)cc3)c2c1] 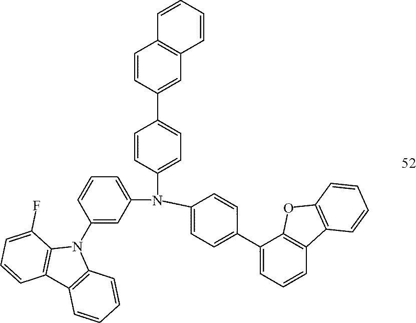 [SMILES: Fc1ccc2c3ccccc3n(-c3ccccc3N(c3ccc(-c4ccc5ccccc5c4)cc3)c3ccc(-c4cccc5c4sc4ccccc45)cc3)c2c1] 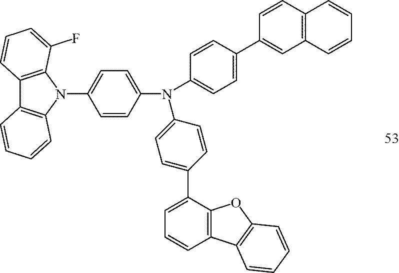 [SMILES: Fc1ccc2c3ccccc3n(-c3cccc(N(c4ccc(-c5ccc6ccccc6c5)cc4)c4ccc(-c5cccc6c5oc5ccccc56)cc4)c3)c2c1] 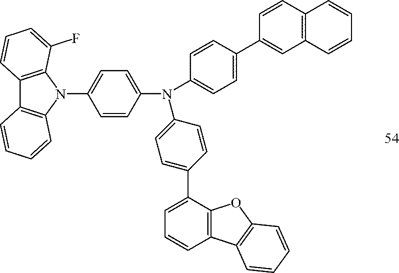 [SMILES: Fc1ccc2c3ccccc3n(-c3cccc(N(c4ccc(-c5ccc6ccccc6c5)cc4)c4ccc(-c5cccc6c5sc5ccccc56)cc4)c3)c2c1] 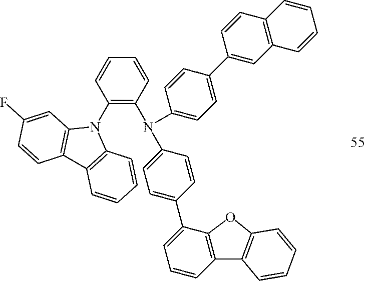 [SMILES: Fc1ccc2c3ccccc3n(-c3ccc(N(c4ccc(-c5ccc6ccccc6c5)cc4)c4ccc(-c5cccc6c5oc5ccccc56)cc4)cc3)c2c1] 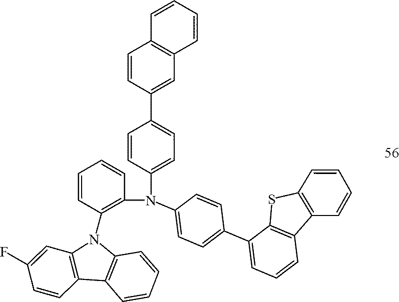 [SMILES: Fc1ccc2c3ccccc3n(-c3ccc(N(c4ccc(-c5ccc6ccccc6c5)cc4)c4ccc(-c5cccc6c5sc5ccccc56)cc4)cc3)c2c1] 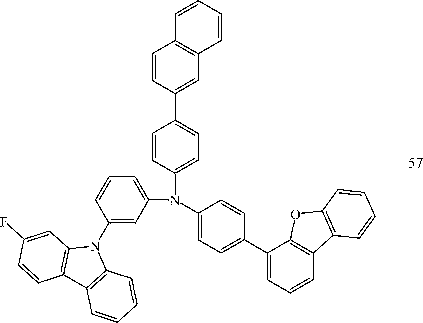 [SMILES: Fc1ccc2c(c1)c1ccccc1n2-c1ccccc1N(c1ccc(-c2ccc3ccccc3c2)cc1)c1ccc(-c2cccc3c2oc2ccccc23)cc1] 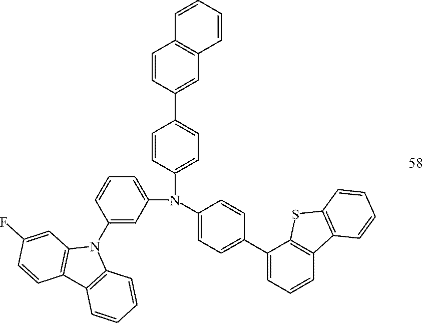 [SMILES: Fc1ccc2c(c1)c1ccccc1n2-c1ccccc1N(c1ccc(-c2ccc3ccccc3c2)cc1)c1ccc(-c2cccc3c2sc2ccccc23)cc1] 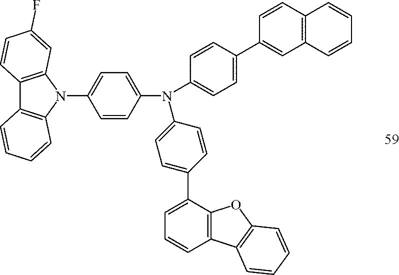 [SMILES: Fc1ccc2c(c1)c1ccccc1n2-c1cccc(N(c2ccc(-c3ccc4ccccc4c3)cc2)c2ccc(-c3cccc4c3oc3ccccc34)cc2)c1] 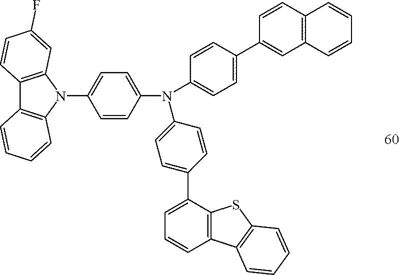 [SMILES: Fc1ccc2c(c1)c1ccccc1n2-c1cccc(N(c2ccc(-c3ccc4ccccc4c3)cc2)c2ccc(-c3cccc4c3sc3ccccc34)cc2)c1] 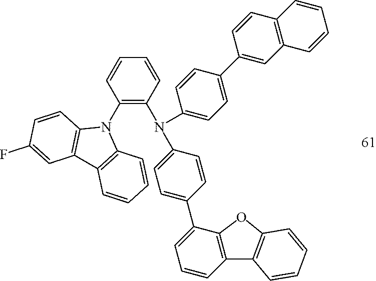 [SMILES: Fc1ccc2c(c1)c1ccccc1n2-c1ccc(N(c2ccc(-c3ccc4ccccc4c3)cc2)c2ccc(-c3cccc4c3oc3ccccc34)cc2)cc1] 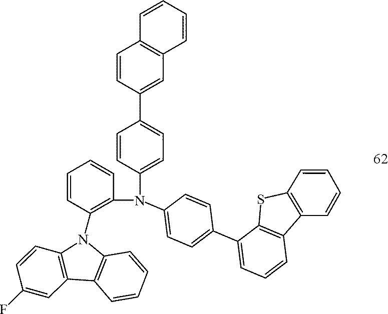 [SMILES: Fc1ccc2c(c1)c1ccccc1n2-c1ccc(N(c2ccc(-c3ccc4ccccc4c3)cc2)c2ccc(-c3cccc4c3sc3ccccc34)cc2)cc1] 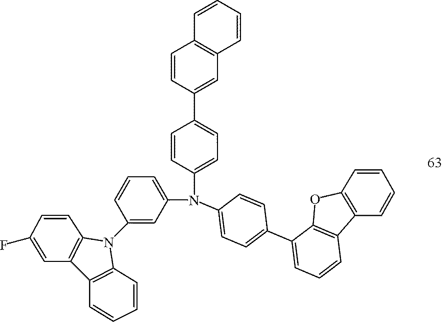 [SMILES: Fc1cccc2c1c1ccccc1n2-c1ccccc1N(c1ccc(-c2ccc3ccccc3c2)cc1)c1ccc(-c2cccc3c2oc2ccccc23)cc1] 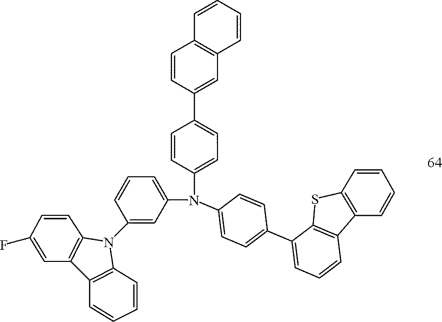 [SMILES: Fc1cccc2c1c1ccccc1n2-c1ccccc1N(c1ccc(-c2ccc3ccccc3c2)cc1)c1ccc(-c2cccc3c2sc2ccccc23)cc1] 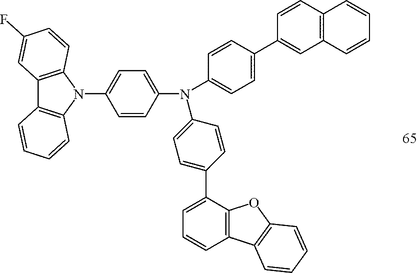 [SMILES: Fc1cccc2c1c1ccccc1n2-c1cccc(N(c2ccc(-c3ccc4ccccc4c3)cc2)c2ccc(-c3cccc4c3oc3ccccc34)cc2)c1] 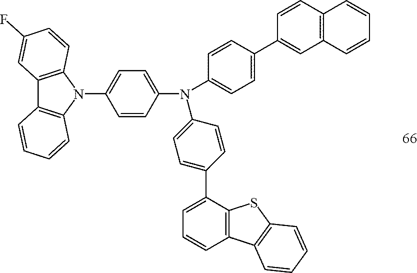 [SMILES: Fc1cccc2c1c1ccccc1n2-c1cccc(N(c2ccc(-c3ccc4ccccc4c3)cc2)c2ccc(-c3cccc4c3sc3ccccc34)cc2)c1] 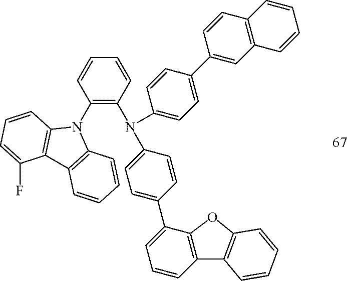 [SMILES: Fc1cccc2c1c1ccccc1n2-c1ccc(N(c2ccc(-c3ccc4ccccc4c3)cc2)c2ccc(-c3cccc4c3oc3ccccc34)cc2)cc1] 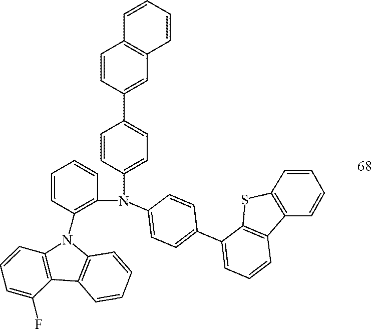 [SMILES: Fc1cccc2c1c1ccccc1n2-c1ccc(N(c2ccc(-c3ccc4ccccc4c3)cc2)c2ccc(-c3cccc4c3sc3ccccc34)cc2)cc1] 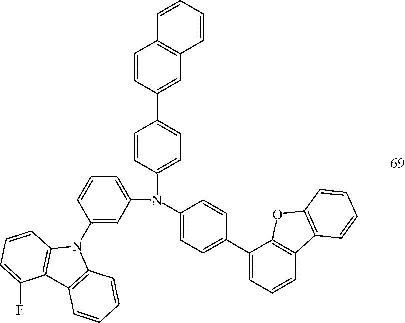 [SMILES: Fc1cccc2c1c1ccccc1n2-c1ccccc1N(c1ccc(-c2ccccc2)cc1)c1ccc(-c2cccc3c2oc2c(-c4ccccc4)cccc23)cc1] 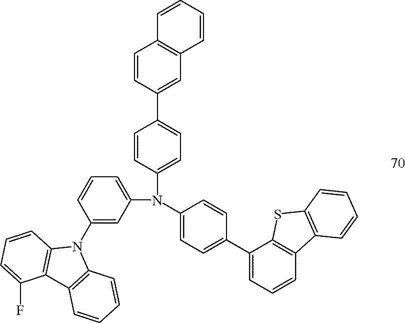 [SMILES: Fc1cccc2c1c1ccccc1n2-c1ccccc1N(c1ccc(-c2ccccc2)cc1)c1ccc(-c2cccc3c2sc2c(-c4ccccc4)cccc23)cc1] 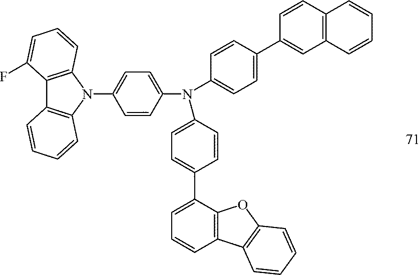 [SMILES: Fc1cccc2c1c1ccccc1n2-c1cccc(N(c2ccc(-c3ccccc3)cc2)c2ccc(-c3cccc4c3oc3c(-c5ccccc5)cccc34)cc2)c1] 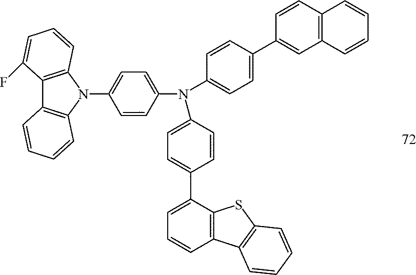 [SMILES: Fc1cccc2c1c1ccccc1n2-c1cccc(N(c2ccc(-c3ccccc3)cc2)c2ccc(-c3cccc4c3sc3c(-c5ccccc5)cccc34)cc2)c1] 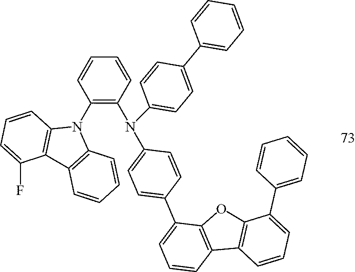 [SMILES: Fc1cccc2c1c1ccccc1n2-c1ccc(N(c2ccc(-c3ccccc3)cc2)c2ccc(-c3cccc4c3oc3c(-c5ccccc5)cccc34)cc2)cc1] 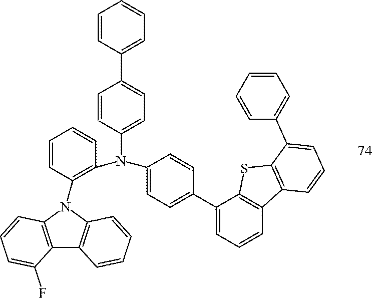 [SMILES: Fc1cccc2c1c1ccccc1n2-c1ccc(N(c2ccc(-c3ccccc3)cc2)c2ccc(-c3cccc4c3sc3c(-c5ccccc5)cccc34)cc2)cc1] 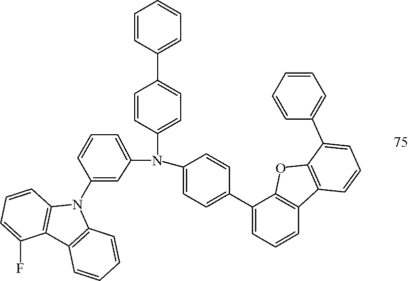 [SMILES: Fc1ccc2c(c1)c1ccccc1n2-c1ccccc1N(c1ccc(-c2ccccc2)cc1)c1ccc(-c2cccc3c2oc2c(-c4ccccc4)cccc23)cc1] 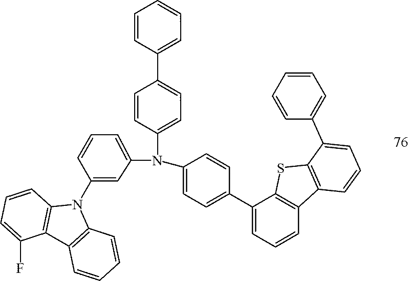 [SMILES: Fc1ccc2c(c1)c1ccccc1n2-c1ccccc1N(c1ccc(-c2ccccc2)cc1)c1ccc(-c2cccc3c2sc2c(-c4ccccc4)cccc23)cc1] 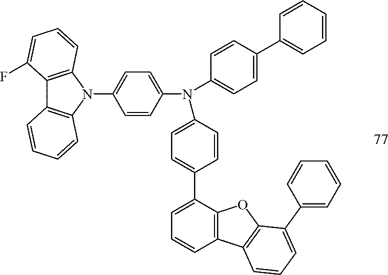 [SMILES: Fc1ccc2c(c1)c1ccccc1n2-c1cccc(N(c2ccc(-c3ccccc3)cc2)c2ccc(-c3cccc4c3oc3c(-c5ccccc5)cccc34)cc2)c1] 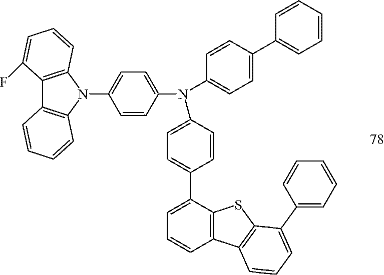 [SMILES: Fc1ccc2c(c1)c1ccccc1n2-c1cccc(N(c2ccc(-c3ccccc3)cc2)c2ccc(-c3cccc4c3sc3c(-c5ccccc5)cccc34)cc2)c1] 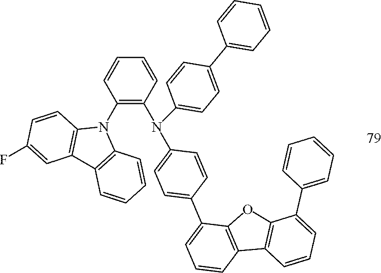 [SMILES: Fc1ccc2c(c1)c1ccccc1n2-c1ccc(N(c2ccc(-c3ccccc3)cc2)c2ccc(-c3cccc4c3oc3c(-c5ccccc5)cccc34)cc2)cc1] 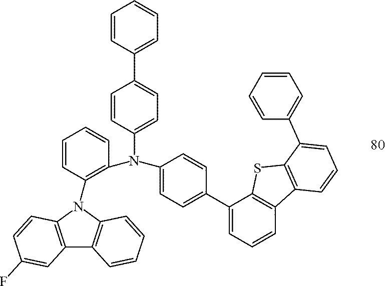 [SMILES: Fc1ccc2c(c1)c1ccccc1n2-c1ccc(N(c2ccc(-c3ccccc3)cc2)c2ccc(-c3cccc4c3sc3c(-c5ccccc5)cccc34)cc2)cc1] 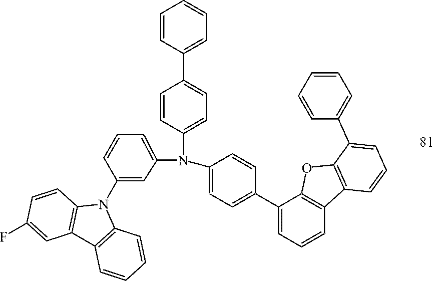 [SMILES: Fc1ccc2c3ccccc3n(-c3ccccc3N(c3ccc(-c4ccccc4)cc3)c3ccc(-c4cccc5c4oc4c(-c6ccccc6)cccc45)cc3)c2c1] 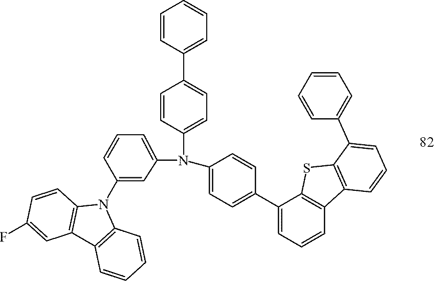 [SMILES: Fc1ccc2c3ccccc3n(-c3ccccc3N(c3ccc(-c4ccccc4)cc3)c3ccc(-c4cccc5c4sc4c(-c6ccccc6)cccc45)cc3)c2c1] 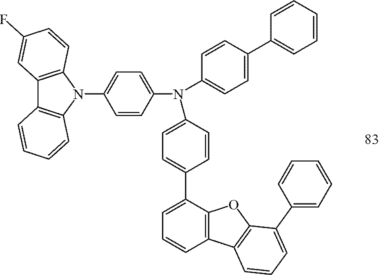 [SMILES: Fc1ccc2c3ccccc3n(-c3cccc(N(c4ccc(-c5ccccc5)cc4)c4ccc(-c5cccc6c5oc5c(-c7ccccc7)cccc56)cc4)c3)c2c1] 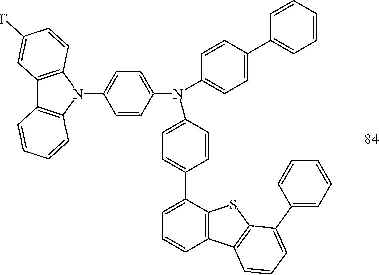 [SMILES: Fc1ccc2c3ccccc3n(-c3cccc(N(c4ccc(-c5ccccc5)cc4)c4ccc(-c5cccc6c5sc5c(-c7ccccc7)cccc56)cc4)c3)c2c1] 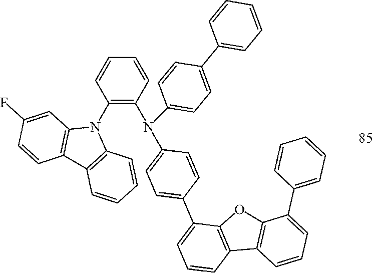 [SMILES: Fc1ccc2c3ccccc3n(-c3ccc(N(c4ccc(-c5ccccc5)cc4)c4ccc(-c5cccc6c5oc5c(-c7ccccc7)cccc56)cc4)cc3)c2c1] 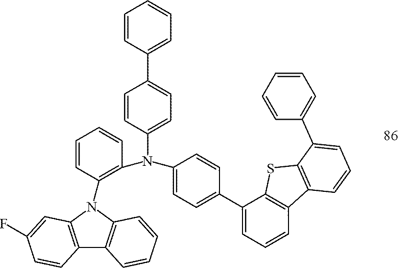 [SMILES: Fc1ccc2c3ccccc3n(-c3ccc(N(c4ccc(-c5ccccc5)cc4)c4ccc(-c5cccc6c5sc5c(-c7ccccc7)cccc56)cc4)cc3)c2c1] 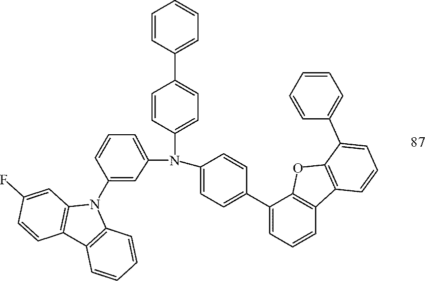 [SMILES: Fc1cccc2c3ccccc3n(-c3ccccc3N(c3ccc(-c4ccccc4)cc3)c3ccc(-c4cccc5c4oc4c(-c6ccccc6)cccc45)cc3)c12] 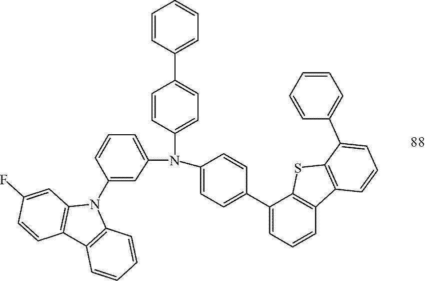 [SMILES: Fc1cccc2c3ccccc3n(-c3ccccc3N(c3ccc(-c4ccccc4)cc3)c3ccc(-c4cccc5c4sc4c(-c6ccccc6)cccc45)cc3)c12] 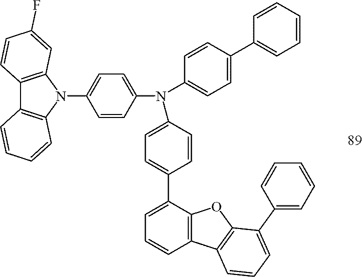 [SMILES: Fc1cccc2c3ccccc3n(-c3cccc(N(c4ccc(-c5ccccc5)cc4)c4ccc(-c5cccc6c5oc5c(-c7ccccc7)cccc56)cc4)c3)c12] 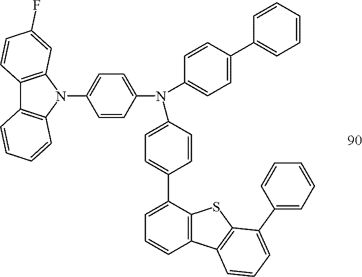 [SMILES: Fc1cccc2c3ccccc3n(-c3cccc(N(c4ccc(-c5ccccc5)cc4)c4ccc(-c5cccc6c5sc5c(-c7ccccc7)cccc56)cc4)c3)c12] 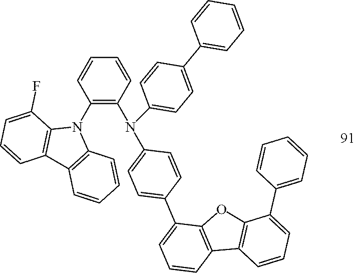 [SMILES: Fc1cccc2c3ccccc3n(-c3ccc(N(c4ccc(-c5ccccc5)cc4)c4ccc(-c5cccc6c5oc5c(-c7ccccc7)cccc56)cc4)cc3)c12] 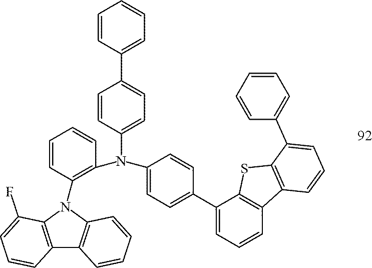 [SMILES: Fc1cccc2c3ccccc3n(-c3ccc(N(c4ccc(-c5ccccc5)cc4)c4ccc(-c5cccc6c5sc5c(-c7ccccc7)cccc56)cc4)cc3)c12] 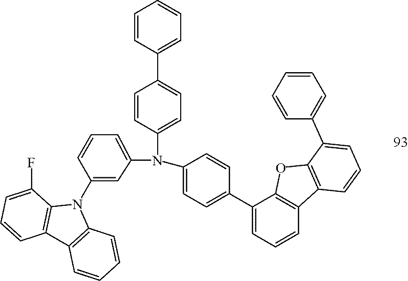 [SMILES: Fc1ccc2c(c1)c1ccccc1n2-c1cccc(-c2ccc(N(c3ccc(-c4ccccc4)cc3)c3ccc(-c4cccc5oc6ccccc6c45)cc3)cc2)c1] 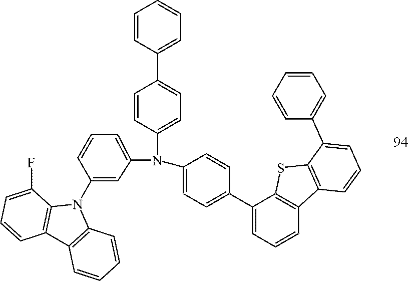 [SMILES: Fc1ccc2c(c1)c1ccccc1n2-c1cccc(-c2ccc(N(c3ccc(-c4ccccc4)cc3)c3ccc(-c4cccc5sc6ccccc6c45)cc3)cc2)c1] 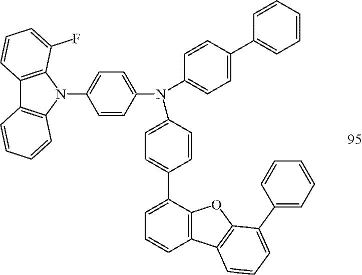 [SMILES: Fc1ccc2c3ccccc3n(-c3cccc(-c4ccc(N(c5ccc(-c6ccccc6)cc5)c5ccc(-c6cccc7oc8ccccc8c67)cc5)cc4)c3)c2c1] 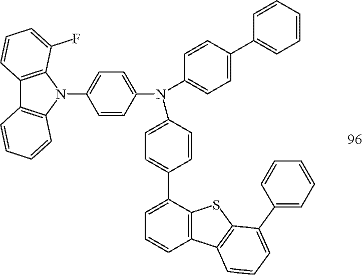 [SMILES: Fc1ccc2c3ccccc3n(-c3cccc(-c4ccc(N(c5ccc(-c6ccccc6)cc5)c5ccc(-c6cccc7sc8ccccc8c67)cc5)cc4)c3)c2c1] 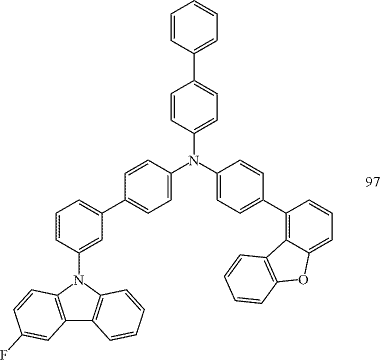 [SMILES: Fc1cccc2c3ccccc3n(-c3cccc(-c4ccc(N(c5ccc(-c6ccccc6)cc5)c5ccc(-c6cccc7oc8ccccc8c67)cc5)cc4)c3)c12] 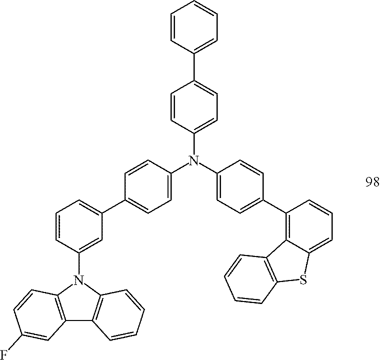 [SMILES: Fc1cccc2c3ccccc3n(-c3cccc(-c4ccc(N(c5ccc(-c6ccccc6)cc5)c5ccc(-c6cccc7sc8ccccc8c67)cc5)cc4)c3)c12] 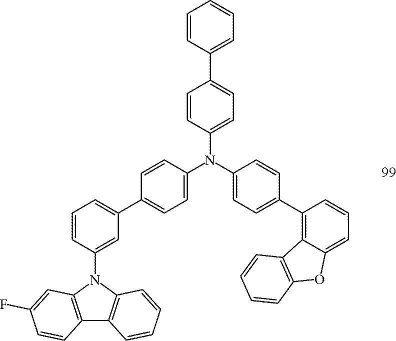 [SMILES: Fc1cccc2c1c1ccccc1n2-c1cccc(-c2ccc(N(c3ccc(-c4ccccc4)cc3)c3ccc(-c4cccc5oc6ccccc6c45)cc3)cc2)c1] 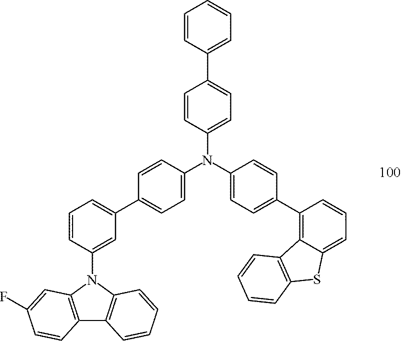 [SMILES: Fc1cccc2c1c1ccccc1n2-c1cccc(-c2ccc(N(c3ccc(-c4ccccc4)cc3)c3ccc(-c4cccc5sc6ccccc6c45)cc3)cc2)c1] 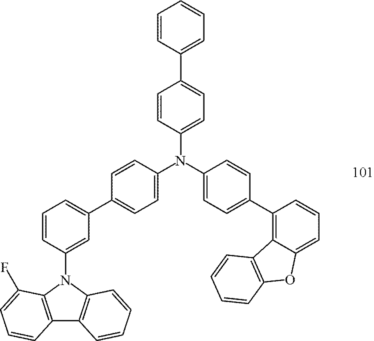 [SMILES: Fc1cccc2c3ccccc3n(-c3cccc(-c4ccc(N(c5ccc(-c6cccc7oc8ccccc8c67)cc5)c5ccccc5-c5ccccc5)cc4)c3)c12] 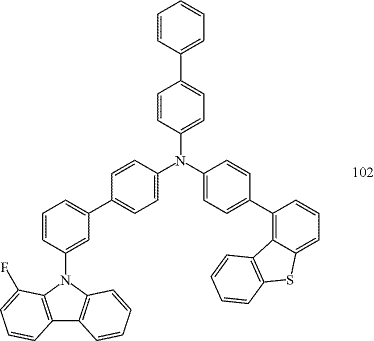 [SMILES: Fc1cccc2c3ccccc3n(-c3cccc(-c4ccc(N(c5ccc(-c6cccc7sc8ccccc8c67)cc5)c5ccccc5-c5ccccc5)cc4)c3)c12] 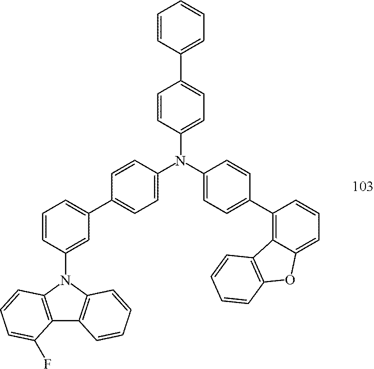 [SMILES: Fc1cccc2c1c1ccccc1n2-c1cccc(-c2ccc(N(c3ccc(-c4cccc5oc6ccccc6c45)cc3)c3ccccc3-c3ccccc3)cc2)c1] 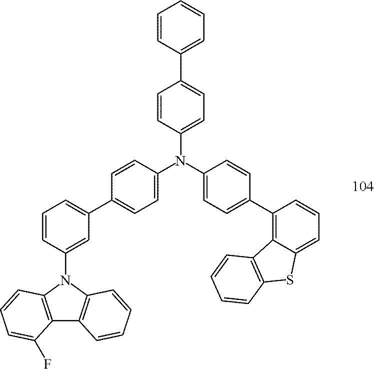 [SMILES: Fc1cccc2c1c1ccccc1n2-c1cccc(-c2ccc(N(c3ccc(-c4cccc5sc6ccccc6c45)cc3)c3ccccc3-c3ccccc3)cc2)c1] 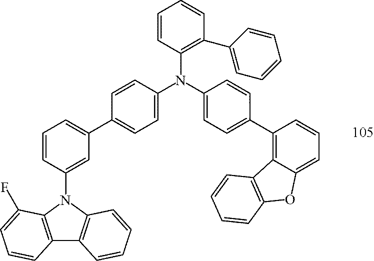 [SMILES: Fc1cccc2c3ccccc3n(-c3cccc(-c4ccc(N(c5ccc(-c6cccc7oc8ccccc8c67)cc5)c5cccc(-c6ccccc6)c5)cc4)c3)c12] 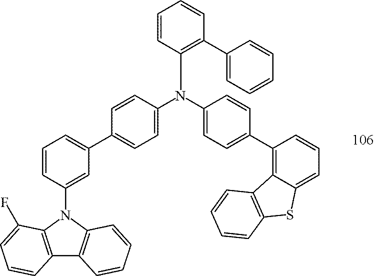 [SMILES: Fc1cccc2c3ccccc3n(-c3cccc(-c4ccc(N(c5ccc(-c6cccc7sc8ccccc8c67)cc5)c5cccc(-c6ccccc6)c5)cc4)c3)c12] 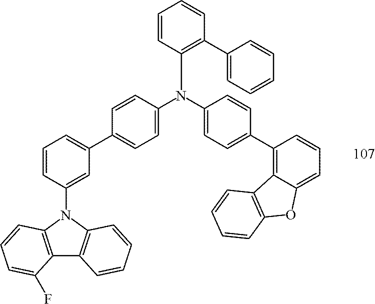 [SMILES: Fc1cccc2c1c1ccccc1n2-c1cccc(-c2ccc(N(c3ccc(-c4cccc5oc6ccccc6c45)cc3)c3cccc(-c4ccccc4)c3)cc2)c1] 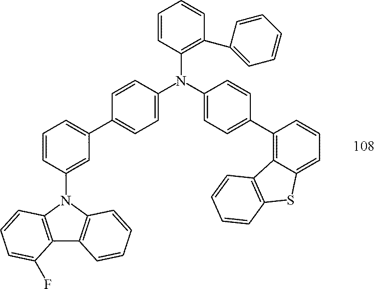 [SMILES: Fc1cccc2c1c1ccccc1n2-c1cccc(-c2ccc(N(c3ccc(-c4cccc5sc6ccccc6c45)cc3)c3cccc(-c4ccccc4)c3)cc2)c1] 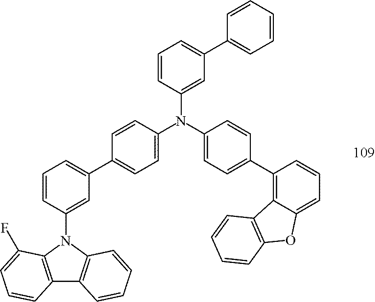 [SMILES: Fc1ccc2c(c1)c1ccccc1n2-c1cccc(-c2ccc(N(c3ccc(-c4cccc5oc6ccccc6c45)cc3)c3cccc(-c4ccccc4)c3)cc2)c1] 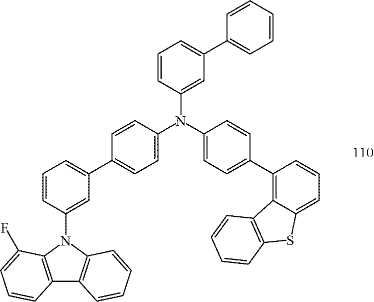 [SMILES: Fc1ccc2c(c1)c1ccccc1n2-c1cccc(-c2ccc(N(c3ccc(-c4cccc5sc6ccccc6c45)cc3)c3cccc(-c4ccccc4)c3)cc2)c1] 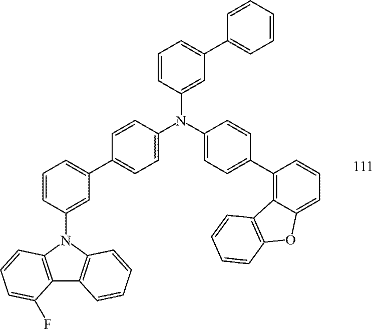 [SMILES: Fc1ccc2c3ccccc3n(-c3cccc(-c4ccc(N(c5ccc(-c6cccc7oc8ccccc8c67)cc5)c5cccc(-c6ccccc6)c5)cc4)c3)c2c1] 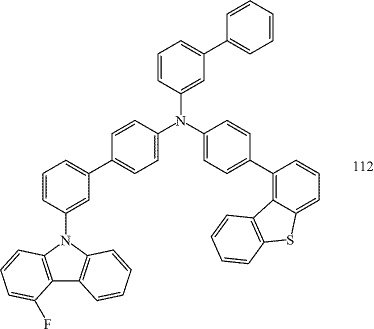 [SMILES: Fc1ccc2c3ccccc3n(-c3cccc(-c4ccc(N(c5ccc(-c6cccc7sc8ccccc8c67)cc5)c5cccc(-c6ccccc6)c5)cc4)c3)c2c1] 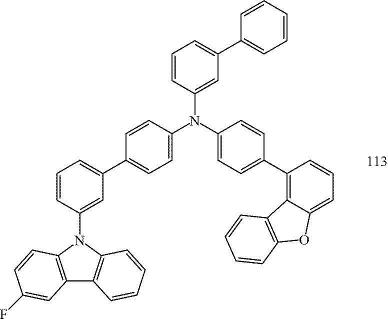 [SMILES: [2H]c1ccc(-c2cccc(N(c3ccc(-c4cccc(-n5c6ccccc6c6cc(F)ccc65)c4)cc3)c3ccc(-c4cccc5oc6ccccc6c45)cc3)c2)cc1] 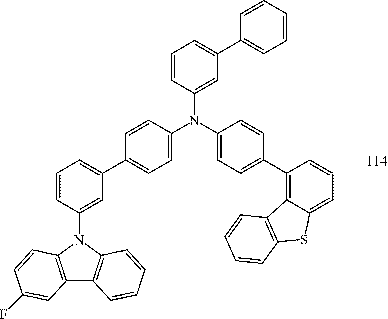 [SMILES: [2H]c1ccc(-c2cccc(N(c3ccc(-c4cccc(-n5c6ccccc6c6cc(F)ccc65)c4)cc3)c3ccc(-c4cccc5sc6ccccc6c45)cc3)c2)cc1] 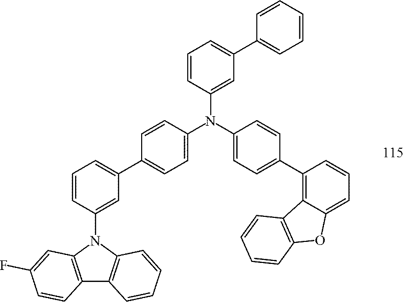 [SMILES: N#Cc1ccc(-c2cccc(N(c3ccc(-c4cccc(-n5c6ccccc6c6ccc(F)cc65)c4)cc3)c3ccc(-c4cccc5oc6ccccc6c45)cc3)c2)cc1] 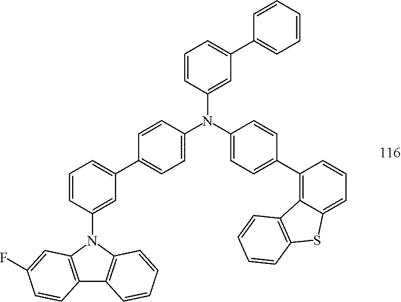 [SMILES: N#Cc1ccc(-c2cccc(N(c3ccc(-c4cccc(-n5c6ccccc6c6ccc(F)cc65)c4)cc3)c3ccc(-c4cccc5sc6ccccc6c45)cc3)c2)cc1] 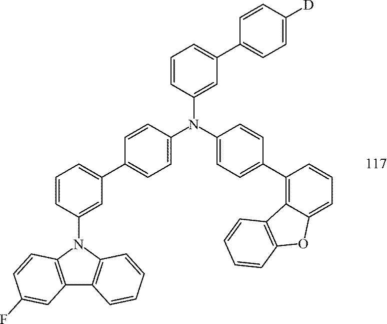 [SMILES: Fc1ccc2c(c1)c1cc(F)ccc1n2-c1cccc(-c2cccc(N(c3ccc(-c4ccccc4)cc3)c3ccc(-c4cccc5c4oc4ccccc45)cc3)c2)c1] 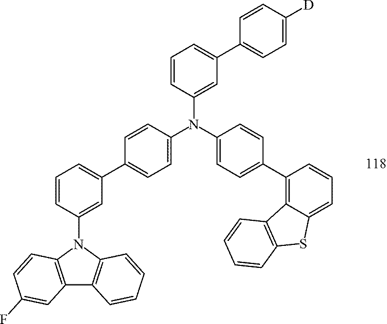 [SMILES: Fc1ccc2c(c1)c1cc(F)ccc1n2-c1cccc(-c2cccc(N(c3ccc(-c4ccccc4)cc3)c3ccc(-c4cccc5c4sc4ccccc45)cc3)c2)c1] 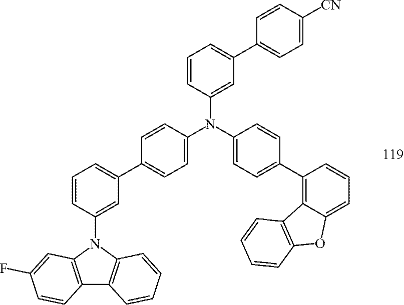 [SMILES: Fc1ccc2c3ccc(F)cc3n(-c3cccc(-c4cccc(N(c5ccc(-c6ccccc6)cc5)c5ccc(-c6cccc7c6oc6ccccc67)cc5)c4)c3)c2c1] 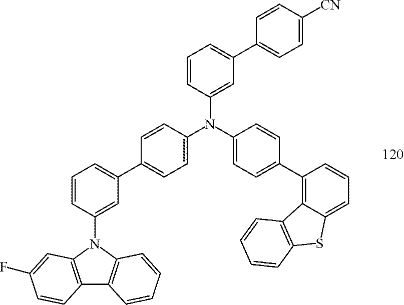 [SMILES: Fc1ccc2c3ccc(F)cc3n(-c3cccc(-c4cccc(N(c5ccc(-c6ccccc6)cc5)c5ccc(-c6cccc7c6sc6ccccc67)cc5)c4)c3)c2c1] 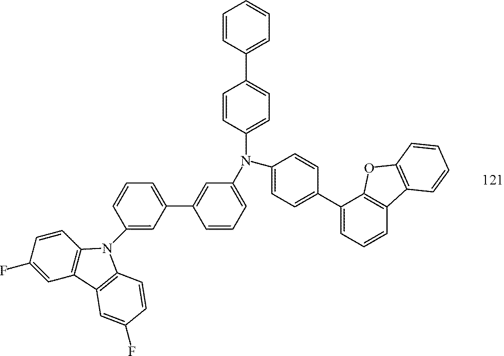 [SMILES: Fc1ccc2c3ccc(F)cc3n(-c3cccc(-c4cccc(N(c5ccc(-c6cccc7c6oc6ccccc67)cc5)c5cccc(-c6ccccc6)c5)c4)c3)c2c1] 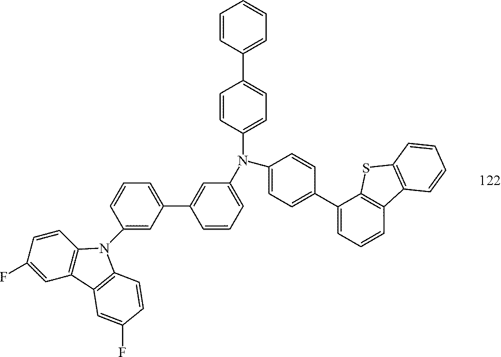 [SMILES: Fc1ccc2c3ccc(F)cc3n(-c3cccc(-c4cccc(N(c5ccc(-c6cccc7c6sc6ccccc67)cc5)c5cccc(-c6ccccc6)c5)c4)c3)c2c1] 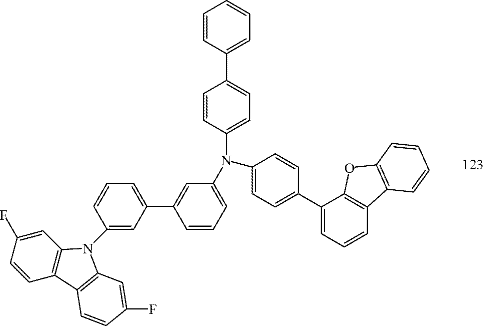 [SMILES: Fc1ccc2c3ccc(F)cc3n(-c3cccc(-c4cccc(N(c5ccc(-c6cccc7c6oc6ccccc67)cc5)c5ccccc5-c5ccccc5)c4)c3)c2c1] 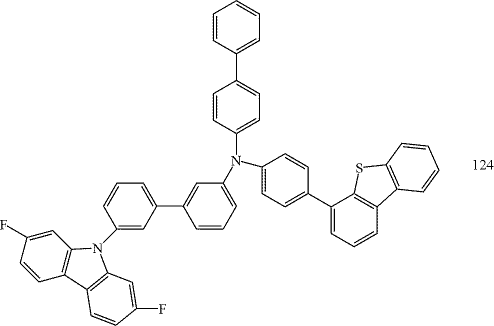 [SMILES: Fc1ccc2c3ccc(F)cc3n(-c3cccc(-c4cccc(N(c5ccc(-c6cccc7c6sc6ccccc67)cc5)c5ccccc5-c5ccccc5)c4)c3)c2c1] 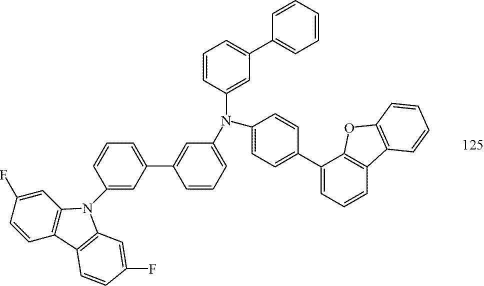 [SMILES: Fc1cc(F)c2c(c1)c1ccccc1n2-c1cccc(-c2cccc(N(c3ccc(-c4ccccc4)cc3)c3ccc(-c4cccc5c4sc4ccccc45)cc3)c2)c1] 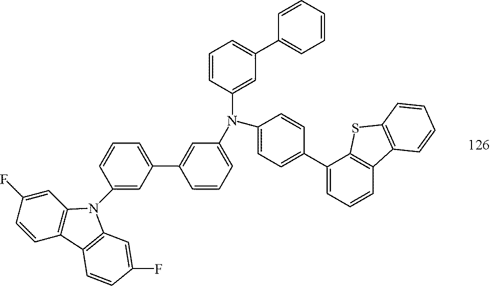 [SMILES: Fc1cc(F)c2c3ccccc3n(-c3cccc(-c4cccc(N(c5ccc(-c6ccccc6)cc5)c5ccc(-c6cccc7c6oc6ccccc67)cc5)c4)c3)c2c1] 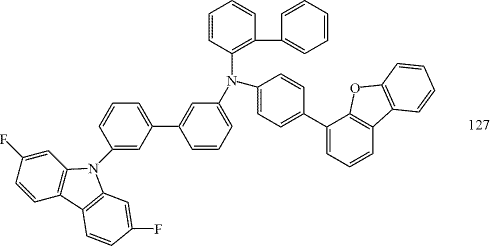 [SMILES: Fc1ccc2c3ccccc3n(-c3cccc(N(c4ccc(-c5cccc6c5oc5ccccc56)cc4)c4ccc(-c5cccc6cccc(-c7ccccc7)c56)cc4)c3)c2c1] 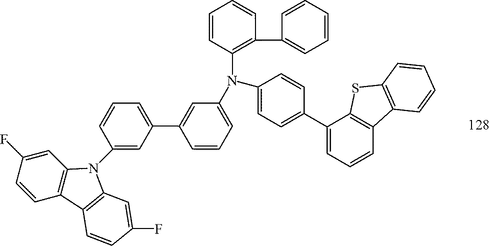 [SMILES: Fc1ccc2c3ccccc3n(-c3cccc(N(c4ccc(-c5cccc6c5sc5ccccc56)cc4)c4ccc(-c5cccc6cccc(-c7ccccc7)c56)cc4)c3)c2c1] 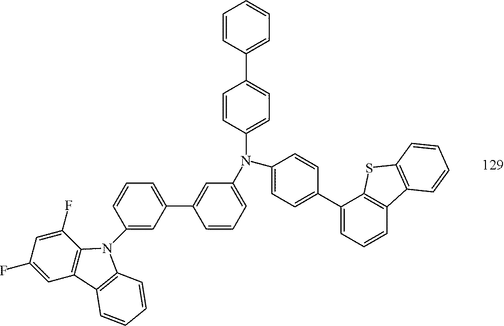 [SMILES: Fc1ccc2c3ccccc3n(-c3cccc(N(c4ccc(-c5cccc6c5oc5ccccc56)cc4)c4ccccc4-c4ccc(-c5cccc6ccccc56)cc4)c3)c2c1] 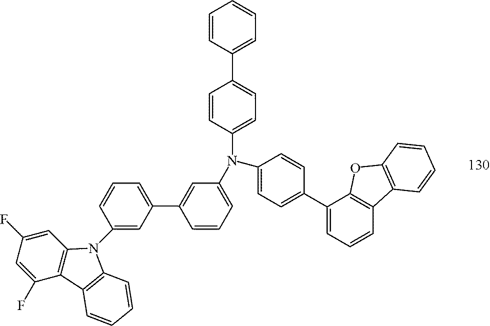 [SMILES: Fc1ccc2c3ccccc3n(-c3cccc(N(c4ccc(-c5cccc6c5sc5ccccc56)cc4)c4ccccc4-c4ccc(-c5cccc6ccccc56)cc4)c3)c2c1] 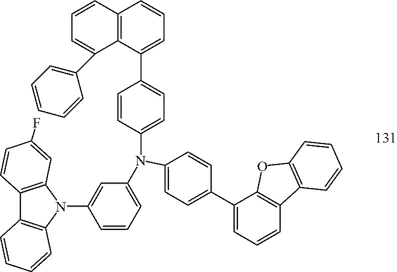 [SMILES: Fc1ccc2c3ccccc3n(-c3cccc(N(c4ccc(-c5cccc6c5oc5ccccc56)cc4)c4ccccc4-c4ccc(-c5ccccc5)c5ccccc45)c3)c2c1] 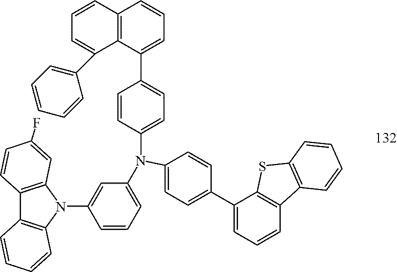 [SMILES: Fc1ccc2c3ccccc3n(-c3cccc(N(c4ccc(-c5cccc6c5sc5ccccc56)cc4)c4ccccc4-c4ccc(-c5ccccc5)c5ccccc45)c3)c2c1] 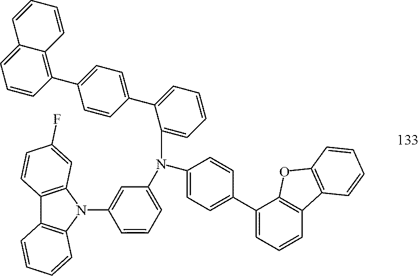 [SMILES: Fc1ccc2c3ccccc3n(-c3cccc(N(c4ccc(-c5cccc6c5oc5ccccc56)cc4)c4ccccc4-c4ccc(-c5ccccc5)cc4)c3)c2c1] 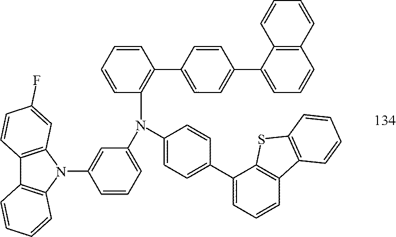 [SMILES: Fc1ccc2c3ccccc3n(-c3cccc(N(c4ccc(-c5cccc6c5sc5ccccc56)cc4)c4ccccc4-c4ccc(-c5ccccc5)cc4)c3)c2c1] 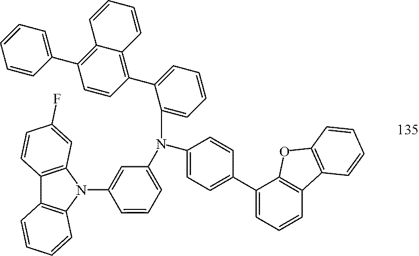 [SMILES: Fc1ccc2c3ccccc3n(-c3cccc(N(c4ccc(-c5cccc6c5oc5ccccc56)cc4)c4ccc5c6ccccc6n(-c6ccccc6)c5c4)c3)c2c1] 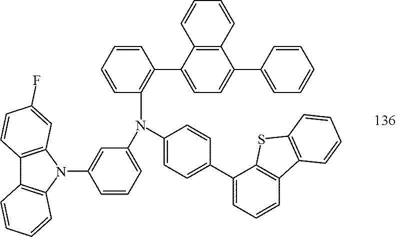 [SMILES: Fc1ccc2c3ccccc3n(-c3cccc(N(c4ccc(-c5cccc6c5sc5ccccc56)cc4)c4ccc5c(c4)c4ccccc4n5-c4ccccc4)c3)c2c1] 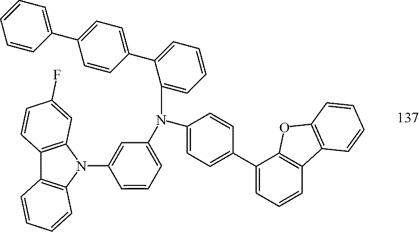 [SMILES: Fc1ccc2c3ccccc3n(-c3ccccc3-c3ccc(N(c4ccc(-c5cccc6oc7ccccc7c56)cc4)c4cccc(-c5ccccc5)c4)cc3)c2c1] 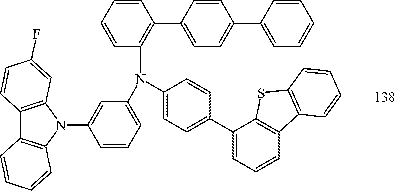 [SMILES: Fc1ccc2c3ccccc3n(-c3ccccc3-c3ccc(N(c4ccc(-c5cccc6oc7ccccc7c56)cc4)c4ccccc4-c4ccccc4)cc3)c2c1] 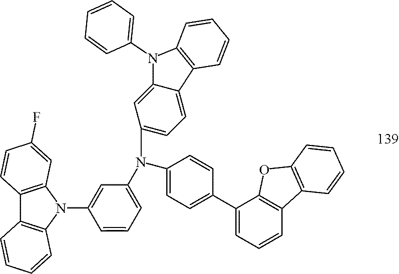 [SMILES: Fc1ccc2c3ccccc3n(-c3ccccc3-c3ccc(N(c4ccc(-c5ccccc5)cc4)c4ccc(-c5cccc6oc7ccccc7c56)cc4)cc3)c2c1] 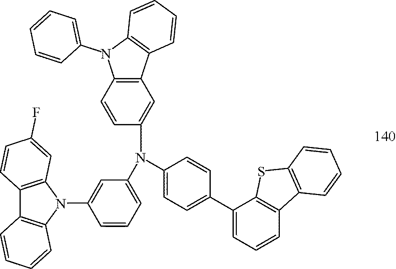 [SMILES: Fc1ccc2c3ccccc3n(-c3ccccc3-c3ccc(N(c4ccc(-c5cccc6sc7ccccc7c56)cc4)c4cccc(-c5ccccc5)c4)cc3)c2c1] 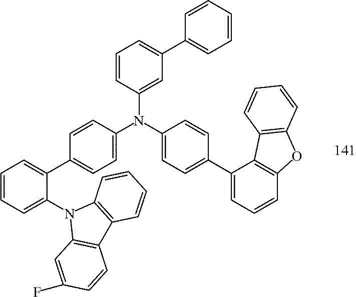 [SMILES: Fc1ccc2c3ccccc3n(-c3ccccc3-c3ccc(N(c4ccc(-c5cccc6sc7ccccc7c56)cc4)c4ccccc4-c4ccccc4)cc3)c2c1] 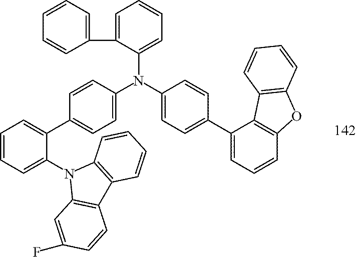 [SMILES: Fc1ccc2c3ccccc3n(-c3ccccc3-c3ccc(N(c4ccc(-c5ccccc5)cc4)c4ccc(-c5cccc6sc7ccccc7c56)cc4)cc3)c2c1] 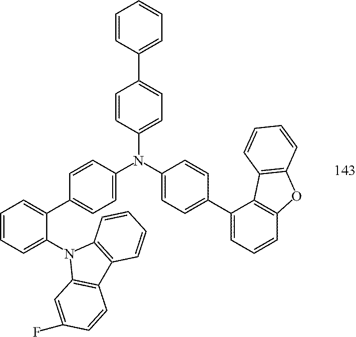 [SMILES: Fc1cccc2c3ccccc3n(-c3ccccc3-c3ccc(N(c4ccc(-c5cccc6oc7ccccc7c56)cc4)c4cccc(-c5ccccc5)c4)cc3)c12] 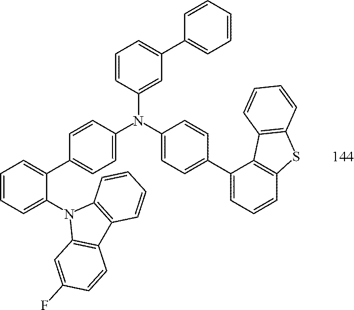 [SMILES: Fc1cccc2c3ccccc3n(-c3ccccc3-c3ccc(N(c4ccc(-c5cccc6oc7ccccc7c56)cc4)c4ccccc4-c4ccccc4)cc3)c12] 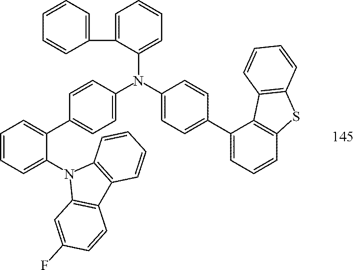 [SMILES: Fc1cccc2c3ccccc3n(-c3ccccc3-c3ccc(N(c4ccc(-c5ccccc5)cc4)c4ccc(-c5cccc6oc7ccccc7c56)cc4)cc3)c12] 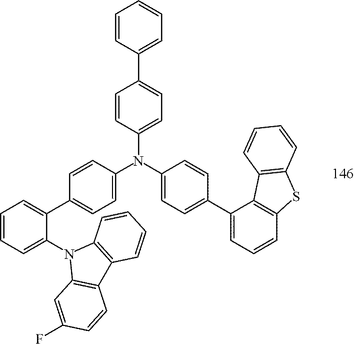 [SMILES: Fc1cccc2c3ccccc3n(-c3ccccc3-c3ccc(N(c4ccc(-c5cccc6sc7ccccc7c56)cc4)c4cccc(-c5ccccc5)c4)cc3)c12] 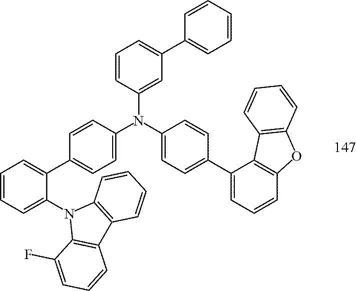 [SMILES: Fc1cccc2c3ccccc3n(-c3ccccc3-c3ccc(N(c4ccc(-c5cccc6sc7ccccc7c56)cc4)c4ccccc4-c4ccccc4)cc3)c12] 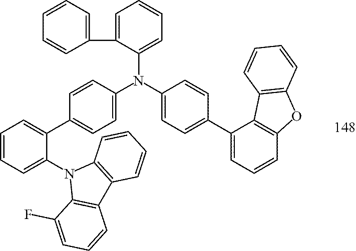 [SMILES: Fc1cccc2c3ccccc3n(-c3ccccc3-c3ccc(N(c4ccc(-c5ccccc5)cc4)c4ccc(-c5cccc6sc7ccccc7c56)cc4)cc3)c12] 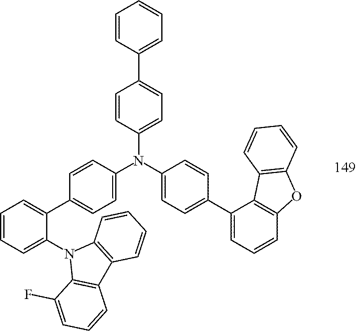 [SMILES: Fc1ccc2c(c1)c1ccccc1n2-c1ccccc1-c1ccc(N(c2ccc(-c3cccc4oc5ccccc5c34)cc2)c2cccc(-c3ccccc3)c2)cc1] 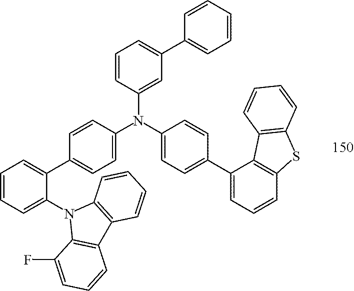 [SMILES: Fc1ccc2c(c1)c1ccccc1n2-c1ccccc1-c1ccc(N(c2ccc(-c3cccc4sc5ccccc5c34)cc2)c2ccccc2-c2ccccc2)cc1] 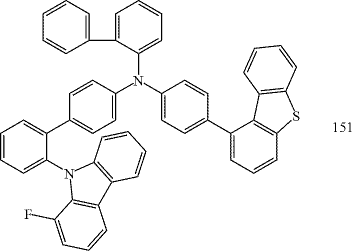 [SMILES: Fc1ccc2c(c1)c1ccccc1n2-c1ccccc1-c1ccc(N(c2ccc(-c3ccccc3)cc2)c2ccc(-c3cccc4oc5ccccc5c34)cc2)cc1] 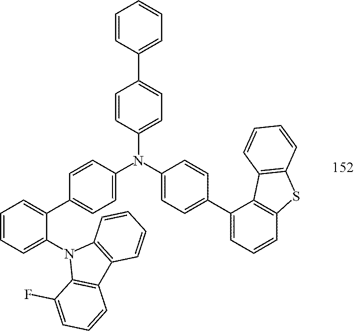 [SMILES: Fc1ccc2c(c1)c1ccccc1n2-c1ccccc1-c1ccc(N(c2ccc(-c3cccc4sc5ccccc5c34)cc2)c2cccc(-c3ccccc3)c2)cc1] 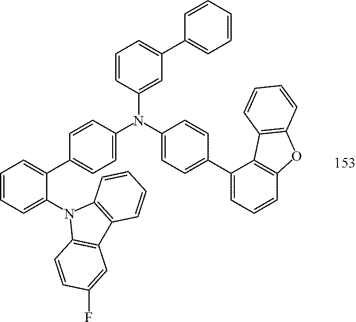 [SMILES: Fc1ccc2c(c1)c1ccccc1n2-c1ccccc1-c1ccc(N(c2ccc(-c3cccc4sc5ccccc5c34)cc2)c2ccccc2-c2ccccc2)cc1] 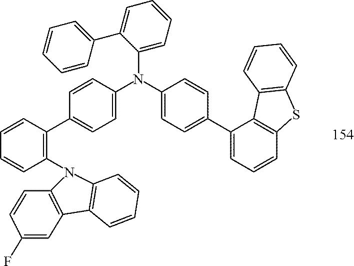 [SMILES: Fc1ccc2c(c1)c1ccccc1n2-c1ccccc1-c1ccc(N(c2ccc(-c3ccccc3)cc2)c2ccc(-c3cccc4sc5ccccc5c34)cc2)cc1] 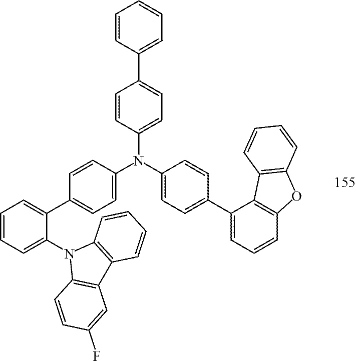 [SMILES: Fc1cccc2c1c1ccccc1n2-c1ccccc1-c1ccc(N(c2ccc(-c3cccc4oc5ccccc5c34)cc2)c2cccc(-c3ccccc3)c2)cc1] 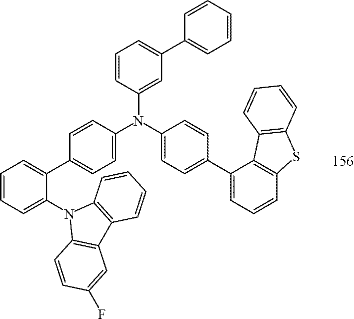 [SMILES: Fc1cccc2c1c1ccccc1n2-c1ccccc1-c1ccc(N(c2ccc(-c3cccc4oc5ccccc5c34)cc2)c2ccccc2-c2ccccc2)cc1] 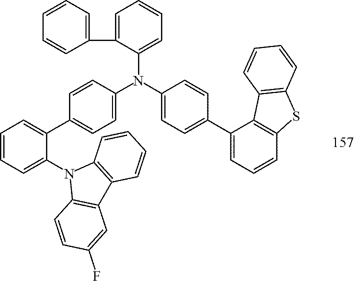 [SMILES: Fc1cccc2c1c1ccccc1n2-c1ccccc1-c1ccc(N(c2ccc(-c3ccccc3)cc2)c2ccc(-c3cccc4oc5ccccc5c34)cc2)cc1] 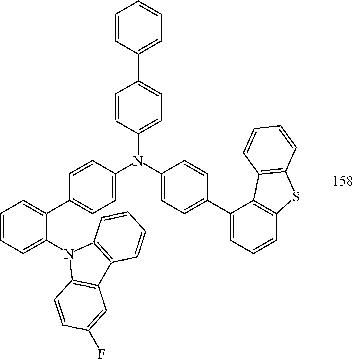 [SMILES: Fc1cccc2c1c1ccccc1n2-c1ccccc1-c1ccc(N(c2ccc(-c3cccc4sc5ccccc5c34)cc2)c2cccc(-c3ccccc3)c2)cc1] 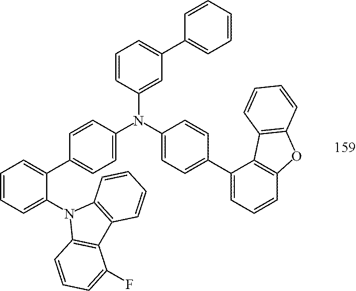 [SMILES: Fc1cccc2c1c1ccccc1n2-c1ccccc1-c1ccc(N(c2ccc(-c3cccc4sc5ccccc5c34)cc2)c2ccccc2-c2ccccc2)cc1] 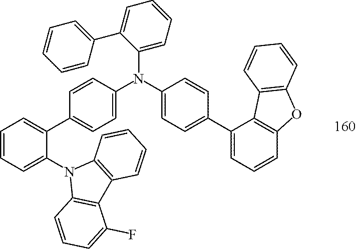 [SMILES: Fc1cccc2c1c1ccccc1n2-c1ccccc1-c1ccc(N(c2ccc(-c3ccccc3)cc2)c2ccc(-c3cccc4sc5ccccc5c34)cc2)cc1] 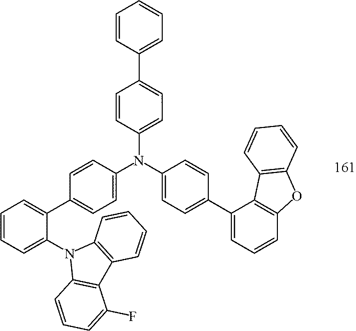 [SMILES: Fc1cccc2c1c1ccccc1n2-c1ccccc1-c1ccc(N(c2ccc(-c3cccc4c3oc3ccccc34)cc2)c2cccc(-c3ccccc3)c2)cc1] 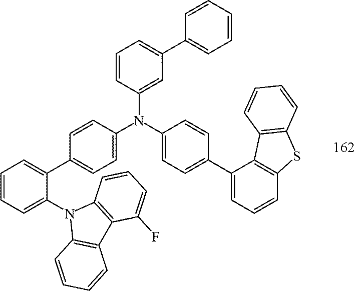 [SMILES: Fc1cccc2c1c1ccccc1n2-c1ccccc1-c1ccc(N(c2ccc(-c3cccc4c3oc3ccccc34)cc2)c2ccccc2-c2ccccc2)cc1] 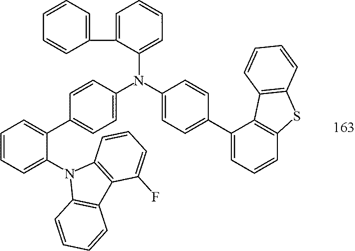 [SMILES: Fc1cccc2c1c1ccccc1n2-c1ccccc1-c1ccc(N(c2ccc(-c3ccccc3)cc2)c2ccc(-c3cccc4c3oc3ccccc34)cc2)cc1] 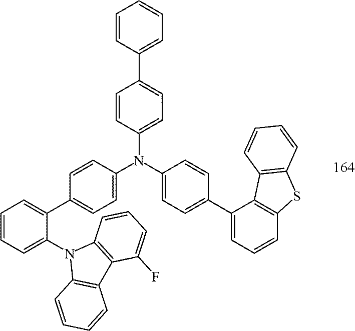 [SMILES: Fc1cccc2c1c1ccccc1n2-c1ccccc1-c1ccc(N(c2ccc(-c3cccc4c3sc3ccccc34)cc2)c2cccc(-c3ccccc3)c2)cc1] 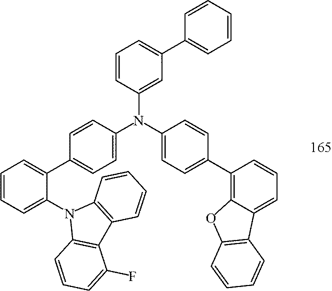 [SMILES: Fc1cccc2c1c1ccccc1n2-c1ccccc1-c1ccc(N(c2ccc(-c3cccc4c3sc3ccccc34)cc2)c2ccccc2-c2ccccc2)cc1] 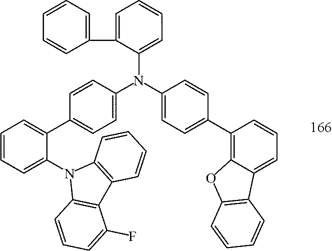 [SMILES: Fc1cccc2c1c1ccccc1n2-c1ccccc1-c1ccc(N(c2ccc(-c3ccccc3)cc2)c2ccc(-c3cccc4c3sc3ccccc34)cc2)cc1] 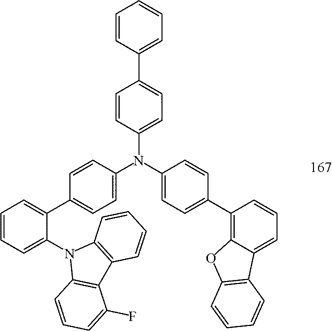 [SMILES: Fc1ccc2c(c1)c1ccccc1n2-c1ccccc1-c1ccc(N(c2ccc(-c3cccc4c3oc3ccccc34)cc2)c2cccc(-c3ccccc3)c2)cc1] 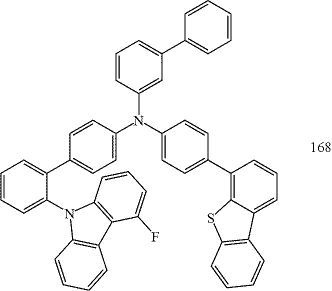 [SMILES: Fc1ccc2c(c1)c1ccccc1n2-c1ccccc1-c1ccc(N(c2ccc(-c3cccc4c3oc3ccccc34)cc2)c2ccccc2-c2ccccc2)cc1] 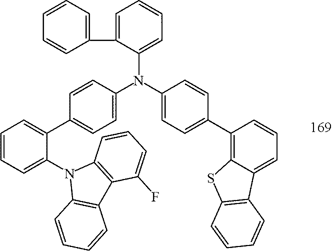 [SMILES: Fc1ccc2c(c1)c1ccccc1n2-c1ccccc1-c1ccc(N(c2ccc(-c3ccccc3)cc2)c2ccc(-c3cccc4c3oc3ccccc34)cc2)cc1] 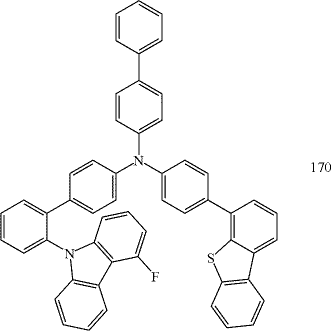 [SMILES: Fc1ccc2c(c1)c1ccccc1n2-c1ccccc1-c1ccc(N(c2ccc(-c3cccc4c3sc3ccccc34)cc2)c2cccc(-c3ccccc3)c2)cc1] 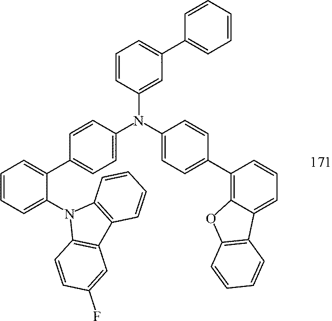 [SMILES: Fc1ccc2c(c1)c1ccccc1n2-c1ccccc1-c1ccc(N(c2ccc(-c3cccc4c3sc3ccccc34)cc2)c2ccccc2-c2ccccc2)cc1] 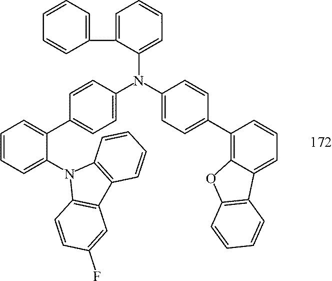 [SMILES: Fc1ccc2c(c1)c1ccccc1n2-c1ccccc1-c1ccc(N(c2ccc(-c3ccccc3)cc2)c2ccc(-c3cccc4c3sc3ccccc34)cc2)cc1] 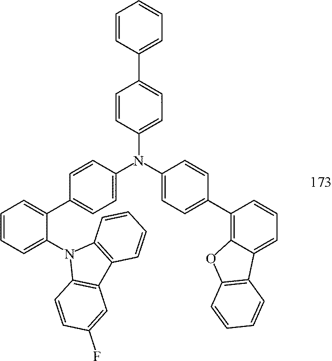 [SMILES: Fc1ccc2c3ccccc3n(-c3ccccc3-c3ccc(N(c4ccc(-c5cccc6c5oc5ccccc56)cc4)c4cccc(-c5ccccc5)c4)cc3)c2c1] 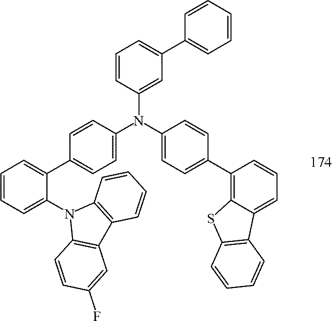 [SMILES: Fc1ccc2c3ccccc3n(-c3ccccc3-c3ccc(N(c4ccc(-c5cccc6c5oc5ccccc56)cc4)c4ccccc4-c4ccccc4)cc3)c2c1] 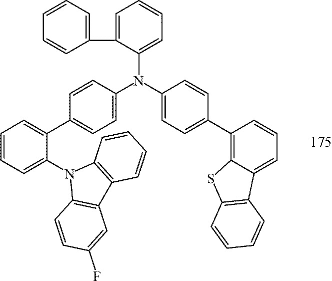 [SMILES: Fc1ccc2c3ccccc3n(-c3ccccc3-c3ccc(N(c4ccc(-c5ccccc5)cc4)c4ccc(-c5cccc6c5oc5ccccc56)cc4)cc3)c2c1] 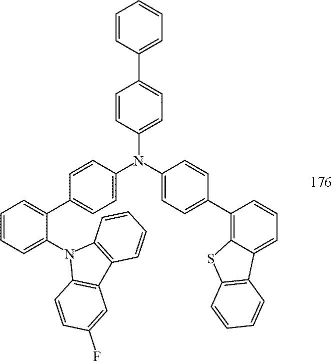 [SMILES: Fc1ccc2c3ccccc3n(-c3ccccc3-c3ccc(N(c4ccc(-c5cccc6c5sc5ccccc56)cc4)c4cccc(-c5ccccc5)c4)cc3)c2c1] 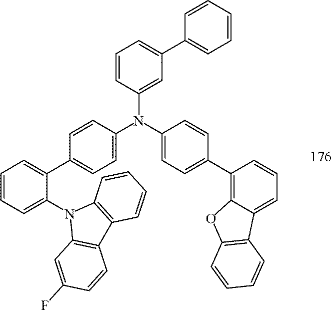 [SMILES: Fc1ccc2c3ccccc3n(-c3ccccc3-c3ccc(N(c4ccc(-c5cccc6c5sc5ccccc56)cc4)c4ccccc4-c4ccccc4)cc3)c2c1] 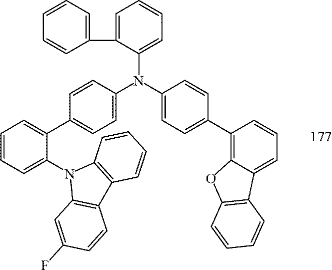 [SMILES: Fc1ccc2c3ccccc3n(-c3ccccc3-c3ccc(N(c4ccc(-c5ccccc5)cc4)c4ccc(-c5cccc6c5sc5ccccc56)cc4)cc3)c2c1] 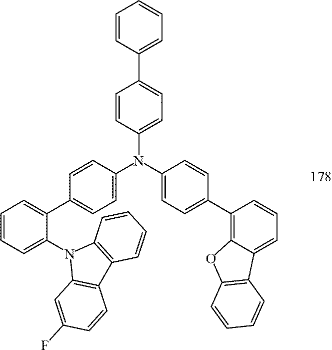 [SMILES: Fc1ccc2c3ccccc3n(-c3ccccc3-c3ccc(N(c4ccc(-c5ccc6c(c5)oc5ccccc56)cc4)c4cccc(-c5ccccc5)c4)cc3)c2c1] 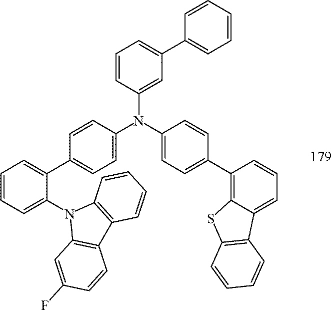 [SMILES: Fc1ccc2c3ccccc3n(-c3ccccc3-c3ccc(N(c4ccc(-c5ccc6c(c5)oc5ccccc56)cc4)c4ccccc4-c4ccccc4)cc3)c2c1] 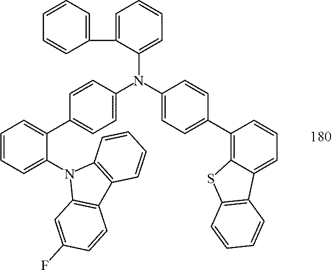 [SMILES: Fc1ccc2c3ccccc3n(-c3ccccc3-c3ccc(N(c4ccc(-c5ccccc5)cc4)c4ccc(-c5ccc6c(c5)oc5ccccc56)cc4)cc3)c2c1] 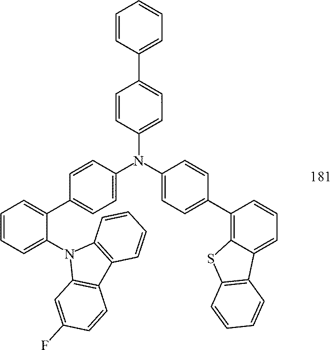 [SMILES: Fc1ccc2c3ccccc3n(-c3ccccc3-c3ccc(N(c4ccc(-c5ccc6c(c5)sc5ccccc56)cc4)c4cccc(-c5ccccc5)c4)cc3)c2c1] 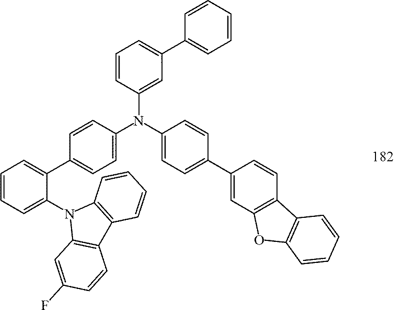 [SMILES: Fc1ccc2c3ccccc3n(-c3ccccc3-c3ccc(N(c4ccc(-c5ccc6c(c5)sc5ccccc56)cc4)c4ccccc4-c4ccccc4)cc3)c2c1] 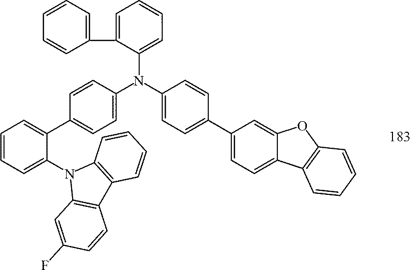 [SMILES: Fc1ccc2c3ccccc3n(-c3ccccc3-c3ccc(N(c4ccc(-c5ccccc5)cc4)c4ccc(-c5ccc6c(c5)sc5ccccc56)cc4)cc3)c2c1] 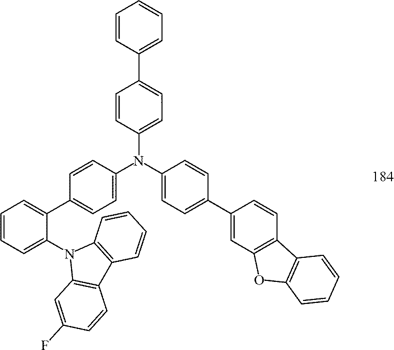 [SMILES: Fc1ccc2c3ccccc3n(-c3ccccc3-c3ccc(N(c4ccc(-c5ccc6oc7ccccc7c6c5)cc4)c4cccc(-c5ccccc5)c4)cc3)c2c1] 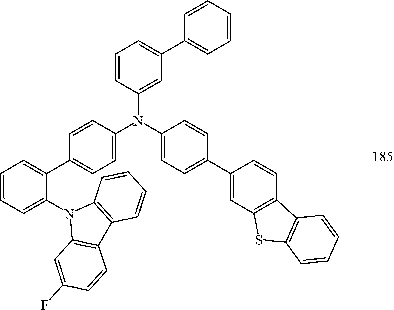 [SMILES: Fc1ccc2c3ccccc3n(-c3ccccc3-c3ccc(N(c4ccc(-c5ccc6oc7ccccc7c6c5)cc4)c4ccccc4-c4ccccc4)cc3)c2c1] 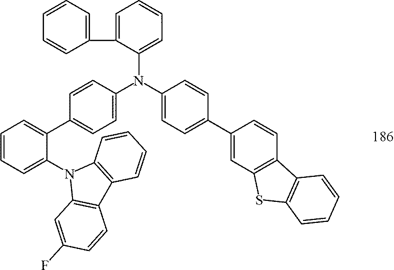 [SMILES: Fc1ccc2c3ccccc3n(-c3ccccc3-c3ccc(N(c4ccc(-c5ccccc5)cc4)c4ccc(-c5ccc6oc7ccccc7c6c5)cc4)cc3)c2c1] 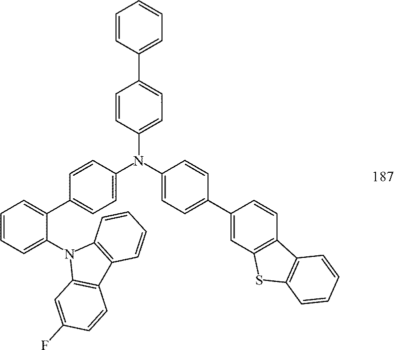 [SMILES: Fc1ccc2c3ccccc3n(-c3ccccc3-c3ccc(N(c4ccc(-c5ccc6sc7ccccc7c6c5)cc4)c4cccc(-c5ccccc5)c4)cc3)c2c1] 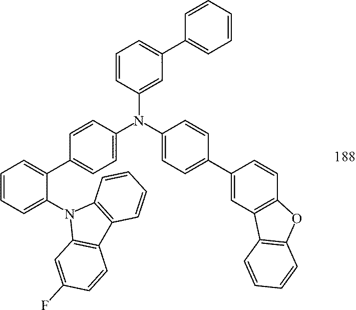 [SMILES: Fc1ccc2c3ccccc3n(-c3ccccc3-c3ccc(N(c4ccc(-c5ccc6sc7ccccc7c6c5)cc4)c4ccccc4-c4ccccc4)cc3)c2c1] 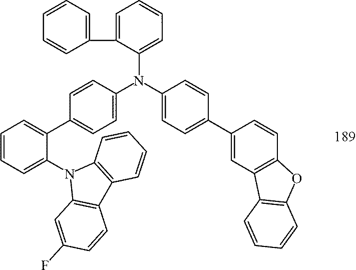 [SMILES: Fc1ccc2c3ccccc3n(-c3ccccc3-c3ccc(N(c4ccc(-c5ccccc5)cc4)c4ccc(-c5ccc6sc7ccccc7c6c5)cc4)cc3)c2c1] 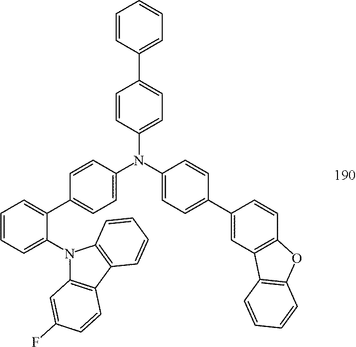 [SMILES: Fc1ccc2c3ccccc3n(-c3ccccc3-c3ccc(N(c4cccc(-c5ccccc5)c4)c4cccc(-c5cccc6c5oc5ccccc56)c4)cc3)c2c1] 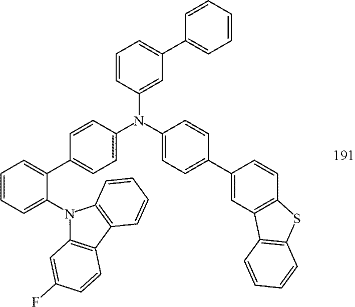 [SMILES: Fc1ccc2c(c1)c1ccccc1n2-c1ccccc1-c1ccc(N(c2cccc(-c3ccccc3)c2)c2cccc(-c3cccc4sc5ccccc5c34)c2)cc1] 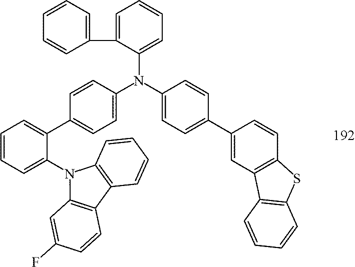 [SMILES: Fc1ccc2c(c1)c1ccc(F)cc1n2-c1ccccc1-c1ccc(N(c2cccc(-c3ccccc3)c2)c2cccc(-c3cccc4c3oc3ccccc34)c2)cc1] 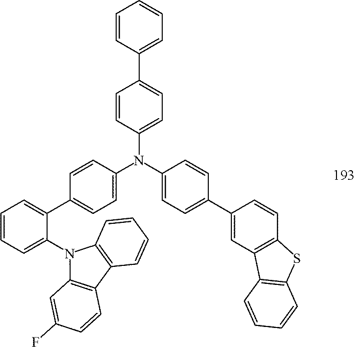 [SMILES: Fc1ccc2c(c1)c1c(F)cccc1n2-c1ccccc1-c1ccc(N(c2cccc(-c3ccccc3)c2)c2cccc(-c3cccc4sc5ccccc5c34)c2)cc1] 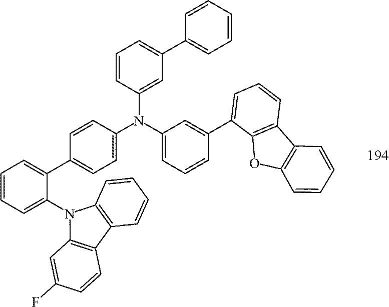 [SMILES: Fc1cc(F)c2c(c1)c1ccccc1n2-c1cccc(-c2ccc(N(c3cccc(-c4cccc5c4oc4ccccc45)c3)c3cccc4ccccc34)cc2)c1] 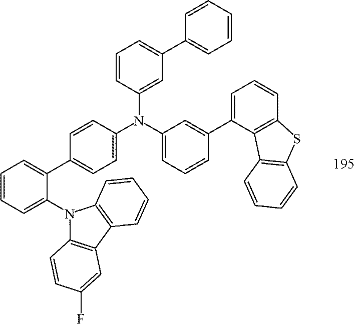 [SMILES: Fc1cc(F)c2c3ccccc3n(-c3cccc(-c4ccc(N(c5cccc(-c6cccc7sc8ccccc8c67)c5)c5ccc6ccccc6c5)cc4)c3)c2c1] 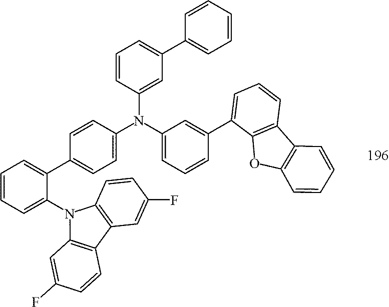 [SMILES: Fc1ccc2c3ccccc3n(-c3ccccc3-c3ccc(N(c4cccc(-c5ccccc5)c4)c4cccc5c4oc4ccccc45)cc3)c2c1] 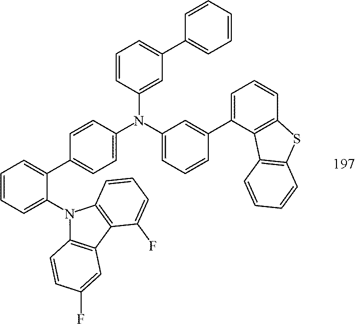 [SMILES: Fc1ccc2c3ccccc3n(-c3ccccc3-c3ccc(N(c4ccc(-c5ccccc5)cc4)c4cccc5c4oc4ccccc45)cc3)c2c1] 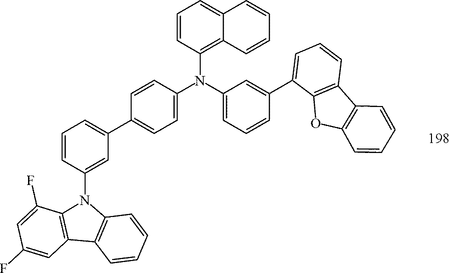 [SMILES: Fc1ccc2c3ccccc3n(-c3cccc(-c4ccc(N(c5ccc(-c6ccccc6)cc5)c5cccc6c5oc5ccccc56)cc4)c3)c2c1] 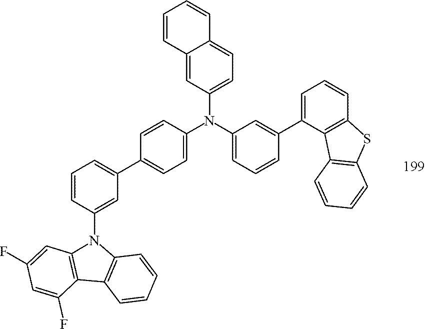 [SMILES: Fc1ccc2c(c1)c1ccccc1n2-c1cccc(-c2ccc(N(c3ccc(-c4ccccc4)cc3)c3cccc4c3oc3ccccc34)cc2)c1] 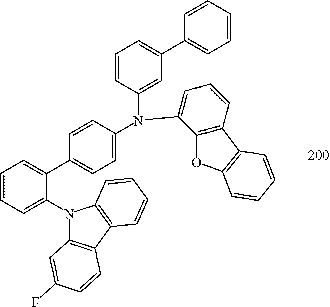 [SMILES: Fc1ccc2c3ccccc3n(-c3ccccc3-c3ccc(N(c4cccc(-c5ccccc5)c4)c4cccc5oc6ccccc6c45)cc3)c2c1] 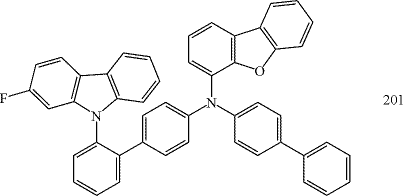 [SMILES: Fc1ccc2c3ccccc3n(-c3ccccc3-c3ccc(N(c4ccc(-c5ccccc5)cc4)c4cccc5oc6ccccc6c45)cc3)c2c1] 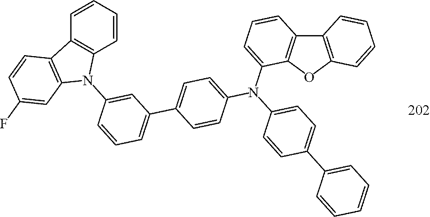 [SMILES: Fc1ccc2c3ccccc3n(-c3cccc(-c4ccc(N(c5ccc(-c6ccccc6)cc5)c5ccc6oc7ccccc7c6c5)cc4)c3)c2c1] 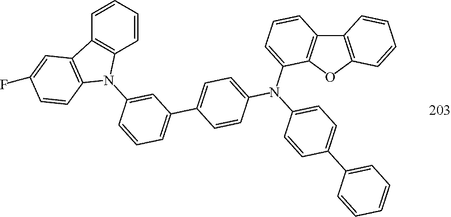 [SMILES: Fc1ccc2c(c1)c1ccccc1n2-c1cccc(-c2ccc(N(c3ccc(-c4ccccc4)cc3)c3cccc4c3oc3ccccc34)cc2)c1] 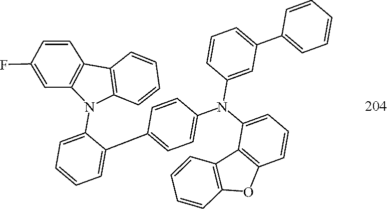 [SMILES: Fc1ccc2c3ccccc3n(-c3ccccc3-c3ccc(N(c4ccc(-c5cccc6c5oc5ccccc56)cc4)c4cccc5c4c4ccccc4n5-c4ccccc4)cc3)c2c1] 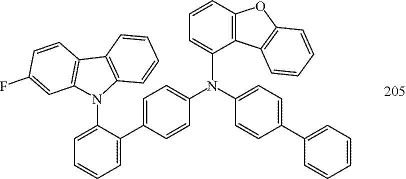 [SMILES: CC1(C)c2ccccc2-c2ccc(N(c3ccc(-c4ccccc4-n4c5ccccc5c5ccc(F)cc54)cc3)c3ccc(-c4cccc5sc6ccccc6c45)cc3)cc21] 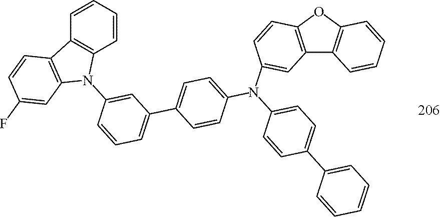 [SMILES: Fc1ccc2c3ccccc3n(-c3ccccc3-c3ccc(N(c4ccc(-c5cccc6c5oc5ccccc56)cc4)c4ccc5sc6ccccc6c5c4)cc3)c2c1] 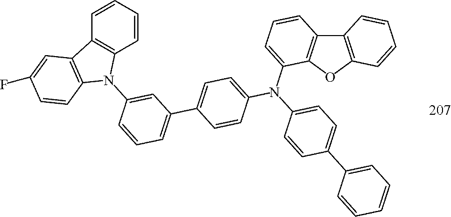 [SMILES: CC1(C)c2ccccc2-c2cc(N(c3ccc(-c4ccccc4-n4c5ccccc5c5cc(F)ccc54)cc3)c3ccc(-c4cccc5sc6ccccc6c45)cc3)ccc21] 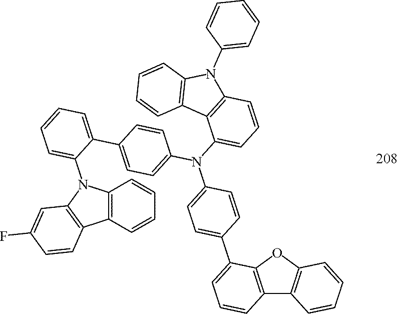 [SMILES: Fc1ccc2c3ccccc3n(-c3cccc(-c4ccc(N(c5ccc(-c6cccc7c6oc6ccccc67)cc5)c5ccc6c7ccccc7n(-c7ccccc7)c6c5)cc4)c3)c2c1] 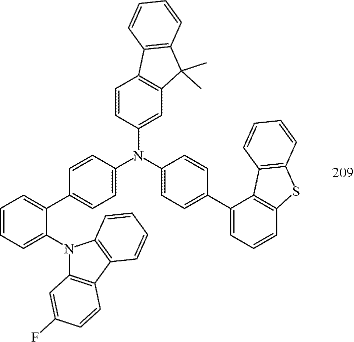 [SMILES: CC1(C)c2ccccc2-c2ccc(N(c3ccc(-c4cccc(-n5c6ccccc6c6ccc(F)cc65)c4)cc3)c3ccc(-c4cccc5c4oc4ccccc45)cc3)cc21] 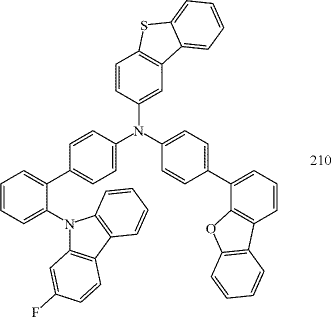 [SMILES: Fc1ccc2c3ccccc3n(-c3cccc(-c4ccc(N(c5ccc(-c6cccc7c6oc6ccccc67)cc5)c5ccc6sc7ccccc7c6c5)cc4)c3)c2c1] 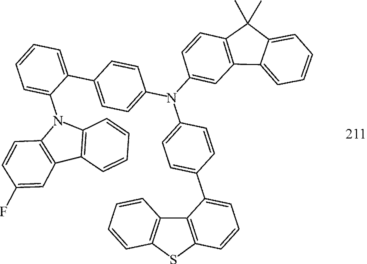 [SMILES: CC1(C)c2ccccc2-c2cc(N(c3ccc(-c4cccc(-n5c6ccccc6c6cc(F)ccc65)c4)cc3)c3ccc(-c4cccc5sc6ccccc6c45)cc3)ccc21] 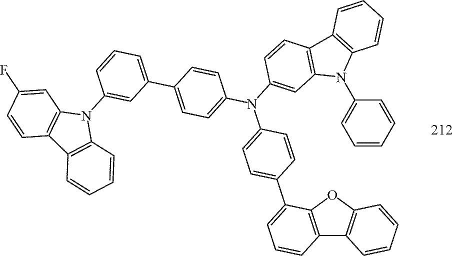 [SMILES: Fc1ccc2c3ccccc3n(-c3cccc(-c4ccc(N(c5ccc(-c6cccc7c6oc6ccccc67)cc5)c5ccc6c7ccccc7n(-c7ccccc7)c6c5)cc4)c3)c2c1] 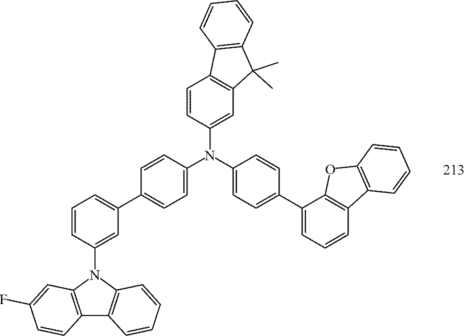 [SMILES: CC1(C)c2ccccc2-c2ccc(N(c3ccc(-c4cccc(-n5c6ccccc6c6ccc(F)cc65)c4)cc3)c3ccc(-c4cccc5oc6ccccc6c45)cc3)cc21] 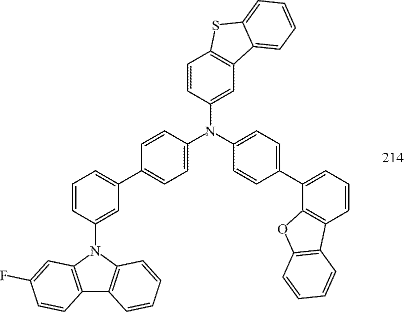 [SMILES: Fc1ccc2c3ccccc3n(-c3cccc(-c4ccc(N(c5ccc(-c6cccc7c6oc6ccccc67)cc5)c5ccc6sc7ccccc7c6c5)cc4)c3)c2c1] 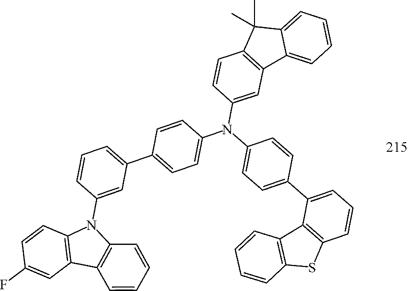 [SMILES: CC1(C)c2ccccc2-c2cc(N(c3ccc(-c4cccc(-n5c6ccccc6c6cc(F)ccc65)c4)cc3)c3ccc(-c4cccc5sc6ccccc6c45)cc3)ccc21] 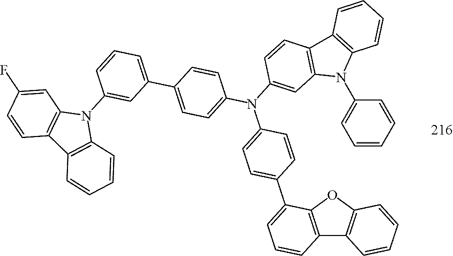 [SMILES: Fc1ccc2c3ccccc3n(-c3cccc(-c4ccc(N(c5ccc(-c6ccccc6)cc5)c5cccc(-c6ccc7c(c6)oc6ccccc67)c5)cc4)c3)c2c1] 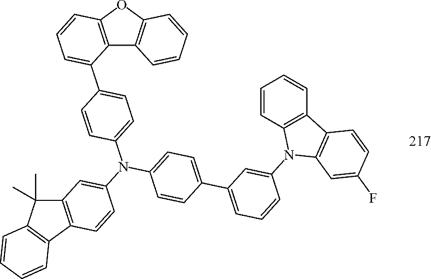 [SMILES: Fc1cccc2c1c1c(F)cccc1n2-c1cccc(-c2ccc(N(c3ccc(-c4ccccc4)cc3)c3ccc(-c4cccc5c4oc4ccccc45)cc3)cc2)c1] 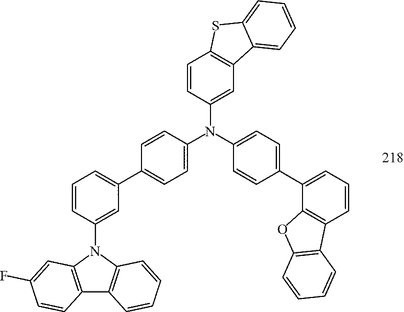 [SMILES: Fc1ccc2c3c(F)cccc3n(-c3cccc(-c4ccc(N(c5ccc(-c6ccccc6)cc5)c5ccc(-c6cccc7c6oc6ccccc67)cc5)cc4)c3)c2c1] 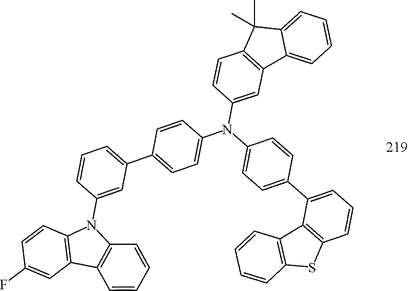 [SMILES: Fc1cccc2c1c1cccc(F)c1n2-c1cccc(-c2ccc(N(c3ccc(-c4ccccc4)cc3)c3ccc(-c4cccc5c4oc4ccccc45)cc3)cc2)c1] 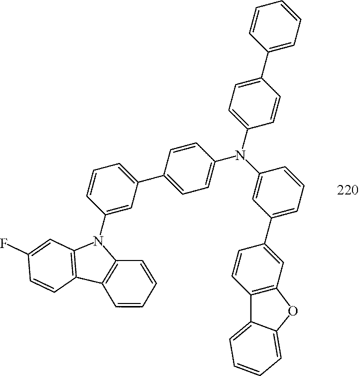 [SMILES: Fc1cc(F)c2c3ccccc3n(-c3cccc(-c4ccc(N(c5ccc(-c6ccccc6)cc5)c5cccc(-c6ccc7c(c6)oc6ccccc67)c5)cc4)c3)c2c1] 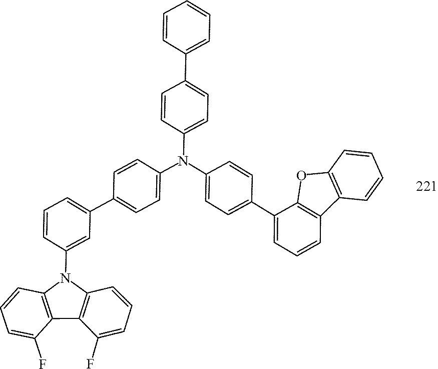 [SMILES: Fc1ccc2c3ccccc3n(-c3cccc(-c4ccc(N(c5ccccc5)c5ccc(-c6cccc7c6oc6ccccc67)cc5)cc4)c3)c2c1] 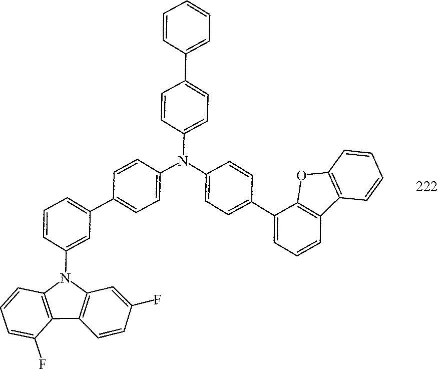 [SMILES: Fc1ccc2c3c(F)cccc3n(-c3cccc(-c4ccc(N(c5ccc(-c6ccccc6)cc5)c5ccc(-c6cccc7c6oc6ccccc67)cc5)cc4)c3)c2c1] 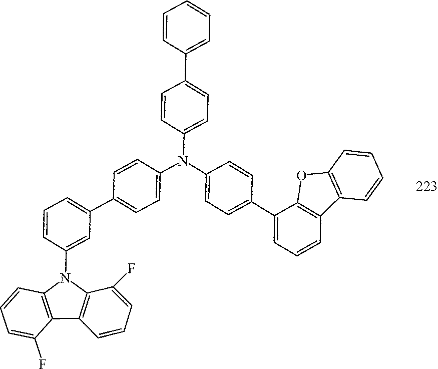 [SMILES: Fc1cccc2c1c1cccc(F)c1n2-c1cccc(-c2ccc(N(c3ccc(-c4ccccc4)cc3)c3ccc(-c4cccc5c4oc4ccccc45)cc3)cc2)c1] 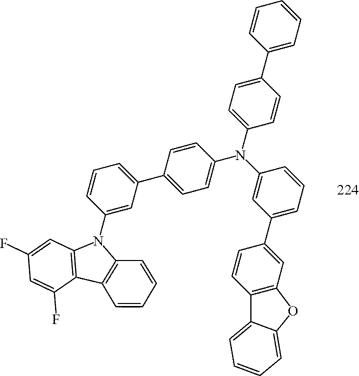 [SMILES: Fc1ccc2c3ccccc3n(-c3cccc(-c4ccc(N(c5ccc(-c6cccc7c6oc6ccccc67)cc5)c5cccc(-c6ccc7ccccc7c6)c5)cc4)c3)c2c1] 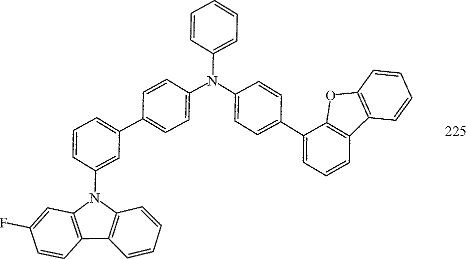 [SMILES: Fc1ccc2c(c1)c1ccccc1n2-c1cccc(-c2ccc(N(c3ccc(-c4cccc5c4oc4ccccc45)cc3)c3ccc4c(-c5ccccc5)cccc4c3)cc2)c1] 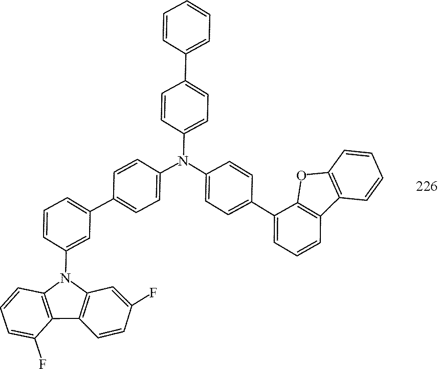 [SMILES: Fc1ccc2c3ccccc3n(-c3ccc4c(-c5ccc(N(c6ccc(-c7ccccc7)cc6)c6cccc7oc8ccccc8c67)cc5)cccc4c3)c2c1] 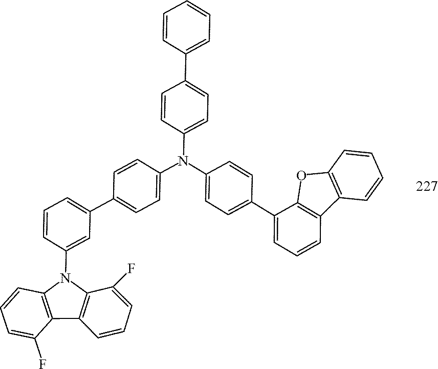 [SMILES: Fc1cccc2c1c1ccccc1n2-c1cccc(-c2ccc(N(c3ccc(-c4ccccc4)cc3)c3ccc(-c4cccc5c4sc4ccccc45)cc3)c3ccccc23)c1] 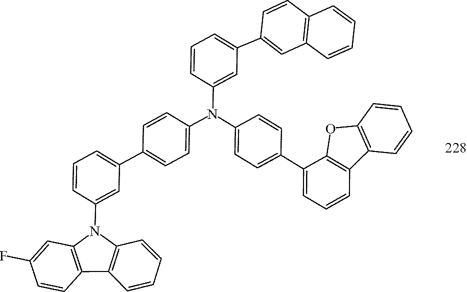 [SMILES: Fc1ccc2c3ccccc3n(-c3cccc(-c4ccc(N(c5cccc(-c6ccccc6)c5)c5cccc6c(-c7cccc8c7oc7ccccc78)cccc56)cc4)c3)c2c1] 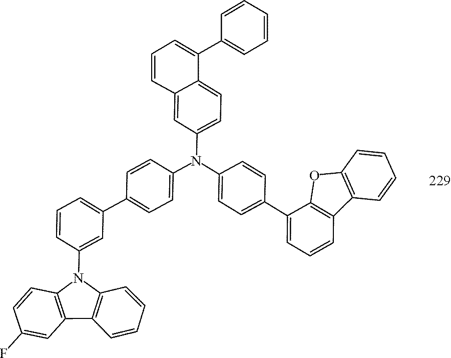 [SMILES: Fc1ccc2c(c1)c1ccccc1n2-c1cccc(-c2ccc(N(C3=CC=C(c4ccccc4)CC3)c3ccc(-c4cccc(-c5cccc6c5sc5ccccc56)c4)cc3)cc2)c1] 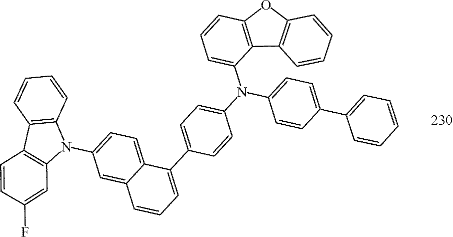 [SMILES: Fc1ccc2c3ccccc3n(-c3cccc(-c4ccc(N(c5ccc(-c6ccccc6)cc5)c5cccc6oc7cc(-c8ccccc8)ccc7c56)cc4)c3)c2c1] 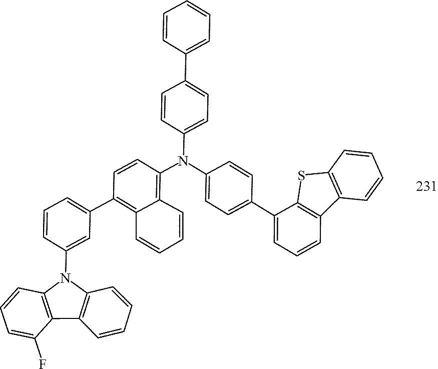 [SMILES: Fc1cccc2c1c1ccccc1n2-c1cccc(-c2ccc(N(c3ccc(-c4ccccc4)cc3)c3ccc(-c4cccc5c4oc4ccccc45)cc3)c3ccccc23)c1] 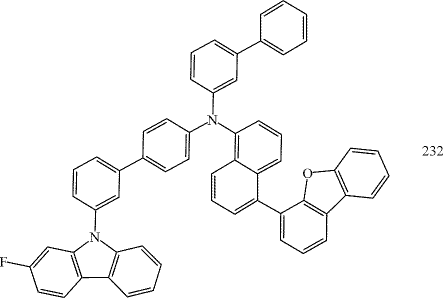 [SMILES: Fc1cccc2c1c1cccc(F)c1n2-c1ccccc1-c1ccc(N(c2ccc(-c3ccccc3)cc2)c2ccc(-c3cccc4c3oc3ccccc34)cc2)cc1] 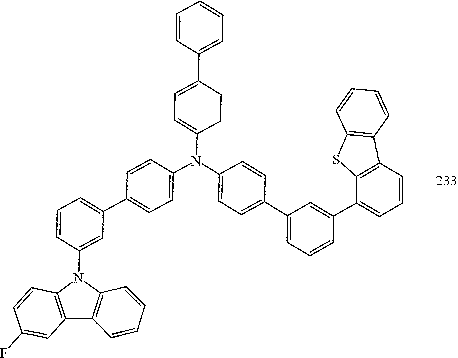 [SMILES: Fc1ccc2c3c(F)cccc3n(-c3ccccc3-c3ccc(N(c4ccc(-c5ccccc5)cc4)c4ccc(-c5cccc6c5sc5ccccc56)cc4)cc3)c2c1] 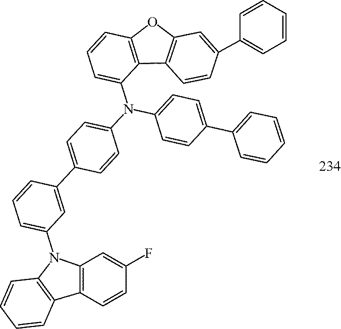 [SMILES: Fc1ccc2c(c1)c1ccc(F)cc1n2-c1ccccc1-c1ccc(N(c2ccc(-c3ccccc3)cc2)c2ccc(-c3cccc4sc5ccccc5c34)cc2)cc1] 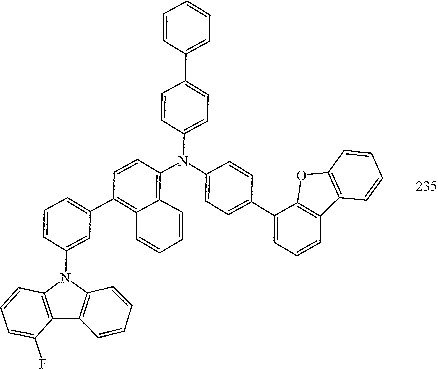 [SMILES: CC1(C)c2ccccc2-c2cc(N(c3ccc(-c4ccccc4-n4c5ccc(F)cc5c5ccc(F)cc54)cc3)c3ccc(-c4cccc5sc6ccccc6c45)cc3)ccc21] 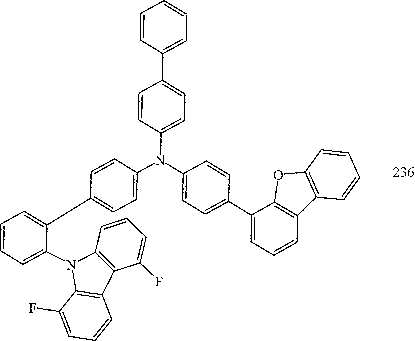 [SMILES: Fc1cccc2c3ccccc3n(-c3cccc(-c4ccc(N(c5ccc(-c6ccccc6)cc5)c5ccc(-c6cccc7c6oc6ccccc67)cc5)cc4)c3)c12] 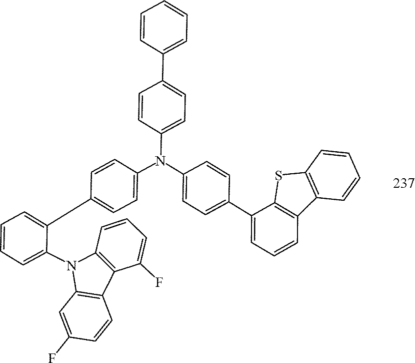 [SMILES: Fc1ccc2c(c1)c1cccc(F)c1n2-c1cccc(-c2ccc(N(c3ccc(-c4cccc5c4oc4ccccc45)cc3)c3cccc(-c4ccccc4)c3)cc2)c1] 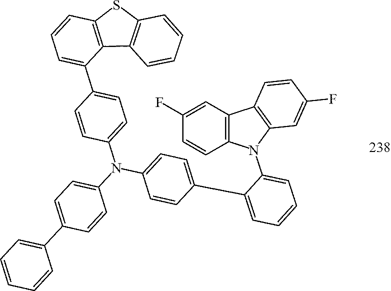 [SMILES: Fc1ccc2c(c1)c1ccc(F)cc1n2-c1cccc(-c2ccc(N(c3ccc(-c4ccccc4)cc3)c3ccc(-c4cccc5oc6ccccc6c45)cc3)cc2)c1] 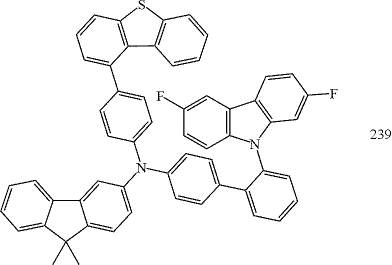 [SMILES: Fc1cc(F)c2c3ccccc3n(-c3cccc(-c4ccc(N(c5ccc(-c6cccc7oc8ccccc8c67)cc5)c5cccc(-c6ccccc6)c5)cc4)c3)c2c1] 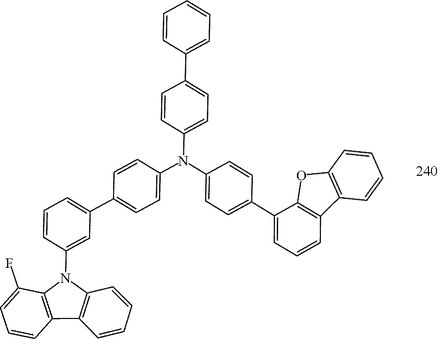 [SMILES: CC(C)(C)c1ccc(N(c2ccc(-c3cccc(-n4c5ccccc5c5cccc(F)c54)c3)cc2)c2ccc(-c3cccc4c3sc3ccccc34)cc2)cc1] 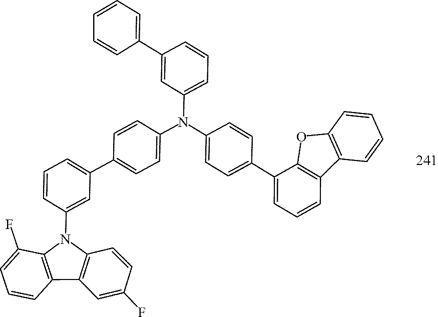 [SMILES: Fc1ccc2c(c1)c1cccc(F)c1n2-c1cccc(-c2ccc(N(c3ccc(-c4cccc5c4sc4ccccc45)cc3)c3cccc(-c4ccccc4)c3)cc2)c1] 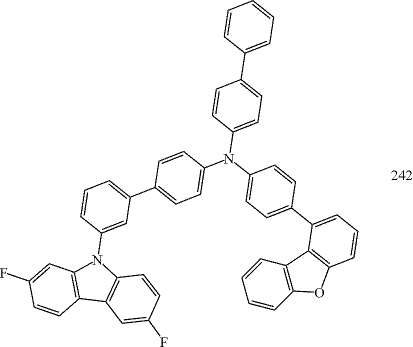 [SMILES: Fc1ccc2c(c1)c1ccc(F)cc1n2-c1cccc(-c2ccc(N(c3ccc(-c4ccccc4)cc3)c3ccc(-c4cccc5sc6ccccc6c45)cc3)cc2)c1] 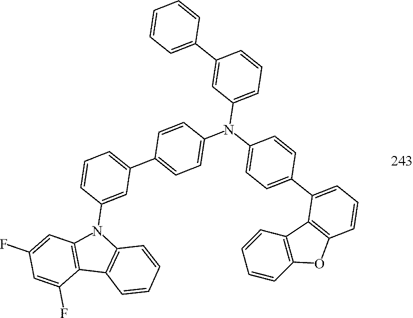 [SMILES: Fc1ccc2c3ccc(F)cc3n(-c3cccc(-c4ccc(N(c5ccc(-c6cccc7sc8ccccc8c67)cc5)c5cccc(C6CCCCC6)c5)cc4)c3)c2c1] 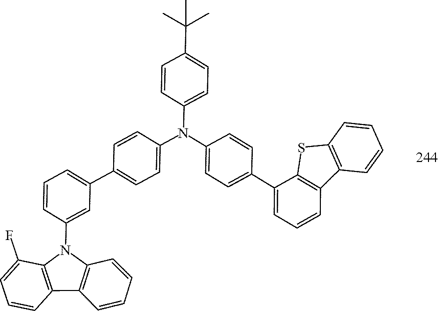 [SMILES: Fc1cc(F)c2c(c1)c1ccccc1n2-c1cccc(-c2ccc(N(c3ccc(-c4ccccc4)cc3)c3ccc(-c4cccc5c4sc4ccccc45)cc3)cc2)c1] 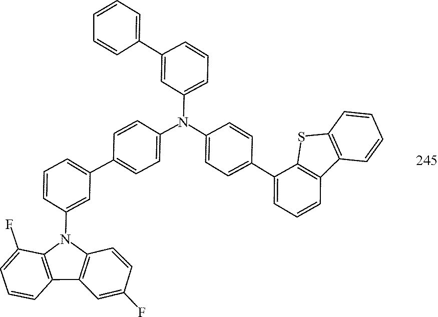 [SMILES: Fc1ccc(F)c2c1c1ccccc1n2-c1cccc(-c2ccc(N(c3ccc(-c4ccccc4)cc3)c3ccc(-c4cccc5sc6ccccc6c45)cc3)cc2)c1] 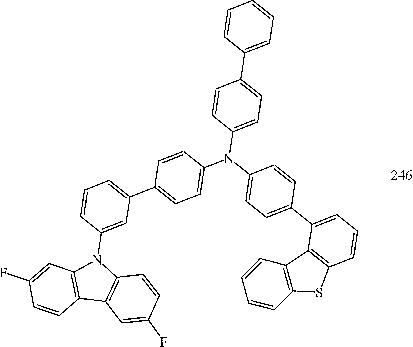 [SMILES: Fc1ccc(F)c2c1c1ccccc1n2-c1cccc(-c2ccc(N(c3ccc(-c4ccccc4)cc3)c3ccc(-c4cccc5sc6ccccc6c45)cc3)cc2)c1] 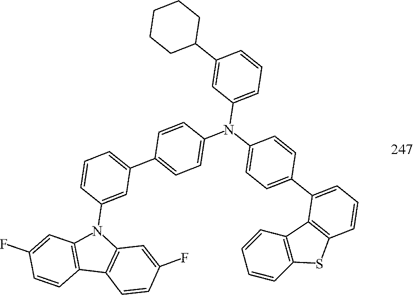 [SMILES: [2H]c1c([2H])c([2H])c(-c2cccc(N(c3ccc(-c4cccc(-n5c6ccccc6c6ccc(F)cc65)c4)cc3)c3ccc(-c4cccc5sc6ccccc6c45)cc3)c2)c([2H])c1[2H]] 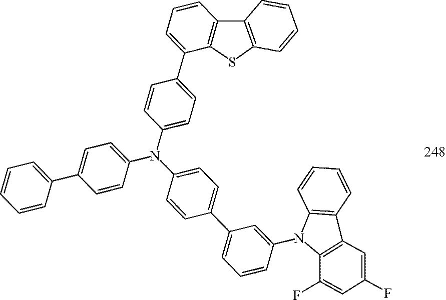 [SMILES: Fc1ccc2c3ccccc3n(-c3cccc(-c4ccc(N(c5ccccc5)c5ccc(-c6cccc7c6oc6cc(-c8ccccc8)ccc67)cc5)cc4)c3)c2c1] 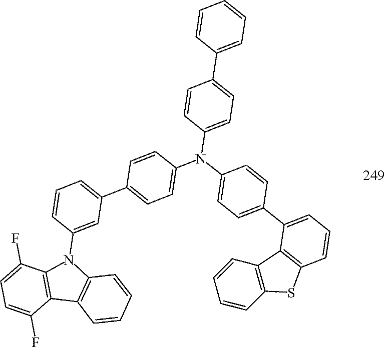 [SMILES: Fc1ccc2c3ccccc3n(-c3cccc(-c4ccc(N(c5ccccc5)c5ccc(-c6cccc7oc8cc(-c9ccccc9)ccc8c67)cc5)cc4)c3)c2c1] 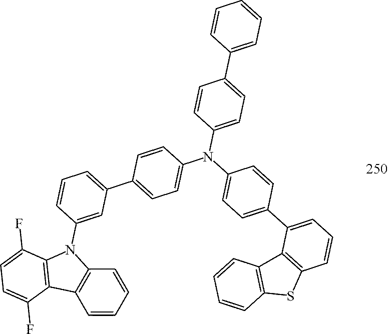 [SMILES: Fc1ccc2c3ccccc3n(-c3cccc(-c4ccc(N(c5ccc(-c6cccc7c6oc6ccccc67)cc5)c5ccc6ccccc6c5)cc4)c3)c2c1] 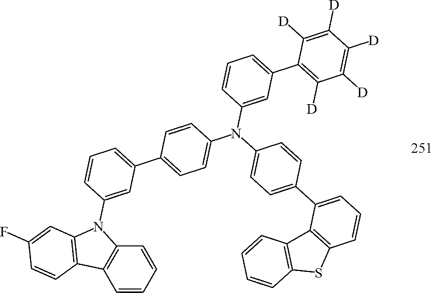 [SMILES: Fc1ccc2c(c1)c1ccccc1n2-c1cccc(-c2ccc(N(c3ccc(-c4cccc5c4oc4ccccc45)cc3)c3cccc4ccccc34)cc2)c1] 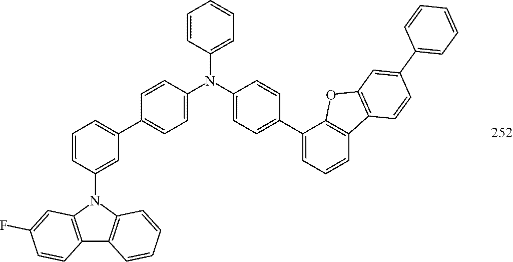 [SMILES: Fc1ccc2c3ccccc3n(-c3cccc(-c4ccc(N(c5cccc(-c6cccc(-c7ccccc7)c6)c5)c5cccc6c5oc5ccccc56)cc4)c3)c2c1] 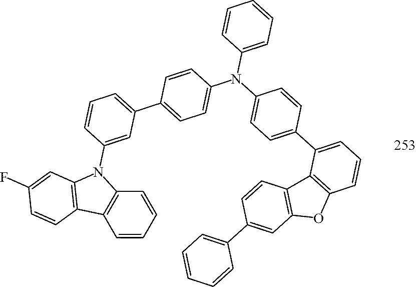 [SMILES: Cc1cccc(-c2cccc(N(c3ccc(-c4cccc(-n5c6ccccc6c6ccc(F)cc65)c4)cc3)c3ccc(-c4cccc5c4oc4ccccc45)cc3)c2)c1] 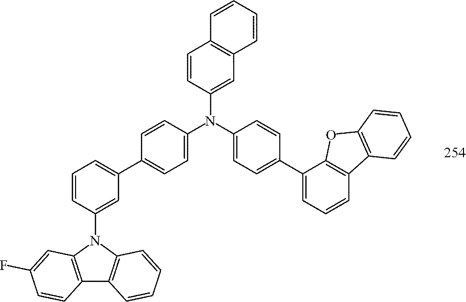 [SMILES: Fc1ccc2c3ccccc3n(-c3cccc(-c4ccc(N(c5ccc(-c6ccccc6F)cc5)c5ccc(-c6cccc7c6oc6ccccc67)cc5)cc4)c3)c2c1] 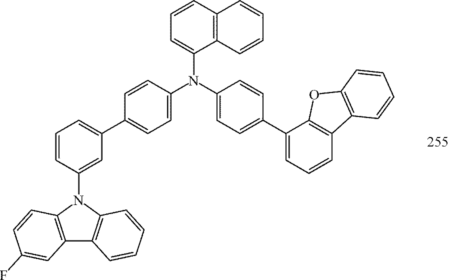 [SMILES: N#Cc1ccccc1-c1ccc(N(c2ccc(-c3ccccc3-n3c4ccccc4c4ccc(F)cc43)cc2)c2ccc(-c3cccc4c3oc3ccccc34)cc2)cc1] 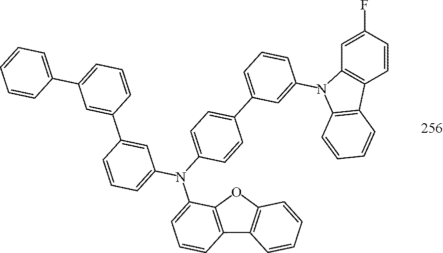 [SMILES: Fc1ccc2c3ccccc3n(-c3cccc(-c4ccc(N(c5ccc(-c6ccccc6)cc5)c5ccccc5-c5cccc6c5sc5ccccc56)cc4)c3)c2c1] 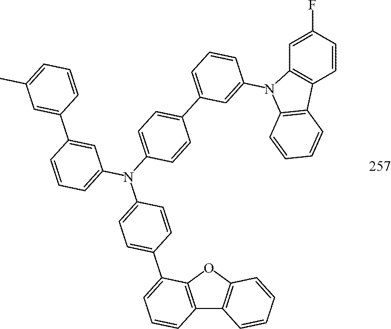 [SMILES: Cc1cc(N(c2ccc(-c3cccc4c3sc3ccccc34)cc2)c2cccc(-c3ccccc3)c2)ccc1-c1cccc(-n2c3ccccc3c3ccc(F)cc32)c1] 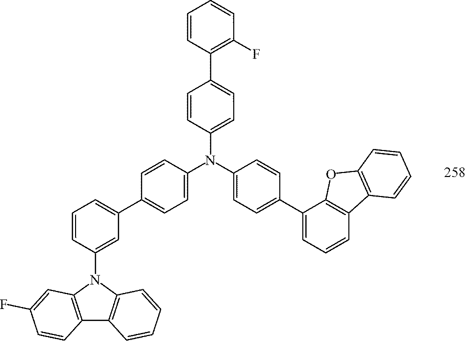 [SMILES: Fc1ccc2c3ccccc3n(-c3cccc(-c4ccc(N(c5ccc(-c6cccc7c6sc6ccccc67)cc5)c5ccc6c(c5)-c5ccccc5C65CCCC5)cc4)c3)c2c1] 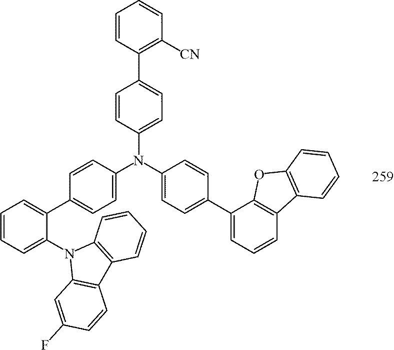 [SMILES: Fc1ccc2c3ccccc3n(-c3ccccc3-c3ccc(N(c4ccc(-c5ccccc5)cc4)c4cccc(-c5ccc6c(c5)sc5ccccc56)c4)cc3)c2c1] 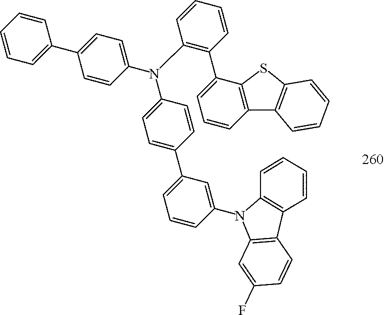 [SMILES: C[Si](C)(C)c1cccc(N(c2ccc(-c3ccccc3-n3c4ccccc4c4cc(F)ccc43)cc2)c2ccc(-c3cccc4oc5ccccc5c34)cc2)c1] 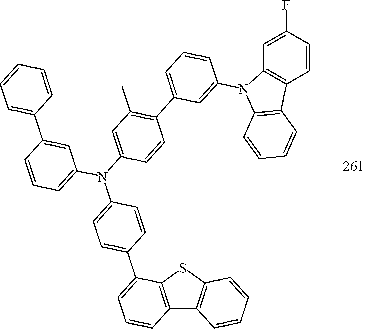 [SMILES: Fc1ccc2c3ccccc3n(-c3ccccc3-c3ccc(N(c4ccc(-c5cccc6oc7ccccc7c56)cc4)c4ccc(C(F)(F)F)cc4)cc3)c2c1] 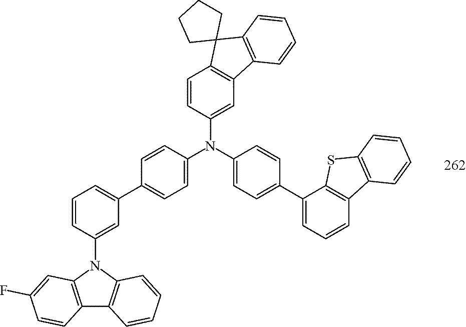 [SMILES: CC(C)(C)c1cccc(-n2c3ccccc3c3ccc(F)cc32)c1-c1ccc(N(c2ccc(-c3ccccc3)cc2)c2ccc(-c3cccc4c3oc3ccccc34)cc2)cc1] 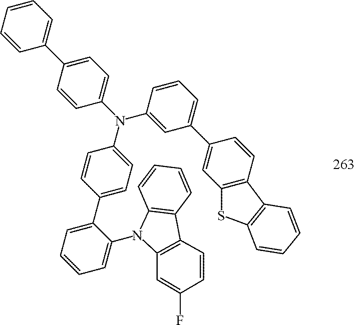 [SMILES: Cc1cc(N(c2ccc(-c3ccccc3)cc2)c2ccc(-c3cccc4c3oc3ccccc34)cc2)ccc1-c1ccccc1-n1c2ccccc2c2ccc(F)cc21] 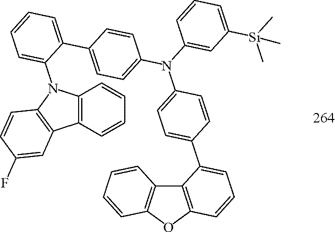 [SMILES: CC(C)(C)c1ccc(N(c2ccc(-c3ccccc3-n3c4ccccc4c4cc(F)ccc43)cc2)c2ccc(-c3cccc4sc5ccccc5c34)cc2)cc1] 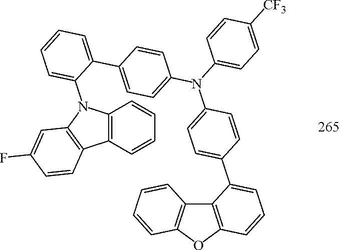 [SMILES: Fc1ccc2c3ccccc3n(-c3ccccc3-c3ccc(N(c4ccc(C(F)(F)F)cc4)c4cccc(-c5cccc6sc7ccccc7c56)c4)cc3)c2c1] 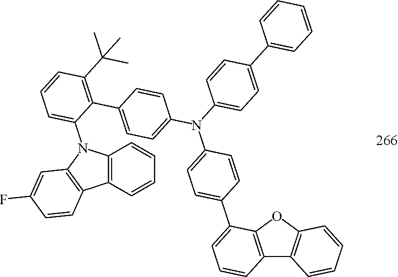 [SMILES: CC(C)(C)c1cccc(-n2c3ccccc3c3ccc(F)cc32)c1-c1ccc(N(c2ccc(-c3ccccc3)cc2)c2ccc(-c3cccc4c3sc3ccccc34)cc2)cc1] 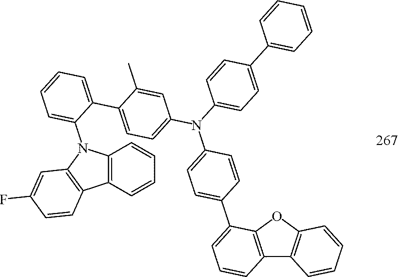 [SMILES: Cc1cc(N(c2ccc(-c3ccccc3)cc2)c2ccc(-c3cccc4c3sc3ccccc34)cc2)ccc1-c1ccccc1-n1c2ccccc2c2ccc(F)cc21] 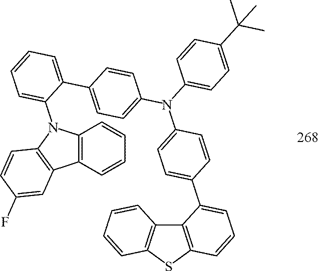 [SMILES: Cc1cccc(N(c2ccc(-c3cccc(-n4c5ccccc5c5cc(F)ccc54)c3)cc2)c2ccc(-c3cccc4oc5ccccc5c34)cc2)c1] 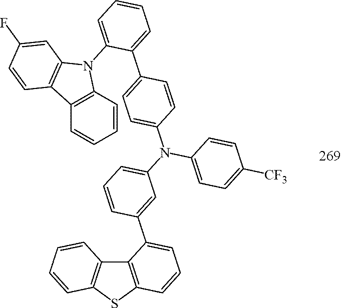 [SMILES: Fc1ccc2c3ccccc3n(-c3ccc(N(c4ccc(-c5cccc6c5oc5ccccc56)cc4)c4cccc(-c5ccc6c(c5)oc5ccccc56)c4)cc3)c2c1] 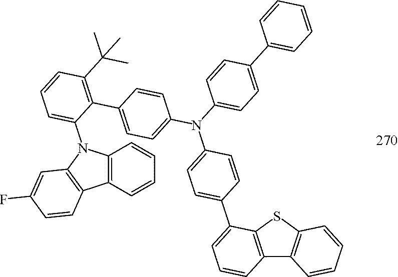 [SMILES: Fc1ccc2c3ccccc3n(-c3ccc(-c4ccccc4)c(-c4ccc(N(c5ccc(-c6ccccc6)cc5)c5ccc(-c6cccc7c6oc6ccccc67)cc5)cc4)c3)c2c1] 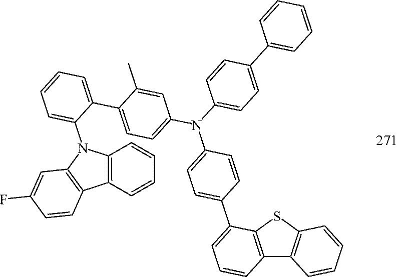 [SMILES: Fc1cccc2c1c1ccccc1n2-c1ccc(N(c2cccc(-c3ccc4c(c3)sc3ccccc34)c2)c2cccc3oc4ccccc4c23)cc1] 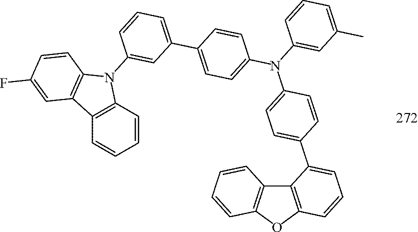 [SMILES: Fc1cccc2c1c1ccccc1n2-c1ccc(N(c2cccc(-c3ccc4c(c3)sc3ccccc34)c2)c2cccc3oc4ccccc4c23)cc1] 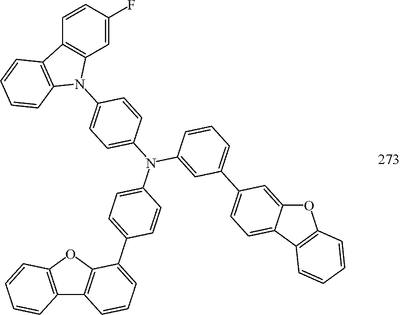 [SMILES: Fc1ccc2c3ccccc3n(-c3cccc(-c4ccc(N(c5ccc(C(F)(F)F)cc5)c5cccc(-c6cccc7sc8ccccc8c67)c5)cc4)c3)c2c1] 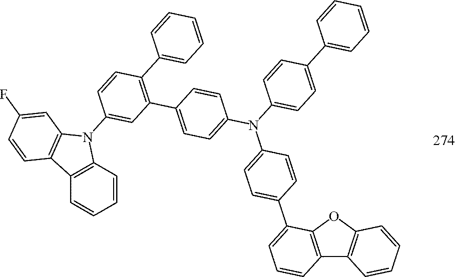 [SMILES: CC(C)(C)c1ccc(-n2c3ccccc3c3ccc(F)cc32)cc1-c1ccc(N(c2ccc(-c3ccccc3)cc2)c2ccc(-c3cccc4c3sc3ccccc34)cc2)cc1] 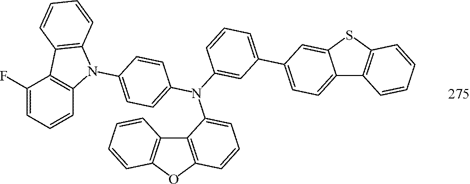 [SMILES: Cc1cc(N(c2ccc(-c3cccc4c3sc3ccccc34)cc2)c2cccc3ccccc23)ccc1-c1cccc(-n2c3ccccc3c3cc(F)ccc32)c1] 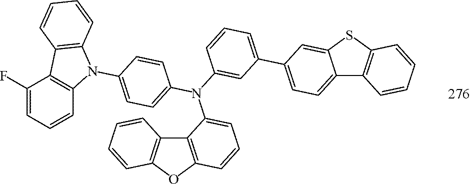 [SMILES: Fc1cccc2c1c1ccccc1n2-c1ccc(N(c2cccc(-c3ccc4c(c3)sc3ccccc34)c2)c2cccc3oc4ccccc4c23)cc1] 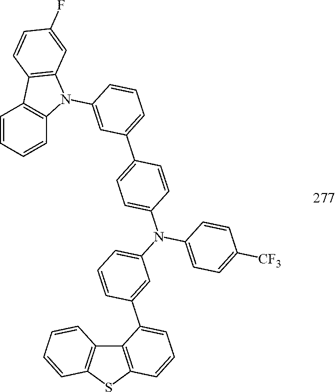 [SMILES: Fc1ccc2c3ccccc3n(-c3cccc(-c4ccc(N(c5ccc(C(F)(F)F)cc5)c5cccc(-c6cccc7sc8ccccc8c67)c5)cc4)c3)c2c1] 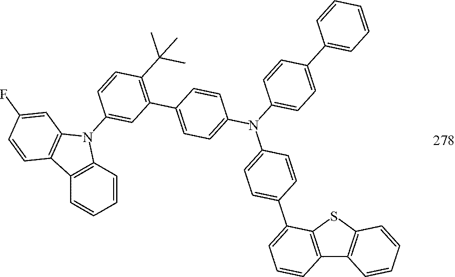 [SMILES: Fc1ccc2c3ccccc3n(-c3cccc(-c4ccc(N(c5ccc(-c6cccc7c6sc6ccccc67)cc5)c5ccc(-c6ccccc6)cn5)cc4)c3)c2c1] 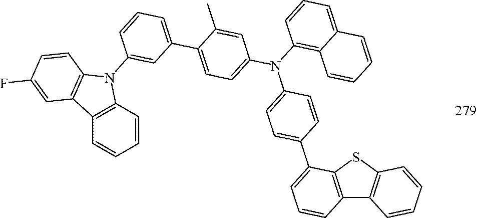 [SMILES: Cc1cc(-c2cccc(-n3c4ccccc4c4cc(F)ccc43)c2)ccc1N(c1ccc(-c2cccc3c2sc2ccccc23)cc1)c1cccc2cccnc12] 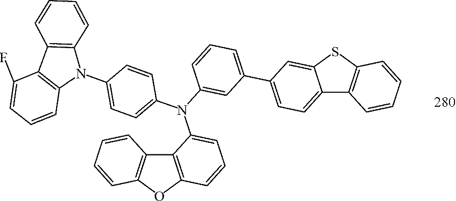 [SMILES: Fc1cccc2c1c1ccccc1n2-c1ccc(N(c2ccc(-c3ccccc3)c(-c3ccc4c(c3)sc3ccccc34)c2)c2cccc3oc4ccccc4c23)cc1] 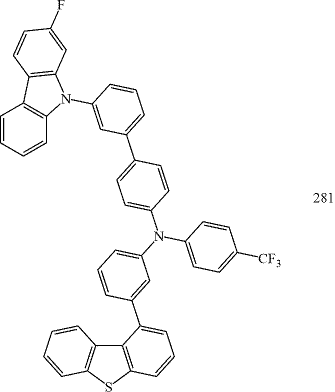 [SMILES: Fc1ccc2c3ccccc3n(-c3ccc(N(c4ccccc4)c4cccc(-c5cccc(-c6cccc7sc8ccccc8c67)c5)c4)cc3)c2c1] 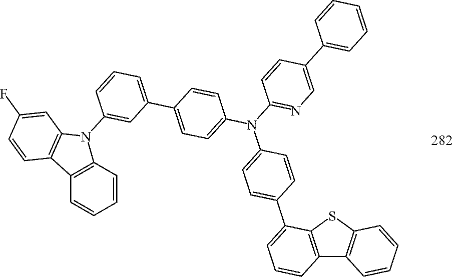 [SMILES: Fc1ccc2c3ccccc3n(-c3cccc(-c4ccc(N(c5ccc(-c6ccccc6)cc5)c5ccc6c(-c7cccc8c7oc7ccccc78)cccc6c5)cc4)c3)c2c1] 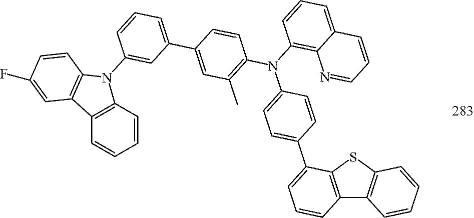 [SMILES: Fc1ccc2c(c1)c1ccccc1n2-c1cccc(-c2ccc(N(c3ccc(-c4cccc5c4oc4ccccc45)cc3)c3cccc4cccnc34)cc2)c1] 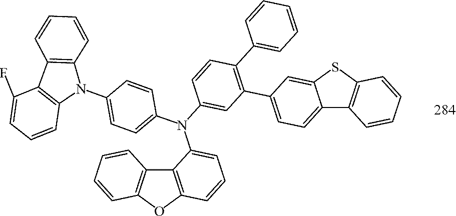 [SMILES: Fc1ccc2c3ccccc3n(-c3cccc(-c4ccc(N(c5ccc(-c6ccccc6)cc5)c5cccc6oc7c(-c8ccccc8)cccc7c56)cc4)c3)c2c1] 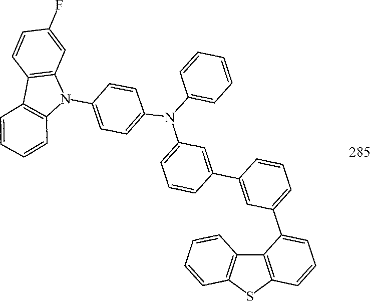 [SMILES: Fc1ccc2c(c1)c1ccccc1n2-c1cccc(-c2ccc(N(c3ccc(-c4ccccc4)cc3)c3cccc4c3oc3c(-c5ccccc5)cccc34)cc2)c1] 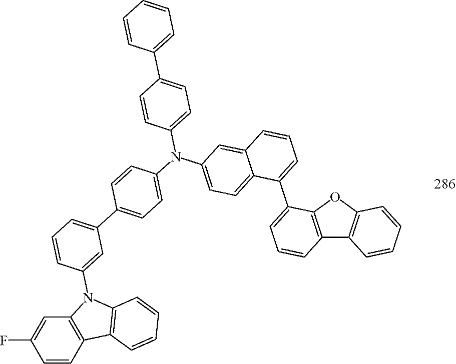 [SMILES: Fc1cccc2c1c1ccccc1n2-c1cccc(-c2ccc(N(c3ccc(-c4ccccc4)cc3)c3cccc4oc5cc(-c6ccccc6)ccc5c34)cc2)c1] 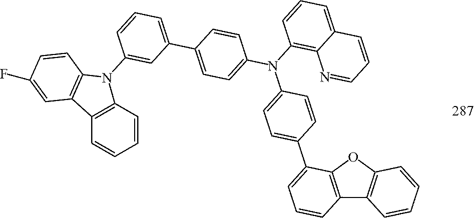 [SMILES: Fc1cccc2c1c1ccccc1n2-c1cccc(-c2ccc(N(c3cccc(-c4ccccc4)c3)c3cccc4oc5c(-c6ccccc6)cccc5c34)cc2)c1] 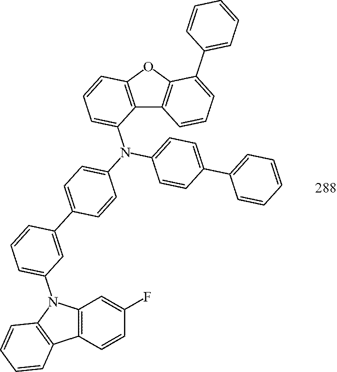 [SMILES: Fc1cccc2c3ccccc3n(-c3ccccc3-c3ccc(N(c4ccc(-c5ccccc5)cc4)c4cccc5oc6c(-c7ccccc7)cccc6c45)cc3)c12] 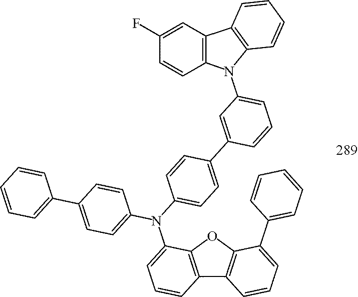 [SMILES: Fc1ccc2c(c1)c1ccccc1n2-c1ccccc1-c1ccc(N(c2ccccc2-c2ccccc2)c2cccc3oc4c(-c5ccccc5)cccc4c23)cc1] 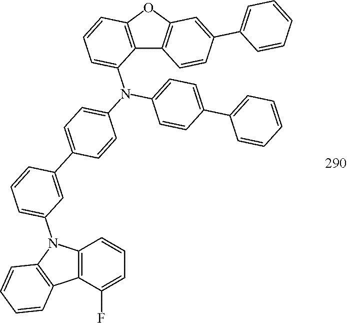 [SMILES: CC1(C)c2ccccc2-c2ccc(N(c3ccc(-c4cccc(-n5c6ccccc6c6ccc(F)cc65)c4)cc3)c3ccc(-c4cccc5c4oc4ccccc45)cc3)cc21] 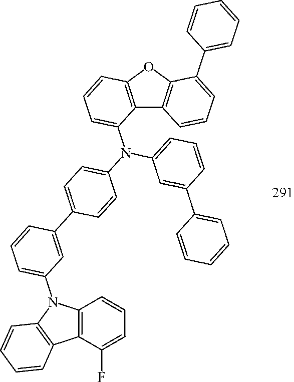 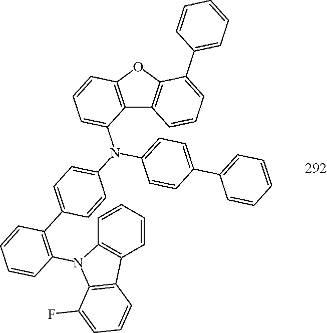 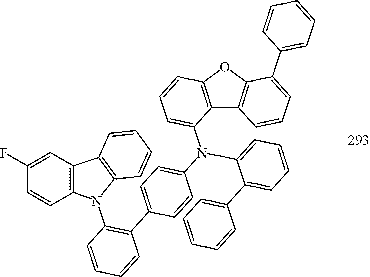 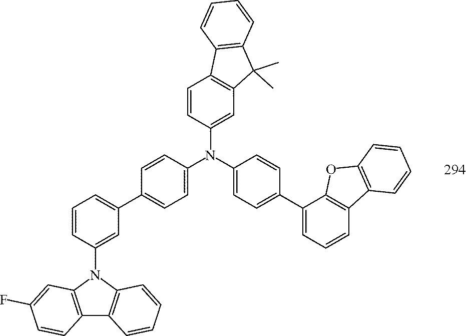
.

14. An electronic element, comprising an anode and a cathode which are oppositely disposed, and a functional layer disposed between the anode and the cathode; wherein the functional layer comprises the nitrogen-containing compound according to claim 1.

15. The electronic element according to claim 14, wherein the functional layer comprises a hole transport layer, and the hole transport layer comprises the nitrogen-containing compound.

16. An electronic device, comprising the electronic element according to claim 14.

17. The nitrogen-containing compound according to claim 3, wherein R₁ and R₂ are the same or different, and are each independently selected from deuterium, fluorine, cyano, aryl with 6 to 12 carbon atoms, trialkylsilyl with 3 to 7 carbon atoms, alkyl with 1 to 4 carbon atoms, fluoroalkyl with 1 to 4 carbon atoms, and cycloalkyl with 5 to 10 carbon atoms.

18. The nitrogen-containing compound according to claim 10, wherein 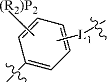
in the formula 1 is selected from 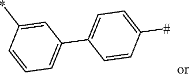 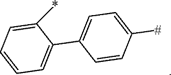
.

19. The electronic element according to claim 14, wherein the electronic element is an organic electroluminescent device or a photoelectric conversion device.

20. The electronic element according to claim 15, wherein the electronic element is the organic electroluminescent device, the hole transport layer comprises a first hole transport layer and a second hole transport layer, the first hole transport layer is closer to the anode than the second hole transport layer, and the second hole transport layer comprises the nitrogen-containing compound.
